(19) United States
(12) Patent Application Publication
(10) Pub. No.: US 2011/0275559 A1
(43) Pub. Date: Nov. 10, 2011

(54) Long-Acting Y2 and/or Y4 Receptor Agonists

(75) Inventors: Søren Østergaard, Bronshoj (DK); Sanne Møller Knudsen, Vaerlose (DK); Jane Spetzler, Bronshoj (DK); Rasmus Jørgensen, Malov (DK); Jacob Kofoed, Vaerlose (DK)

(73) Assignee: Novo Nordisk A/S, Bagsvaerd (DK)

(21) Appl. No.: 12/993,015

(22) PCT Filed: May 18, 2009
(86) PCT No.: PCT/EP09/55989

§ 371 Date: Feb. 23, 2011

Related U.S. Application Data
(60) Provisional application No. 61/055,310, filed on May 22, 2008.
(60) Provisional application No. 61/157,761, filed on Mar. 5, 2009.

(30) Foreign Application Priority Data
May 16, 2008 (EP) 08156360.3
Mar. 5, 2009 (EP) 09154461.9

(51) Int. Cl. A61K 38/22; A61P 3/04; C07K 14/575

(52) U.S. Cl. 514/5.2; 530/324; 514/9.7; 514/5.3

(57) ABSTRACT

The present invention relates to a PYY or PP peptide derivative or analogue thereof derivatised with one or more serum albumin binding side chains comprising a dis-tal tetrazole or carboxylic acid group. Moreover, the invention relates to compositions hereof and methods of treatment of conditions responsive to Y receptor modulation.

### FIELD OF THE INVENTION

This invention relates to the field of therapeutic peptides, i.e. to new protracted peptide derivatives such as Peptide YY (PYY) and Pancreatic Polypeptide (PP) derivatives.

### BACKGROUND OF THE INVENTION

PYY is released during a meal from L-cells in the distal small intestine and the colon. PYY activates both the Y1, Y2, and Y5 receptor subtypes. The peptide PYY is known to have peripheral effects in the gastrointestinal (GI) tract and also act centrally as a satiety signal. PYY is released as PYY(1-36) but is cleaved to PYY(3-36) which constitutes approx. 50% of the circulating PYY. The enzyme responsible for the degradation is dipeptidyl peptidase IV (DPPIV). PYY(3-36) displays selectivity for the Y2 receptor over the Y1, Y4, and Y5 receptors.

PP is a hormone secreted from the endocrine cells in pancreatic islets and release is stimulated by food intake. It acts preferably as an agonist of the Y4 receptor but also displays some affinity for the Y5 receptor. PP is known to reduce food-intake and potentially increase energy expenditure. The Y2 and the Y4 receptor subtypes are considered to be important regulators of food intake.

Agonists that are selective for only the Y2 or the Y4 over the Y1 and Y5 receptors or agonists that are selective for both Y2 and Y4 receptors over the Y1 and Y5 receptors are considered beneficial for treatment of conditions such as obesity. In the design of such peptide drugs is important that the agonistic effect on the Y1 is relatively low, since this will lead to unwanted side effects (e.g., increased blood pressure). Furthermore, activation of the Y5 receptor is unwanted as this will increase food intake. However, the Y5 receptor is expressed in areas of the CNS where circulating peptides are not expected to gain access.

Accordingly, PYY and PP are not optimal for use as pharmaceutical drugs due to the relative broad receptor binding specificity. PYY will act on the Y1 and Y5 receptors in addition to the Y2 receptor and PP will act on the Y5 receptor in addition to the Y4 receptor. Additionally, both PYY(3-36) and PP are rapidly degraded and display suboptimal pharmacokinetic properties, thus the peptides have to be administered at least once-daily or twice-daily. The half life of PYY(3-36) has been reported to be <30 minutes in pigs (Ito T et al, Journal of Endocrinology (2006), 191, pp 113-119) and the half-life of PP has been reported to be 7 minutes in man (Adrian T. E. et al, Gut (1978), 19, pp 907-909).

For the treatment of conditions, such as obesity, responsive to Y receptor modulation it would be attractive to use PYY or PP analogues which are specific for the Y receptor subtypes Y2 or Y4 alone, or analogues which act on both of the receptor subtypes Y2 and Y4 simultaneously and importantly also display protracted pharmaco kinetic properties and as such can be used in a dosing regime with lower frequency of administration than the human PYY, PYY(3-36), or PP peptides.

### DESCRIPTION OF THE DRAWINGS

FIG. 1A: Effect on food intake (BioDAQ) in C57BL mice after administration of PYY(3-36) and PYY analogues. Compounds tested are vehicle, SEQ ID NO: 1, SEQ ID NO: 3, and SEQ ID NO: 4. Dosage was 1 μmol/kg s.c. oid.

FIG. 1B: Effect on food intake (BioDAQ) in C57BL mice after administration of PYY analogues as described for FIG. 1A but represented using a different statistical method.

FIG. 2: Effect on food intake (BioDAQ) in lean C57BL mice after administration of SEQ ID NO: 2 (hPP(1-36)) and the PP analogues SEQ ID NO: 29 and SEQ ID NO: 30 at a dosage of 1 μmol/kg s.c.

FIG. 3: Effect on food intake (BioDAQ) in C57BL mice after administration of SEQ ID NO: 43 at a dosage of 0.03 and 0.1 μmol/kg s.c.

FIG. 4: Effect on food intake (BioDAQ) in lean C57BL mice after administration of SEQ ID NO: 23 at a dosage of 0.3 and 1.0 μmol/kg s.c.

FIG. 5: Effect on food intake (BioDAQ) in lean C57BL mice after administration of SEQ ID NO: 40 at a dosage of 0.1, 0.3 and 1.0 μmol/kg s.c.

FIG. 6: Change in body weight in ob/ob mice after administration of SEQ ID NO:3 at a dosage of 0.3 and 1.0 μmol/kg s.c.

FIG. 7: Percent weight change from baseline in ob/ob mice at day 14 of treatment with SEQ ID NO: 3 at a dosage of 0.3 and 1.0 μmol/kg s.c.

FIG. 8: Determination of pharmaco kinetic profile in mini-pigs. Compound tested is SEQ ID NO: 3. Dosage was 6 nmol/kg i.v.

FIG. 9: Effect on food intake (BioDAQ) in lean C57BL mice after administration of SEQ ID NO: 57, SEQ ID NO: 58 and SEQ ID NO: 59 at a dosage of 1.0 μmol/kg s.c.

FIG. 10: Effect on food intake (BioDAQ) in lean C57BL mice after administration of SEQ ID NO: 43, SEQ ID NO: 46 and SEQ ID NO: 55 at a dosage of 1.0 μmol/kg s.c.

FIG. 11: Effect on food intake after single s.c. administration of SEQ ID NO: 57, SEQ ID NO: 58 and SEQ ID NO: 59 at a dosage of 1.0 μmol/kg in lean rats before onset of dark.

### SUMMARY OF THE INVENTION

The present invention relates to a PYY or PP peptide derivative or analogue thereof, wherein at least one amino acid residue and/or the N- and/or C-terminus of the peptide backbone is derivatised with a serum albumin binding side chain defined by A-B-C-D-, A-C-D-, A-B-C-, or A-C-, wherein

### A- is

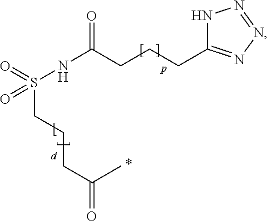

wherein p is selected from the group consisting of 10, 11, 12, 13, 14, 15 and 16 and d is selected from the group consisting of 0, 1, 2, 3, 4 and 5,
and -B- is selected from the group consisting of

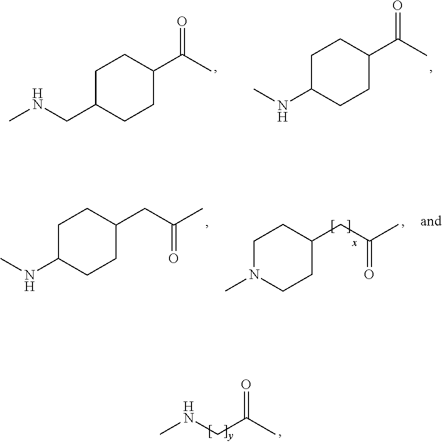

wherein x is selected from the group consisting of 0, 1, 2, 3 and 4, and y is selected from the group consisting of 1, 2, 3, 4, 5, 6, 7, 8, 9, 10, 11 and 12;

### or A- is

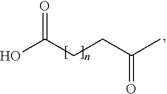

wherein n is selected from the group consisting of 12, 13, 14, 15, 16 17, 18 and 19, and -B- is selected from the group consisting of

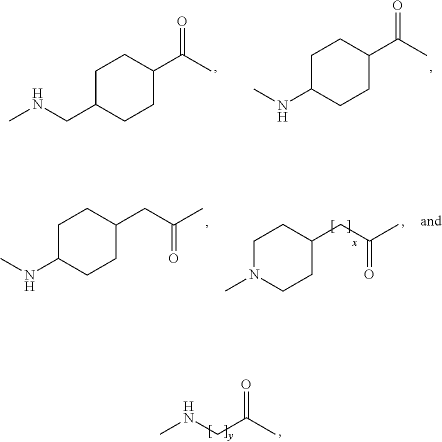

wherein x is selected from the group consisting of 0, 1, 2, 3 and 4; and
-C- is selected from the group consisting of

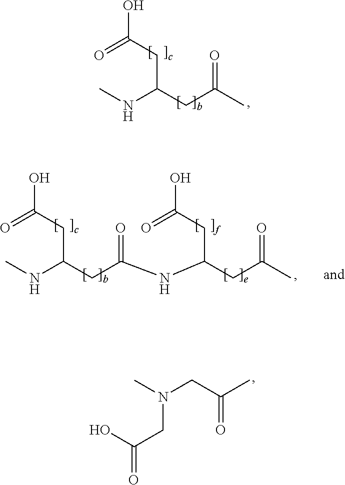

wherein b and e are each independently selected from the group consisting of 0, 1, and 2, and c and f are each independently selected from the group consisting of 0, 1, and 2 with the proviso that when
c is 0 b is 1 or 2,
c is 1 or 2 b is 0,
f is 0 e is 1 or 2,
f is 1 or 2 e is 0, and
with the proviso that when A- is

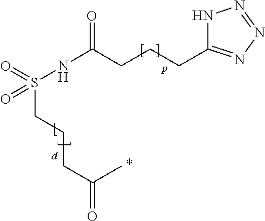

-C- may be deleted; and -D- is attached to said amino acid residue and is a spacer.

In one aspect the invention relates to a composition comprising a PYY or PP peptide derivative or analogue thereof as defined herein and one or more pharmaceutical excipients.

In one aspect the invention relates to a method of treatment of a condition responsive to Y receptor modulation by administration of a PYY or PP peptide derivative or analogue thereof as defined in any of the preceding embodiments.

In one aspect the invention relates to the use of a PYY or PP peptide derivative or analogue thereof as defined herein for the preparation of a medicament for the treatment of a condition responsive to Y receptor modulation, such as obesity or obesity-related diseases, e.g., reduction of food intake and/or increase in energy expenditure.

In one aspect the invention relates to the use of a PYY or PP peptide derivative or analogue thereof as defined herein for administration in a mammal, wherein said derivative shows protracted properties compared to the human PP and PYY compounds.

### DETAILED DESCRIPTION OF THE INVENTION

Protracted pharmaco kinetic properties can be accomplished by attaching the peptide drug of interest to serum albumin in vivo. This attachment can be either covalent or non-covalent. By attaching fatty acids or analogues thereof to the peptide of interest it can bind non-covalently to albumin. This invention describes the design of peptides with attached novel side chains that strongly bind to albumin and prolong the duration of action of the peptide drug with the result that the peptide drug only has to be dosed once-daily or alternatively once-weekly.

The fatty acid albumin binders described herein are structurally different compared to previously published fatty acid albumin binders since these novel fatty acid analogues display a distal carboxylic group or a tetrazole group. This increases the albumin binding more than 10-fold compared to fatty acids that display a methyl group. This leads to peptide analogues that are considerably more protracted and will display once-weekly dosing profile.

Acylated peptides have previously been described, such as Levemir® (WO 95/07931) and Liraglutide (WO 98/08871). However, analogues of PYY or PP suitable for administration with lower frequency than once-daily administration would require the binding to serum albumin to be higher than exemplified with the above mentioned protein and peptide.

In one aspect the invention provides a PYY or PP peptide derivative or analogue thereof with an improved PK profile. In one aspect the invention provides a PYY or PP peptide derivative or analogue thereof with an albumin binding side chain, optionally attached via a suitable spacer, displaying protracted properties making them suitable for administration with a frequency of once-daily or lower, such as in a once-weekly, twice-monthly, or once-monthly dosing regime. The albumin binding handle of this invention has a distal carboxylic acid or tetrazole group. In one aspect the albumin binding handle comprises a fatty di-acid. In one aspect the albumin binding handle is a fatty di-acid.

In one aspect the invention provides a PYY or PP peptide derivative or analogue thereof with high affinity albumin binding effect. In one aspect high affinity albumin binding effect is defined as at least 10 times, such as at least 20 times, at least 50 times, or at least 100 times higher albumin binding of the PYY or PP peptide derivative or analogue thereof according to the invention relative to human PYY, PYY(3-36), or PP peptide or non-acylated analogues hereof.

In one aspect the invention provides a PYY or PP peptide derivative with improved bioavailability compared to other analogues described elsewhere in literature, such as human PYY, PYY(3-36), or PP peptide or non-acylated analogues hereof. In one aspect the invention provides a PYY or PP peptide derivative with improved oral bioavailability as opposed to other analogues describe elsewhere in the literature, such as human PYY, PYY(3-36), or PP peptide or non-acylated analogues hereof.

In one aspect the invention provides a PYY or PP peptide derivative or analogue thereof with improved enzymatic stability as opposed to other analogues described elsewhere in the literature, such as human PYY, PYY(3-36), or PP peptide or non-acylated analogues hereof.

The term “agonist” means any compound that activates the target receptor and elicits one or more of the in vivo or in vitro effects elicited by the endogenous agonist for said receptor.

“Protracted properties” of a peptide is prolonged action of duration of the peptide which results in dosing with lower frequency, e.g., once-daily or alternatively once-weekly dosing. The protracted properties of PYY or PP peptide derivatives or analogues thereof according to the invention could manifest as prolonged plasma half life or prolonged biological activity compared to the human PYY, PYY(3-36), or PP peptide or non-acylated analogues hereof. In one aspect the protraction of compounds of the invention is determined by monitoring the concentration thereof in plasma after s.c. or i.v. administration to animals, such as healthy pigs, using methods as described herein, such as the mini-pig PK assay. For comparison also the concentration in plasma of human PYY, PYY(3-36), PP peptide or non-acylated analogues hereof after s.c. or i.v. administration is followed. The protraction of other PYY, PYY(3-36), or PP compounds of the invention can be determined in the same way. In one aspect the protraction of compounds of the invention is determined by monitoring the duration of effect of the compounds in a biological assay such as an assay for food intake in mice, e.g. fasting induced refeeding assay, following s.c. administration of the compounds. For comparison also the duration of effect of human PYY(3-36), PP peptide or non-acylated analogues hereof after s.c. administration is followed.

The terms “human PYY” and “hPYY” or “human PP” and “hPP” are intended to mean PYY(1-36) according to SEQ ID NO: 1, or alternatively PYY(3-36) according to SEQ ID NO: 1 and with a deletion of the N terminal amino acids in position 1 and 2, and PP(1-36) according to SEQ ID NO: 2, respectively. In one aspect the term PYY is intended to refer to human PYY. In one aspect the term PP is intended to refer to human PP.

### Peptide YY (PYY) and Pancreatic Peptide (PP)

Peptide YY (PYY) and pancreatic peptide (PP) both belong to a group of peptides of the PP-fold family to which neuropeptide Y (NPY) also belongs. They are all homologous and naturally secreted as 36 amino acid peptides with a C-terminal amide. They are characterised by a common three-dimensional fold, the PP-fold, which is considered as a very stable structure. The amino acid sequence of human PYY(1-36) and human PP(1-36) are shown in SEQ ID NO: 1 and SEQ ID NO: 2, respectively.

The determinants for specificity of PYY towards the Y2 receptor is mainly located in the C-terminal part of the peptide. The determinants for specificity of PYY towards the Y1 receptor is located at both the N- and C-terminal. The peptide PYY(3-36) which is naturally occurring is relatively selective towards the Y2 over the Y1 and this peptide is currently in clinical trials.

PP is selective towards the Y4 receptor and the determinants for this specificity is mainly located in the N-terminal part. The C-terminal part of PP differs mainly by one important residue compared to PYY. In PP the position 34 is a proline residue (Pro34) while in PYY this residue is a Gln (Gln34). It is known that mutating the Pro34 to a Gln34, PP will become Y2 selective in addition to Y4 specificity (J. Jorgensen et al, 1990, Eur. J. Pharm 186, 105-114). This dual-acting mechanism has been shown to give beneficial effects on appetite regulation and is thereby a potential treatment of obesity.

### PP-Fold Peptide Receptors

In one aspect this invention relates to PYY or PP peptide derivatives or analogues thereof which are selective for the Y4 receptor over the Y1, Y2 and Y5 receptors and have protracted pharmaco kinetic properties. In one aspect this invention relates to PYY or PP peptide derivatives or analogues thereof which are selective for the Y2 receptor over the Y1, Y4, and Y5 receptors and have protracted pharmaco kinetic properties. In one aspect this invention relates to PYY or PP peptide derivatives or analogues thereof which are selective for the Y2 and Y4 receptors over the Y1 and Y5 receptors and have protracted pharmaco kinetic properties.

In one aspect peptides being “selective” for specific receptors over other receptors refers to peptides that display at least 10 fold, such as at least 20 fold, at least 50 fold, or at least 100 fold higher potency for one Y receptor over other Y receptors as measured in vitro in an assay for receptor function, such as an assay for calcium mobilization, and compared by EC50 values.

PP-fold peptides or analogues thereof have been suggested for use in the treatment of obesity and associated diseases based on the demonstrated effects of certain of the these peptides in animal models and in man and on the fact that obese people have low basal levels of PP and PYY as well as lower meal responses of these peptides. Furthermore, both Y2 and Y4 agonists have been demonstrated to have anti-secretory and pro-absorptive effects in the gastrointestinal (GI) tract. The potential use of Y2 and Y4 agonists in the treatment of a number of gastrointestinal disorders has been suggested

For the treatment of conditions responsive to Y4 receptor modulation, such as obesity and intestinal hyper-secretion, it would be desirable to use protracted Y4 receptor selective agonists. The relatively short half-life of PP limits the therapeutic use of this peptide as a steady exposure level would require frequent dosing which would be highly inconvenient for the patients. As Y1 receptor activation can cause cardiovascular side effects and Y2 receptor activation can cause dose limiting nausea and vomiting it would be desirable to retain the Y4 receptor selectivity of the PP.

For treatment of conditions responsive to Y2 receptor modulation such as obesity and intestinal hyper-secretion it would be desirable to use protracted Y2 receptor selective agonists. The relatively short half-life of PYY(3-36) limits the therapeutic use of this peptide as a steady exposure level would require frequent dosing which would be highly inconvenient for the patients. As Y1 and Y5 receptor activation can cause cardiovascular side effects and Y4 receptor activation could cause so far unknown side effects it would be desirable to retain the Y2 receptor selectivity of the PYY(3-36).

For the treatment of conditions responsive to both Y2 and Y4 receptor modulation, such as obesity and intestinal hyper-secretion it would be desirable to use protracted dual acting Y2 and Y4 receptor selective agonists as an additive effect could be obtained from simultaneous activation of the Y2 and Y4 receptors compared to activation of the Y2 or Y4 receptors alone.

### Analogues of PYY or PP Peptide

The term “analogue” as used herein referring to a peptide means a modified peptide wherein one or more amino acid residues of the peptide have been substituted by other amino acid residues and/or wherein one or more amino acid residues have been deleted from the peptide and/or wherein one or more amino acid residues have been added to the peptide and/or wherein one or more amino acid residues of the peptide have been modified. Such addition or deletion of amino acid residues can take place at the N-terminal of the peptide and/or at the C-terminal of the peptide. A simple nomenclature is used to describe the compounds according to the invention, for example, [Gln34]hPP(2-36) designates an analogue of the human PP, wherein the naturally occurring proline in position 34 has been substituted with Gln and the naturally occurring alanine in position 1 has been deleted. The peptide may be derived from vertebrates, such as human, mouse, sheep, goat, cow, or horse. The term “vertebrate” means members of the subphylum Vertebrata, a primary division of the phylum Chordata that includes the fish, amphibians, reptiles, birds, and mammals, all of which are characterized by a segmented spinal column and a distinct well-differentiated head. The term “mammal” means humans as well as all other warm-blooded members of the animal kingdom possessed of a homeostatic mechanism in the class Mammalia, e.g., companion mammals, zoo mammals, and food-source mammals. Some examples of companion mammals are canines (e.g., dogs), felines (e.g., cats) and horses; some examples of food-source mammals are pigs, cattle, sheep, and the like. In one aspect the mammal is a human or a companion mammal. In one aspect the mammal is a human, male or female.

The term “polypeptide” and “peptide” as used herein means a compound composed of at least five constituent amino acids connected by peptide bonds. All amino acids for which the optical isomer is not stated is to be understood to mean the L-isomer. However, also contemplated within the scope of the invention are D-amino acid residues of one or more of the amino acids.

The constituent amino acids of the peptides according to the invention may be from the group of the amino acids encoded by the genetic code and they may be natural amino acids which are not encoded by the genetic code, as well as synthetic amino acids. Natural amino acids which are not encoded by the genetic code are e.g., γ-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), β-alanine, 3-aminomethyl benzoic acid, anthranilic acid.

The 22 proteinogenic amino acids are: Alanine, Arginine, Asparagine, Aspartic acid, Cysteine, Cystine, Glutamine, Glutamic acid, Glycine, Histidine, Hydroxyproline, Isoleucine, Leucine, Lysine, Methionine, Phenylalanine, Proline, Serine, Threonine, Tryptophan, Tyrosine, Valine.

Thus a non-proteinogenic amino acid is a moiety which can be incorporated into a peptide via peptide bonds but is not a proteogenic amino acid. Examples are γ-carboxyglutamate, ornithine, phosphoserine, the D-amino acids such as D-alanine and D-glutamine, Synthetic non-proteogenic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), 3-aminomethyl benzoic acid, anthranilic acid, des-amino-Histidine, the beta analogues of amino acids such as β-alanine etc., D-histidine, desamino-histidine, 2-amino-histidine, β-hydroxy-histidine, homohistidine, Nα-acetylhistidine, α-fluoromethyl-histidine, α-methyl-histidine, 3-pyridylalanine, 2-pyridylalanine or 4-pyridylalanine, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid.

Unnatural amino acids for use in the invention include, e.g., thiotyrosine, ornithine, 3-mercaptophenylalanine, 3- or 4-aminophenylalanine, 3- or 4-acetylphenylalanine, 2- or 3- hydroxyphenylalanine (o- or m-tyrosine), hydroxymethylglycine, aminoethylglycine, 1-methyl-1-mercaptoethylglycine, aminoethylthioethylglycine and mercaptoethylglycine. Many of the unnatural amino acids useful in the present invention are commercially available. Others may be prepared by methods known in the art.

In one aspect, the peptides of the invention are at least 34 amino acids in length. In other embodiments, the peptides may be at least 21, 22, 23, 24, 25, 26, 27, 28, 29, 30, 31, 32, or 33 amino acids in length. Further, In one aspect, the peptides of the invention include only natural L-amino acid residues and/or modified natural L-amino acid residues. Alternatively, In one aspect, the peptides of the invention do not include unnatural amino acid residues.

In embodiments of the invention a maximum of 17 amino acids have been modified as compared to PYY (SEQ ID NO: 1) or PP (SEQ ID NO: 2). In embodiments of the invention a maximum of 15 amino acids have been modified as compared to PYY (SEQ ID NO: 1) or PP (SEQ ID NO: 2). In embodiments of the invention a maximum of 10 amino acids have been modified as compared to PYY (SEQ ID NO: 1) or PP (SEQ ID NO: 2). In embodiments of the invention a maximum of 8 amino acids have been modified as compared to PYY (SEQ ID NO: 1) or PP (SEQ ID NO: 2). In embodiments of the invention a maximum of 7 amino acids have been modified as compared to PYY (SEQ ID NO: 1) or PP (SEQ ID NO: 2). In embodiments of the invention a maximum of 6 amino acids have been modified as compared to PYY (SEQ ID NO: 1) or PP (SEQ ID NO: 2). In embodiments of the invention a maximum of 5 amino acids have been modified as compared to PYY (SEQ ID NO: 1) or PP (SEQ ID NO: 2). In embodiments of the invention a maximum of 4 amino acids have been modified as compared to PYY (SEQ ID NO: 1) or PP (SEQ ID NO: 2). In embodiments of the invention a maximum of 3 amino acids have been modified as compared to PYY (SEQ ID NO: 1) or PP (SEQ ID NO: 2). In embodiments of the invention a maximum of 2 amino acids have been modified as compared to PYY (SEQ ID NO: 1) or PP (SEQ ID NO: 2). In embodiments of the invention 1 amino acid has been modified as compared to PYY (SEQ ID NO: 1) or PP (SEQ ID NO: 2).

In yet another embodiment, peptides of the invention may exhibit at least 60%, 65%, 70%, 80%, or 90% sequence identity to a PYY(1-36), PYY(3-36), or PP(1-36) over the entire length of the PYY(1-36), PYY(3-36), or PP(1-36) respectively. In yet another embodiment, peptides of the invention may exhibit at least 50%, 60%, 65%, 70%, 80%, or 90% sequence identity to a NPY. As an example of a method for determination of sequence identity between two analogues the two peptides [Gln34]PP(1-36) and PP(1-36) are aligned. The sequence identity of Gln34 analogue relative to PP(1-36) is given by the number of aligned identical residues minus the number of different residues divided by the total number of residues in PP(1-36). Accordingly, in said example the sequence identity is (36-1)/36.

In one aspect, the present invention relates to peptides comprising at least two PP-fold motifs, wherein the at least two PP-fold motifs include at least the N-terminal polyproline PP-fold motif and the C-terminal tail PP-fold motif, and the PP-fold peptide does not include any unnatural amino acid residues.

In one aspect, the peptides of the invention include PYY or PP peptide derivatives or analogues thereof. In one aspect of the invention, the peptides of the invention include PP-fold chimeric peptides comprising a fragment of a PP, PYY or NPY peptide covalently linked to at least one additional fragment of a PP, PYY or NPY peptide, wherein each PP, PYY or NPY fragment includes a PP-fold motif.

More particularly, in one aspect, the present invention relates to PYY or PP peptide derivatives or analogues thereof including one or more amino acid sequence modifications. Such modifications include substitutions, insertions, and/or deletions, alone or in combination. In a specific aspect, the PYY or PP peptides derivatives or analogues thereof of the invention include one or more modifications of a “non-essential” amino acid residue. In the context of the invention, a “non-essential” amino acid residue is a residue that can be altered, i.e., deleted or substituted, in the human PYY or PP amino acid sequence without abolishing or substantially reducing the PYY or PP peptide derivative or analogue thereof activity of the PYY or PP analogue peptide, respectively.

In one aspect of the invention, the C-terminal of the derivative according to the invention may be terminated as either an acid or amide. In a specific aspect, the C-terminal of the derivative of the invention is an amide.

Substitutions. In one aspect, the PYY or PP peptide derivatives or analogues thereof of the invention may have one or more substitutions in the amino acid sequence of human PYY or PP, respectively, alone or in combination with one or more insertions or deletions. In one aspect, the substitution does not abolish or substantially reduce the PYY or PP peptide derivative or analogue thereof activity of the PYY or PP analogue peptide, respectively. In one aspect, the present invention relates to PYY or PP peptide derivatives or analogues thereof that have a single substitution, or consecutive or non-consecutive substitution of more than one amino acid residues in the amino acid sequence of human PYY or PP, respectively. In one aspect, the PYY or PP peptide derivatives or analogues thereof of the invention include one, two, or three amino acid substitutions.

In one aspect, the amino acid residues of human PYY at the helical C-terminus region of PYY (e.g., residues 20, 24, 25, 27 and 29), the tail end residues (32-36), and/or the N-terminus prolines at position 5 and 8 are not substituted. In one aspect, amino acid residues are not substituted at positions 32 through 36 of human PYY. In one aspect, amino acid residues of human PYY are not substituted at one or more amino acid sequence positions selected from: 5, 7, 8, 20, 24, 25, 27, 29, 32, 33, 34, 35, 36, and any combination thereof.

In one aspect amino acids may be substituted by conservative substitution. The term “conservative substitution” as used herein denotes that one or more amino acids are replaced by another, biologically similar residue. Examples include substitution of amino acid residues with similar characteristics, e.g. small amino acids, acidic amino acids, polar amino acids, basic amino acids, hydrophobic amino acids and aromatic amino acids. For example, in a preferred embodiment of the invention Met residues are substituted with norleucine (Nle) or with leucine, isoleucine or valine, which—as opposed to Met—are not readily oxidised. Another example of a conservative substitution with a residue normally not found in endogenous, mammalian peptides and proteins would be the conservative substitution of Arg or Lys with for example, ornithine, canavanine, aminoethylcysteine or other basic amino acid. For further information concerning phenotypically silent substitutions in peptides and proteins, see, for example, Bowie et. al. Science 247, 1306-1310, 1990. Conservatively substituted analogues of the invention may have, for example, up to 10 conservative substitutions, or In one aspect up to 5, or in yet another embodiment 3 or fewer.

In one aspect, the PYY or PP peptide derivatives or analogues thereof of the invention may include substitutions of one or more unnatural and/or non-amino acids, e.g., amino acid mimetics, into the sequence of PYY or PP, respectively. In a preferred embodiment, the non-amino acids inserted into the sequence of PYY or PP may be beta-turn mimetics or linker molecules, such as —NH—X—CO—, wherein X=(CH2)n (where n can be 2-20) or —NH—CH2CH2(-O—CH2CH2-O-)m-CH2-CO— (where m=1-5). Preferred linker molecules include aminocaproyl (“Aca”), beta-alanyl, and 8-amino-3,6- dioxaoctanoyl. beta-turn mimetics are available commercially (BioQuadrant Inc, Quebec, Canada) and have been described in literature (Hanessian et ah, Tetrahedron 12789-854 (1997); Gu et ah, Tetrahedron Letters 44: 5863-6 (2003); Bourguet et al., Bioorganic and Medicinal Chemistry Letters 13: 1561-4 (2003); Grieco et ah, Tetrahedron Letters 43: 6297-9 (2002); Souers et ah, Tetrahedron 57: 7431-48 (2001); Tsai et ah, Bioorganic and Medicinal Chemistry 7: 29-38 (1999); Virgilio et ah, Tetrahedron 53: 6635-44 (1997)).

Deletions and Truncations. In one aspect, the PYY or PP peptide derivatives or analogues thereof of the invention may have one or more amino acid residues deleted from the amino acid sequence of human PYY or PP, respectively, alone or in combination with one or more insertions or substitutions. In one aspect, the PYY or PP peptide derivatives or analogues thereof of the invention may have one or more amino acid residues deleted from the N-terminus or C-terminus of human PYY or PP, respectively. In one aspect, the PYY or PP peptide derivatives or analogues thereof of the invention may have one or more amino acid residues deleted at amino acid positions 2 through 35 of human PYY or PP, respectively. Such deletions may include more than one consecutive or non-consecutive deletions at amino acid positions 2 through 35 of human PYY or PP. In a preferred embodiment, the amino acid residues at positions 24 through 36 of human PYY or PP are not deleted.

In one aspect, the PP-fold peptides of the invention may include N or C-terminal truncations, or internal deletions at amino acid positions 2 to 35 so long as at least one biological activity of a native PP-fold peptide is retained. In preferred embodiments, the amino acid residues at positions 5 through 8 and 24 through 36, more specifically 5 through 8 and 32 through 35 are not deleted.

Insertions. In one aspect, the PYY or PP peptide derivatives or analogues thereof of the invention may have one or more amino acid residues inserted into the amino acid sequence of human PYY or PP, respectively, alone or in combination with one or more deletions and/or substitutions. In one aspect, the present invention relates to PYY or PP peptide derivatives or analogues thereof that have a single insertion, or consecutive or non-consecutive insertions of more than one amino acid residues into the amino acid sequence of human PYY or PP. In yet a further embodiment, one or more amino acids may be inserted at the N-terminal or C-terminal end of the peptide analogue. In yet a further embodiment, amino acid residues are not inserted at positions 24 through 36 of human PYY or PP, respectively.

In one aspect, the PYY or PP peptide derivatives or analogues thereof of the invention may include insertions of one or more unnatural amino acids and/or non-amino acids into the sequence of PYY or PP, respectively. In yet another embodiment, the unnatural amino acids inserted into the sequence of human PYY or PP may be beta-turn mimetics or linker molecules. Examples of linker molecules include aminocaproyl (“Aca”), beta-alanyl, and 8-amino-3,6-dioxaoctanoyl.

In one aspect the invention relates to PYY or PP mimetics characterised by a deletion of the residues 5-24 which are substituted by a linker such as but not restricted to: aminocaproyl (“Aca”), beta-alanyl, and 8-amino-3,6-dioxaoctanoyl. In addition these mimics are stabilised, e.g., by a S—S by Cys in position 2 and a D-Cys in position 27.

In yet another embodiment PP-fold peptides are stabilised by a lactam bridge between a Lys and a Glu. As an example, but not restricted hereto, is a Lys in position 28 and Glu in position 32. In one aspect of the invention, the analogue of a PYY or PP peptide includes combinations of the above-described modifications, i.e., deletion, truncation, insertion, and substitution. In one aspect of the invention, the analogue of a PYY or PP peptide includes one, two, or three amino acid substitutions.

In the PP sequence Asp10 is particularly prone to cyclisation in solution to form a cyclic imidate which ring opens to form mixtures of the alpha and beta-aspartate with concomitant scrambling of stereochemistry. In peptide pairs (v), (vi) and (viii) of the invention that residue has been replaced by Glu. This substitution preserves the special electrostatic potential distribution within the peptides and thereby the overall stability of the peptide as well as its solubility. Since Glu in position 10 does not undergo analogues cyclisation/ring opening to form gamma-Glu it has the beneficial effect of improving the bulk and the solution stability of the peptide as a pharmaceutical agent compared to its Asp 10 counterparts. Improved solution stability leads to increased synthetic yields and reduces the requirement for troublesome, costly and waste producing purification of the desired product from the closely related beta-Asp impurity. In one aspect an albumin binding handle according to the invention may be attached to Asp10 in the PP sequence.

In the PYY or PP peptides of this invention Met may be substituted with a residue that is not prone to this alteration. For example, the Met 17 and Met 30 residues in the human PP sequence can potentially undergo oxidation upon storage in solution. Specifically, Met may be substituted with Nle which prevents oxidation at this position and preserves the aliphatic side chain structure as Nle is a bio-isostere for Met in the PYY or PP peptides of this invention. Also Leu, Ile and Val may be used as isosteres for Met. In addition the aliphatic alpha-helix promoting amino acids 1-aminocyclohexyl) carboxylic acid or 1-aminocyclopentyl) carboxylic acid may be used as a substitute for Met.

In one aspect of the invention enzymatic degradation of human PP is prevented by removal of Ala1 from the PP sequence, i.e. forming the analogue PP(2-36), whereby improving the stability of the peptide both in solution and as lyophilates and therefore improving their properties as pharmaceuticals. Alternatively, the Ala2 from the PP sequence may be substituted with the closely related Aib also improving the stability against DPPIV enzymatic cleavage.

The various stability improving modifications presented above, taken singly or together represent a significant advance in the pharmaceutical properties of these peptides. Improved stability both during synthesis, leading to higher yields and less purification, and prolonged shelf life of the lyophilate and the solutions of these peptides reduces significantly the environmental burden of the production (and reducing the necessity for remanufacture) of peptides of this invention by reducing the use of raw materials, solvents, utilities and therefore also the production of waste products.

The term “DPP-IV protected” as used herein referring to a polypeptide means a polypeptide which has been chemically modified in order to render said derivative resistant to the plasma peptidase dipeptidyl aminopeptidase-4 (DPP-IV). The DPP-IV enzyme in plasma is known to be involved in the degradation of several peptide hormones, e.g. PYY, PP, etc. Thus, a considerable effort is being made to develop analogues and derivatives of the polypeptides susceptible to DPP-IV mediated hydrolysis in order to reduce the rate of degradation by DPP-IV.

In one aspect of the invention, the PYY or PP derivative is a DPPIV protected PYY or PP derivative. In one aspect of the invention, the said PYY or PP derivative is stabilised against DPP-IV degradation relatively to the stability of PYY or PP. In one aspect a derivative according to the invention is a DPP-IV protected derivative which is more resistant to DPP-IV than PYY or PP.

Resistance of a peptide to degradation by dipeptidyl aminopeptidase IV is determined by the following degradation assay:

Aliquots of the peptide (5 nmol) are incubated at 37° C. with 1 μl of purified dipeptidyl aminopeptidase IV corresponding to an enzymatic activity of 5 mU for 10-180 minutes in 100 μl of 0.1 M triethylamine-HCl buffer, pH 7.4. Enzymatic reactions are terminated by the addition of 5 μl of 10% trifluoroacetic acid, and the peptide degradation products are separated and quantified using HPLC analysis. One method for performing this analysis is: The mixtures are applied onto a Vydac C18 widepore (30 nm pores, 5 μm particles) 250×4.6 mm column and eluted at a flow rate of 1 ml/min with linear stepwise gradients of acetonitrile in 0.1% trifluoroacetic acid (0% acetonitrile for 3 min, 0-24% acetonitrile for 17 min, 24-48% acetonitrile for 1 min) according to Siegel et al., Regul. Pept. 1999; 79:93-102 and Mentlein et al. Eur. J. Biochem. 1993; 214:829-35. Peptides and their degradation products may be monitored by their absorbance at 220 nm (peptide bonds) or 280 nm (aromatic amino acids), and are quantified by integration of their peak areas related to those of standards. The rate of hydrolysis of a peptide by dipeptidyl aminopeptidase IV is estimated at incubation times which result in less than 10% of the peptide being hydrolysed.

Alternatively, the resistance of a peptide to degradation by dipeptidyl aminopeptidase IV is determined by the following degradation assay: Aliquots of the peptide (4 nmol) are incubated at 37° C. with 10.9 mU of purified dipeptidyl aminopeptidase IV for 22 hours in 40 μl of 0.085 M Tris-HCl buffer, pH 8.0, in presence or absence of 1.6% human serum albumin. After 0, 4, and 22 hours samples of 10 μl are taken and enzymatic reactions are terminated by mixing with 100 μl of 1% trifluoroacetic acid. The peptide degradation products are separated and quantified using HPLC analysis. One method for performing this analysis is: The mixtures are applied onto an Agilent Zorbax 300SB-C18 (5 μm particles) 150×2.1 mm column and eluted at a flow rate of 0.5 ml/min with a linear gradient from 0.1% trifluoroacetic acid to 100% acetonitrile with 0.07% TFA in 30 minutes. Peptides and their degradation products are monitored by their absorbance at 214 nm, and are quantified by integration of their peak areas. The stability of a peptide against dipeptidyl aminopeptidase IV is determined as the peak area of the intact peptide relative to the sum of the peak areas of the intact peptide and the degradation product lacking the two aminoterminal amino acids after cleavage.

### Derivatives of PYY or PP Peptide or Analogues Thereof

The term “derivative” as used herein in relation to a peptide means a chemically modified peptide or an analogue thereof, wherein at least one substituent is not present in the unmodified peptide or an analogue thereof, i.e. a peptide which has been covalently modified. Typical modifications are amides, carbohydrates, alkyl groups, acyl groups, esters and the like. In one aspect derivatives of PYY or PP are derived from a vertebrate or analogues thereof as described herein modified with an albumin binding handle. The albumin binding handle may occur singularly at the N- or C-terminus or at the side chains of amino acid residues within the sequence of the PYY or PP peptide derivatives or analogues thereof. Alternatively, there may be multiple sites of derivatization along the PYY or PP analogue peptide. Substitution of one or more amino acids with lysine, aspartic acid, glutamic acid, or cysteine may provide additional sites for derivatization. Alternatively, the PYY or PP peptide derivatives or analogues thereof may be conjugated to one, two, or three albumin binding handles molecules.

Any amino acid position in the PYY or PP peptide or analogue thereof may be derivatised. In one aspect of the invention, the amino acid residue which is derivatised comprises an amino group. In one aspect, the derivatised amino acid residue comprises an amino group. In one aspect, the derivatised amino acid residue comprises a primary amino group in a side chain. In one aspect, the derivatised amino acid residue is lysine. In one aspect of the invention, the derivatised amino acid residue is cysteine. In one aspect of the invention, one amino acid residue is derivatised. In yet one aspect of the invention, the derivative according to the invention is only derivatised in one position, e.g. only one amino acid residue is derivatised.

In one aspect the amino terminal position of the PP peptide or an analogue thereof may be derivatised, wherein said position is relative to the PP(1-36) peptide. In one aspect the amino terminal position of the PP peptide or an analogue thereof may be acylated, wherein said position is relative to the PP(1-36) peptide. In one aspect the amino terminal position of the PP peptide or an analogue thereof may be derivatised with an albumin binding group comprising CH₃(CH₂)ᵣCO—, wherein r is 16 or 18, wherein said position is relative to the PP(1-36) peptide.

In one aspect position 18 of the PP peptide or an analogue thereof may be derivatised, wherein said position is relative to the PP(1-36) peptide. In one aspect position 18 of the PP peptide or an analogue thereof may be acylated, wherein said position is relative to the PP(1-36) peptide. In one aspect position 18 of the PP peptide or an analogue thereof may be derivatised with an albumin binding group comprising CH₃(CH₂)ᵣCO—, wherein r is 16 or 18, wherein said position is relative to the PP(1-36) peptide.

In one aspect the amino terminal position of the PYY peptide or an analogue thereof may be derivatised. In one aspect the amino terminal position of the PYY peptide or an analogue thereof may be acylated. In one aspect the amino terminal position of the PYY peptide or an analogue thereof may be derivatised with an albumin binding group comprising CH₃(CH₂)ᵣCO—, wherein r is 16 or 18. In one aspect the amino terminal position of PYY(3-36) or an analogue thereof may be derivatised with an albumin binding group comprising CH₃(CH₂)ᵣCO—, wherein r is 16 or 18.

In one aspect position 18 of the PYY peptide or an analogue thereof may be derivatised, wherein said position is relative to the PYY(1-36) peptide. In one aspect position 18 of the PYY peptide or an analogue thereof may be acylated, wherein said position is relative to the PYY(1-36) peptide. In one aspect position 18 of the PYY peptide or an analogue thereof may be derivatised with an albumin binding group comprising CH₃(CH₂)ᵣCO—, wherein r is 16 or 18, wherein said position is relative to the PYY(1-36) peptide.

In one aspect position 19 of the PYY peptide or an analogue thereof may be derivatised, wherein said position is relative to the PYY(1-36) peptide. In one aspect position 19 of the PYY peptide or an analogue thereof may be acylated, wherein said position is relative to the PYY(1-36) peptide. In one aspect position 19 of the PYY peptide or an analogue thereof may be derivatised with an albumin binding group comprising CH₃(CH₂)ᵣCO—, wherein r is 16 or 18, wherein said position is relative to the PYY(1-36) peptide.

In one aspect position 22 of the PYY peptide or an analogue thereof may be derivatised, wherein said position is relative to the PYY(1-36) peptide. In one aspect position 22 of the PYY peptide or an analogue thereof may be acylated, wherein said position is relative to the PYY(1-36) peptide. In one aspect position 22 of the PYY peptide or an analogue thereof may be derivatised with an albumin binding group comprising CH₃(CH₂)ᵣCO—, wherein r is 16 or 18, wherein said position is relative to the PYY(1-36) peptide.

In one aspect position 23 of the PYY peptide or an analogue thereof may be derivatised, wherein said position is relative to the PYY(1-36) peptide. In one aspect position 23 of the PYY peptide or an analogue thereof may be acylated, wherein said position is relative to the PYY(1-36) peptide. In one aspect position 23 of the PYY peptide or an analogue thereof may be derivatised with an albumin binding group comprising CH₃(CH₂)ᵣCO—, wherein r is 16 or 18, wherein said position is relative to the PYY(1-36) peptide.

Examples of amino acid residues comprising an amino group is lysine, ornithine, Epsilon-N-alkylated lysine such as Epsilon-N methyllysine, O-aminoethylserine, O-aminopropylserine or longer O alkylated serines containing a primary or secondary amino group in the side chain. In one aspect of the invention, the derivatised amino acid residue comprises a primary amino group in a side chain. Examples of amino acid residues comprising a primary amino group is lysine ornithine, O-aminoethylserine, O-aminopropylserine or longer O alkylated serines containing a primary amino group in the side chain.

An example of a method for determination of albumin binding is as follows: Serum albumin binding could be measured by using columns with immobilised serum albumin from human or other species. The affinity of a given peptide can be measured by an altered elution time from the column and the relative affinities between different albumin binding peptides can be established by comparing the elution time profiles. In another method serum albumin peptides can be biotinylated and the binding of the peptide can be determined by enzyme linked immuno assay (ELISA) technique using microtiter plate with immobilised albumin. The visualisation of the binding is done by using avidin or streptavidin conjugated to either horseradish peroxidise or alkaline phosphatase. The relative affinities of different albumin binding peptides can be measured. Other affinity experiments that may be used in the measurement of albumin binding include Biacore analysis and microcalorimetry.

In one aspect of the invention, the albumin binding residue is a lipophilic residue. In one aspect, the lipophilic residue is attached to a lysine residue optionally via a spacer by conjugation chemistry such as by alkylation, acylation, ester formation, or amide formation or to a cysteine residue by maleimide coupling. The term “spacer” as used herein means a molecular unit separates a peptide and an albumin binding handle. In one aspect the term “spacer” as used herein means a spacer that separates a peptide and an albumin binding residue with a chemical moiety which comprises at least 5 non-hydrogen atoms where 30-50% of these are either N or O.

In one aspect of the invention, the albumin binding residue is negatively charged at physiological pH. In one aspect of the invention, the albumin binding residue comprises a group which can be negatively charged. One preferred group which can be negatively charged is a carboxylic acid group.

In one aspect of the invention, the albumin binding residue is selected from the group consisting of a straight chain alkyl group, a branched alkyl group, a group which has an ω-carboxylic acid group, and a partially or completely hydrogenated cyclopentanophenanthrene skeleton.

In one aspect of the invention, the albumin binding residue is a cibacronyl residue.

In one aspect of the invention, the albumin binding residue has from 6 to 40 carbon atoms, from 8 to 26 carbon atoms or from 8 to 20 carbon atoms.

In one aspect of the invention, the albumin binding residue is an acyl group selected from the group comprising CH₃(CH₂)ᵣCO—, wherein r is an integer from 4 to 38, specifically an integer from 4 to 24, more preferred selected from the group comprising CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

In one aspect of the invention, the albumin binding residue is an acyl group of a straight-chain or branched alkane α,ω-dicarboxylic acid.

In one aspect of the invention, a peptide derivative comprising a peptide wherein at least one amino acid residue, such as lysine, and/or the N- and/or C-terminus of the peptide backbone is derivatised with either A-B-C-D-, A-C-D-, A-B-C-, or A-C-, wherein

### A- is

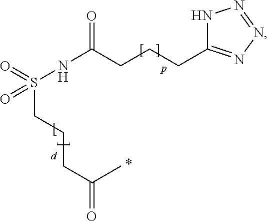

wherein p is selected from the group consisting of 10, 11, 12, 13, 14, 15 and 16 and d is selected from the group consisting of 0, 1, 2, 3, 4 and 5, and -B- is selected from the group consisting of

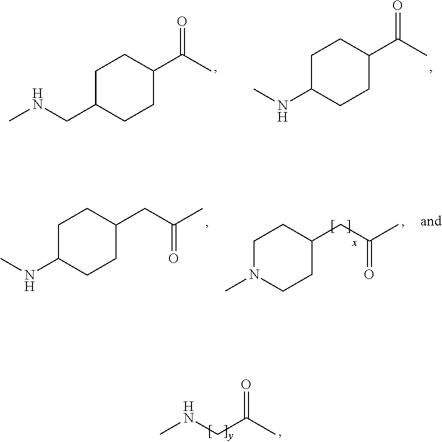

wherein x is selected from the group consisting of 0, 1, 2, 3 and 4, and y is selected from the group consisting of 1, 2, 3, 4, 5, 6, 7, 8, 9, 10, 11 and 12;

### or A- is

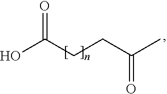

wherein n is selected from the group consisting of 12, 13, 14, 15, 16 17, 18 and 19, and -B- is selected from the group consisting of

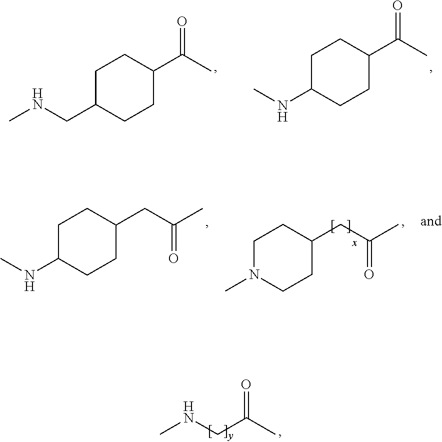

wherein x is selected from the group consisting of 0, 1, 2, 3 and 4;
and -C- is selected from the group consisting of

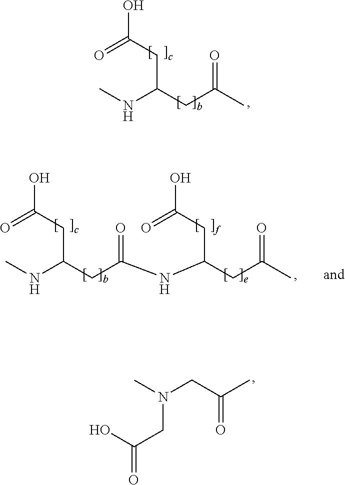

wherein b and e are each independently selected from the group consisting of 0, 1, and 2, and c and f are each independently selected from the group consisting of 0, 1, and 2 with the proviso that when
c is 0 b is 1 or 2,
c is 1 or 2 b is 0,
f is 0 e is 1 or 2,
f is 1 or 2 e is 0; and
-D- is attached to said amino acid residue and is a spacer.

In one aspect the invention relates to a PYY or PP peptide derivative or analogue thereof according to any of the preceding embodiments, wherein the peptide is selected from the group consisting of

a PP analogue according to formula I

(I)
Z-Ala-Pro-Leu-Glu-Pro-Val-Tyr-Pro-Gly-Xaa₁₀-Xaa₁₁-
Xaa₁₂-Xaa₁₃-Xaa₁₄-Xaa₁₅-Xaa₁₆-Xaa₁₇-Xaa₁₈-Xaa₁₉-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Thr-Arg-Xaa₃₄-Arg-Xaa₃₆,

wherein
Z is the side chain A-B-C-D-, A-C-D-, A-B-C-, or A-C- attached to the N-terminal amino group, or not present when A-B-C-D-, A-C-D-, A-B-C-, A-C- is attached to the side chain of an amino acid,
Ala in position 1 may be deleted,
Xaa₁₀ is Asp, Asn, 2,3-diaminopropionic acid, 2,4-diaminobutyric acid, ornithine, or Lys,
Xaa₁₁ is Asp, Asn, 2,3-diaminopropionic acid, 2,4-diaminobutyric acid, ornithine, or Lys,
Xaa₁₂ is Ala, 2,3-diaminopropionic acid, 2,4-diaminobutyric acid, ornithine, or Lys,
Xaa₁₃ is Thr, 2,3-diaminopropionic acid, 2,4-diaminobutyric acid, ornithine, or Lys,
Xaa₁₄ is Pro or hydroxyproline,
Xaa₁₅ is Glu, 2,3-diaminopropionic acid, 2,4-diaminobutyric acid, ornithine, or Lys,
Xaa₁₆ is Gln, 2,3-diaminopropionic acid, 2,4-diaminobutyric acid, ornithine, or Lys,
Xaa₁₇ is Leu, Val, Ile, homoleucine, norleucine, (1-aminocyclopentyl) carboxylic acid, (1-aminocyclohexyl) carboxylic acid, or 1-aminobutyric acid,
Xaa₁₈ is Ala, 2,3-diaminopropionic acid, 2,4-diaminobutyric acid, ornithine, or Lys,
Xaa₁₉ is Gln, 2,3-diaminopropionic acid, 2,4-diaminobutyric acid, ornithine, or Lys,
Xaa₂₀ is Tyr, Phe, or 3-pyridylalanine,
Xaa₂₁ is Ala, 2,3-diaminopropionic acid, 2,4-diaminobutyric acid, ornithine, or Lys,
Xaa₂₂ is Ala, 2,3-diaminopropionic acid, 2,4-diaminobutyric acid, ornithine, or Lys,
Xaa₂₃ is Asp, 2,3-diaminopropionic acid, 2,4-diaminobutyric acid, ornithine, or Lys,
Xaa₂₄ is Leu, Val, Ile, homoleucine, norleucine, (1-aminocyclopentyl) carboxylic acid, (1-aminocyclohexyl) carboxylic acid, or 1-aminobutyric acid,
Xaa₂₅ is Arg, 2,3-diaminopropionic acid, 2,4-diaminobutyric acid, ornithine, or Lys,
Xaa₂₆ is Arg, His, 2,3-diaminopropionic acid, 2,4-diaminobutyric acid, ornithine, or Lys,
Xaa₂₇ is Tyr, Phe, homoPhe, or 3-pyridylalanine,
Xaa₂₈ is Ile, Val, Leu, homoleucine, norleucine, (1-aminocyclopentyl) carboxylic acid, (1-aminocyclohexyl) carboxylic acid, or 1-aminobutyric acid,

### Xaa₂₉ is Asn, Gln, or Lys,

Xaa₃₀ is Met, Leu, Val, Ile, homoleucine, norleucine, (1-aminocyclopentyl) carboxylic acid, (1-aminocyclohexyl) carboxylic acid, or 1-aminobutyric acid,
Xaa₃₁ is Leu, Val, Ile, homoleucine, norleucine, (1-aminocyclopentyl) carboxylic acid, (1-aminocyclohexyl) carboxylic acid, or 1-aminobutyric acid,
Arg in position 33 may be substituted with Lys,

### Xaa₃₄ is Gln, Asn, or His,

Arg in position 35 may be substituted with Lys,
Xaa₃₆ is Tyr, 3-pyridylalanine;
a PYY analogue according to formula II

(II)
Z-Tyr-Pro-Xaa₃-Xaa₄-Pro-Glu-Ala-Pro-Gly-Xaa₁₀-
Xaa₁₁-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Thr-Arg-Xaa₃₄-Arg-
Xaa₃₆,

wherein
Z is the side chain A-B-C-D-, A-C-D-, A-B-C-, or A-C- attached to the N-terminal amino group, or not present when A-B-C-D-, A-C-D-, A-B-C-, A-C- is attached to the side chain of an amino acid,
Tyr-Pro in position 1 and 2 may be deleted,
Tyr in position 1 may substituted with Ala or be deleted,
Xaa₃ is Ile, Val, Leu (1-aminocyclopentyl) carboxylic acid, (1-aminocyclohexyl) carboxylic acid, or 1-aminobutyric acid,
Xaa₄ is Glu, 2,3-diaminopropionic acid, 2,4-diaminobutyric acid, ornithine, or Lys, Glu in position 6 may be substituted with Val,
Ala in position 7 may be substituted with Tyr,
Xaa₁₀ is Glu, 2,3-diaminopropionic acid, 2,4-diaminobutyric acid, ornithine, or Lys,
Xaa₁₁ is Asp, 2,3-diaminopropionic acid, 2,4-diaminobutyric acid, ornithine, or Lys,
Xaa₁₂ is Ala, 2,3-diaminopropionic acid, 2,4-diaminobutyric acid, ornithine, or Lys,
Xaa₁₃ is Ser, 2,3-diaminopropionic acid, 2,4-diaminobutyric acid, ornithine, or Lys,
Xaa₁₄ is Pro, hydroxyproline, or Lys,
Xaa₁₅ is Glu, 2,3-diaminopropionic acid, 2,4-diaminobutyric acid, ornithine, or Lys,
Xaa₁₆ is Glu, 2,3-diaminopropionic acid, 2,4-diaminobutyric acid, ornithine, or Lys,
Xaa₁₇ is Leu, Val, Ile, homoleucine, norleucine, (1-aminocyclopentyl) carboxylic acid, (1-aminocyclohexyl) carboxylic acid, or 1-aminobutyric acid,
Xaa₁₈ is Asn, 2,3-diaminopropionic acid, 2,4-diaminobutyric acid, ornithine, or Lys,
Xaa₁₉ is Arg, 2,3-diaminopropionic acid, 2,4-diaminobutyric acid, ornithine, or Lys,
Xaa₂₀ is Tyr, Phe, 3-pyridylalaine, 2,3-diaminopropionic acid, 2,4-diaminobutyric acid, ornithine, or Lys,
Xaa₂₁ is Tyr, Phe, 3-pyridylalaine, 2,3-diaminopropionic acid, 2,4-diaminobutyric acid, ornithine, or Lys,
Xaa₂₂ is Asp, 2,3-diaminopropionic acid, 2,4-diaminobutyric acid, ornithine, or Lys,
Xaa₂₃ is Ala, 2,3-diaminopropionic acid, 2,4-diaminobutyric acid, ornithine, or Lys,
Xaa₂₄ is Leu, Ile, Val, homoleucine, norleucine, (1-aminocyclopentyl) carboxylic acid, (1-aminocyclohexyl) carboxylic acid, 1-aminobutyric acid, or Lys,
Xaa₂₅ is Arg, 2,3-diaminopropionic acid, 2,4-diaminobutyric acid, ornithine, or Lys,
Xaa₂₆ is His, Arg, 2,3-diaminopropionic acid, 2,4-diaminobutyric acid, ornithine, or Lys,
Xaa₂₇ is Tyr, Phe, homoPhe, or 3-pyridylalanine,
Xaa₂₈ is Ile, Val, Leu, homoleucine, norleucine, (1-aminocyclopentyl) carboxylic acid, (1-aminocyclohexyl) carboxylic acid, 1-aminobutyric acid, or Lys,

### Xaa₂₉ is Asn, Gln, or Lys,

Xaa₃₀ is Met, Leu, Val, Ile, homoleucine, norleucine, (1-aminocyclopentyl) carboxylic acid, (1-aminocyclohexyl) carboxylic acid, 1-aminobutyric acid, or Lys,
Xaa₃₁ is Leu, Val, Ile, homoleucine, norleucine, (1-aminocyclopentyl) carboxylic acid, (1-aminocyclohexyl) carboxylic acid, 1-aminobutyric acid, or Lys,
Thr in position 32 may be substituted with Lys,

### Xaa₃₄ is Gln, Asn, or His,

Xaa₃₆ is Tyr, 3-pyridylalanine, or Lys,
wherein the compound is modified with a serum albumin binding side chain comprising a distal carboxylic acid or tetrazole group.

In one aspect said the N-terminus is an amino group and/or said C-terminus is a carboxylic acid group.

In one aspect of the invention -D- is a spacer providing distance of the albumin handles to the peptide and may be selected from the group consisting of one or more consecutive PEG molecules, one or more consecutive glycine or other small polar residues. In one aspect said spacer may be one or more consecutive 8-amino-3,6-dioxaoctanoic acid (Oeg) molecules or other spacers of the PEG type. In one aspect said spacer may be a peptide and may be one or more consecutive Gly molecules forming a glycine polymer. In one aspect the spacer may be composed of several polar or hydrophilic amino acids. As an example but not restricted to is (Ser-Gly)n where n is an integer; n=1-20 or 1-10 or 1-5. In one aspect the spacer may be composed of non-alfa-amino acids such as beta-alanine or 8-amino-caprylic acid or combinations thereof.

In one aspect, D is selected from the group consisting of

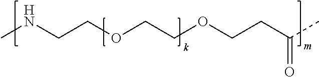

and wherein k is selected from the group consisting of 0, 1, 2, 3, 4, 5, 11 and 27, and m is selected from the group consisting of 0, 1, 2, 3, 4, 5 and 6.

In one aspect, A-B-C-D- is selected and combined from

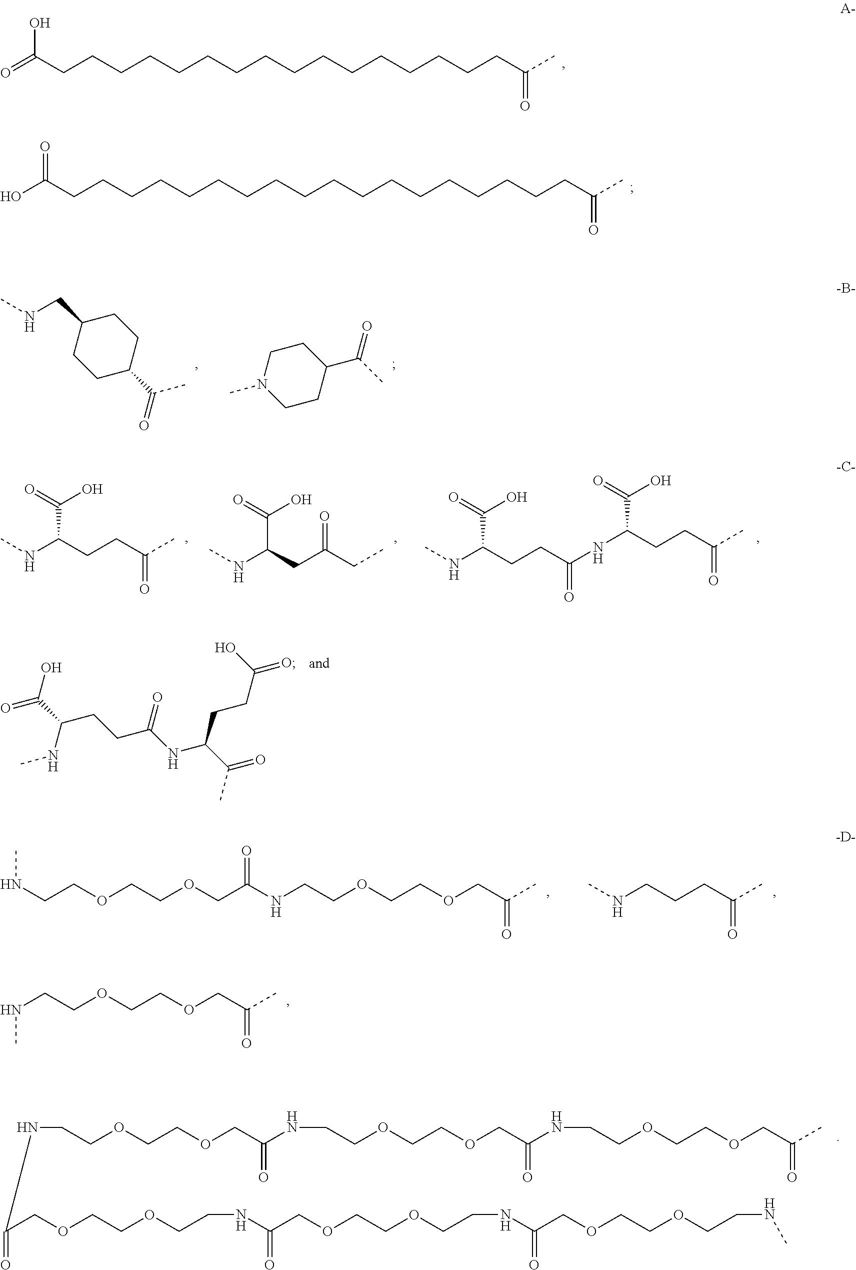

In one aspect, A-B-C-D- is selected and combined from

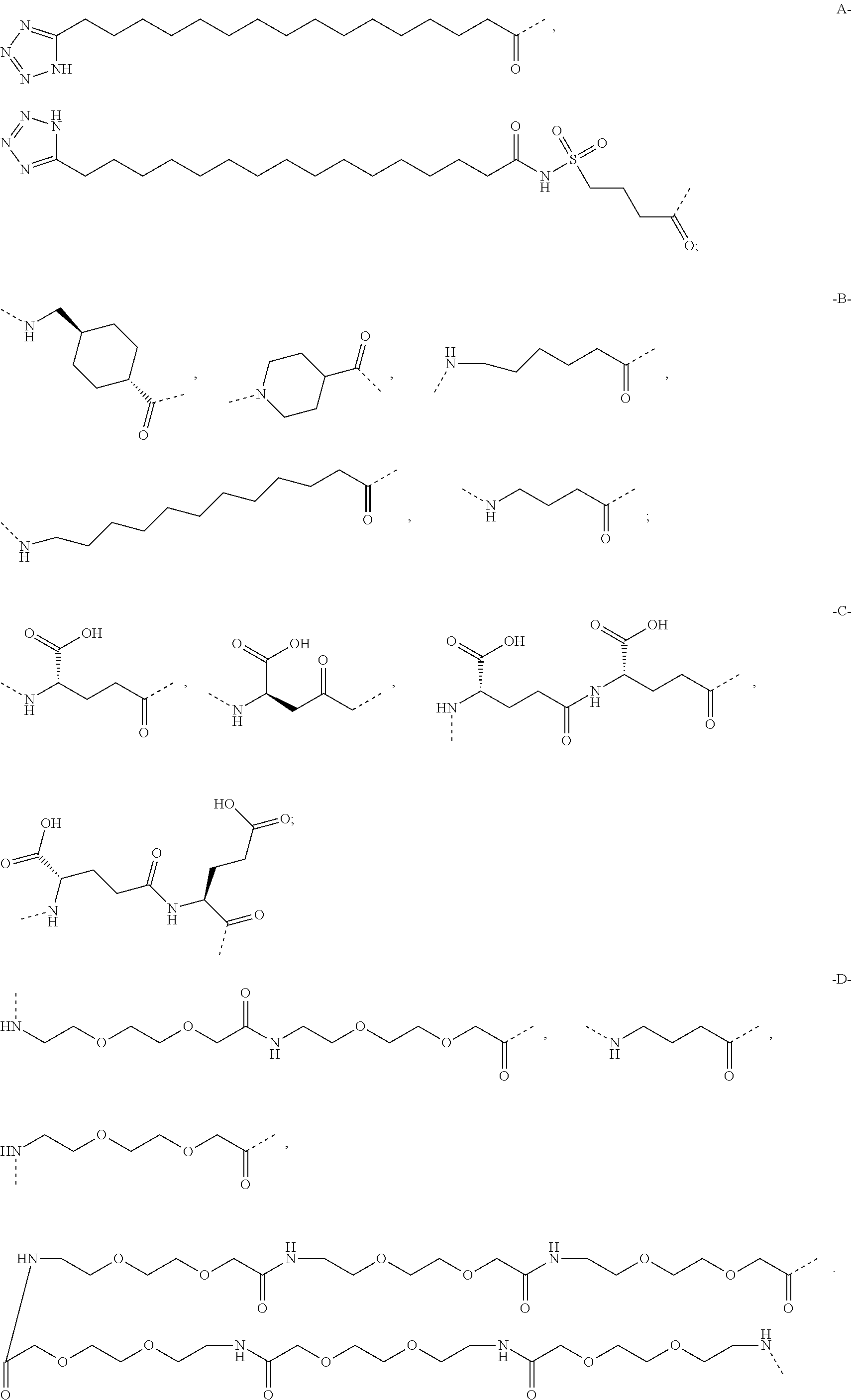

In one aspect, A-B-C-D- is selected from the group consisting of

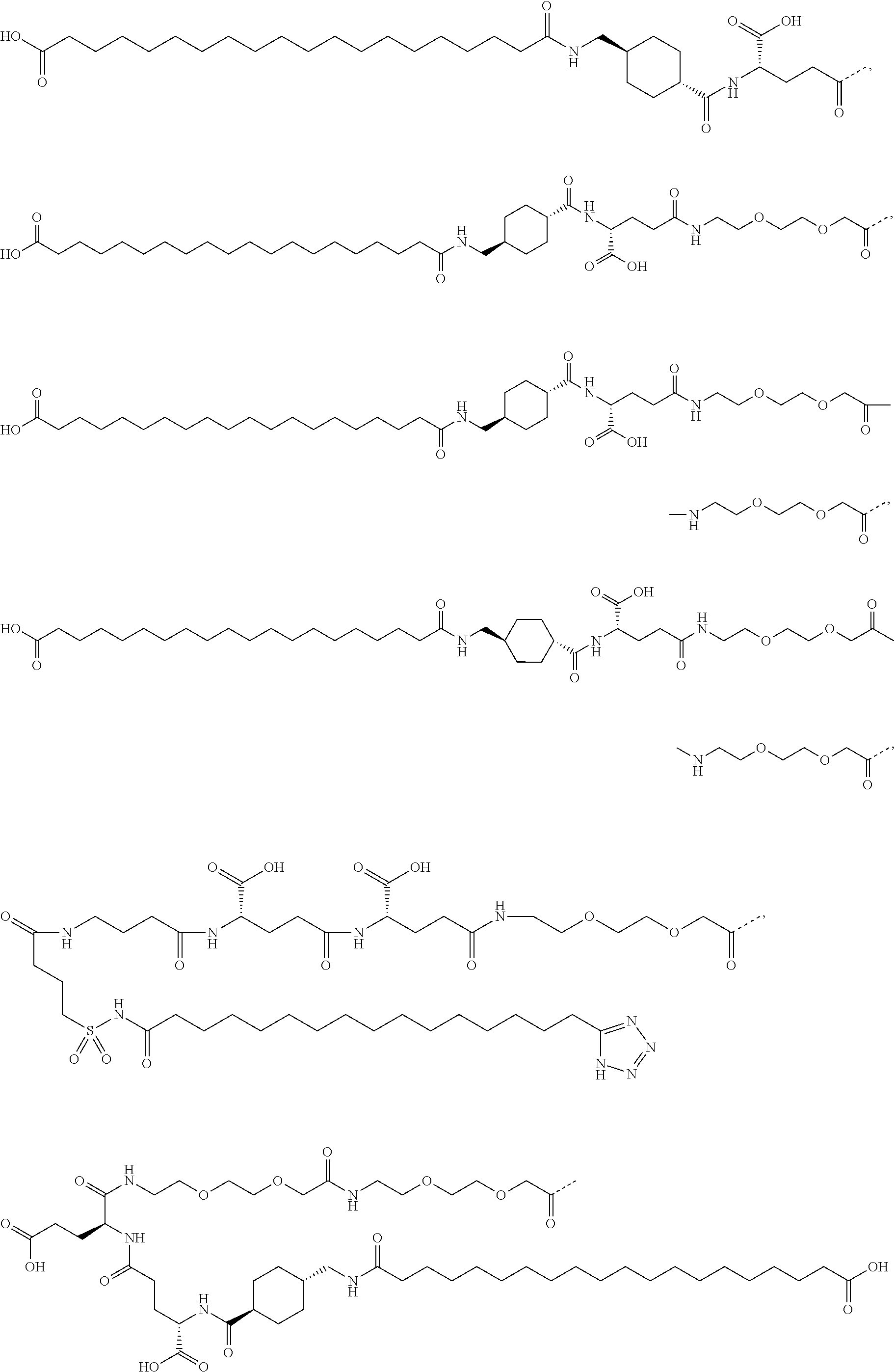

In one aspect, the invention relates to a PYY or PP analogue or derivative thereof, wherein A-B-C-D- is 2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl. In one aspect, the invention relates to a PYY or PP analogue or derivative thereof, wherein A-B-C-D- is 2-(2-{2-[2-(2-{2-[(S)-4-Carboxy-4-(17-carboxyheptadecanoylamino)butyrylamino]ethoxy}ethoxy)-acetylamino]ethoxy}ethoxy)acetyl. In one aspect, the invention relates to a PYY or PP analogue or derivative thereof, wherein A-B-C-D- is [4-(16-(1H-Tetrazol-5-yl)hexadecanoylsulfamoyl)butyryl]ethoxy}ethoxy)acetylamino]ethoxy}ethoxy)acetyl].

In one aspect, the invention relates to a PYY or PP analogue or derivative thereof, wherein at least one amino acid residue and/or the N terminal amino group of the peptide backbone is derivatised with A-B-C-D-, and where the derivative binds to albumin.

In one aspect, A-B-C-D is composed of an albumin binding fragment A-B-C- and a hydrophilic spacer, D.

Attaching fatty di-acids, e.g., hexadecanedioic acid, octadecanedioic acid, or dodecanedioic acid introduces an additional negative charge in the distal end of the fatty acid. This increases the affinity to serum albumin. The di-acid may be attached to a spacer such as a negatively charged amino acid, e.g., L-gamma-glutamate but not restricted hereto as such. The fatty di-acid may also be attached to a hydrophobic spacer such as tranexamic acid and isonipecotinic acid but not restricted as such.

In one aspect the combined di-acid (A-B-C- or A-C-) and spacer (-D-) may be separated with one or more consecutive spacers such as 8-amino-3,6-dioxaoctanoic acid (Oeg).

In one aspect, the PYY or PP peptide derivatives or analogues thereof of the invention retain at least about 25%, specifically about 30%, 40%, 50%, 60%, 70%, 80%, 90%, 95%, 98%, or 99% percent of the biological activity of human PYY or PP, respectively, with regard to the reduction of food intake, the effect on body weight, gastric emptying, change in respiratory quotient, and/or the effect on intestinal electrolyte secretion. In one aspect, the PYY or PP peptide derivatives or analogues thereof of the invention exhibit improved PYY or PP peptide derivative or analogue thereof activity, respectively. In one aspect, the PYY or PP peptide derivatives or analogues thereof of the invention exhibits at least about 110%, 125%, 130%, 140%, 150%, 200%, or more of the biological activity of human PYY or PP, respectively, with regard to the reduction of food intake, the effect on body weight, gastric emptying, change in respiratory quotient, and/or the effect on intestinal electrolyte secretion. Methods for measuring said biological effects are provided in later sections of this document. In one aspect the PYY or PP peptide analogues or derivatives thereof have a potency in one of the assays described herein (such as food intake, the effect on body weight, gastric emptying, change in respiratory quotient, and/or the effect on intestinal electrolyte secretion) which is equal to or greater than the potency of human PYY or PP in that same assay. Alternatively, PYY or PP peptide analogues or derivatives thereof may exhibit improved ease of manufacture, stability, and/or ease of formulation, as compared to human PYY or PP.

In one aspect, the PYY or PP peptide analogues or derivatives exhibit improved protracted properties in vivo compared to human PYY or PP.

The albumin binding handle may be linked to an amino, carboxyl, or thiol group, and may be linked by N or C termini, or at the side chains of lysine, aspartic acid, glutamic acid, or cysteine. Alternatively, the albumin binding handle may be linked with diamine and dicarboxylic groups.

PYY or PP peptide derivatives or analogues thereof of the invention also include PYY or PP peptide derivatives or analogues thereof with chemical alterations to one or more amino acid residues. Such chemical alterations include amidation, glycosylation, acylation, sulfation, phosphorylation, acetylation, and cyclization. The chemical alterations may occur singularly at the N- or C-terminus or at the side chains of amino acid residues within the sequence of the PYY or PP peptide derivatives or analogues thereof. In one aspect, the C-terminus of these peptides may have a free —OH or —NH₂ group. In one aspect, the N-terminal end may be capped with an isobutyloxycarbonyl group, an isopropyloxycarbonyl group, an n-butyloxycarbonyl group, an ethoxycarbonyl group, an isocaproyl group (isocap), an octanyl group, an octyl glycine group (G(Oct)), an 8-aminooctanic acid group or a Fmoc group. In one aspect, cyclization can be through the formation of disulfide bridges or lactam bridge between a Lys and Glu or a Lys and Asp. Alternatively, there may be multiple sites of chemical alteration along the PYY or PP analogue peptide.

In one aspect, the present invention relates to a derivative of PYY or PP or analogue thereof which has substantially improved terminal half-life in rodent and in a non-rodent model relative to any one of PYY, PYY(3-36), or PP.

In one aspect of this invention, the terminal half-life in rodent or in a non-rodent model is improved at least 3 fold relative to any one of PYY, PYY(3-36), or PP. In one aspect of this invention, the terminal half-life in a non-rodent model is improved at least 6 fold relative to any one of PYY, PYY(3-36), or PP. In one aspect of this invention, the terminal half-life in a non-rodent model is improved at least 10 fold relative to any one of PYY, PYY(3-36), or PP. In one aspect of this invention, the terminal half-life in a non-rodent model is improved at least 50 fold relative to any one of PYY, PYY(3-36), or PP. In one aspect the present invention relates to a derivative of PYY or PP or analogue thereof, wherein said derivative or analogue shows an improvement of terminal half-life compared to human PYY(3-36) in the range of 5-500, such as 10-500, 20-500, 50-500, 10-400, 20-400, 50-400, 100-500, 100-400 or 200-500 fold determined in vivo using a non-rodent model. In one aspect the present invention relates to a derivative of PYY or PP or analogue thereof, wherein said derivative shows an improvement of terminal half-life compared to human PP in the range of 50-5000, such as 100-5000, 200-5000, 500-5000, 100-4000, 200-4000, 500-4000, 1000-5000, 1000-4000 or 2000-5000 fold determined in vivo using a non-rodent model.

In one aspect, the present invention relates to a derivative of PYY or PP or analogue thereof which has substantially improved terminal half-life in a non-rodent model relative to any one of PYY, PYY(3-36), or PP and wherein the binding to the Y2 and/or Y4 receptors has at least the same level of potency as any one of PYY, PYY(3-36), or PP. In one aspect, the present invention relates to a derivative of PYY or PP or analogue thereof which has substantially improved terminal half-life in a non-rodent model relative to any one of PYY, PYY(3-36), or PP and wherein the binding to the Y2 and/or Y4 receptors has at least 50%, such as 60%, 70%, 80% or 80% potency as any one of PYY, PYY(3-36), or PP.

In one aspect, the present invention relates to a derivative of PYY or PP or analogue thereof which has an in vivo half-life of at least 10 h after i.v. administration to rats.

In one aspect, the present invention relates to a derivative of PYY or PP or analogue thereof which has an in vivo half-life of at least 10 h, such as at least 20 h, at least 30 h, at least 40 h, at least 50 h, at least 100 h, at least 150 h, at least 200 h, at least 250 h, at least 300 h, or at least 350 h after s.c. or i.v. administration to mini pigs, and alternatively an in vivo half-life of at least 80 h after s.c. or i.v. administration to mini pigs.

In one aspect, the present invention relates to a derivative of PYY or PP or analogue thereof which can be formulated into particles suitable for pulmonary administration.

In one aspect, the present invention relates to a derivative of PYY or PP or analogue thereof which is chemically and physically stable at neutral pH, most specifically in the range 6-8.

In embodiments of the invention a combination of the above features is achieved.

A range of albumin binding residues are known among linear and branched lipohophillic moieties containing 4-40 carbon atoms having a distal acidic group.

In the formulas herein the terminal dashed bonds from the attached groups, A, B, C, and D, are to be regarded as attachment bonds and not ending in methylene groups unless stated. In the compounds according to the invention the groups A, B, C, and/or D are attached to each other by amide bonds.

In one aspect the invention relates to a PYY or PP peptide derivate or analogue thereof according to the invention, wherein the peptide may be truncated by deletion of a consecutive sequence of one or more amino acids from the N-terminal end. In one aspect, in said PYY or PP peptide derivate or analogue thereof, the consecutive sequence of one or more amino acids is selected from position 1 to 25 in PYY or position 1 to 2 in PP.

In one aspect the invention relates to a PYY or PP peptide derivate or analogue thereof according to the invention, wherein the serum albumin binding side chain is attached to the side chain of an amino acid of the peptide backbone.

In one aspect the invention relates to a PYY or PP peptide derivate or analogue thereof according to the invention, wherein the serum albumin binding side chain is attached to an amino group of the side chain of an amino acid of the peptide backbone.

In one aspect the invention relates to a PYY or PP peptide derivate or analogue thereof according to the invention, wherein the serum albumin binding side chain is attached to an amino group of the side chain of an amino acid of the peptide backbone selected from the group consisting of 2,3-diaminopropionic acid, 2,4-diaminobutyric acid, ornithine, and Lys.

In one aspect the invention relates to a PYY or PP peptide derivative or analogue thereof according to the invention, wherein the spacer, -D-, comprises one or more 8-amino-3,6-dioxaoctanoic acid (Oeg) molecules.

In one aspect the invention relates to a PYY or PP peptide derivative or analogue thereof according to the invention, wherein said derivative is selective for the Y2 and/or Y4 receptors over the Y1 receptor.

In one aspect the invention relates to a PYY or PP peptide derivative or analogue thereof according to the invention, wherein said derivative is selective for the Y2 and/or Y4 receptors over the Y5 receptor.

In one aspect the invention relates to a PYY or PP peptide derivative or analogue thereof according to the invention, wherein said derivative is suitable for administration in a once-daily dosing regime.

In one aspect the invention relates to a PYY or PP peptide derivative or analogue thereof according to the invention, wherein said derivative is suitable for administration in a once-weekly dosing regime.

In one aspect the invention relates to a PYY or PP peptide derivative or analogue thereof according to the invention, wherein said derivative is suitable for administration in a twice-monthly dosing regime.

In one aspect the invention relates to a PYY or PP peptide derivative or analogue thereof according to the invention, wherein said derivative is suitable for administration in a once-monthly dosing regime.

In one aspect the invention relates to a PYY or PP peptide derivative or analogue thereof according to the invention, wherein said derivative shows improved PK profile compared to human PYY or PP.

In one aspect the invention relates to a PYY or PP peptide derivative or analogue thereof according to the invention, wherein said derivative shows protracted properties compared to human PYY or PP.

In one aspect the invention relates to a PYY or PP peptide derivative or analogue thereof according to the invention, wherein said derivative shows improved half life in vivo compared to human PYY or PP.

In one aspect the invention relates to a PYY or PP peptide derivative or analogue thereof according to the invention, wherein a therapeutically effective dose of said derivative causes less side effects compared to human PYY or PP.

In one aspect PYY or PP peptide derivatives according to the invention may be selected from the group consisting of compounds shown in Table A, with the proviso that the compound is not SEQ ID NO: 1, SEQ ID NO: 2 or SEQ ID NO: 73. In Table A SEQ ID NO: 1 is human PYY(3-36), SEQ ID NO: 2 is human PP(1-36) and SEQ ID NO: 73 is [Leu17,Leu30]hPP(2-36).

TABLE A
List of Compounds

SEQ ID NO: 1
Name: hPYY(3-36)
Structure: IKPEAPGEDASPEELNRYYASLRHYLNLVTRQRY
SEQ ID NO: 2
Name: hPP(1-36)
Structure: APLEPVYPGDNATPEQMAQYAADLRRYINMLTRPRY
SEQ ID NO: 3 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys13]hPYY(3-36) Structure: 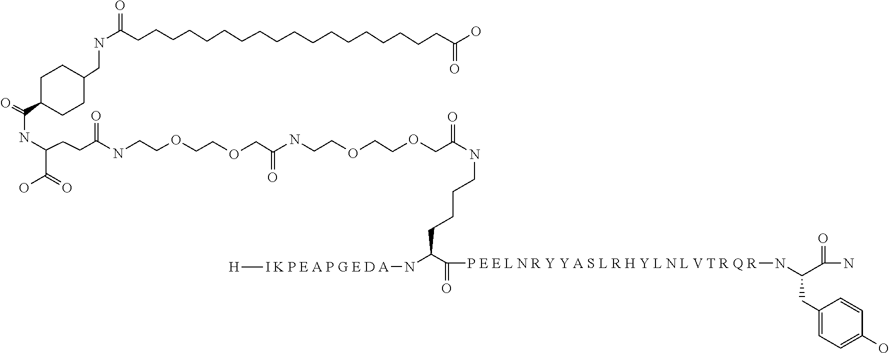
SEQ ID NO: 4 Name: N-epsilon13-[2-(2-{2-[2-(2-{2-[(S)-4-Carboxy-4-({trans-4-[(19)- carboxynonadecanoylamino)methyl]cyclohexanecarbonyl}amino)butyrylamino]- ethoxy}ethoxy)acetylamino]ethoxy}ethoxy)acetyl][Lys13,Leu17,Leu30,Gln34]hPP(1-36) Structure: 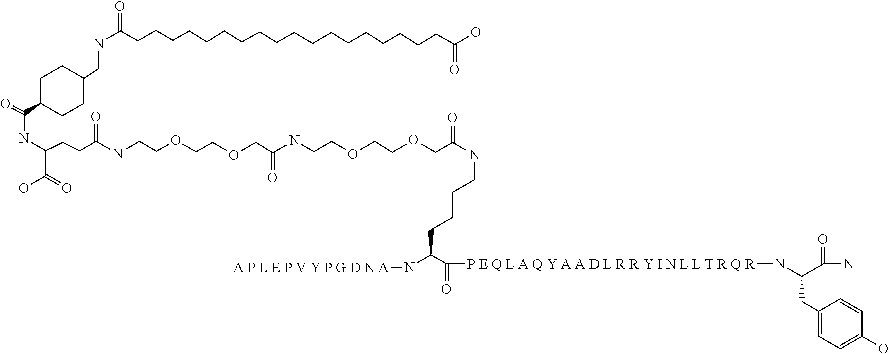
SEQ ID NO: 5 Name: N-epsilon13-[2-(2-{2-[2-(2-{2-[(S)-4-Carboxy-4-(17- carboxyheptadecanoylamino)butyrylamino]ethoxy}ethoxy)- acetylamino]ethoxy}ethoxy)acetyl][Lys13]hPYY(3-36) Structure: 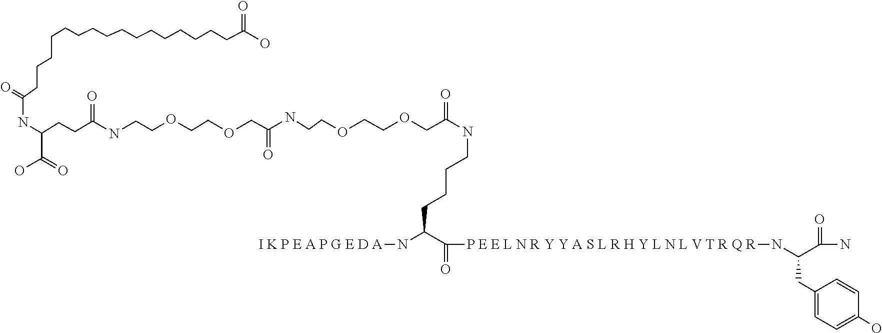
SEQ ID NO: 6 Name: N-epsilon10-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys10,Leu17,Leu30]hPP2-36 Structure: 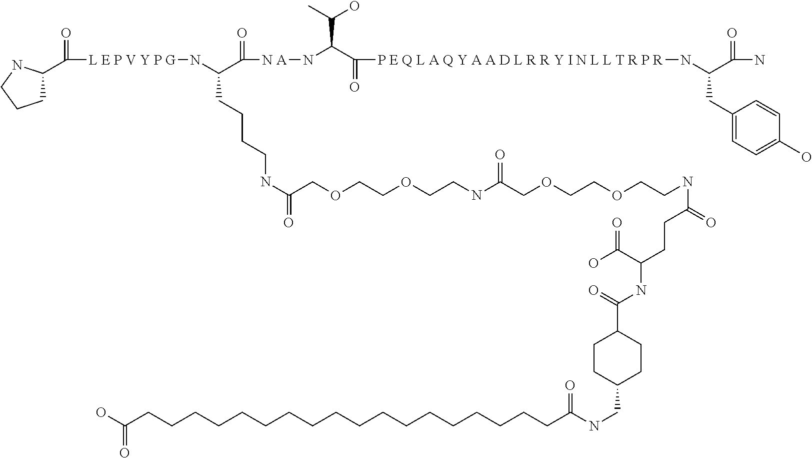
SEQ ID NO: 7 Name: N-epsilon10-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys10,Leu17,Leu30]hPP2-36 Structure: 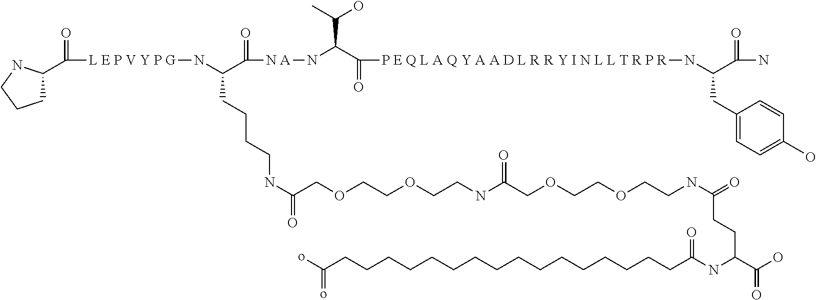
SEQ ID NO: 8 Name: N-epsilon11-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys11]hPYY(3-36) Structure: 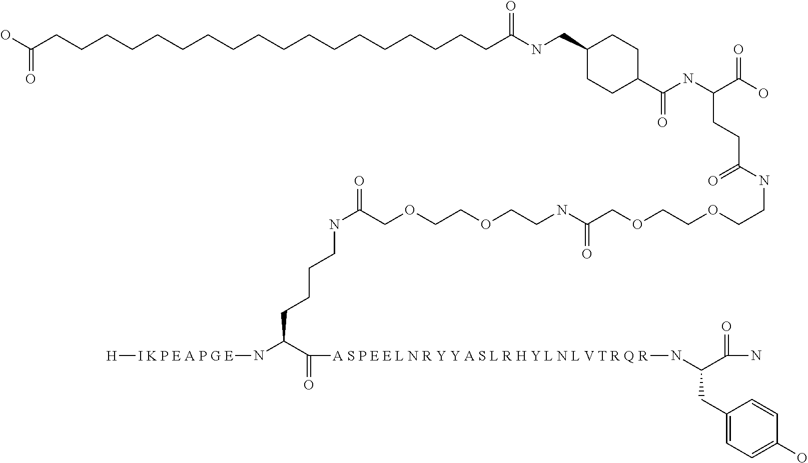
SEQ ID NO: 9 Name: N-epsilon11-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys11]hPYY(3-36) Structure: 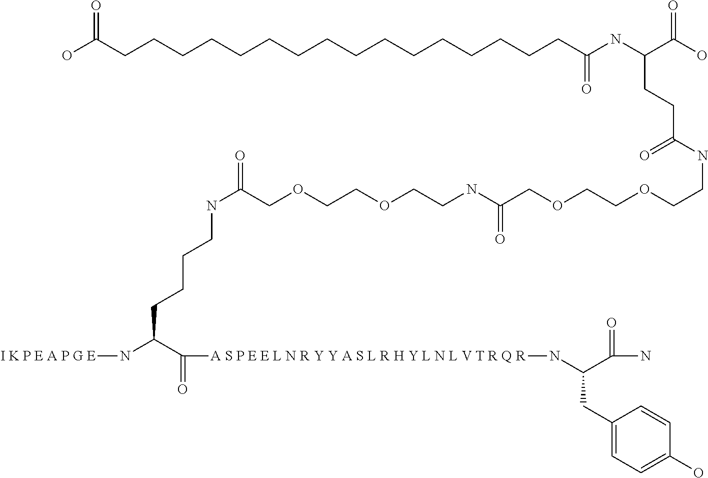
SEQ ID NO: 10 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys13,Leu17,Nle30,Gln34]hPP(1-36) Structure: 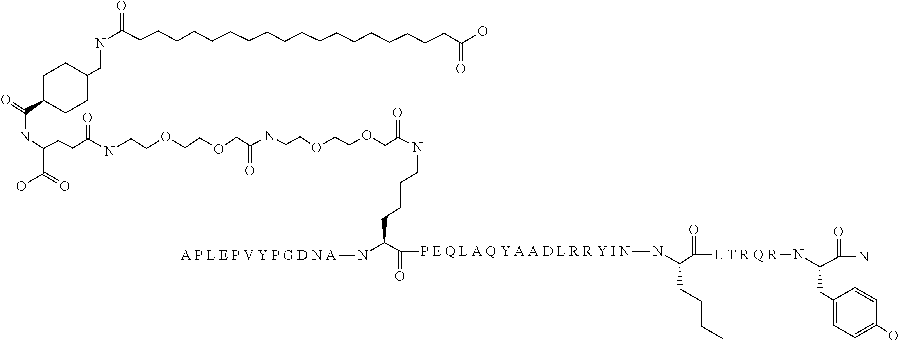
SEQ ID NO: 11 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys13]hPYY2-36 Structure: 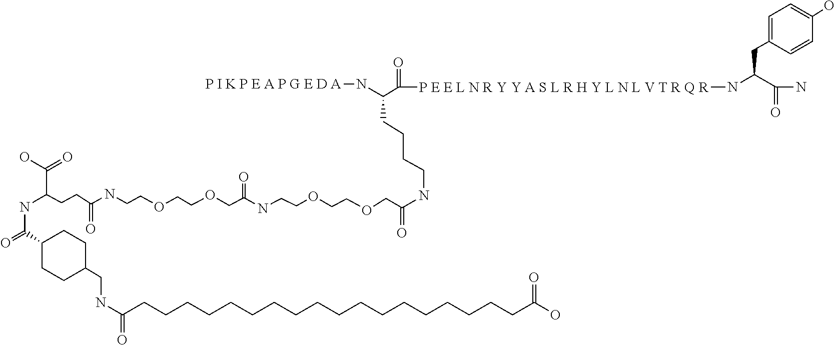
SEQ ID NO: 12 Name: N-epsilon4-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl]hPYY(3-36) Structure: 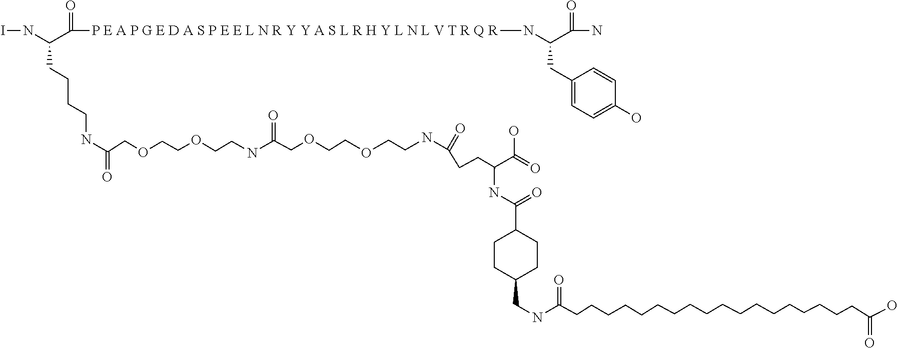
SEQ ID NO: 13 Name: N-epsilon4-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl]hPYY(3-36) Structure: 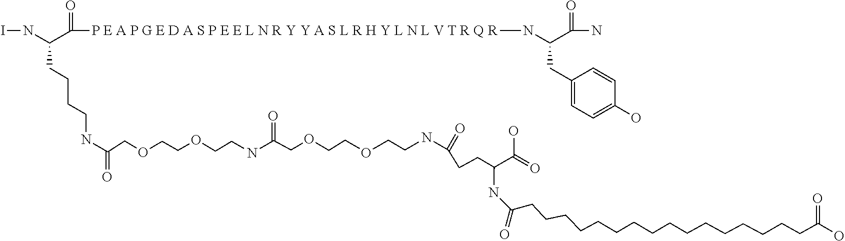
SEQ ID NO: 14 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]-eth- oxy}ethoxy)acetylamino]ethoxy}ethoxy)acetyl][Asn10,Asp11,Lys13,Leu17,Leu30,Val31]hPP(1-36) Structure: 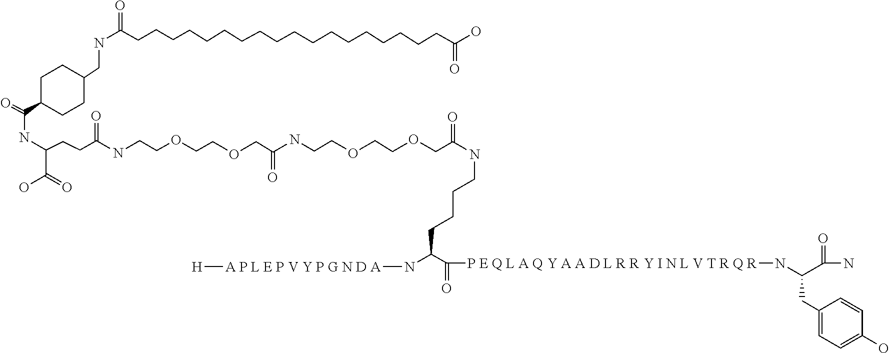
SEQ ID NO: 15 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]-eth- oxy}ethoxy)acetylamino]ethoxy}ethoxy)acetyl][Lys13,Leu17,Leu28,Val30,Gln34]hPP(1-36) Structure: 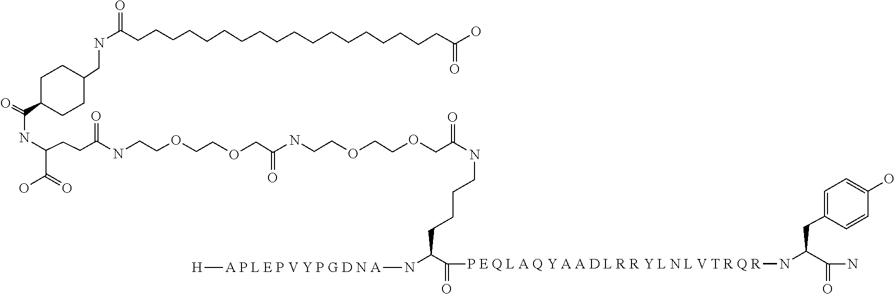
SEQ ID NO: 16 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]-eth- oxy}ethoxy)acetylamino]ethoxy}ethoxy)acetyl][Lys13,Leu17,Val28,Leu30,Gln34]hPP(1-36) Structure: 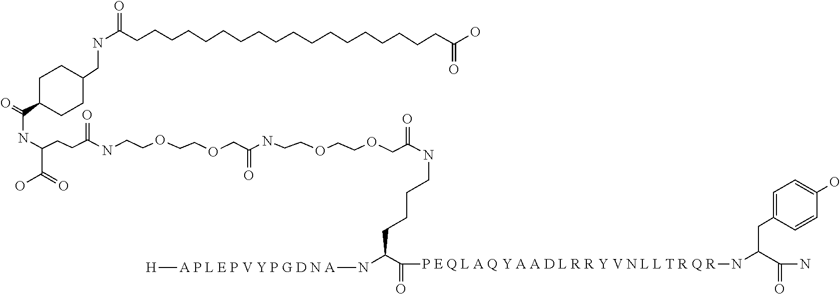
SEQ ID NO: 17 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys13,Leu17,Val30,Gln34]hPP(1-36) Structure: 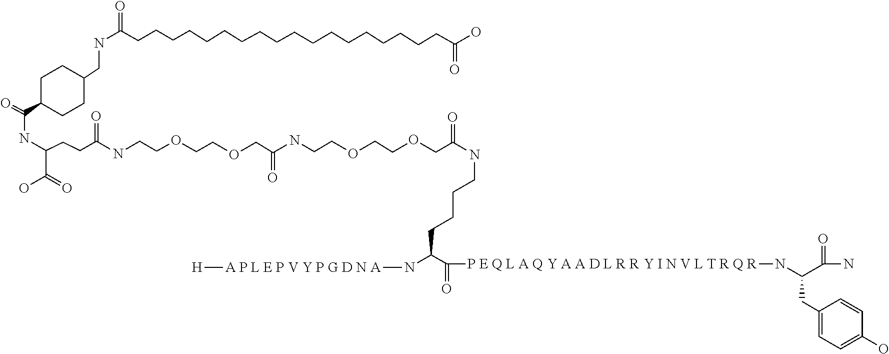
SEQ ID NO: 18 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys13,Leu17,Gln29,Leu30]hPP(1-36) Structure: 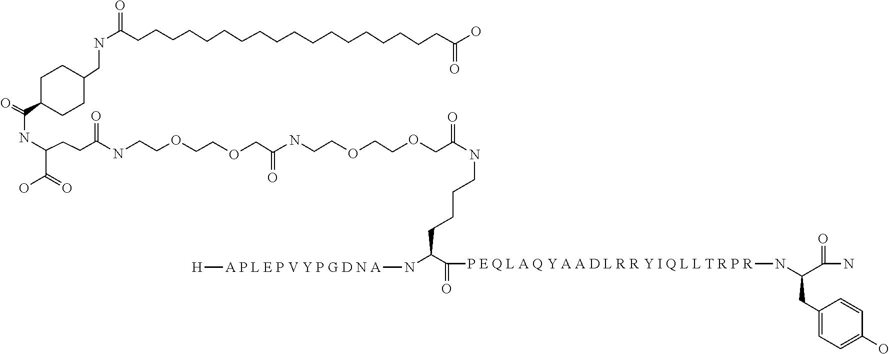
SEQ ID NO: 19 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys13,Arg26]hPPY(3-36) Structure: 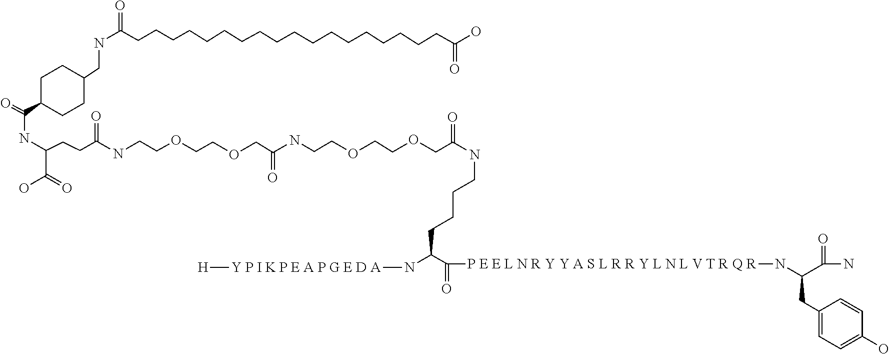
SEQ ID NO: 20 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]-eth- oxy}ethoxy)acetylamino]ethoxy}ethoxy)acetyl][Ala1,Leu3,Glu4,Val6,Tyr7,Lys13,Arg26]hPYY(1-36) Structure: 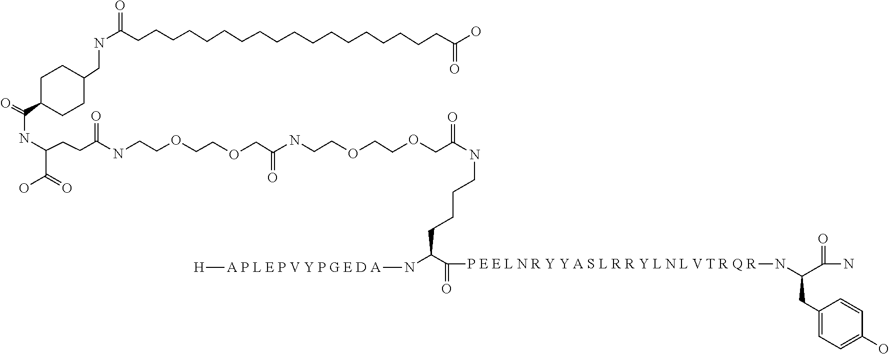
SEQ ID NO: 21 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Ala1,Glu4,Lys13,Arg26]hPYY(1-36) Structure: 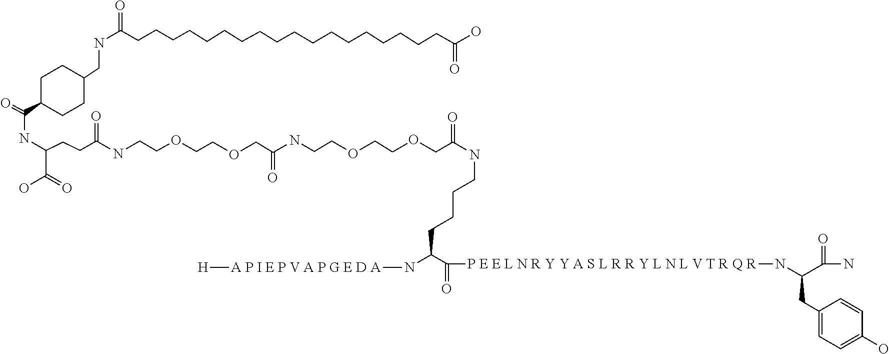
SEQ ID NO: 22 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]-eth- oxy}ethoxy)acetylamino]ethoxy)acetyl][Ala1,Glu4,Tyr7,Lys13,Arg26]hPYY(1-36) Structure: 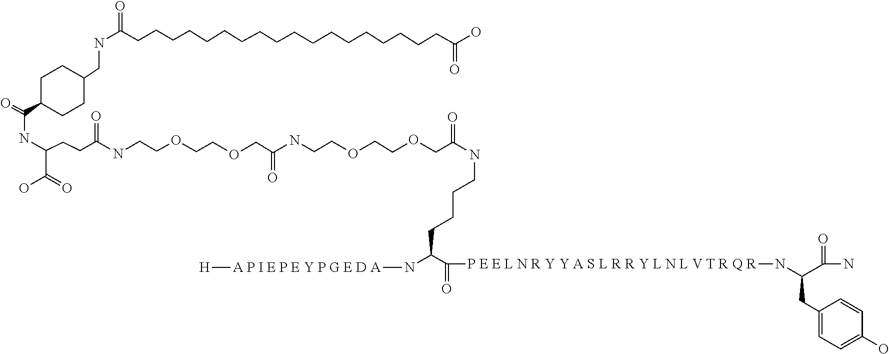
SEQ ID NO: 23 Name: N-alfa-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl]hPYY(3-36) Structure: 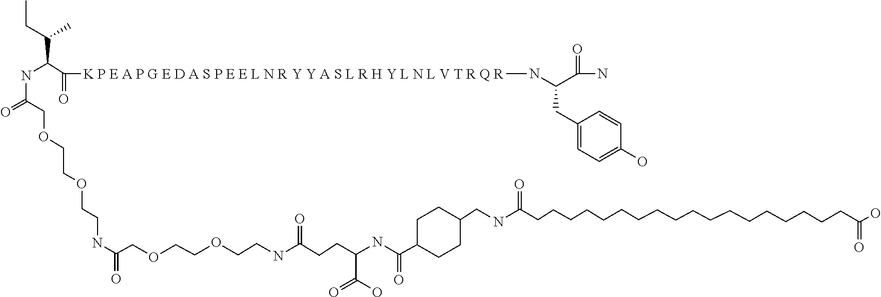
SEQ ID NO: 24 Name: N-epsilon13-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys13]hPP(1-36) Structure: 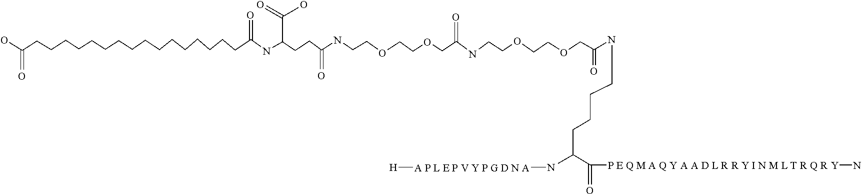
SEQ ID NO: 25 Name: N-epsilon4-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys4]hPYY(3-36) Structure: 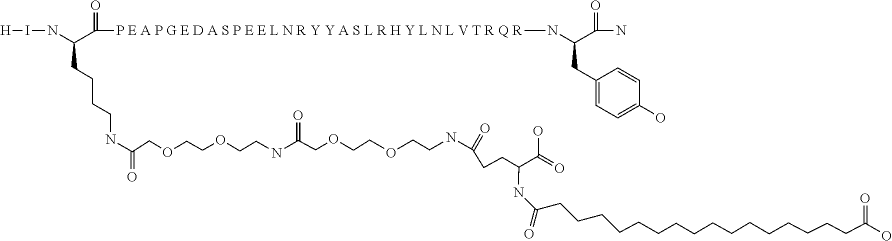
SEQ ID NO: 26 Name: N-epsilon13-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys13,Gln34]hPP(1-36) Structure: 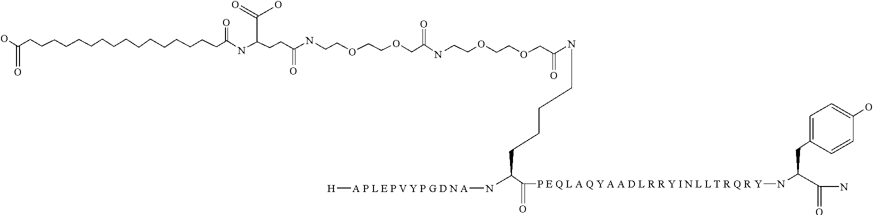
SEQ ID NO: 27 Name: N-epsilon11-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys11]hPYY(3-36) Structure: 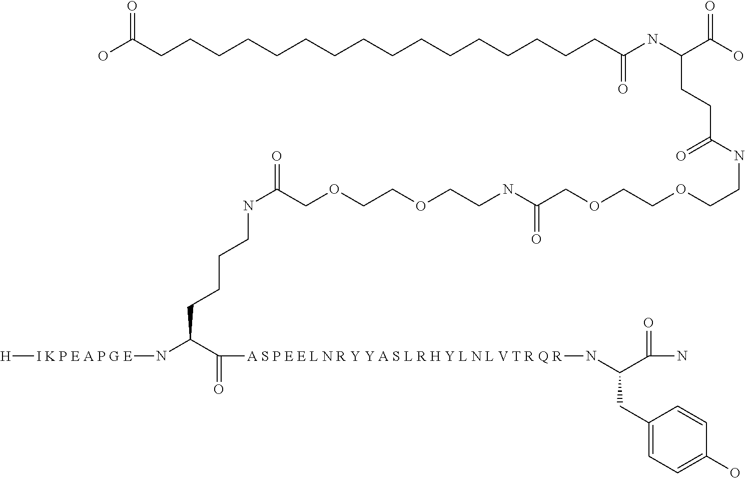
SEQ ID NO: 28 Name: N-epsilon11-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys11]hPYY(3-36) Structure: 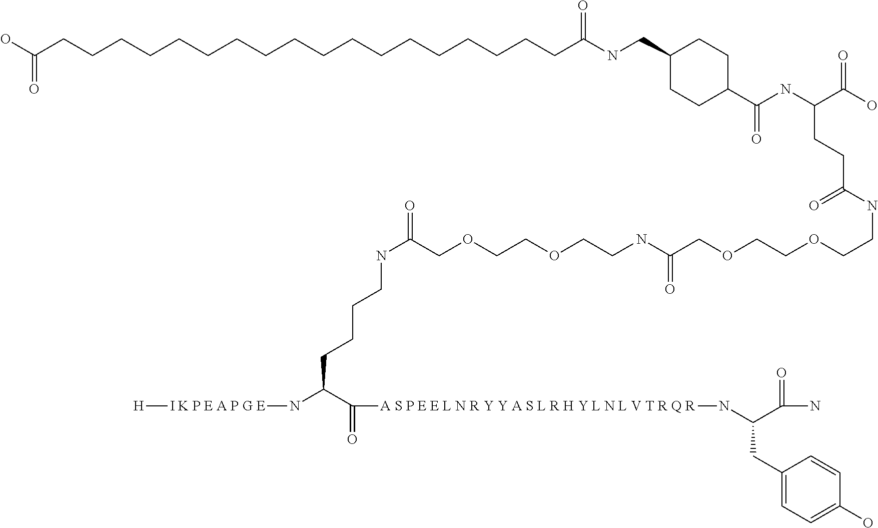
SEQ ID NO: 29 Name: N-epsilon11-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys11,Leu17,Leu30]hPP2-36 Structure: 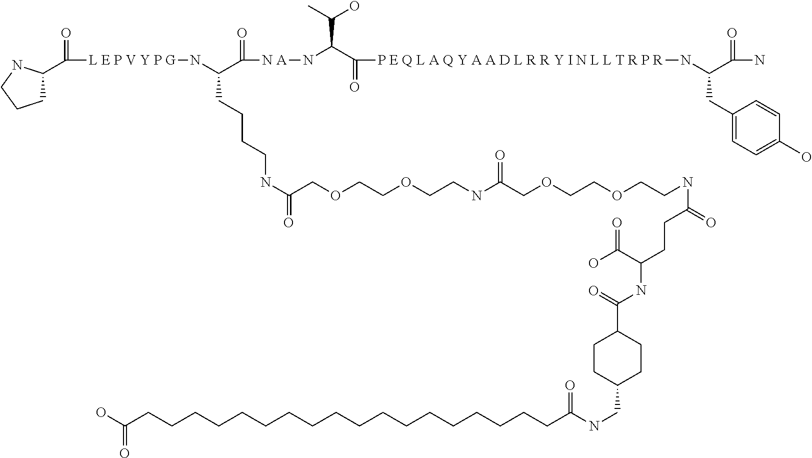
SEQ ID NO: 30 Name: N-epsilon11-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys11,Leu17,Leu30]hPP2-36 Structure: 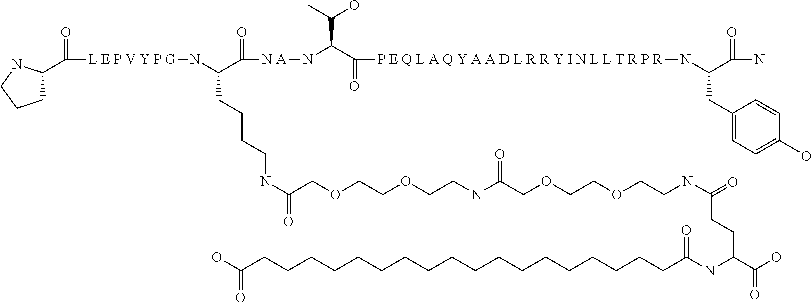
SEQ ID NO: 31 Name: N-epsilon13-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys13]hPP(1-36) Structure: 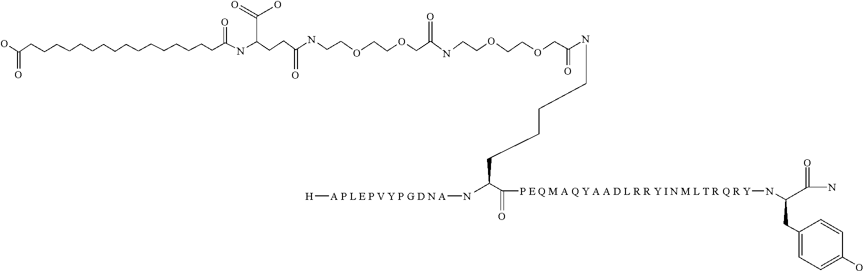
SEQ ID NO: 32 Name: N-epsilon18-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys18,Leu17,Leu30]hPP2-36 Structure: 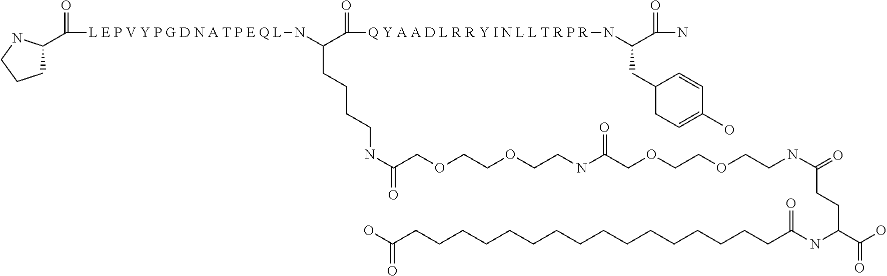
SEQ ID NO: 33 Name: N-alfa-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl]hPYY18-36 Structure: 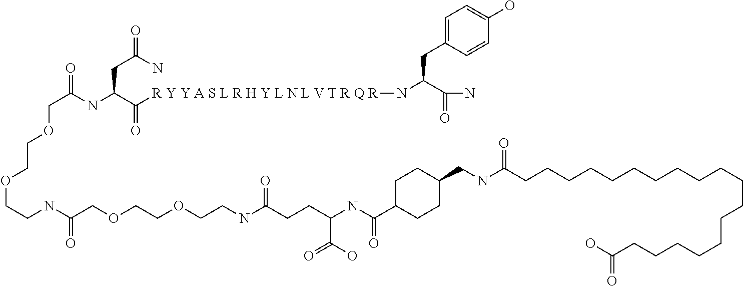
SEQ ID NO: 34 Name: N-epsilon25-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys25]hPYY(3-36) Structure: 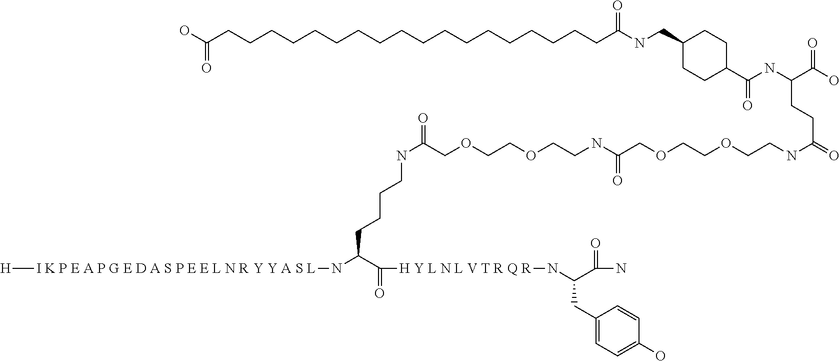
SEQ ID NO: 35 Name: N-epsilon24-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys24]hPYY(3-36) Structure: 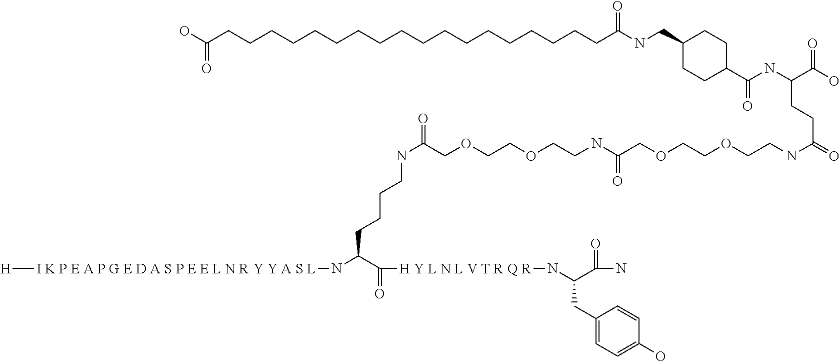
SEQ ID NO: 36 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys13,Leu17,Leu30]hPP(1-36) Structure: 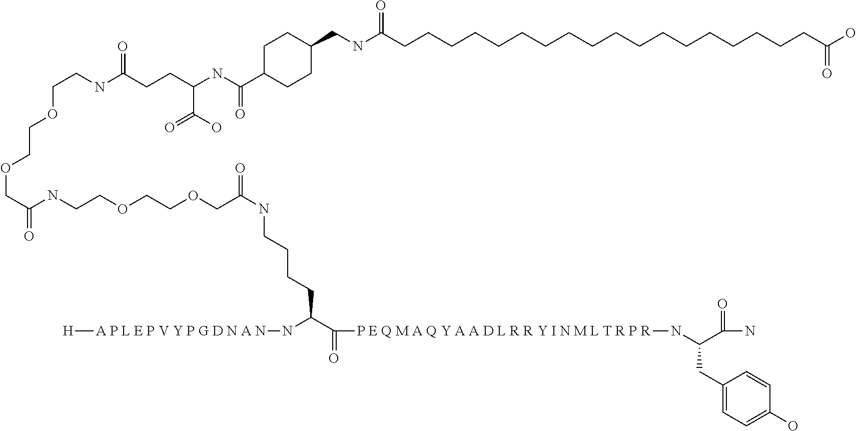
SEQ ID NO: 37 Name: N-epsilon25-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys17,Leu25,Leu30]hPP(1-36) Structure: 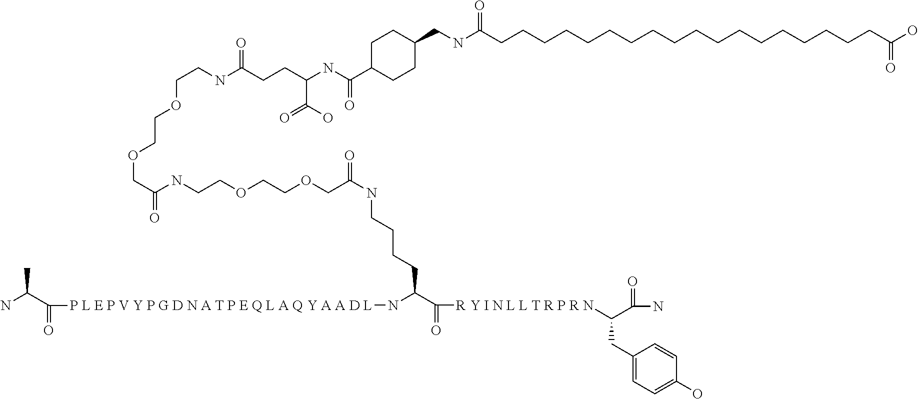
SEQ ID NO: 38 Name: N-epsilon15-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys15,Leu17,Leu30]hPP(1-36) Structure: 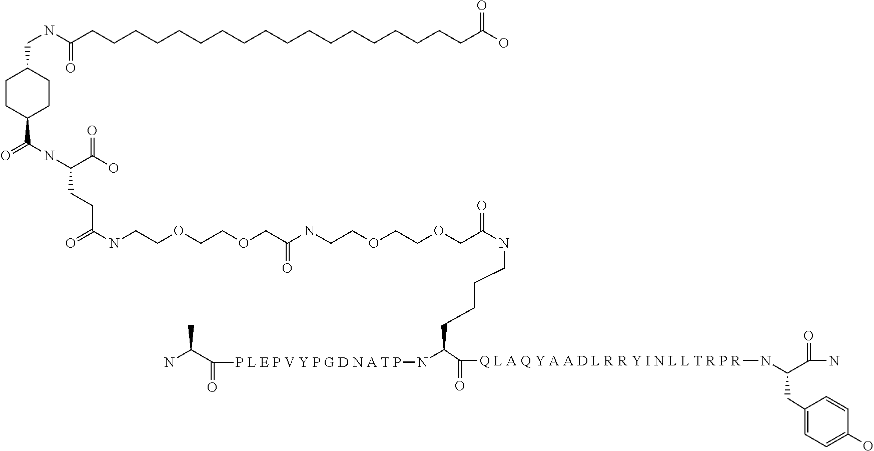
SEQ ID NO: 39 Name: N-epsilon10-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys10,Leu17,Leu30,Gln34]hPP2-36 Structure: 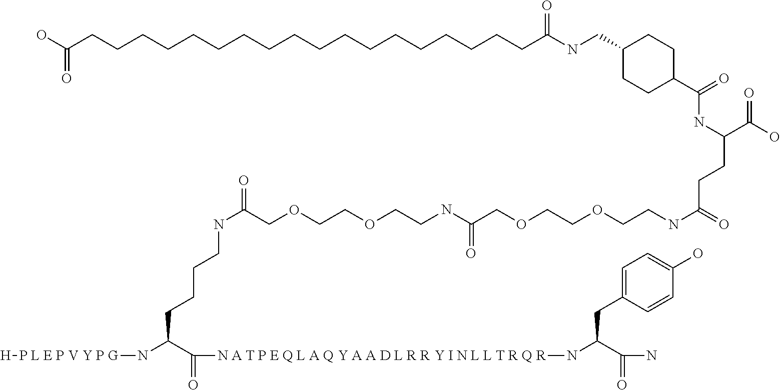
SEQ ID NO: 40 Name: N-epsilon19-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys19]hPYY(3-36) Structure: 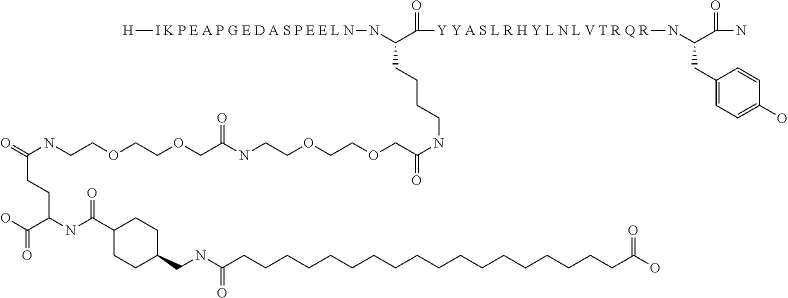
SEQ ID NO: 41 Name: N-epsilon33-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Leu17,Leu30,Lys33]hPP2-36 Structure: 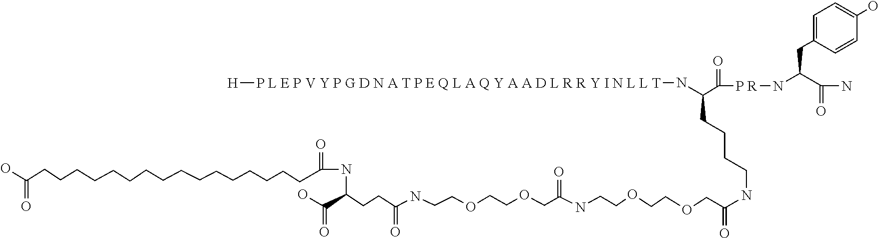
SEQ ID NO: 42 Name: N-epsilon33-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Leu17,Leu30,Lys33]hPP2-36 Structure: 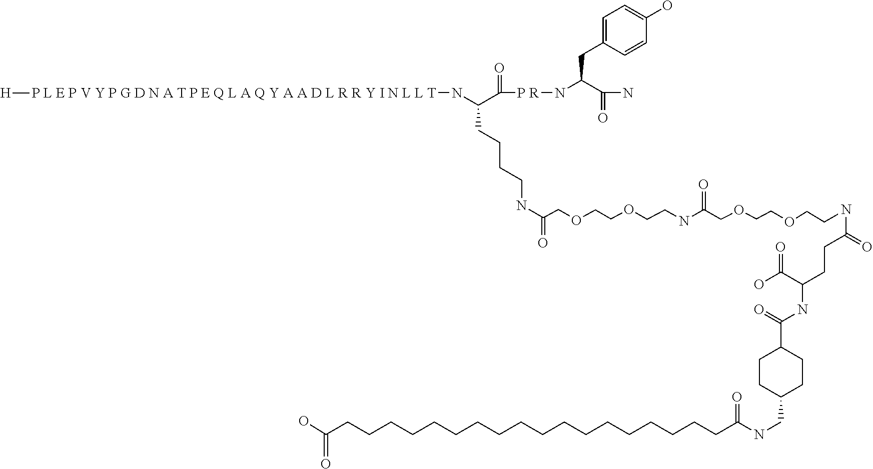
SEQ ID NO: 43 Name: N-epsilon18-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Leu17,Lys18,Leu30]hPP2-36 Structure: 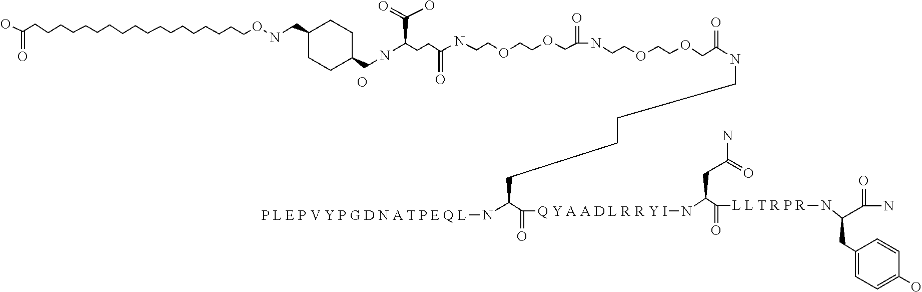
SEQ ID NO: 44 Name: N-epsilon29-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Leu17,Lys29,Leu30]hPP2-36 Structure: 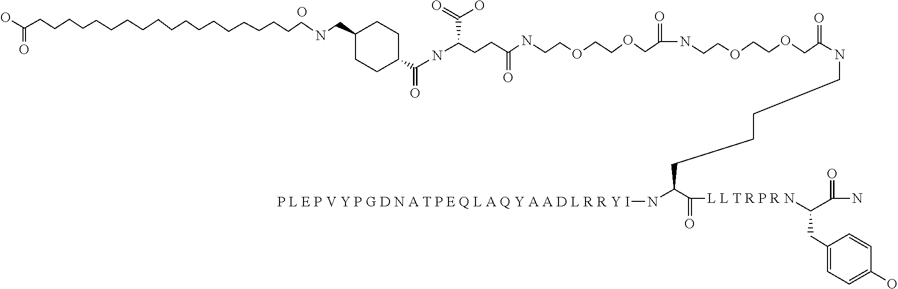
SEQ ID NO: 45 Name: N-epsilon26-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Leu17,Lys26,Leu30]hPP2-36 Structure: 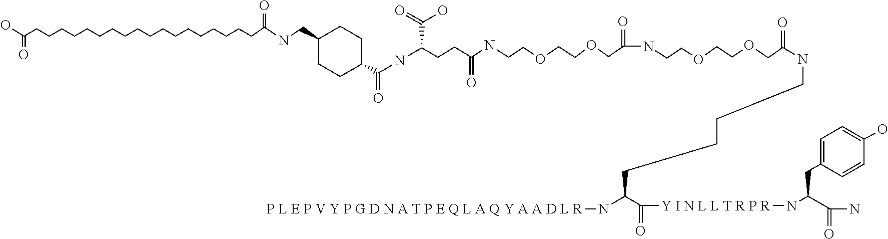
SEQ ID NO: 46 Name: N-epsilon26-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Leu17,Lys26,Leu30]hPP2-36 Structure: 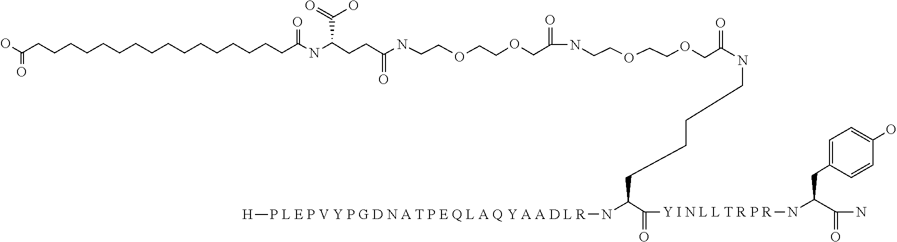
SEQ ID NO: 47 Name: N-epsilon35-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Leu17,Leu30,Lys35]hPP2-36 Structure: 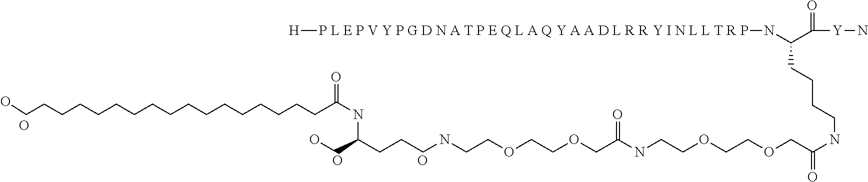
SEQ ID NO: 48 Name: N-epsilon35-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Leu17,Leu30,Lys35]hPP2-36 Structure: 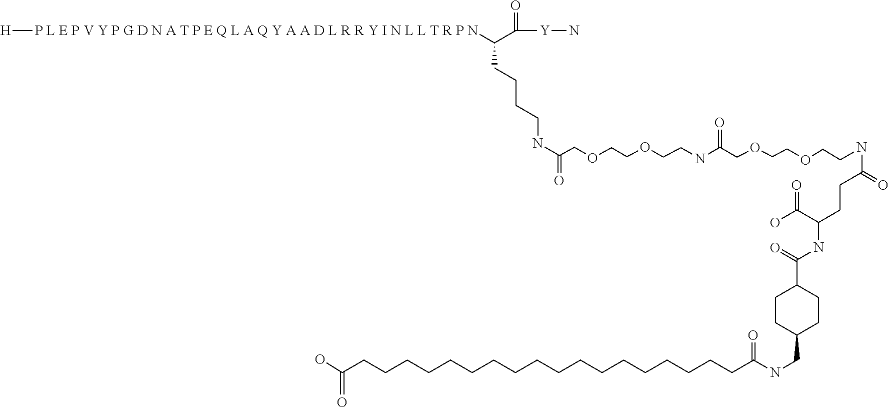
SEQ ID NO: 49 Name: N-epsilon25-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Leu17,Lys25,Leu30]hPP2-36 Structure: 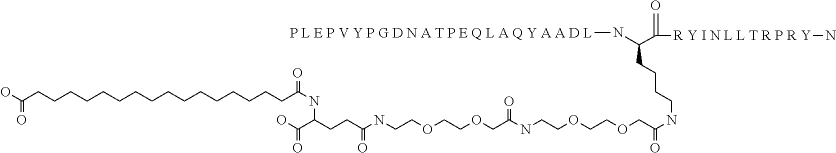
SEQ ID NO: 50 Name: N-epsilon25-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Leu17,Lys25,Leu30]hPP2-36 Structure: 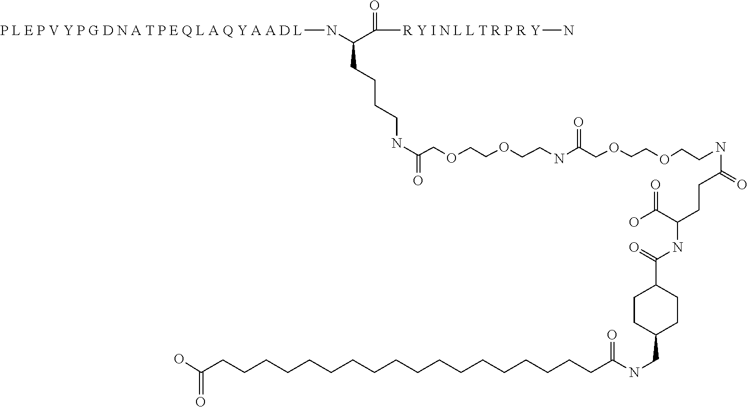
SEQ ID NO: 51 Name: N-epsilon13-[4-(16-(1H-Tetrazol-5-yl)hexadecanoylsulfamoyl)butyryl] ethoxy}ethoxy)acetylamino]ethoxy}ethoxy)acetyl][Lys13]PYY(3-36) Structure: 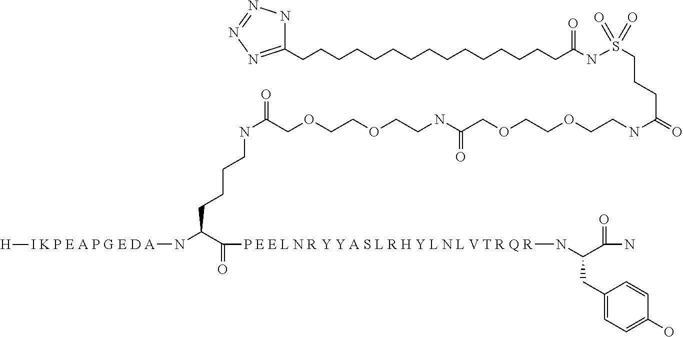
SEQ ID NO: 52 Name: N-epsilon25-[4-(16-(1H-Tetrazol-5-yl)hexadecanoylsulfamoyl)butyryl] ethoxy}ethoxy)acetylamino]ethoxy}ethoxy)acetyl][Lys25]PYY(3-36) Structure: 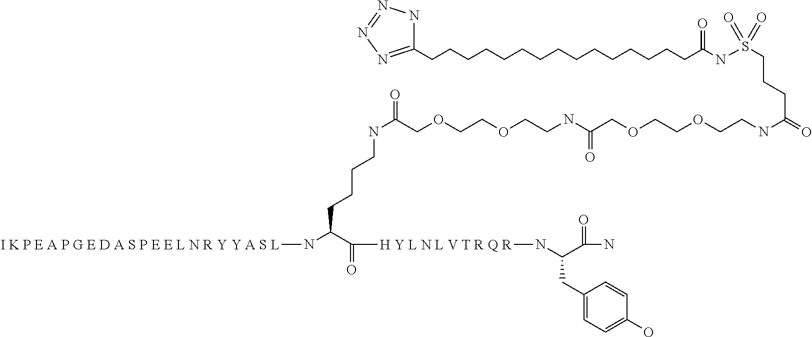
SEQ ID NO: 53 Name: N-alfa-[4-(16-(1H-Tetrazol-5-yl)hexadecanoylsulfamoyl)butyryl] ethoxy}ethoxy)acetylamino]ethoxy}ethoxy)acetyl]PYY(3-36) Structure: 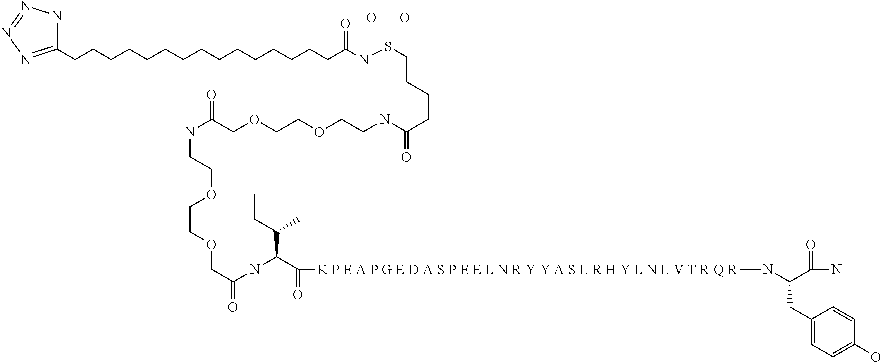
SEQ ID NO: 54 Name: N-alfa-[4-(16-(1H-Tetrazol-5-yl)hexadecanoylsulfamoyl)butyryl] ethoxy}ethoxy)acetylamino]ethoxy}ethoxy)acetyl]PYY(3-36) Structure: 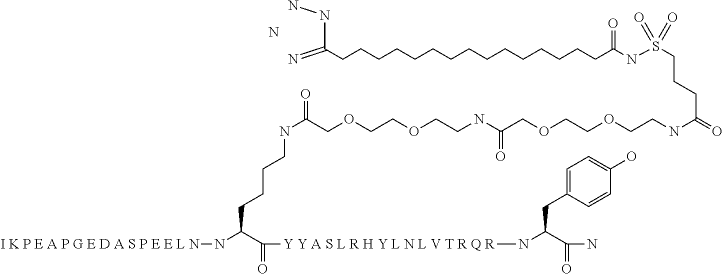
SEQ ID NO: 55 Name: N-alfa-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Leu17,Leu30]hPP2-36 Structure: 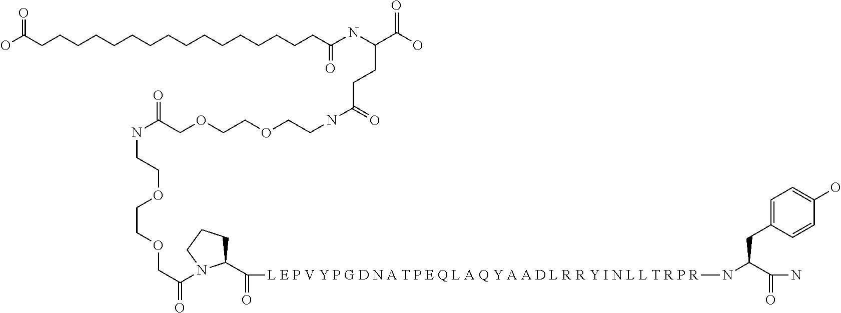
SEQ ID NO: 56 Name: N-alfa-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Leu17,Leu30]hPP2-36 Structure: 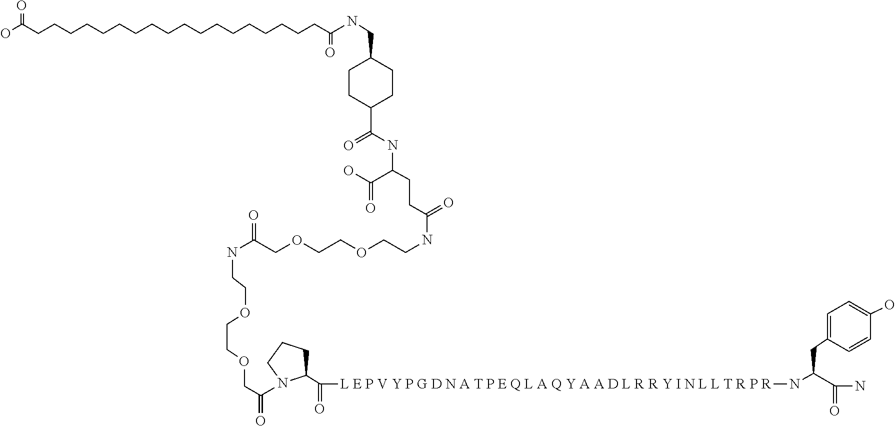
SEQ ID NO: 57 Name: N-epsilon18-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys18]hPYY3-36 Structure: 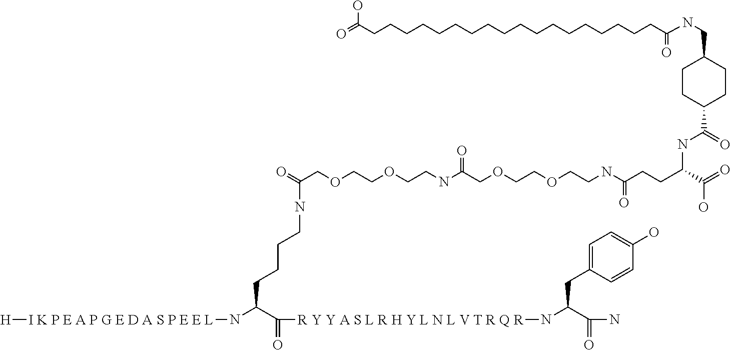
SEQ ID NO: 58 Name: N-epsilon22-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys22]hPYY3-36 Structure: 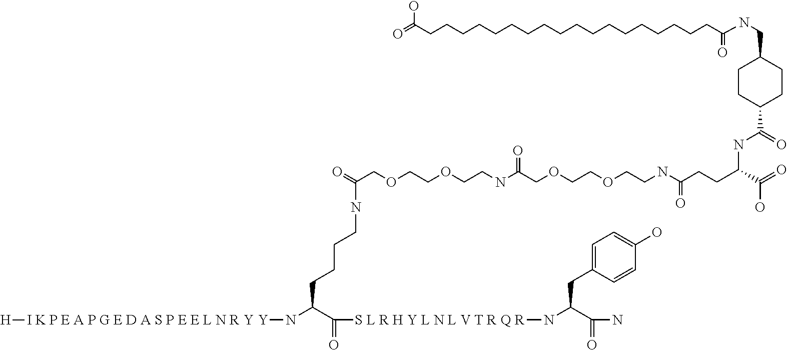
SEQ ID NO: 59 Name: N-epsilon26-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys26]hPYY3-36 Structure: 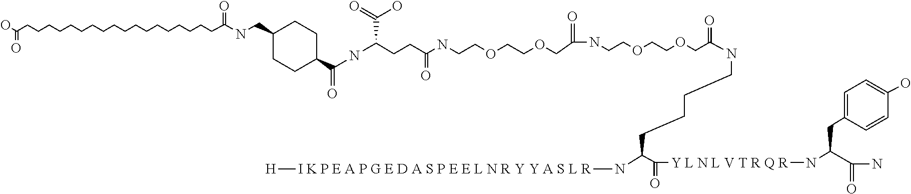
SEQ ID NO: 60 Name: N-epsilon29-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys29]hPYY3-36 Structure: 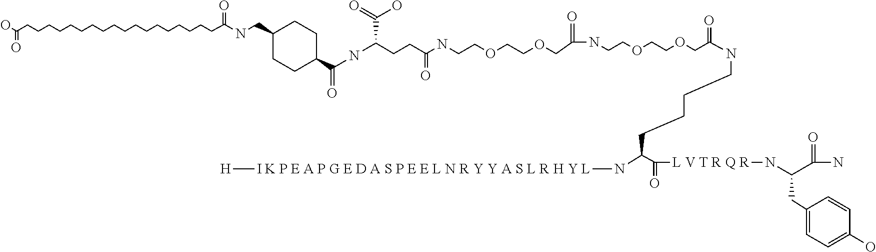
SEQ ID NO: 61 Name: N-epsilon36-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Lys36]hPYY3-36 Structure: 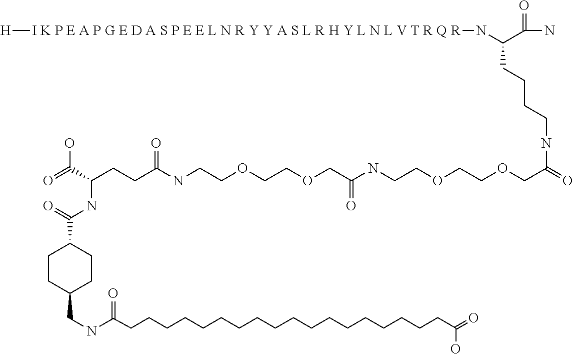
SEQ ID NO: 62 Name: N-epsilon21-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys21]hPYY3-36 Structure: 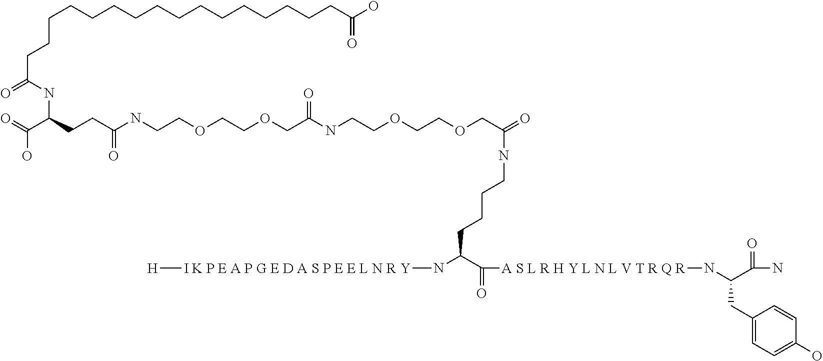
SEQ ID NO: 63 Name: N-epsilon30-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys30]hPYY3-36 Structure: 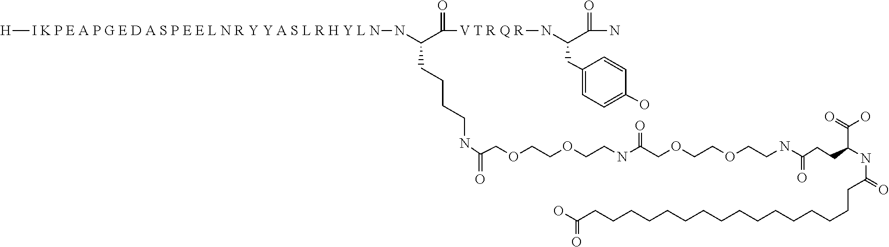
SEQ ID NO: 64 Name: N-epsilon31-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys31]hPYY3-36 Structure: 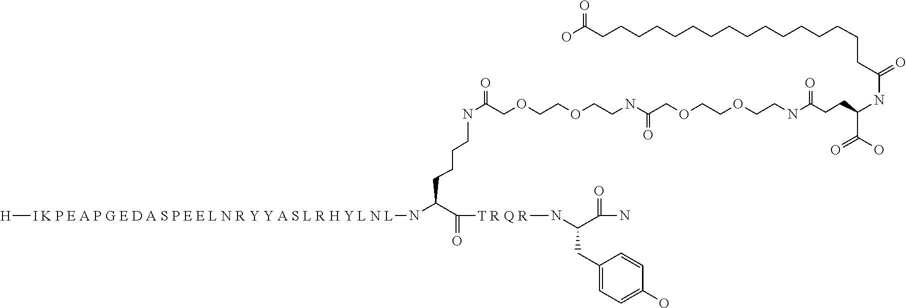
SEQ ID NO: 65 Name: N-epsilon14-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys14]hPYY3-36 Structure: 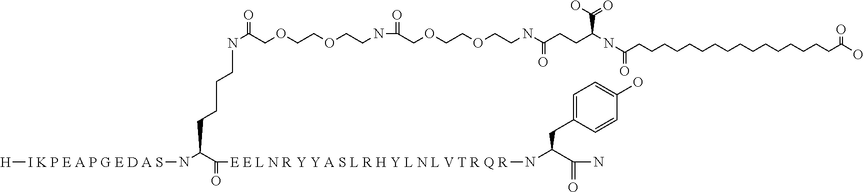
SEQ ID NO: 66 Name: N-epsilon15-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys15]hPYY3-36 Structure: 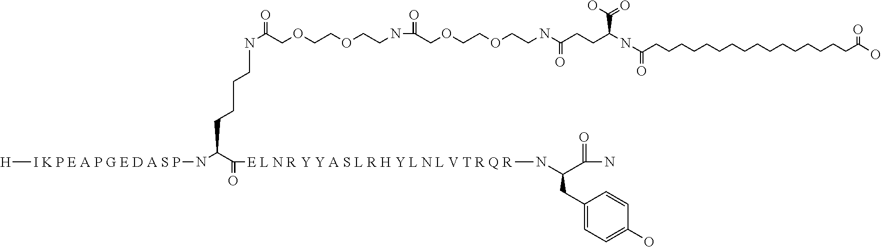
SEQ ID NO: 67 Name: N-epsilon16-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys16]hPYY3-36 Structure: 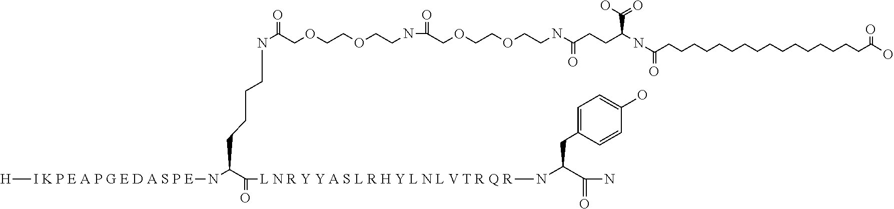
SEQ ID NO: 68 Name: N-epsilon20-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys20]hPYY3-36 Structure: 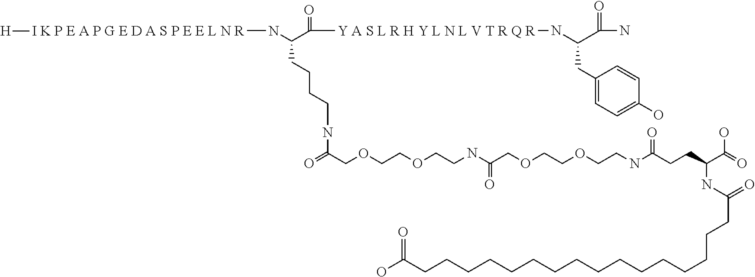
SEQ ID NO: 69 Name: N-epsilon28-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys28]hPYY3-36 Structure: 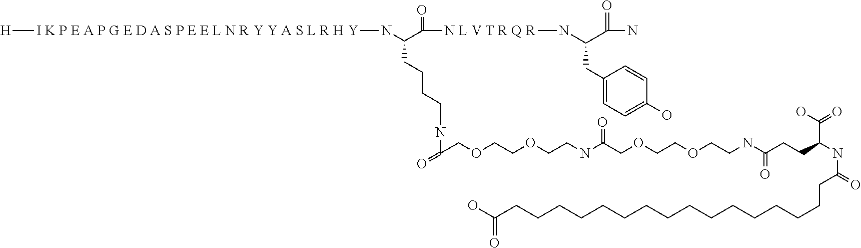
SEQ ID NO: 70 Name: N-epsilon32-[2-(2-{2-[2-(2-{2-[(S)-4-Carboxy-4-(17- carboxyheptadecanoylamino)butyrylamino]ethoxy}ethoxy)acetylamino]- ethoxy}ethoxy)acetyl][Lys32]hPYY3-36 Structure: 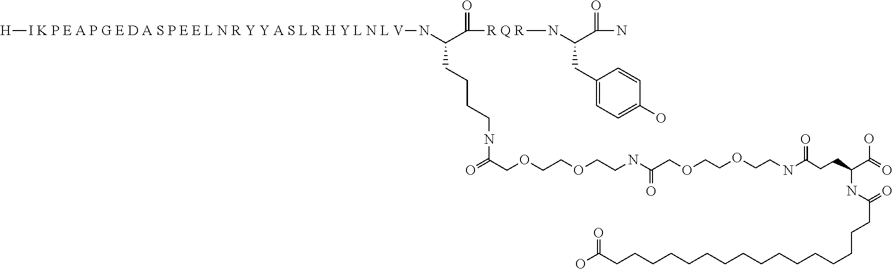
SEQ ID NO: 71 Name: N-epsilon25-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Leu17,Lys25,Leu30]hPP1-36 Structure: 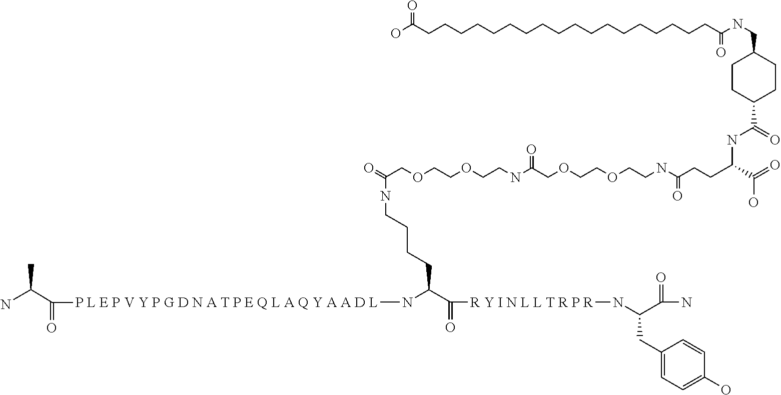
SEQ ID NO: 72 Name: N-epsilon15-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Leu17,Lys15,Leu30]hPP1-36 Structure: 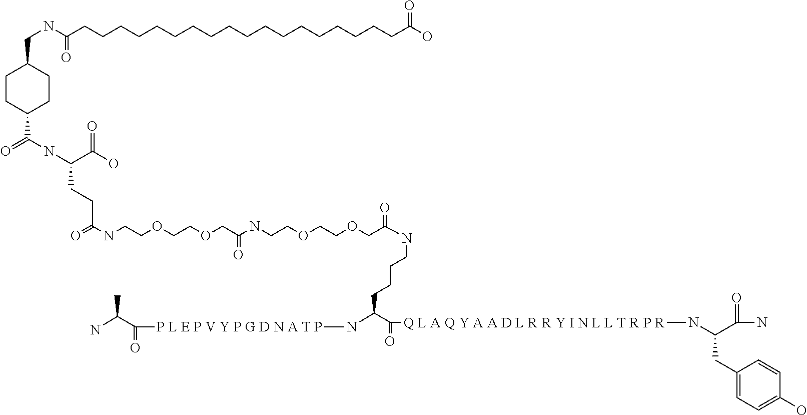
SEQ ID NO: 73 Name: [Leu17,Leu30]hPP2-36 Structure: 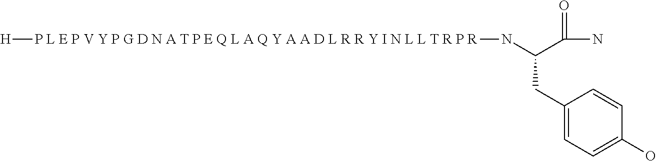
SEQ ID NO: 74 Name: N-epsilon13-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Leu17,Lys13,Gln34]hPP1-36 acid Structure: 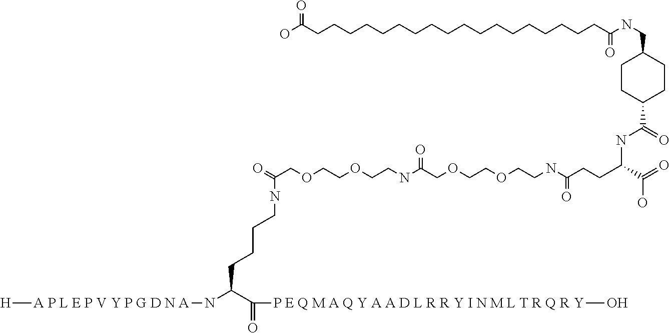
SEQ ID NO: 75 Name: N-alfa-[2-(2-{2-[2-(2-{2-[(S)-4-Carboxy-4-({trans-4-[(19- carboxynonadecanoylamino)methyl]cyclohexanecarbonyl}amino)butyrylamino]- ethoxy}ethoxy)acetylamino]ethoxy}ethoxy)acetyl][Leu17,Leu30]hPP1-36 Structure:

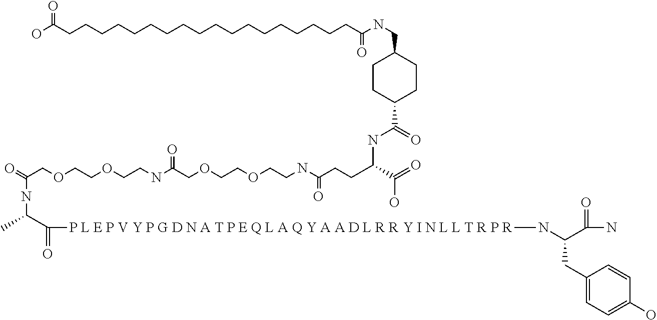

In one aspect PYY or PP peptide derivatives according to the invention are selected from the group consisting of SEQ ID NO: 3, SEQ ID NO: 12, SEQ ID NO: 19, SEQ ID NO: 20, SEQ ID NO: 21, SEQ ID NO: 22, SEQ ID NO: 23, SEQ ID NO: 27, SEQ ID NO: 28, SEQ ID NO: 33, SEQ ID NO: 34, SEQ ID NO: 35, SEQ ID NO: 40, SEQ ID NO: 51, SEQ ID NO: 52, SEQ ID NO: 53, SEQ ID NO: 54, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 66, SEQ ID NO: 67, SEQ ID NO: 68, SEQ ID NO: 69, and SEQ ID NO: 70. In one aspect PYY or PP peptide derivatives according to the invention is selected from the group consisting of SEQ ID NO: 4, SEQ ID NO: 14, SEQ ID NO: 15, SEQ ID NO: 16, SEQ ID NO: 17, SEQ ID NO: 18, SEQ ID NO: 24, SEQ ID NO: 29, SEQ ID NO: 30, SEQ ID NO: 31, SEQ ID NO: 32, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 55, SEQ ID NO: 56, SEQ ID NO: 71, SEQ ID NO: 72, SEQ ID NO: 74, SEQ ID NO: 75. In one aspect PYY or PP peptide derivatives according to the invention are selected from the group consisting of SEQ ID NO: 23, SEQ ID NO: 57, SEQ ID NO: 58, SEQ ID NO: 43, and SEQ ID NO: 55.

### Clinical Indications

The present invention provides a method of treating a disease, condition or disorder modulated by an Y2 and/or Y4 receptor agonist in mammals, which comprises peripherally administering to a mammal in need of such treatment a therapeutically effective amount of a PYY or PP peptide derivative or analogue thereof of the invention. The PYY or PP peptide derivative or analogue thereof of the invention may be used alone or in combination with at least one additional pharmaceutical agent that is useful in the treatment of the disease, condition or disorder or a co-morbidity of the disease, condition or disorder. Diseases, conditions, or disorders modulated by an Y2 and/or Y4 receptor agonist in mammals include obesity and being overweight. Co-morbidities of such diseases, conditions, or disorders would likely be incidentally improved by treatment of such diseases, conditions, or disorders. Further provided is a method of treating obesity in a mammal in need of such treatment, which comprises peripherally administering to the mammal a therapeutically effective amount of a PYY or PP peptide derivative or analogue thereof of the present invention.

As used herein, the term “therapeutically effective amount” of a compound refers to an amount sufficient to cure, alleviate, or partially arrest the clinical manifestations of a given disease and/or its complications with respect to appropriate control values determined prior to treatment or in a vehicle-treated group. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

The terms “treatment”, “treating” and other variants thereof as used herein refer to the management and care of a patient for the purpose of combating a condition, such as a disease or a disorder. The terms are intended to include the full spectrum of treatments for a given condition from which the patient is suffering, such as administration of the active compound(s) in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition, or disorder, and includes the administration of the active compound(s) in question to prevent the onset of symptoms or complications. The terms “treating”, “treat”, or “treatment” embrace both preventative, i.e., prophylactic, and palliative treatment. Also provided is a method of reducing weight or promoting weight loss (including preventing or inhibiting weight gain) in a mammal which comprises peripherally administering to the mammal a weight- controlling or weight-reducing amount of a PYY or PP peptide derivative or analogue thereof of the present invention.

Also provided is a method of reducing food intake in a mammal which comprises peripherally administering to the mammal a food-intake-reducing amount of a PYY or PP peptide derivative or analogue thereof of the present invention.

Also provided is a method of inducing satiety in a mammal which comprises peripherally administering to the mammal a satiety-inducing amount of a PYY or PP peptide derivative or analogue thereof of the invention.

Also provided is a method of reducing caloric intake in a mammal which comprises peripherally administering to the mammal a calorie-intake-reducing amount of a PYY or PP peptide derivative or analogue thereof of the invention.

Also provided is a method of reducing nutrient availability by administration of a therapeutically effective amount of a PYY or PP peptide derivative or analogue thereof of the present invention. In one aspect a method of inhibition of food intake, slowing of gastric emptying, inhibition of gastric acid secretion, and inhibition of pancreatic enzyme secretion by administration of a therapeutically effective amount of a PYY or PP peptide derivative or analogue thereof of the present invention is provided. In one aspect a method of treating or preventing metabolic diseases such as type 1, type 2, or gestational diabetes mellitus, obesity and other manifestations of insulin-resistance syndrome (Syndrome X) by administration of a therapeutically effective amount of a PYY or PP peptide derivative or analogue thereof of the present invention is provided.

In one aspect, a method is disclosed herein for altering energy metabolism in a subject. The method includes administering a therapeutically effective amount of an agonist of the invention to the subject, thereby altering energy expenditure. Energy is burned in all physiological processes. The body can alter the rate of energy expenditure directly, by modulating the efficiency of those processes, or changing the number and nature of processes that are occurring. For example, during digestion the body expends energy moving food through the bowel, and digesting food, and within cells, the efficiency of cellular metabolism can be altered to produce more or less heat. In one aspect a method is disclosed herein for any and all manipulations of the accurate circuitry described in this application, which alter food intake coordinately and reciprocally alter energy expenditure. Energy expenditure is a result of cellular metabolism, protein synthesis, metabolic rate, and calorie utilization. Thus, in this embodiment, peripheral administration results in increased energy expenditure, and decreased efficiency of calorie utilization. In one aspect, a therapeutically effective amount of a receptor agonist according to the invention is administered to a subject, thereby increasing energy expenditure.

In one aspect of the invention, methods for treating or preventing obesity are provided, wherein the method comprises administering a therapeutically or prophylactically effective amount of a PYY or PP peptide derivative or analogue thereof to a subject in need thereof. In a preferred embodiment, the subject is an obese or overweight subject. While “obesity” is generally defined as a body mass index over 30, for purposes of this disclosure, any subject, including those with a body mass index of less than 30, who needs or wishes to reduce body weight is included in the scope of “obese.” Subjects who are insulin resistant, glucose intolerant, or have any form of diabetes mellitus (e.g., type 1, 2 or gestational diabetes) can benefit from this method. In one aspect of the invention, methods of reducing food intake, reducing nutrient availability, causing weight loss, affecting body composition, and altering body energy content or increasing energy expenditure, treating diabetes mellitus, and improving lipid profile (including reducing LDL cholesterol and triglyceride levels and/or changing HDL cholesterol levels) are provided, wherein the methods comprise administering to a subject an effective amount of a PYY or PP peptide derivative or analogue thereof of the invention. In a preferred embodiment, the methods of the invention are used to treat or prevent conditions or disorders which can be alleviated by reducing nutrient availability in a subject in need thereof, comprising administering to said subject a therapeutically or prophylactically effective amount of a PYY or PP peptide derivative or analogue thereof of the invention. Such conditions and disorders include, but are not limited to, hypertension, dyslipidemia, cardiovascular disease, eating disorders, insulin-resistance, obesity, and diabetes mellitus of any kind.

Without intending to be limited by theory, it is believed that the effects of peripherally-administered PYY or PP peptide derivative or analogue thereof of the present invention in the reduction of food intake, in the delay of gastric emptying, in the reduction of nutrient availability, and in the causation of weight loss are determined by interactions with one or more unique receptor classes in, or similar to, those in the PP family. More particularly, it appears that a receptor or receptors similar to the PYY-preferring (or Y7) receptors are involved.

Additional assays useful to the invention include those that can determine the effect of PP-fold compounds, such as PYY or PP peptide derivatives or analogues thereof, on body weight and/or body composition. An exemplary assay can be one that involves utilization of a diet-induced obese (DIO) mouse model for metabolic disease: 125 female CBA mice may be ordered from Charles River, Japan. At 5 weeks of age they arrive at Animal Unit, Novo Nordisk. The mice are on reversed day/night cycle. Mice #1-100 have ad libitum access to high fat diet D12309, Research Diet (60% kcal from fat). This diet has previously been shown to be effective in inducing obesity in CBA mice. In a first embodiment mice #101-125 are fed control diet (D12310) containing 11% kcal from fat. In a second embodiment mice #101-125 are fed control diet (D12450B) containing 10% kcal from fat. The mice are weighed on a weekly basis. When High fat fed mice (D12492 or D12309) have gained sufficient weight as compared to low fat diet mice (appr 15-20% overweight) they are used in the study. Based on body weight, outliers are removed and the remaining mice divided into groups aiming at obtaining similar body weights in the groups. Before starting the study all mice are scanned for body composition (NMR scan). One week before starting the study the mice are weighed daily to get a stable baseline and to acclimatize them to the procedure. The mice are divided into groups as follows;

Group 1 (n=10): s.c. dosing of PYY analogue (dosis 0.3 μmol/kg, 10 ml/kg)

Group 2 (n=10): s.c. dosing of PYY analogue (dosis 1 μmol/kg, 10 ml/kg)

Group 3 (n=10): s.c. dosing of PP analogue (dosis 0.3 μmol/kg, 10 ml/kg)

Group 4 (n=10): s.c. dosing of PP analogue (dosis 1 μmol/kg, 10 ml/kg)

Group 5 (n=10): s.c. dosing of human PYY(3-36) (dosis 1 μmol/kg, 10 ml/kg)

Group 6 (n=10): s.c. dosing of human PP (dosis 1 μmol/kg, 10 ml/kg)

Group 7 (n=10): s.c. dosing of vehicle

Group 8 (n=10): Low fat group as reference

One or more PYY or PP peptide derivatives or analogue thereof as well as control compounds, such as human PYY, PYY(3-36), and human PP, are dissolved in 50 mM NaH2PO4, 165 mM NaCl, pH=7.4. Dosing is performed once daily at the same time point every day, shortly before lights off. As an alternative to s.c. administration some or all of the compounds can be delivered via Alzet osmotic minipumps. The pumps can be set to deliver any amount of the compounds, e.g., 1 μmol/kg/24 hours. The mice are dosed for 3 weeks. Body weight for all mice is recorded daily in combination with dosing. After 1 week and 3 weeks of treatment, the mice are scanned for body composition using a QNMR system (Echo Medical Systems, Houston, Tex.). Thereafter the mice are euthanized with cervical dislocation. Data are analysed in Graph Pad Prism. Statistical significance is assessed by comparing the groups with ANOVA followed by Tukey's post-hoc test. A p-value <0.05 is considered statistically significant.

In another assay ob/ob mice are used to analyze the effect of compounds of the invention on body weight and body composition. This assay is similar to the above described assay for DIO mice except that ob/ob mice (Taconic, Hudson, N.Y.) are used. These mice are maintained on a regular diet (Altromin 1324, Brogaarden, Denmark).

Respiratory quotient (RQ, defined as CO2 production divided by O2 consumption) and metabolic rate can be determined using whole-animal indirect calorimetry (Oxymax, Columbus Instruments, Columbus, Ohio). The mice can be euthanized by isoflurane overdose, and an index of adiposity (bilateral epididymal fat pad weight) measured. In the methods of the invention, preferred PP-fold peptides of the invention are those having a potency in one of the assays described herein (specifically food intake, gastric emptying, pancreatic secretion, weight reduction or body composition assays).

Additional assays useful in determining effect of PP-fold peptides are assays measuring acute food intake, such as the Fasting-induced refeeding assay:

Acute food intake in mice: Lean C57BL male mice are obtained from Charles River, Japan. They are maintained on a 12:12 light:dark cycle (lights off at 10:00 AM, lights on at 10:00 PM), fed pelleted D12450B rodent diet (Research Diets, Inc., New Brunswick, N.J.), and allowed water ad libitum. The mice arrive at 7-8 weeks of age and are acclimatized in the BioDAQ system a minimum of two weeks prior to study. On the day of study, mice are 9-12 weeks old. They are fasted overnight (20-24 h) with free access to water. The day of the study, mice are dosed with s.c. injection (dose volume=10 mL/kg), returned to their cage, and pre-weighed food is immediately placed in the cage. The dosing vehicle used may be: 50 mM K2HPO4, 0.05% tween 80, pH=8.0 and dose is calculated for the test compound on a molar basis. Assay design:
The mice are fasted from 2:00 PM the day before dosing
The mice are weighed and dosed 30 minutes before the light is turned off at 10:00 AM.
The mice are dosed with 10 ml/kg s.c.
The mice are dosed once and the food-intake is monitored using the BioDAQ system (Research Diets, Inc., New Brunswick, N.J.) for 24 hours
   The BioDAQ system consists of 32 mouse boxes each having a food-tray with a sensitive weight. When the mice eat the weight reduction of the content of the food-tray is registered. Data is registered each time there is a change in the weight of the individual food-tray. Cumulative food intake is calculated by subtracting the food weight at each time point from the starting food weight.

Acute food intake in rats: Lean male Sprague Dawley rats (˜180 g) are obtained from Taconic, Europe. Immediately after arrival and two weeks before dosing the rats are housed in reversed light cycle (dark from 10 am to 10 pm, 2 in each cage). The rats are fed regular diet (Altromin 1324, Brogaarden, Denmark). One week before dosing, rats are moved to the FeedWin system, where the rats are placed in individual cages for acclimatisation. The FeedWin system (Ellegårds Systems, Faaborg, Denmark) contains 32 stations for individual and continuous registration of food and water intakes. One station is defined by 1 cage with a metal lid plus 2 scales, one for food-intake and one for water-intake. Food and water intake is estimated by measurements of the disappearance of preloaded amounts of food and water that are placed on the 2 scales on each side of the cage. The day of the study rats are dosed before onset of dark with a s.c. injection (dose volume=1-2 mL/kg) and returned to their cage. After dosing water and food intake will be registered by the FeedWin system. Data will be collected each 15 minutes for 48 hours. Food consumptions for each group are calculated for the requested periods.

Acute food intake in pigs: Young female Landrace Yorkshire Duroc pigs are obtained from Gundsoegaard, Denmark. The animals are housed in a group for one week during acclimatisation. The animals are fed ad libitum with pig diet (Prima Antonio) at all times both during the acclimatisation and the experimental period. For measurement of individual food intake, the animals are placed in individual pens. Food intake is monitored on line by logging the weight of food every 15 minutes using the Mpigwin system (Ellegårds Systems, Faaborg, Denmark). The first day of the study (Monday morning) pigs are dosed with a s.c. injection (dose volume=0.025-0.04 mL/kg) and food intake is monitored for five days until end of study (Friday afternoon). Food consumptions for each group are calculated for the requested periods.

An assay useful for measuring PK of the compounds of the invention is the mini-pig PK assay. Five male Göttingen mini-pigs weighing approximately 18 to 22 kg from Ellegaard Göttingen Minipigs A/S, Denmark were included in the study. The mini-pigs had two central venous catheters inserted which were used for intra venous (i.v.) dosing and bloodsampling. Compound was dissolved in 50 mM K2HPO4, 0.05% tween 80, pH=8.0 to a concentration of 180 nmol/ml. The pigs were dosed with 6 nmol compound/kg body weight. Blood samples were taken at the following time points: pre-dose, 30 minutes, 1, 2, 4, 8, 24, 48, 72, 96, 120, 168 and 240 hours post dosing. The blood samples were collected into test tubes containing EDTA buffer for stabilization and kept on ice for max. 20 minutes before centrifugation. The centrifugation procedure to separate plasma was; 4° C., 3000 rpm for 10 minutes. Plasma were collected and immediately transferred to Micronic tubes stored at −20° C. until assayed.

An additional mini-pig PK assay was used for measuring PK of the compounds of the invention. Mini-pigs weighing 15 to 35 kg from Ellegaard Göttingen Minipigs A/S were included in the studies. The animals had two central venous catheters inserted which were used for intra venous (i.v.) dosing and blood sampling. Compounds were dissolved in 10 mM Na₂HPO₄, 150 mM NaCl, 0.01% tween 80, pH=4.0 to concentrations in the range of 40 nmol/ml to 200 nmol/ml. The mini-pigs were dosed i.v. with 10 nmol compound/kg body weight, occasionally other doses such as 4 nmol/kg, 30 nmol/kg or 50 nmol/kg were administered. Each compound was dosed to 3 or 4 mini-pigs, and two compounds may be given simultaneously to the same animal. Blood were sampled pre-dose and 12 times during the first 10 hours post-dose. Blood were furthermore sampled once daily until 13 days post dosing. The blood samples were collected into test tubes containing EDTA buffer, trasylol and Val-Pyr for stabilization and kept on ice for max. 20 minutes. Samples were centrifuged at 4° C., 2000G for 10 minutes to separate plasma. Plasma were collected and immediately transferred to Micronic tubes stored at −20° C. until assayed.

Plasma samples were analysed by LC-MS on an LTQ-Orbitrap (ThermoFisher Scientific, Bremen) to which Accela HPLC pumps and an autosampler were connected (both from ThermoFisher). The mass spectrometer was equipped with an electrospray interface, which was operated in positive ionisation mode. Analysis was conducted in selected ion monitoring mode at m/z 829.8±1.5 Da. The compound was detected at 829.4529 Da, which corresponded to [M+6H]6+ with an accuracy of 3.6 ppm. For quantification purposes, the six most intense isotope peaks were extracted with an accuracy of 5 ppm. HPLC was performed on a Jupiter Proteo column (4μ) 90A (50×2.0 mm ID). Mobile phases consisted of A. 0.1% formic acid and B. 0.1% formic acid in acetonitrile. A gradient was run from 10% B to 20% B from 0 to 0.2 min and then from 20% B to 34% B from 0.2 min to 6 min. The flow rate was 0.3 ml/min. For analysis of plasma samples, 30 μl plasma was precipitated with 90 μl ethanol. To 100 μl of the supernatant, 20 μl 95% acetonitrile (containing 5% formic acid) and 200 μl heptane were added. The heptane phase was removed after 5 min and the remaining solution was analysed by LC-MS as described above. For construction of plasma standards, compound was spiked to plasma (minipig) at the following concentrations: 1 nM, 2 nM, 5 nM, 10 nM, 20 nM, 50 nM, 100 nM, 200 nM. The plasma standards were treated as the samples. The lower limit of quantification was estimated to 2 nM.

Non-compartmental analysis (NCA): Plasma concentration-time profiles were analyzed by non-compartmental pharmacokinetics analysis (NCA) using WinNonlin Professional 5.0 (Pharsight Inc., Mountain View, Calif., USA). NCA was performed using the individual plasma concentration-time profiles from each animal.

An exemplary assay for measurement of gastric emptying is described in the materials and methods section page 1326 under the headline “Gastric emptying” in (Asakawa A et al, Characterization of the effects of pancreatic polypeptide in the regulation of energy balance, Gastroenterology, 2003,124,1325-1336).

Appetite can be measured by any means known to one of skill in the art. For example, in humans, decreased appetite can be assessed by a psychological assessment. In such an embodiment, administration of the receptor agonist results in a change in perceived hunger, satiety, and/or fullness. Hunger can be assessed by any means known to one of skill in the art. In one aspect, hunger is assessed using psychological assays, such as by an assessment of hunger feelings and sensory perception using e.g. a questionnaire.

In addition to the amelioration of hypertension in subjects in need thereof as a result of reduced food intake, weight loss, or treating obesity, compounds of the invention may be used to treat hypotension.

Compounds of the invention may also be useful for potentiating, inducing, enhancing or restoring glucose responsiveness in pancreatic islets or cells. These actions may be useful for treating or preventing conditions associated with metabolic disorders such as those described above and in U.S. patent application no. US20040228846. Assays for determining such activity are known in the art. For example, in published U.S. patent application no. US20040228846 (incorporated by reference in its entirety), assays are described for islet isolation and culture as well as determining fetal islet maturation. In the examples of patent application US20040228846, intestine-derived hormone peptides including pancreatic peptide (PP), neuropeptide Y (NPY), neuropeptide K (NPK), PYY, secretin, glucagon-like peptide-1 (GLP-1) and bombesin were purchased from Sigma. Collagenase type XI was obtained from Sigma. RPMI 1640 culture medium and fetal bovine serum were obtained from Gibco. A radioimmunoassay kit containing anti-insulin antibody ([125I]-RIA kit) was purchased from Linco, St Louis. Post-partem rat islets were obtained from P-02 year old rats. Adult rat islets were obtained from 6-8 week old rats. Fetal rat islets were obtained as follows. Pregnant female rats were sacrificed on pregnancy day e21. Fetuses were removed from the uterus. 10-14 pancreata were dissected from each litter and washed twice in Hanks buffer. The pancreas were pooled, suspended in 6 ml 1 mg/ml collagenase (Type XI, Sigma) and incubated at 37° C. for 8-10 minutes with constant shaking. The digestion was stopped by adding 10 volumes of ice-cold Hanks buffer followed by three washes with Hanks buffer. The islets were then purified by Ficoll gradient and cultured in 10% fetal bovine serum (FBS)/RPMI medium with or without addition of 1 μM IBMX. At the end of five days, 20 islets were hand picked into each tube and assayed for static insulin release. Generally, islets were first washed with KRP buffer and then incubated with 1 ml of KRP buffer containing 3 mM (low) glucose for 30 minutes at 37 Degrees Centigrade with constant shaking. After collecting the supernatant, the islets were then incubated with 17 mM (high) glucose for one hour at 37 Degrees Centigrade The insulin released from low or high glucose stimulation were assayed by radioimmunoassay (RIA) using the [125I]-RIA kit. E21 fetal islets were cultured for 5 days in the presence of 200 ng/ml PYY, PP, CCK, NPK, NPY, Secretin, GLP-I or Bombesin.

An exemplary in vivo assay is also provided using the Zucker Diabetic Fatty (ZDF) male rat, an inbred (>F30 Generations) rat model that spontaneously expresses diabetes in all fa/fa males fed a standard rodent diet Purina 5008. In ZDF fa-fa males, hyperglycemia begins to develop at about seven weeks of age and glucose levels (fed) typically reach 500 mg/DL by 10 to 11 weeks of age. Insulin levels (fed) are high during the development of diabetes. However, by 19 weeks of age insulin drops to about the level of lean control litter mates. Triglyceride and cholesterol levels of obese rats are normally higher than those of leans. In the assay, three groups of 7- week old ZDF rats, with 6 rats per group, received the infusion treatment by Alzet pump for 14 days: 1) vehicle control, 2) and 3), PYY with two different doses, 100 μmol/kg/h and 500 μmol/kg/h respectively. Four measurements were taken before the infusion and after the infusion at day 7 and day 14: 1) plasma glucose level, 2) plasma insulin level, and 3) plasma triglycerides (TG) level, as well as oral glucose tolerance (OGTT) test. Accordingly, these assays can be used with compounds of the invention to test for desired activity.

Compounds of the invention may be used in the treatment of anxiety. A method of measuring for an effect of an administrated peptide on anxiety-like behaviour by the elevated plus maze test is described in the material and methods section page 1327 under the headline “Repeated administrations” in (Asakawa A et al, Characterization of the effects of pancreatic polypeptide in the regulation of energy balance, Gastroenterology, 2003, 124, 1325-1336).

Compounds of the invention may be used in the treatment of rhinitis of any origin. A method of measuring for an effect of an administrated peptide on nasal blood flow as a marker for rhinitis is described in page 1725 line 11 of (Cervin A et al, Functional effects of neuropeptide Y receptors on blood flow and nitric oxide levels in the human nose. Am J Respir Crit. Care Med. 1999 November; 160(5 Pt 1):1724-8).

Compounds of the invention may be useful for promoting wound healing. Compounds of the invention may be useful in decreasing time of recreation after surgery of any kind including, but not limited to, dental surgery and cosmetic surgery. Compounds of the invention may be useful for promoting arteriogenesis in the treatment of diseases where this is desirable including, but not limited to, peripheral arterial disease.

The compounds of the invention exhibit a broad range of biological activities, some related to their antisecretory and antimotility properties. The compounds may suppress gastrointestinal secretions by direct interaction with epithelial cells or, perhaps, by inhibiting secretion of hormones or neurotransmitters which stimulate intestinal secretion. Anti-secretory properties include inhibition of gastric and/or pancreatic secretions and can be useful in the treatment or prevention of diseases and disorders including gastritis, acute pancreatitis, Barrett's esophagus, and Gastroesophageal Reflux Disease.

Compounds of the invention are useful in the treatment of any number of gastrointestinal disorders (see e.g., Harrison's Principles of Internal Medicine, McGraw-Hill Inco, New York, 12th Ed.) that are associated with excess intestinal electrolyte and water secretion as well as decreased absorption, e.g., infectious diarrhoea, inflammatory diarrhoea, short bowel syndrome, or the diarrhoea which typically occurs following surgical procedures, e.g., ileostomy. Examples of infectious diarrhoea include, without limitation, acute viral diarrhoea, acute bacterial diarrhoea (e.g., salmonella, Campylobacter, and Clostridium or due to protozoal infections), or traveller's diarrhoea (e.g., Norwalk virus or rotavirus). Examples of inflammatory diarrhoea include, without limitation, malabsorption syndrome, tropical sprue, chronic pancreatitis, Crohn's disease, diarrhoea, and irritable bowel syndrome. It has also been discovered that the peptides of the invention can be used to treat an emergency or life-threatening situation involving a gastrointestinal disorder, e.g., after surgery or due to cholera. A method of measuring intestinal electrolyte secretion is described on page 1250 of (Eto B et al Comparison of the antisecretory effect of endogenous forms of peptide YY on fed and fasted rat jejunum. Peptides. 1997; 18(8):1249-55).

Compounds of the invention may also be useful for treating or preventing intestinal damage as opposed to merely treating the symptoms associated with the intestinal damage (for example, diarrhoea). Such damage to the intestine may be, or a result of, chemotherapy-induced diarrhoea, ulcerative colitis, inflammatory bowel disease, bowel atrophy, loss bowel mucosa, and/or loss of bowel mucosal function (see WO 03/105763, incorporated herein by reference in its entirety). Assays for such activity, as described in WO 03/105763, include 11 week old male HSD rats, ranging 250-300 grams housed in a 12:12 lightdark cycle, and allowed ad libitum access to a standard rodent diet (Teklad L M 485, Madison, Wis.) and water. The animals were fasted for 24 hours before the experiment. A simple and reproducible rat model of chronic colonic inflammation has been previously described by Morris G P, et al., “Hapten-induced model of chronic inflammation and ulceration in the rat colon.” Gastroenterology. 1989; 96:795-803. It exhibits a relatively long duration of inflammation and ulceration, affording an opportunity to study the pathophysiology of colonic inflammatory disease in a specifically controlled fashion, and to evaluate new treatments potentially applicable to inflammatory bowel disease in humans. Rats were anesthetized with 3% isofluorane and placed on a regulated heating pad set at 37 Degrees Centigrade A gavage needle was inserted rectally into the colon 7 cm. The hapten trinitrobenzenesulfonic acid (TNBS) dissolved in 50% ethanol (v/v) was delivered into the lumen of the colon through the gavage needle at a dose of 30 mg/kg, in a total volume of 0 0.4-0.6 mL, as described in Mazelin, et al., “Protective role of vagal afferents in experimentally-induced colitis in rats.” Juton Nery Syst. 1998; 73:38 45. Control groups received saline solution (NaCl 0.9%) intracolonically. Four days after induction of colitis, the colon was resected from anesthetized rats, which were then euthanized by decapitation. Weights of excised colon and spleen were measured, and the colons photographed for scoring of gross morphologic damage. Inflammation was defined as regions of hyperemia and bowel wall thickening.

The Y4 receptor selective agonists of the invention are of value in the treatment of constipation. The frequency of bowel movements, a measure of constipation, can be measured by any means known to one of skill in the art.

Y4 selective agonists are also of value in the treatment of diarrhoea or hypersecretion from intestinal stomia, and in the treatment of nausea or emesis, or as antinausea or antiemetic agents or co-treatment with drugs prone to cause nausea and/or emesis.

The Y4 selective compounds of the present invention, and PP itself, are also useful for the treatment or protection against emesis and nausea.

In one aspect of the invention an acute test may be performed where a compound of the invention is administered to ensure that these compounds have the intended effect in the subject to be treated before a chronic treatment is started. Through these means it is ensured that only subjects who are susceptible to treatment with a compound of the invention are treated with these compounds.

In one aspect the invention relates to a method of treatment of a condition responsive to Y receptor modulation by administration of a PYY or PP peptide derivative or analogue thereof as defined herein. In one aspect the invention, in said method of treatment, the condition responsive to Y receptor modulation is obesity. In one aspect the invention, in said method of treatment, the condition is obesity-related diseases, such as reduction of food intake, Syndrome X (metabolic syndrome), diabetes, type 2 diabetes mellitus or Non Insulin Dependent Diabetes Mellitus (NIDDM), hyperglycemia, insulin resistance, impaired glucose tolerance, cardiovascular disease, hypertension, atherosclerosis, coronary artery disease, myocardial infarction, peripheral vascular disease, stroke, thromboembolic diseases, hypercholesterolemia, hyperlipidemia, gallbladder disease, osteoarthritis, sleep apnea, reproductive disorders such as polycystic ovarian syndrome, or cancer of the breast, prostate, or colon. In one aspect the invention, in said method of treatment, the condition is a disease associated with excess intestinal electrolyte and water secretion or decreased absorption, e.g., infectious diarrhoea, inflammatory diarrhoea, short bowel syndrome, or the diarrhoea which typically occurs following surgical procedures, e.g., ileostomy. Examples of infectious diarrhoea include, without limitation, acute viral diarrhoea, acute bacterial diarrhoea (e.g., salmonella, Campylobacter, and Clostridium or due to protozoal infections), or traveller's diarrhoea (e.g., Norwalk virus or rotavirus). Examples of inflammatory diarrhoea include, without limitation, malabsorption syndrome, tropical sprue, chronic pancreatitis, Crohn's disease, diarrhoea, and irritable bowel syndrome. In one aspect the invention, in said method of treatment, the condition is a condition characterized by damage to the intestine such as chemotherapy-induced diarrhoea, ulcerative colitis, inflammatory bowel disease, bowel atrophy, loss bowel mucosa, and/or loss of bowel mucosal function. In one aspect the invention, in said method of treatment, the condition is an intestinal inflammatory condition such as ulcerative colitis, Crohns disease or irritable bowel syndrome. In one aspect the invention, in said method of treatment, the condition is allergic or non-allergic rhinitis. In one aspect the invention, in said method of treatment, the condition responsive is anxiety. In one aspect the invention, in said method of treatment, the administration regime is selected from the group consisting of once-daily, once-weekly, twice-monthly, or once-monthly. In one aspect the invention, in said method of treatment, said derivative shows improved PK profile compared to human PYY, PYY(3-36), or PP. In one aspect the invention, in said method of treatment, said derivative shows protracted properties compared to human PYY, PYY(3-36), or PP. In one aspect the invention, in said method of treatment, said derivative shows improved half life in vivo compared to human PYY, PYY(3-36) or PP. In one aspect the invention, in said method of treatment, a therapeutically effective dose of said derivative causes less side effects compared to human PYY, PYY(3-36), or PP.

In one aspect the invention relates to the use of a PYY or PP peptide derivative or analogue thereof as defined herein for the preparation of a medicament for the treatment of a condition responsive to Y receptor modulation, such as obesity or obesity-related diseases, e.g., reduction of food intake.

In one aspect the PYY or PP peptide derivative or analogue thereof provides a reduction of food intake of at least 5%, such as at least 10%, 15%, 20%, 25% or 30% compared to vehicle. In one aspect the PYY or PP peptide derivative or analogue thereof provides a reduction of food intake in the range of 5-30%, such as at least 5-20%, 5-15% or 10-20% compared to vehicle.

In one aspect the PYY or PP peptide derivative or analogue thereof provides a reduction of body weight of at least 5%, such as at least 10%, 15%, 20%, 25% or 30% compared to vehicle. In one aspect the PYY or PP peptide derivative or analogue thereof provides a reduction of body weight in the range of 5-30%, such as at least 5-20%, 5-15% or 10-20% compared to vehicle.

In one aspect the invention relates to the use of a PYY or PP peptide derivative or analogue thereof as defined herein for administration in a mammal, wherein said derivative shows protracted properties compared to the human PP and PYY compounds.

### Measurement of In Vitro Effect of PP-Fold Peptides on Y Receptor Activity

Measurement of Calcium Mobilization Using Cells Co-Expressing Y Receptors and a Chimeric G-Protein:

Potency of test compounds on the human Y receptors is determined by performing dose-response experiments in CHO cells stably transfected with a human Y receptor as well as a promiscuous G protein, Gqi5, which ensures that the Y receptor couples through a Gq pathway leading to an increase in calcium mobilization which is measured using a FLIPR (FLIPRtetra from Molecular Devices, CA, USA).

A. Preparation of Cells
1. Adherent Y receptor and Gqi5 double stable CHO cells maintained in DMEM-F12 with appropriate antibiotics are plated in 96-well poly-D-lysine-coated microplates to near confluence and grown overnight.

B. Preparation of Reagents
2. A 250 mM (100×) stock of probenecid acid (Invitrogen #P36400) is made and dissolved in 1 ml assay buffer.
3. The dye loading solution is prepared (for one microplate): 10 ml of assay buffer and 100 μl of probenecid acid (final concentration: 2.5 mM) stock solution is added to a vial of dye loading mix (provided in the kit). Vortex vigorously.

C. Assay
4. 100 μl of the dye loading solution is added to each well of a 96-well plate containing cells in 100 μl media (or other buffers depending on the ligand to be tested). The cells are placed in the 37° C./5% CO₂ incubator. The cells are incubated for 1 hour prior to the assay.
5. During the incubation of cells in dye mixture, a solution is prepared of receptor agonist (5×) in HBSS/HEPES: Hanks' Balanced Salt Solution (1×) with 20 mM HEPES, 0.01% NaN₃, pH 7.4 (with 0.1% BSA if using peptide as ligand). A serial dilution is made in 96-well compound plate (VWR #62409-108, NUNC, V-bottom).
6. 1 hour after the addition of dye mixture to the cells, fluorescence is measured using a FLIPR (FLIPRtetra from Molecular Devices, CA, USA).
   Determinations are made in triplicates. EC50 values were calculated using a standard pharmacological data handling software, Prism 5.0 (graphPad Sofware, San Diego, USA).

### Measuring Y2 or Y4 Receptor Activity Using ACTOne Based FLIPR Assay:

ACTOne™ is an easily scaleable cAMP biosensor HTS platform for measurement of Gs and Gi coupled 7™ receptor signalling from BD Biosciences (San Jose, Calif.). The cells express a biosensor developed around a modified rat olfactory cyclic nucleotide gated (CNG) calcium channel—a fairly non-discriminatory ion channel that responds to cAMP and cGMP. The CNG has been engineered to be cAMP selective and thus function as a cAMP responsive biosensor that signals through calcium or membrane potential responsive dyes. Y2 or Y4 receptor expressing ACTOne HEK-293 cells are obtained from BD Biosciences. The cells are loaded with a calcium responsive dye that only distributes in the cytoplasm. Probenecid, an inhibitor of the organic anion transporter is added to prevent the dye from leaving the cell. A phosphodiesterase inhibitor is added to prevent formatted cAMP from being degraded. Isoproterenol (an β1/β2 agonist) is added to activate the adenylate cyclase. When an Y2 or Y4 receptor agonist is added, the adenylate cyclase is inactivated. The decreased calcium concentration in the cytoplasm is then detected as an increase in fluorescence. Together with the test substance, Isoproterenol at a concentration matching EC₈₀, is added to all wells.
The cells are plated out in Greiner 384-well plates. 25 μl cell suspension containing 560 cells per μl are added to all wells using the Multidrop™ (384-Multidrop from Labsystems, Finland).
The cell plates are then incubated in the incubator over night at 37° C. with 5% CO₂ in stacks of up to 9 plates.
The cell plates are loaded with 25 μl probe from the FLIPR calcium4 kit (Molecular Devices, CA, USA) using the Multidrop™.
The cell plates are returned to the incubator and incubated for 60 min. at 37° C. in stacks of up to 9 plates.
The cell plates are then left at room temperature for 60 min., before use, without stacking the plates. The plates are covered with tinfoil to avoid light (the dye can be excited by the daylight, which results in higher baseline and variation).
The FLIPR (FLIPRtetra from Molecular Devices, CA, USA) adds 1 μl sample and 1 μl isoproterenol (0.05 μM final concentration) at the same time.
The fluorescence signal from the wells is measured 330 seconds after sample addition on the FLIPR.

### Administration and Pharmaceutical Compositions

Another object of the present invention is to provide a pharmaceutical formulation comprising a derivative according to the present invention which is present in a concentration from 0.1 mg/ml to 25 mg/ml, and wherein said formulation has a pH from 3.0 to 9.0. The formulation may further comprise a buffer system, preservative(s), tonicity agent(s), chelating agent(s), stabilizers and surfactants. The term “pharmaceutical composition” as used herein means a product comprising an active derivative according to the invention together with pharmaceutical excipients such as buffer, preservative, and optionally a tonicity modifier and/or a stabilizer. Thus a pharmaceutical composition is also known in the art as a pharmaceutical formulation.

In one aspect the invention relates to a composition comprising a PYY or PP peptide derivative or analogue thereof as defined herein and one or more pharmaceutical excipients.

In one aspect of the invention, the pharmaceutical formulation is an aqueous formulation, i.e. formulation comprising water. Such formulation is typically a solution or a suspension. In one aspect of the invention, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In one aspect, the pharmaceutical formulation is a freeze-dried formulation, whereto the physician or the patient adds solvents and/or diluents prior to use.

In one aspect, the pharmaceutical formulation is a dried formulation (e.g. freeze-dried or spray-dried) ready for use without any prior dissolution.

In one aspect, the invention relates to a pharmaceutical formulation comprising an aqueous solution of a derivative according to the present invention, and a buffer, wherein said derivative is present in a concentration from 0.1 mg/ml or above, and wherein said formulation has a pH from about 3.0 to about 9.0.

In one aspect of the invention, the pH of the formulation is from about 7.0 to about 9.5. In one aspect of the invention, the pH of the formulation is from about 3.0 to about 7.0. In one aspect of the invention, the pH of the formulation is from about 5.0 to about 7.5. In one aspect of the invention, the pH of the formulation is from about 7.5 to about 9.0. In one aspect of the invention, the pH of the formulation is from about 7.5 to about 8.5. In one aspect of the invention, the pH of the formulation is from about 6.0 to about 7.5. In one aspect of the invention, the pH of the formulation is from about 6.0 to about 7.0. In one aspect, the pharmaceutical formulation is from 8.0 to 8.5.

In one aspect of the invention, each administered dose contains from 0.01 mg-10 mg of active derivative. In one aspect, the dose administered contains more than 0.05 mg active derivative. In one aspect, the dose administered contains more than 0.1 mg active derivative. In one aspect, the dose administered contains up to 10 mg active derivative. In one aspect, the dose administered contains up to 9 mg active derivative. In one aspect, the dose administered contains up to 8 mg active derivative. In one aspect, the dose administered contains up to 7 mg active derivative. In one aspect, the dose administered contains up to 6 mg active derivative. In one aspect, the dose administered contains up to 5 mg active derivative. In one aspect, the dose administered contains from 0.2 mg to 5 mg active derivative.

In one aspect of the invention, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative aspect of the invention.

In one aspect of the invention, the formulation further comprises a pharmaceutically acceptable preservative. In one aspect of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In one aspect, the preservative is phenol or m-cresol. In one aspect of the invention, the preservative is present in a concentration from 0.1 mg/ml to 20 mg/ml. In one aspect of the invention, the preservative is present in a concentration from 0.1 mg/ml to 5 mg/ml. In one aspect of the invention, the preservative is present in a concentration from 5 mg/ml to 10 mg/ml. In one aspect of the invention, the preservative is present in a concentration from 10 mg/ml to 20 mg/ml. Each one of these specific preservatives constitutes an alternative aspect of the invention. The use of a preservative in pharmaceutical compositions is well-known to the skilled person. For convenience reference is made to Remington: The Science and Practice of Pharmacy, 19^{th} edition, 1995.

In one aspect of the invention, the formulation further comprises an isotonic agent. In one aspect of the invention, the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. In one aspect, the isotoncity agent is propyleneglycol. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alfa and beta HPCD, soluble starch, hydroxyethyl starch and carboxymethylcellulose-Na may be used. In one aspect, the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one aspect, the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one aspect, the sugar or sugar alcohol concentration is between about 1 mg/ml and about 150 mg/ml. In one aspect of the invention, the isotonic agent is present in a concentration from 1 mg/ml to 50 mg/ml. In one aspect of the invention, the isotonic agent is present in a concentration from 1 mg/ml to 7 mg/ml. In one aspect of the invention, the isotonic agent is present in a concentration from 5 mg/ml to 7 mg/ml. In one aspect of the invention, the isotonic agent is present in a concentration from 8 mg/ml to 24 mg/ml. In one aspect of the invention, the isotonic agent is present in a concentration from 25 mg/ml to 50 mg/ml. Each one of these specific isotonic agents constitutes an alternative aspect of the invention. The use of an isotonic agent in pharmaceutical compositions is well-known to the skilled person. For convenience reference is made to Remington: The Science and Practice of Pharmacy, 19^{th} edition, 1995.

In one aspect of the invention, the formulation further comprises a chelating agent. In one aspect of the invention the chelating agent is selected from salts of ethylenediaminetetraacetic acid (EDTA), citric acid, and aspartic acid, and mixtures thereof. In one aspect of the invention the chelating agent is present in a concentration from 0.1 mg/ml to 5 mg/ml. In one aspect of the invention the chelating agent is present in a concentration from 0.1 mg/ml to 2 mg/ml. In one aspect of the invention the chelating agent is present in a concentration from 2 mg/ml to 5 mg/ml. Each one of these specific chelating agents constitutes an alternative aspect of the invention. The use of a chelating agent in pharmaceutical compositions is well-known to the skilled person. For convenience reference is made to Remington: The Science and Practice of Pharmacy, 19^{th} edition, 1995.

In one aspect of the invention, the formulation further comprises a stabilizer. The use of a stabilizer in pharmaceutical compositions is well-known to the skilled person. For convenience reference is made to Remington: The Science and Practice of Pharmacy, 19^{th} edition, 1995.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one aspect, amino acids to use in preparing the compositions of the invention are those carrying a charged side chain, such as arginine, lysine, aspartic acid, and glutamic acid. Any stereoisomer (i.e., L, D, or a mixture thereof) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one aspect the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In one aspect of the invention the amino acids or amino acid analogues are used in a concentration, which is sufficient to prevent or delay aggregation of the protein.

In one aspect of the invention, methionine (or other sulphuric amino acids or amino acid analogues) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L or D) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In one aspect of the invention, the formulation further comprises a stabilizer selected from the group of high molecular weight polymers or low molecular compounds.

In one aspect of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative aspect of the invention.

The pharmaceutical compositions may also comprise additional stabilizing agents, which further enhance stability of a therapeutically active polypeptide therein.

Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In one aspect of the invention, the formulation further comprises a surfactant. In one aspect of the invention, the pharmaceutical composition comprises two different surfactants. The term “surfactant” as used herein refers to any molecules or ions that are comprised of a water-soluble (hydrophilic) part, the head, and a fat-soluble (lipophilic) segment. Surfactants accumulate specifically at interfaces, which the hydrophilic part is orientated towards the water (hydrophilic phase) and the lipophilic part towards the oil- or hydrophobic phase (i.e. glass, air, oil etc.). The concentration at which surfactants begin to form micelles is known as the critical micelle concentration or CMC. Furthermore, surfactants lower the surface tension of a liquid. Surfactants are also known as amphipathic compounds. The term “detergent” is a synonym used for surfactants in general.

Anionic surfactants may be selected from the group of: Chenodeoxycholic acid, Chenodeoxycholic acid sodium salt, Cholic acid, Dehydrocholic acid, Deoxycholic acid, Deoxycholic acid methyl ester, Digitonin, Digitoxigenin, N,N-Dimethyldodecylamine N-oxide, Docusate sodium, Glycochenodeoxycholic acid sodium, Glycocholic acid hydrate, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine sodium salt, N-Lauroylsarcosine, N-Lauroylsarcosine, Lithium dodecyl sulfate, Lugol, 1-Octanesulfonic acid sodium salt, 1-Octanesulfonic acid sodium salt, Sodium 1-butanesulfonate, Sodium 1-decanesulfonate, Sodium 1-dodecanesulfonate, Sodium 1-heptanesulfonate, Sodium 1-heptanesulfonate, Sodium 1-nonanesulfonate, Sodium 1-propanesulfonate monohydrate, Sodium 2-bromoethanesulfonate, Sodium cholate hydrate, ox or sheep bile, Sodium cholate hydrate, Sodium choleate, Sodium deoxycholate, Sodium dodecyl sulfate, Sodium dodecyl sulfate, Sodium hexanesulfonate, Sodium octyl sulfate, Sodium pentanesulfonate, Sodium taurocholate, Taurochenodeoxycholic acid sodium salt, Taurodeoxycholic acid sodium salt monohydrate, Taurolithocholic acid 3-sulfate disodium salt, Tauroursodeoxycholic acid sodium salt, Trizma® dodecyl sulfate, DSS (docusate sodium, CAS registry no [577-11-7]), docusate calcium, CAS registry no [128-49-4]), docusate potassium, CAS registry no [749]-09-0]), SDS (sodium dodecyl sulfate or sodium lauryl sulfate), Dodecylphosphocholine (FOS-Choline-12), Decylphosphocholine (FOS-Choline-10), Non-ylphosphocholine (FOS-Choline-9), dipalmitoyl phosphatidic acid, sodium caprylate, and/or Ursodeoxycholic acid.

Cationic surfactants may be selected from the group of: Alkyltrimethylammonium bromide, Benzalkonium chloride, Benzalkonium chloride, Benzyldimethylhexadecylammonium chloride, Benzyldimethyltetradecylammonium chloride, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Hexadecyltrimethylammonium bromide, Hexadecyltrimethylammonium bromide, Polyoxyethylene(10)-N-tallow-1,3-diaminopropane, Thonzonium bromide, and/or Trimethyl(tetradecyl)ammonium bromide.

Nonionic surfactants may be selected from the group of: BigCHAP, Bis(polyethylene glycol bis[imidazoyl carbonyl]), block copolymers as polyethyleneoxide/polypropyleneoxide block copolymers such as poloxamers, poloxamer 188 and poloxamer 407, Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Dodecanoyl-N-methylglucamide, alkyl-polyglucosides, ethoxylated castor oil, Heptaethylene glycol monodecyl ether, Heptaethylene glycol monododecyl ether, Heptaethylene glycol monotetradecyl ether, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O-(N-heptylcarbamoyl)-beta-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-β-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tetradecyl-β-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-100, Triton® X-114, Triton® X-165 solution, Triton® X-305 solution, Triton® X-405, Triton® X-45, Triton® X-705-70, TWEEN® 20, TWEEN® 40, TWEEN® 60, TWEEN® 6, TWEEN® 65, TWEEN® 80, TWEEN® 81, TWEEN® 85, Tyloxapol, sphingophospholipids (sphingomyelin), and sphingoglycolipids (ceramides, gangliosides), phospholipids, and/or n-Undecyl β-D-glucopyranoside.

Zwitterionic surfactants may be selected from the group of: CHAPS, CHAPSO, 3-(Decyldimethylammonio)propanesulfonate inner salt, 3-(Dodecyldimethylammonio)-propanesulfonate inner salt, 3-(Dodecyldimethylammonio)propanesulfonate inner salt, 3-(N,N-Dimethylmyristylammonio)propanesulfonate, 3-(N,N-Dimethyloctadecylammonio)-propanesulfonate, 3-(N,N-Dimethyloctylammonio)propanesulfonate inner salt, 3-(N,N-Dimethylpalmitylammonio)propanesulfonate, N-alkyl-N,N-dimethylammonio-1-propanesulfonates, 3-cholamido-1-propyldimethylammonio-1-propanesulfonate, Dodecylphosphocholine, myristoyl lysophosphatidylcholine, Zwittergent 3-12 (N-dodecyl-N,N-dimethyl-3-ammonio-1-propanesulfonate), Zwittergent 3-10 (3-(Decyldimethylammonio)propanesulfonate inner salt), Zwittergent 3-08 (3-(Octyldimethylammonio)propanesulfonate), glycerophospholipids (lecithins, kephalins, phosphatidyl serine), glycero-glycolipids (galactopyranoside), alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, lysophosphatidylserine and lysophosphatidylthreonine, acylcarnitines and derivatives, N^{beta}-acylated derivatives of lysine, arginine or histidine, or side-chain acylated derivatives of lysine or arginine, N^{beta}-acylated derivatives of dipeptides comprising any combination of lysine, arginine or histidine and a neutral or acidic amino acid, N^{beta}-acylated derivative of a tripeptide comprising any combination of a neutral amino acid and two charged amino acids, or the surfactant may be selected from the group of imidazoline derivatives, long-chain fatty acids and salts thereof C₆-C₁₂ (eg. oleic acid and caprylic acid), N-Hexadecyl-N,N-dimethyl-3-ammonio-1-propanesulfonate, anionic (alkyl-aryl-sulphonates) monovalent surfactants, palmitoyl lysophosphatidyl-L-serine, lysophospholipids (e.g. 1-acyl-sn-glycero-3-phosphate esters of ethanolamine, choline, serine or threonine), or mixtures thereof.

The term “alkyl-polyglucosides” as used herein in relates to a straight or branched C₅₋₂₀-alkyl, -alkenyl or -alkynyl chain which is substituted by one or more glucoside moieties such as maltoside, saccharide etc. In one aspect these alkylpolyglucosides include C₆₋₁₈-alkyl-polyglucosides. In one aspect these alkylpolyglucosides includes the even numbered carbon-chains such as C₆, C₈, C₁₀, C₁₂, C₁₄, C₁₆, C₁₈ and C₂₀ alkyl chain. In one aspect the glucoside moieties include pyranoside, glucopyranoside, maltoside, maltotrioside and sucrose. In one aspect of the invention, less than 6 glucosid moieties are attached to the alkyl group. In one aspect of the invention, less than 5 glucosid moieties are attached to the alkyl group. In one aspect of the invention, less than 4 glucosid moieties are attached to the alkyl group. In one aspect of the invention, less than 3 glucosid moieties are attached to the alkyl group. In one aspect of the invention, less than 2 glucosid moieties are attached to the alkyl group. In one aspect alkyl-polyglucosides are alkyl glucosides such n-decyl β-D-glucopyranoside, decyl 13-D-maltopyranoside, dodecyl β-D-glucopyranoside, n-dodecyl β-D-maltoside, n-dodecyl β-D-maltoside, n-dodecyl β-D-maltoside, tetradecyl β-D-glucopyranoside, decyl β-D-maltoside, hexadecyl β-D-maltoside, decyl β-D-maltotrioside, dodecyl β-D-maltotrioside, tetradecyl β-D-maltotrioside, hexadecyl β-D-maltotrioside, n-dodecyl-sucrose, n-decylsucrose, sucrose monocaprate, sucrose monolaurate, sucrose monomyristate, and sucrose monopalmitate.

The use of a surfactant in pharmaceutical compositions is well-known to the skilled person. For convenience reference is made to Remington: The Science and Practice of Pharmacy, 19^{th} edition, 1995.

In one aspect of the invention, the formulation further comprises protease inhibitors such as EDTA (ethylenediamine tetraacetic acid) and benzamidineHCl, but other commercially available protease inhibitors may also be used. The use of a protease inhibitor is particular useful in pharmaceutical compositions comprising zymogens of proteases in order to inhibit autocatalysis.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a derivative according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, chewing gum, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the derivative of the present invention, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for administration of derivatives of the present invention, optionally using a device well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more specifically, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension or a powder for the administration of the derivative of the present invention in the form of a nasal or pulmonal liquid or powder spray. As a still further option, the pharmaceutical compositions containing the derivative of the invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is specifically a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one aspect of the invention, the pharmaceutical formulation comprising the derivative of the present invention is stable for more than 6 weeks of usage and for more than 3 years of storage.

In one aspect of the invention, the pharmaceutical formulation comprising the derivative of the present invention is stable for more than 4 weeks of usage and for more than 3 years of storage.

In one aspect of the invention, the pharmaceutical formulation comprising the derivative of the present invention is stable for more than 4 weeks of usage and for more than two years of storage.

In one aspect of the invention, the pharmaceutical formulation comprising the derivative of the present invention is stable for more than 2 weeks of usage and for more than two years of storage.

The treatment with a derivative according to the present invention may also be combined with a second or more pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Examples of these pharmacologically active substances are: Insulin, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as anti-hyperlipidemic agents as HMG CoA inhibitors (statins), Gastric Inhibitory Polypeptides (GIP analogues), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, PYY agonists, Y2 receptor agonists, Y4 receptor agonists, mixed Y2/Y4 receptor agonists, Glucagon-Like Peptide-1 (GLP-1) receptor agonists, amylin receptor agonists, MC4 (melanocortin 4) agonists, orexin antagonists or agonists, TNF (tumor necrosis factor) agonists, CRF (corticotropin releasing factor) agonists, CRF BP (corticotropin releasing factor binding protein) antagonists, urocortin agonists, β3 agonists, oxyntomodulin and analogues, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists, Gastric Inhibitory Polypeptide agonists or antagonists (GIP analogues), gastrin and gastrin analogues.

It should be understood that any suitable combination of the derivatives according to the invention with one or more of the above-mentioned compounds and optionally one or more further pharmacologically active substances are considered to be within the scope of the present invention.

The PYY or PP peptide derivative or analogue thereof as well as compositions according to the invention can be administered by any route, including the enteral (e.g. oral administration) or parenteral route. In one aspect, the parenteral route is preferred and includes intravenous, intraarticular, intraperitoneal, subcutaneous, intramuscular, intrasternal injection and infusion as well as administration by the sublingual, transdermal, topical, transmucosal including nasal route, or by inhalation such as, e.g., pulmonary inhalation. The PYY or PP peptide derivative or analogue thereof may be administered to an animal including a mammal, such as, e.g., a human, by any convenient administration route, such as, e.g., the oral, buccal, nasal, ocular, pulmonary, topical, transdermal, vaginal, rectal, ocular, parenteral (including inter alia subcutaneous, intramuscular, and intravenous cf. above), route in a dose that is effective for the individual purposes. A person skilled in the art will know how to choose a suitable administration route. In one aspect, the administration is via the parenteral administration route. In one aspect, the PYY or PP peptide derivative or analogue thereof are administered subcutaneously and/or nasally. It is well known in the art that subcutaneous injections can be easily self-administered.

The term “peripheral administration” means administration outside of the central nervous system. Peripheral administration does not include direct administration to the brain. Peripheral administration includes, but is not limited to intravenous, intravascular, intramuscular, subcutaneous, pulmonary, oral, sublingual, enteral, rectal, transdermal, or intra-nasal administration.

As used herein, the term “solvate” refers to a complex of defined stoichiometry formed between a solute (in casu, a compound according to the present invention) and a solvent. Solvents may include, by way of example, water, ethanol, or acetic acid.

The PYY or PP peptide derivative or analogue thereof can be administered as such dispersed in a suitable vehicle or they can be administered in the form of a suitable composition. Such compositions are also within the scope of the invention. In the following are described suitable pharmaceutical compositions.

The PYY or PP peptide derivative or analogue thereof according to the invention may be in the form of a pharmaceutical composition comprising the specific PYY or PP peptide derivative or analogue thereof together with one or more physiologically or pharmaceutically acceptable excipients.

The term “pharmaceutically acceptable” as used herein means suited for normal pharmaceutical applications, i.e. giving rise to no serious adverse events in patients etc.

The term “excipient” as used herein means the chemical compounds which are normally added to pharmaceutical compositions, e.g. buffers, tonicity agents, preservatives and the like.

The pharmaceutical composition comprising a PYY or PP peptide derivative or analogue thereof according to the invention may be in the form of a solid, semi-solid or fluid composition.

Fluid compositions, which are sterile solutions or dispersions can be utilized by for example intravenous, intramuscular, intrathecal, epidural, intraperitoneal or subcutaneous injection of infusion. The PYY or PP peptide derivative or analogue thereof may also be prepared as a sterile solid composition, which may be dissolved or dispersed before or at the time of administration using e.g. sterile water, saline or other appropriate sterile injectable medium. The fluid form of the composition may be a solution, an emulsion including nano- emulsions, a suspension, a dispersion, a liposomal composition, a mixture, a spray, or an aerosol (the two latter types are especially relevant for nasal administration).

Suitable mediums for solutions or dispersions are normally based on water or pharmaceutically acceptable solvents e.g. like an oil (e.g. sesame or peanut oil) or an organic solvent like e.g. propanol or isopropanol. A composition according to the invention may comprise further pharmaceutically acceptable excipients such as, e.g., pH adjusting agents, osmotically active agents e.g. in order to adjust the isotonicity of the composition to physiologically acceptable levels, viscosity adjusting agents, suspending agents, emulsifiers, stabilizers, preservatives, antioxidants etc. In one aspect, the medium is water.

Compositions for nasal administration may also contain suitable non-irritating vehicles such as, e.g., polyethylene glycols, glycofurol, etc. as well as absorption enhancers well known by a person skilled in the art (e.g. with reference to Remington's Pharmaceutical Science).

For parenteral administration, in one aspect the PYY or PP peptide derivative or analogue thereof can be formulated generally by mixing it at the desired degree of purity, in a unit dosage injectable form (solution, suspension, or emulsion), with a pharmaceutically acceptable excipient or carrier, i.e., one that is non-toxic to recipients at the dosages and concentrations employed and is compatible with other ingredients of the composition.

Generally, the formulations are prepared by contacting the PYY or PP peptide derivative or analogue thereof uniformly and intimately with liquid carriers or finely divided solid carriers or both. Then, if necessary, the product is shaped into the desired formulation. Specifically the carrier is a parenteral carrier, more specifically a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes. Due to the amphiphatic nature of the PYY or PP peptide derivative or analogue thereof described herein suitable forms also include micellar formulations, liposomes and other types of formulations comprising one or more suitable lipids such as, e.g., phospholipids and the like. In one aspect, they are suspended in an aqueous carrier, for example, in an isotonic buffer solution at a pH of about 3.0 to about 8.0, specifically at a pH of about 3.5 to about 7.4, 3.5 to 6.0, or 3.5 to about 5.

The compositions may also be designed to controlled or prolonged delivery of the PYY or PP peptide derivative or analogue thereof after administration in order to obtain a less frequent administration regimen. Normally a dosage regimen including 1-2 daily administrations is considered suitable, but within the scope of the present invention is also included other administration regimens such as, e.g., more frequent and less frequent. In order to achieve a prolonged delivery of the PYY or PP peptide derivative or analogue thereof, a suitable vehicle including e.g. lipids or oils may be employed in order to form a depot at the administration site from which the receptor agonist is slowly released into the circulatory system, or an implant may be used. Suitable compositions in this respect include liposomes and biodegradable particles into which the receptor agonist has been incorporated.

In those situations where solid compositions are required, the solid composition may be in the form of tablets such as, e.g. conventional tablets, effervescent tablets, coated tablets, melt tablets or sublingual tablets, pellets, powders, granules, granulates, particulate material, solid dispersions or solid solutions. A semi-solid form of the composition may be a chewing gum, an ointment, a cream, a liniment, a paste, a gel or a hydrogel. Other suitable dosages forms of the pharmaceutical compositions according to the invention may be vagitories, suppositories, plasters, patches, tablets, capsules, sachets, troches, devices etc. The dosage form may be designed to release the compound freely or in a controlled manner e.g. with respect to tablets by suitable coatings.

The pharmaceutical composition may comprise a therapeutically effective amount of a PYY or PP peptide derivative or analogue thereof according to the invention. The content of a PYY or PP peptide derivative or analogue thereof of the invention in a pharmaceutical composition of the invention is e.g. from about 0.1 to about 100% w/w of the pharmaceutical composition.

The pharmaceutical compositions may be prepared by any of the method well known to a person skilled in pharmaceutical formulation.

In pharmaceutical compositions, the PYY or PP peptide derivative or analogue thereof are normally combined with a pharmaceutical excipient, i.e. a therapeutically inert substance or carrier. The carrier may take a wide variety of forms depending on the desired dosage form and administration route. The pharmaceutically acceptable excipients may be e.g. fillers, binders, disintegrants, diluents, glidants, solvents, emulsifying agents, suspending agents, stabilizers, enhancers, flavours, colours, pH adjusting agents, retarding agents, wetting agents, surface active agents, preservatives, antioxidants etc. Details can be found in pharmaceutical handbooks such as, e.g., Remington's Pharmaceutical Science or Pharmaceutical Excipient Handbook.

In one aspect compositions according to this invention will influence the physical state, stability, rate of in vivo release, and rate of in vivo clearance of the present PYY analogue peptides. See, e.g., Remington's Pharmaceutical Sciences 1435-712, 18th ed, Mack Publishing Co., Easton, Pa. (1990).

More particularly, administration of the pharmaceutical compositions according to the present invention may be via any common route so long as the target tissue is available via that route. In one aspect, the pharmaceutical compositions may be introduced into the subject by any conventional peripheral method, e.g., by intravenous, intradermal, intramusclar, intramammary, intraperitoneal, intrathecal, retrobulbar, intrapulmonary (e.g., term release); by oral, sublingual, nasal, anal, vaginal, or transdermal delivery, or by surgical implantation at a particular site. The treatment may consist of a single dose or a plurality of doses over a period of time. Controlled continual release of the compositions of the present invention is also contemplated.

The formulation may be liquid or may be solid, such as lyophilized, for reconstitution. Aqueous compositions of the present invention comprise an effective amount of the PYY or PP peptide derivative or analogue thereof, dissolved or dispersed in a pharmaceutically acceptable carrier or aqueous medium. The use of such media and agents for pharmaceutically active substances is well known in the art. Except insofar as any conventional media or agent is incompatible with the active ingredient, its use in therapeutic compositions is contemplated. Supplementary active ingredients also can be incorporated into the compositions.

The PYY or PP peptide derivative of analogue thereof of the invention may be prepared for administration as solutions of free base, or pharmacologically acceptable salts in water suitably mixed with surface active agents (e.g., sorbitan monooleate, polyoxyethylene sorbitain monolaurate (Tween 20), polyoxyethylene sorbitan monooleate (Tween 80), lecithin, polyoxyethylene-polyoxypropylene copolymers (Pluronics), hydroxypropylcellulose) or complexation agents (e.g., hydroxypropyl-b-cyclodextrin, sulfobutyether-b-cyclodextrin (Captisol), polyvinylpyrrolidone). Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like. Such products are readily prepared by procedures well known to those skilled in the art. Dispersions also can be prepared in glycerol, liquid polyethylene glycols, and mixtures thereof and in oils. Under ordinary conditions of storage and use, these preparations contain a preservative to prevent the growth of microorganisms.

In one aspect, the pharmaceutical compositions of the present invention are formulated so as to be suitable for parenteral administration, e.g., via injection or infusion. In one aspect, the PYY or PP peptide derivative of analogue thereof is suspended in an aqueous carrier, for example, in an buffer solution at a pH of about 3.0 to about 8.0, specifically at a pH of about 3.5 to about 7.4, about 3.5 to about 6.0, about 3.5 to about 5.0 or about 3.7 to about 4.7. Useful buffers include sodium acetate/acetic acid, sodium lactate/lactic acid, ascorbic acid, sodium citrate-citric acid, sodium bicarbonate/carbonic acid, sodium succinate/succinic acid, Histidine, Sodium benzoate/benzoic acid, and sodium phosphates, and Tris(hydroxymethyl)arninomehane. A form of repository or “depot” slow release preparation may be used so that therapeutically effective amounts of the preparation are delivered into the bloodstream over many hours or days following trans-dermal injection or delivery.

The pharmaceutical compositions suitable for injectable use include sterile aqueous solutions or dispersions and sterile powders for the extemporaneous preparation of sterile injectable solutions or dispersions. In all cases, the form should be sterile and should be fluid to the extent that is easily syringable. It is also desirable for the PYY or PP peptide derivative of analogue thereof of the invention to be stable under the conditions of manufacture and storage and must be preserved against the contaminating action of microorganisms, such as bacteria and fungi. The carrier can be a solvent or dispersion medium containing, for example, water, ethanol, polyol (e.g., sorbitol, glycerol, propylene glycol, and liquid polyethylene glycol, and the like), dimethylacetamide, cremorphor EL, suitable mixtures thereof, and oils (e.g., soybean, sesame, castor, cottonseed, ethyl oleate, isopropyl myristate, glycofurol, corn). The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, meta-cresol, benzyl alcohol, parabens (methyl, propyl, butyl), chlorobutanol, phenol, phenylmercuric salts (acetate, borate, nitrate), sorbic acid, thimerosal, and the like. In many cases, it will be beneficial to include tonicity agents (for example, sugars, sodium chloride). Prolonged absorption of the injectable compositions can be brought about by the use in the compositions of agents delaying absorption (for example, aluminum monostearate and gelatin).

Sterile injectable solutions may be prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum-drying and freeze-drying techniques that yield a powder of the active ingredient plus any additional desired ingredient from a previously sterile-filtered solution thereof. In general, the PYY or PP peptide derivative of analogue thereof may be formulated into a stable, safe pharmaceutical composition for administration to a patient. Pharmaceutical formulations contemplated for use in the methods of the invention may comprise approximately 0.01 to 20% (w/v), specifically 0.05 to 10%, of the PYY or PP peptide derivative of analogue thereof. The PYY or PP peptide derivative of analogue thereof may be in an acetate, phosphate, citrate or glutamate buffer allowing a pH of the final composition of about 3.0 to about 7.0 containing carbohydrate or polyhydric alcohol as tonicity modifier and, optionally, approximately 0.005 to 5.0% (w/v) of a preservative selected from the group consisting of m-cresol, benzyl alcohol, methyl, ethyl, propyl and butyl parabens and phenol. Such a preservative is generally included if the formulated peptide is to be included in a multiple use product.

In one aspect of the present invention, a pharmaceutical formulation of the present invention may contain a range of concentrations of PYY or PP peptide derivative of analogue thereof, e.g., between about 0.01% to about 98% w/w, or between about 1 to about 98% w/w, or specifically between 80% and 90% w/w, or specifically between about 0.01% to about 50% w/w, or more specifically between about 10% to about 25% w/w in this aspect. A sufficient amount of water for injection may be used to obtain the desired concentration of solution. The pharmaceutical formulations described herein may be lyophilized.

Generally, a therapeutically or prophylactically effective amount of the PYY or PP peptide derivative of analogue thereof will be determined by the age, weight, and condition or severity of the diseases or metabolic conditions or disorders of the recipient. See, e.g., Remington's Pharmaceutical Sciences 697-773. See also Wang and Hanson, Parenteral Formulations of Proteins and Peptides: Stability and Stabilizers, Journal of Parenteral Science and Technology, Technical Report No. 10, Supp. 42:2 S (1988). Typically, a dosage of between about 0.001 μg/kg body weight/day to about 1000 μg/kg body weight/day, may be used, but more or less, as a skilled practitioner will recognize, may be used. Dosing may be one, two, three, four or more times daily, or less frequently, such as once a week, once a month, or once a quarter, depending on the formulation, and may be in conjunction with other compositions as described herein. It should be noted that the present invention is not limited to the dosages recited herein.

Appropriate dosages may be ascertained through the use of established assays for determining level of metabolic conditions or disorders in conjunction with relevant dose-response data. The final dosage regimen will be determined by the attending physician, considering factors that modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

The frequency of dosing will depend on the pharmacokinetic parameters of the agents and the routes of administration. The optimal pharmaceutical formulation will be determined by one of skill in the art depending on the route of administration and the desired dosage. See, e.g., Remington's Pharmaceutical Sciences, supra, pages 1435-1712. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

It will be appreciated that the pharmaceutical compositions and treatment methods of the invention may be useful in fields of human medicine and veterinary medicine. Thus the subject to be treated may be a mammal, specifically human or other animal. For veterinary purposes, subjects include for example, farm animals including cows, sheep, pigs, horses and goats, companion animals such as dogs and cats, exotic and/or zoo animals, laboratory animals including mice, rats, rabbits, guinea pigs and hamsters; and poultry such as chickens, turkeys, ducks and geese.

The present PYY or PP peptide derivatives or analogues thereof and compositions containing them are also useful in the manufacture of a medicament for the therapeutic applications mentioned herein.

In one aspect, the present invention relates to the use of a derivative according to the invention for the preparation of a medicament.

### Syntheses

PYY or PP peptide derivatives or analogues thereof of the invention may be synthesized by standard solid phase peptide synthesis (SPPS), using either an automated peptide synthesizer, or traditional bench synthesis. The solid support can be, for example, Tentagel S RAM, chlorotrityl (CI) or Wang (OH) resin, all of which are readily available commercially. The active amino or hydroxyl groups of those resins react readily with the carboxyl group of an N-Fmoc amino acid, thereby covalently binding it to the polymer via a linkage to a linker attached to the resin. The resin-bound Fmoc-amino acid may be deprotected by exposure to a mixture of 20% piperidine in N-methylpyrrolidinone (NMP) which readily cleaves the Fmoc-group. The subsequent amino acid is coupled using a coupling reagent and followed by another deprotection of the Fmoc-group. Examples of reagents facilitating the coupling of incoming amino acids to the resin-bound amino acid chain are: diisopropylcarbodiimide (DIC), tetra-methyluronium hexafluorophosphate (HATU), O-(1H-benzotriazole-1-yl)- N,N,N\N′-tetramethyluronium hexafluorophosphate (HBTU), O-(1H-benzotriazole-1-yl)-N,N,N′,N′-tetramethyluronium tetrafluoroborate (TBTU), 1H-hydroxybenzotriazole (HOBt).

The SPPS is continued a stepwise manner until the desired sequence is obtained. At the end of the synthesis, the resin-bound protected peptide is deprotected cleaving the protection groups on the side chains and also cleaving the peptide from the resin. This is done with trifluoroacetic acid (TFA) containing scavengers such as triisopropylsilane (TIPS). The peptide is then precipitated in diethylether and isolated. Peptide synthesis by solution chemistry rather than solid phase chemistry is also feasible.

It may be desirable to purify the PP-fold peptides generated by the present invention. Peptide purification techniques are well known to those of skill in the art. These techniques involve, at one level, the crude fractionation of the cellular milieu to peptide and non-peptide fractions. Having separated the peptide from other proteins, the peptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography, polyacrylamide gel electrophoresis, and isoelectric focusing. A particularly efficient method of purifying peptides is reverse phase HPLC, followed by characterization of purified product by liquid chromatography/mass spectrometry (LC/MS) and Matrix-Assisted Laser Desorption Ionization (MALDI) mass spectrometry. Additional confirmation of purity is obtained by determining amino acid analysis.

In one aspect the present invention concern the purification, and in one aspect, the substantial purification, of a peptide derivative according to the invention. The term “purified peptide” as used herein, is intended to refer to a composition, isolatable from other components, wherein the peptide is purified to any degree relative to its naturally obtainable state. A purified peptide therefore also refers to a peptide, free from the environment in which it may naturally occur. Generally, “purified” will refer to a peptide composition that has been subjected to fractionation to remove various other components, and which composition substantially retains its expressed biological activity. Where the term “substantially purified” is used, this designation will refer to a composition in which the peptide forms the major component of the composition, such as constituting about 50%, about 60%, about 70%, about 80%, about 90%, about 95% or more of the peptides in the composition.

Various techniques suitable for use in peptide purification will be well known to those of skill in the art. These include, for example, precipitation with ammonium sulphate, PEG, antibodies, and the like; heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the peptides always be provided in their most purified state. Indeed, it is contemplated that less substantially purified products will have utility in certain aspects. Partial purification may be accomplished by using fewer purification steps in combination, or by utilizing different fopins of the same general purification scheme. For example, it is appreciated that a cation-exchange column chromatography performed, utilizing an HPLC apparatus, will generally result in a greater “-fold” purification than the same technique utilizing a low pressure chromatography system. Methods exhibiting a lower degree of relative purification may have advantages in total recovery of protein product, or in maintaining the activity of an expressed protein.

One may optionally purify and isolate such PYY or PP peptides according to the invention from other components obtained in the process. Methods for purifying a peptide can be found in U.S. Pat. No. 5,849,883. These documents describe specific exemplary methods for the isolation and purification of G-CSF compositions that may be useful in isolating and purifying PYY or PP peptides according to the invention. Given the disclosure of these patents, it is evident that one of skill in the art would be well aware of numerous purification techniques that may be used to purify PYY or PP peptides according to the invention from a given source.

Also it is contemplated that a combination of anion exchange and immunoaffinity chromatography may be employed to produce purified PP-fold peptide compositions of the present invention.

### Embodiments of the Invention

1. A PYY or PP peptide derivative or analogue thereof, wherein at least one amino acid residue and/or the N- and/or C-terminus of the peptide backbone is derivatised with a serum albumin binding side chain defined by A-B-C-D-, A-C-D-, A-B-C-, or A-C-, wherein

### A- is

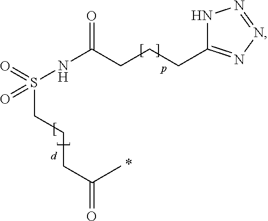

wherein p is selected from the group consisting of 10, 11, 12, 13, 14, 15 and 16 and d is selected from the group consisting of 0, 1, 2, 3, 4 and 5,
and -B- is selected from the group consisting of

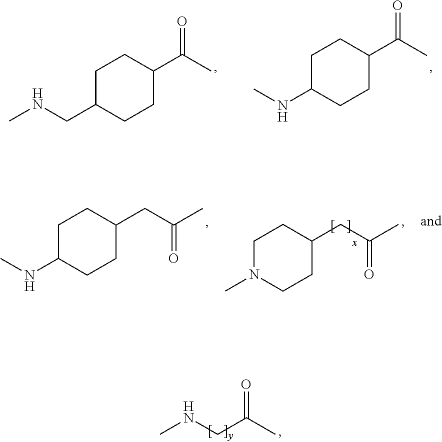

wherein x is selected from the group consisting of 0, 1, 2, 3 and 4, and y is selected from the group consisting of 1, 2, 3, 4, 5, 6, 7, 8, 9, 10, 11 and 12;

### or A- is

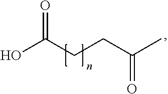

wherein n is selected from the group consisting of 12, 13, 14, 15, 16 17, 18 and 19,
and -B- is selected from the group consisting of

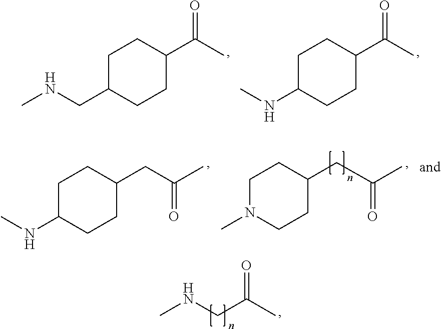

wherein x is selected from the group consisting of 0, 1, 2, 3 and 4; and
-C- is selected from the group consisting of

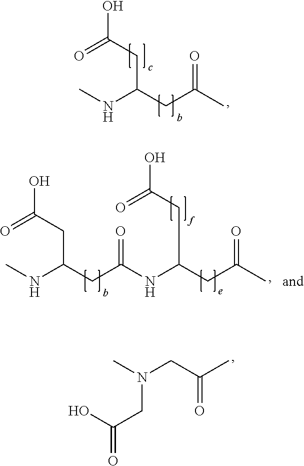

wherein b and e are each independently selected from the group consisting of 0, 1, and 2, and c and f are each independently selected from the group consisting of 0, 1, and 2 with the proviso that when
c is 0 b is 1 or 2,
c is 1 or 2 b is 0,
f is 0 e is 1 or 2,
f is 1 or 2 e is 0, and
with the proviso that when A- is

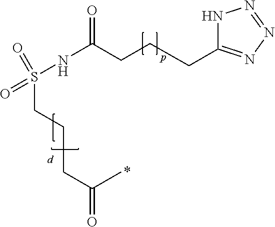

-C- may be deleted; and -D- is attached to said amino acid residue and is a spacer.
2. A PYY or PP peptide derivative or analogue thereof, wherein at least one amino acid residue and/or the N- and/or C-terminus of the peptide backbone is derivatised with a serum albumin binding side chain defined by A-B-C-D-, A-C-D-, A-B-C-, or A-C-, wherein

### A- is

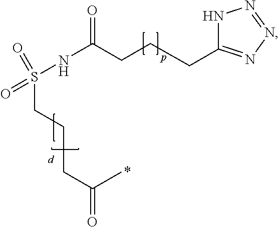

wherein p is selected from the group consisting of 10, 11, 12, 13, 14, 15 and 16 and d is selected from the group consisting of 0, 1, 2, 3, 4 and 5,
and -B- is selected from the group consisting of

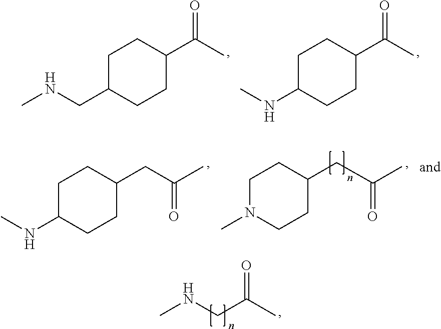

wherein x is selected from the group consisting of 0, 1, 2, 3 and 4, and y is selected from the group consisting of 1, 2, 3, 4, 5, 6, 7, 8, 9, 10, 11 and 12;

### or A- is

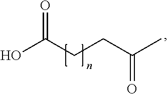

wherein n is selected from the group consisting of 12, 13, 14, 15, 16 17, 18 and 19,
and -B- is selected from the group consisting of

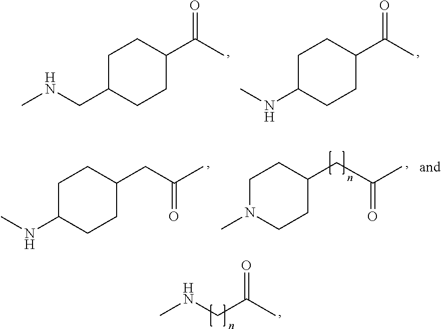

wherein x is selected from the group consisting of 0, 1, 2, 3 and 4; and
-C- is selected from the group consisting of

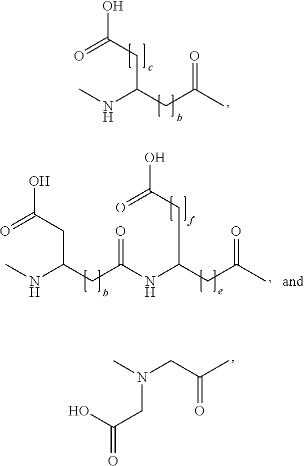

wherein b and e are each independently selected from the group consisting of 0, 1, and 2, and c and f are each independently selected from the group consisting of 0, 1, and 2 with the proviso that when
c is 0 b is 1 or 2,
c is 1 or 2 b is 0,
f is 0 e is 1 or 2,
f is 1 or 2 e is 0; and
-D- is attached to said amino acid residue and is a spacer.
3. A PYY or PP peptide derivative or analogue thereof according to any of the preceding embodiments, wherein the peptide is derived from a vertebrate.
4. A PYY or PP peptide derivative or analogue thereof according to any of the preceding embodiments, wherein the peptide is selected from the group consisting of
a PP analogue according to formula I

(I)
Z-Ala-Pro-Leu-Glu-Pro-Val-Tyr-Pro-Gly-Xaa₁₀-Xaa₁₁-
Xaa₁₂-Xaa₁₃-Xaa₁₄-Xaa₁₅-Xaa₁₆-Xaa₁₇-Xaa₁₈-Xaa₁₉-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Thr-Arg-Xaa₃₄-Arg-Xaa₃₆,

wherein
Z is the side chain A-B-C-D-, A-C-D-, A-B-C-, or A-C- attached to the N-terminal amino group, or not present when A-B-C-D-, A-C-D-, A-B-C-, A-C- is attached to the side chain of an amino acid,
Ala in position 1 may be deleted,
Xaa₁₀ is Asp, Asn, 2,3-diaminopropionic acid, 2,4-diaminobutyric acid, ornithine, or Lys,
Xaa₁₁ is Asp, Asn, 2,3-diaminopropionic acid, 2,4-diaminobutyric acid, ornithine, or Lys,
Xaa₁₂ is Ala, 2,3-diaminopropionic acid, 2,4-diaminobutyric acid, ornithine, or Lys,
Xaa₁₃ is Thr, 2,3-diaminopropionic acid, 2,4-diaminobutyric acid, ornithine, or Lys,
Xaa₁₄ is Pro or hydroxyproline,
Xaa₁₅ is Glu, 2,3-diaminopropionic acid, 2,4-diaminobutyric acid, ornithine, or Lys,
Xaa₁₆ is Gln, 2,3-diaminopropionic acid, 2,4-diaminobutyric acid, ornithine, or Lys,
Xaa₁₇ is Leu, Val, Ile, homoleucine, norleucine, (1-aminocyclopentyl) carboxylic acid, (1-aminocyclohexyl) carboxylic acid, or 1-aminobutyric acid,
Xaa₁₈ is Ala, 2,3-diaminopropionic acid, 2,4-diaminobutyric acid, ornithine, or Lys,
Xaa₁₉ is Gln, 2,3-diaminopropionic acid, 2,4-diaminobutyric acid, ornithine, or Lys,
Xaa₂₀ is Tyr, Phe, or 3-pyridylalanine,
Xaa₂₁ is Ala, 2,3-diaminopropionic acid, 2,4-diaminobutyric acid, ornithine, or Lys,
Xaa₂₂ is Ala, 2,3-diaminopropionic acid, 2,4-diaminobutyric acid, ornithine, or Lys,
Xaa₂₃ is Asp, 2,3-diaminopropionic acid, 2,4-diaminobutyric acid, ornithine, or Lys,
Xaa₂₄ is Leu, Val, Ile, homoleucine, norleucine, (1-aminocyclopentyl) carboxylic acid, (1-aminocyclohexyl) carboxylic acid, or 1-aminobutyric acid,
Xaa₂₅ is Arg, 2,3-diaminopropionic acid, 2,4-diaminobutyric acid, ornithine, or Lys,
Xaa₂₆ is Arg, His, 2,3-diaminopropionic acid, 2,4-diaminobutyric acid, ornithine, or Lys,
Xaa₂₇ is Tyr, Phe, homoPhe, or 3-pyridylalanine,
Xaa₂₈ is Ile, Val, Leu, homoleucine, norleucine, (1-aminocyclopentyl) carboxylic acid, (1-aminocyclohexyl) carboxylic acid, or 1-aminobutyric acid,

### Xaa₂₉ is Asn, Gln, or Lys,

Xaa₃₀ is Met, Leu, Val, Ile, homoleucine, norleucine, (1-aminocyclopentyl) carboxylic acid, (1-aminocyclohexyl) carboxylic acid, or 1-aminobutyric acid,
Xaa₃₁ is Leu, Val, Ile, homoleucine, norleucine, (1-aminocyclopentyl) carboxylic acid, (1-aminocyclohexyl) carboxylic acid, or 1-aminobutyric acid,
Arg in position 33 may be substituted with Lys,

### Xaa₃₄ is Gln, Asn, or His,

Arg in position 35 may be substituted with Lys,
Xaa₃₆ is Tyr, 3-pyridylalanine;
a PYY analogue according to formula II

(II)
Z-Tyr-Pro-Xaa₃-Xaa₄-Pro-Glu-Ala-Pro-Gly-Xaa₁₀-
Xaa₁₁-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Thr-Arg-Xaa₃₄-Arg-
Xaa₃₆,

wherein
Z is the side chain A-B-C-D-, A-C-D-, A-B-C-, or A-C- attached to the N-terminal amino group, or not present when A-B-C-D-, A-C-D-, A-B-C-, A-C- is attached to the side chain of an amino acid,
Tyr-Pro in position 1 and 2 may be deleted,
Tyr in position 1 may be substituted with Ala or may be deleted,
Xaa₃ is Ile, Val, Leu, (1-aminocyclopentyl) carboxylic acid, (1-aminocyclohexyl) carboxylic acid, or 1-aminobutyric acid,
Xaa₄ is Glu, 2,3-diaminopropionic acid, 2,4-diaminobutyric acid, ornithine, or Lys,
Glu in position 6 may be substituted with Val,
Ala in position 7 may be substituted with Tyr,
Xaa₁₀ is Glu, 2,3-diaminopropionic acid, 2,4-diaminobutyric acid, ornithine, or Lys,
Xaa₁₁ is Asp, 2,3-diaminopropionic acid, 2,4-diaminobutyric acid, ornithine, or Lys,
Xaa₁₂ is Ala, 2,3-diaminopropionic acid, 2,4-diaminobutyric acid, ornithine, or Lys,
Xaa₁₃ is Ser, 2,3-diaminopropionic acid, 2,4-diaminobutyric acid, ornithine, or Lys,
Xaa₁₄ is Pro, hydroxyproline, or Lys,
Xaa₁₅ is Glu, 2,3-diaminopropionic acid, 2,4-diaminobutyric acid, ornithine, or Lys,
Xaa₁₆ is Glu, 2,3-diaminopropionic acid, 2,4-diaminobutyric acid, ornithine, or Lys,
Xaa₁₇ is Leu, Val, Ile, homoleucine, norleucine, (1-aminocyclopentyl) carboxylic acid, (1-aminocyclohexyl) carboxylic acid, or 1-aminobutyric acid,
Xaa₁₈ is Asn, 2,3-diaminopropionic acid, 2,4-diaminobutyric acid, ornithine, or Lys,
Xaa₁₉ is Arg, 2,3-diaminopropionic acid, 2,4-diaminobutyric acid, ornithine, or Lys,
Xaa₂₀ is Tyr, Phe, 3-pyridylalaine, 2,3-diaminopropionic acid, 2,4-diaminobutyric acid, ornithine, or Lys,
Xaa₂₁ is Tyr, Phe, 3-pyridylalaine, 2,3-diaminopropionic acid, 2,4-diaminobutyric acid, ornithine, or Lys,
Xaa₂₂ is Asp, 2,3-diaminopropionic acid, 2,4-diaminobutyric acid, ornithine, or Lys,
Xaa₂₃ is Ala, 2,3-diaminopropionic acid, 2,4-diaminobutyric acid, ornithine, or Lys,
Xaa₂₄ is Leu, Ile, Val, homoleucine, norleucine, (1-aminocyclopentyl) carboxylic acid, (1-aminocyclohexyl) carboxylic acid, 1-aminobutyric acid, or Lys,
Xaa₂₅ is Arg, 2,3-diaminopropionic acid, 2,4-diaminobutyric acid, ornithine, or Lys,
Xaa₂₆ is H is, Arg, 2,3-diaminopropionic acid, 2,4-diaminobutyric acid, ornithine, or Lys,
Xaa₂₇ is Tyr, Phe, homoPhe, or 3-pyridylalanine,
Xaa₂₈ is Ile, Val, Leu, homoleucine, norleucine, (1-aminocyclopentyl) carboxylic acid, (1-aminocyclohexyl) carboxylic acid, 1-aminobutyric acid, or Lys,

### Xaa₂₉ is Asn, Gln, or Lys,

Xaa₃₀ is Met, Leu, Val, Ile, homoleucine, norleucine, (1-aminocyclopentyl) carboxylic acid, (1-aminocyclohexyl) carboxylic acid, 1-aminobutyric acid, or Lys,
Xaa₃₁ is Leu, Val, Ile, homoleucine, norleucine, (1-aminocyclopentyl) carboxylic acid, (1-aminocyclohexyl) carboxylic acid, 1-aminobutyric acid, or Lys,
Thr in position 32 may be substituted with Lys,

### Xaa₃₄ is Gln, Asn, or His,

Xaa₃₆ is Tyr, 3-pyridylalanine, or Lys;
wherein the compound is modified with a serum albumin binding side chain comprising a distal carboxylic acid or tetrazole group.
5. A PYY or PP peptide derivative or analogue thereof according to any of the preceding embodiments, wherein the peptide is selected from the group consisting of
a PP analogue according to formula I

(I)
Z-Ala-Pro-Leu-Glu-Pro-Val-Tyr-Pro-Gly-Xaa₁₀-Xaa₁₁-
Xaa₁₂-Xaa₁₃-Xaa₁₄-Xaa₁₅-Xaa₁₆-Xaa₁₇-Xaa₁₈-Xaa₁₉-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Thr-Arg-Xaa₃₄-Arg-Xaa₃₆,

wherein
Z is the side chain A-B-C-D-, A-C-D-, A-B-C-, or A-C- attached to the N-terminal amino group, or not present when A-B-C-D-, A-C-D-, A-B-C-, A-C- is attached to the side chain of an amino acid,
Xaa₁₀ is Asp, 2,3-diaminopropionic acid, 2,4-diaminobutyric acid, ornithine, or Lys,
Xaa₁₁ is Asn, 2,3-diaminopropionic acid, 2,4-diaminobutyric acid, ornithine, or Lys,
Xaa₁₂ is Ala, 2,3-diaminopropionic acid, 2,4-diaminobutyric acid, ornithine, or Lys,
Xaa₁₃ is Thr, 2,3-diaminopropionic acid, 2,4-diaminobutyric acid, ornithine, or Lys,
Xaa₁₄ is Pro or hydroxyproline,
Xaa₁₅ is Glu, 2,3-diaminopropionic acid, 2,4-diaminobutyric acid, ornithine, or Lys,
Xaa₁₆ is Gln, 2,3-diaminopropionic acid, 2,4-diaminobutyric acid, ornithine, or Lys,
Xaa₁₇ is Leu, Val, Ile, homoleucine, norleucine, (1-aminocyclopentyl) carboxylic acid, (1-aminocyclohexyl) carboxylic acid, or 1-aminobutyric acid,
Xaa₁₈ is Ala, 2,3-diaminopropionic acid, 2,4-diaminobutyric acid, ornithine, or Lys,
Xaa₁₉ is Gln, 2,3-diaminopropionic acid, 2,4-diaminobutyric acid, ornithine, or Lys,
Xaa₂₀ is Tyr, Phe, or 3-pyridylalanine,
Xaa₂₁ is Ala, 2,3-diaminopropionic acid, 2,4-diaminobutyric acid, ornithine, or Lys,
Xaa₂₂ is Ala, 2,3-diaminopropionic acid, 2,4-diaminobutyric acid, ornithine, or Lys,
Xaa₂₃ is Asp, 2,3-diaminopropionic acid, 2,4-diaminobutyric acid, ornithine, or Lys,
Xaa₂₄ is Leu, Val, Ile, homoleucine, norleucine, (1-aminocyclopentyl) carboxylic acid, (1-aminocyclohexyl) carboxylic acid, or 1-aminobutyric acid,
Xaa₂₅ is Arg, 2,3-diaminopropionic acid, 2,4-diaminobutyric acid, ornithine, or Lys,
Xaa₂₆ is Arg, His, 2,3-diaminopropionic acid, 2,4-diaminobutyric acid, ornithine, or Lys,
Xaa₂₇ is Tyr, Phe, homoPhe, or 3-pyridylalanine,
Xaa₂₈ is Ile, Val, Leu, homoleucine, norleucine, (1-aminocyclopentyl) carboxylic acid, (1-aminocyclohexyl) carboxylic acid, or 1-aminobutyric acid,

### Xaa₂₉ is Asn or Gln,

Xaa₃₀ is Met, Leu, Val, Ile, homoleucine, norleucine, (1-aminocyclopentyl) carboxylic acid, (1-aminocyclohexyl) carboxylic acid, or 1-aminobutyric acid,
Xaa₃₁ is Leu, Val, Ile, homoleucine, norleucine, (1-aminocyclopentyl) carboxylic acid, (1-aminocyclohexyl) carboxylic acid, or 1-aminobutyric acid,

### Xaa₃₄ is Gln, Asn, or H is,

Xaa₃₆ is Tyr, 3-pyridylalanine;
a PYY analogue according to formula II

(II)
Z-Tyr-Pro-Xaa₃-Xaa₄-Pro-Glu-Ala-Pro-Gly-Xaa₁₀-
Xaa₁₁-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Thr-Arg-Xaa₃₄-Arg-
Xaa₃₆,

wherein
Z is the side chain A-B-C-D-, A-C-D-, A-B-C-, or A-C- attached to the N-terminal amino group, or not present when A-B-C-D-, A-C-D-, A-B-C-, A-C- is attached to the side chain of an amino acid,
Tyr-Pro in position 1 and 2 may be deleted,
Xaa₃ is Ile, Val, Leu (1-aminocyclopentyl) carboxylic acid, (1-aminocyclohexyl) carboxylic acid, or 1-aminobutyric acid,
Xaa₄ is Glu, 2,3-diaminopropionic acid, 2,4-diaminobutyric acid, ornithine, or Lys,
Xaa₁₀ is Glu, 2,3-diaminopropionic acid, 2,4-diaminobutyric acid, ornithine, or Lys,
Xaa₁₁ is Asp, 2,3-diaminopropionic acid, 2,4-diaminobutyric acid, ornithine, or Lys,
Xaa₁₂ is Ala, 2,3-diaminopropionic acid, 2,4-diaminobutyric acid, ornithine, or Lys,
Xaa₁₃ is Ser, 2,3-diaminopropionic acid, 2,4-diaminobutyric acid, ornithine, or Lys,
Xaa₁₄ is Pro or hydroxyproline,
Xaa₁₅ is Glu, 2,3-diaminopropionic acid, 2,4-diaminobutyric acid, ornithine, or Lys,
Xaa₁₆ is Glu, 2,3-diaminopropionic acid, 2,4-diaminobutyric acid, ornithine, or Lys,
Xaa₁₇ is Leu, Val, Ile, homoleucine, norleucine, (1-aminocyclopentyl) carboxylic acid, (1-aminocyclohexyl) carboxylic acid, or 1-aminobutyric acid,
Xaa₁₈ is Asn, 2,3-diaminopropionic acid, 2,4-diaminobutyric acid, ornithine, or Lys,
Xaa₁₉ is Arg, 2,3-diaminopropionic acid, 2,4-diaminobutyric acid, ornithine, or Lys,
Xaa₂₀ is Tyr, Phe, 3-pyridylalaine, 2,3-diaminopropionic acid, 2,4-diaminobutyric acid, ornithine, or Lys,
Xaa₂₁ is Tyr, Phe, 3-pyridylalaine, 2,3-diaminopropionic acid, 2,4-diaminobutyric acid, ornithine, or Lys,
Xaa₂₂ is Asp, 2,3-diaminopropionic acid, 2,4-diaminobutyric acid, ornithine, or Lys,
Xaa₂₃ is Ala, 2,3-diaminopropionic acid, 2,4-diaminobutyric acid, ornithine, or Lys,
Xaa₂₄ is Leu, Ile, Val, homoleucine, norleucine, (1-aminocyclopentyl) carboxylic acid, (1-aminocyclohexyl) carboxylic acid, or 1-aminobutyric acid,
Xaa₂₅ is Arg, 2,3-diaminopropionic acid, 2,4-diaminobutyric acid, ornithine, or Lys,
Xaa₂₆ is His, Arg, 2,3-diaminopropionic acid, 2,4-diaminobutyric acid, ornithine, or Lys,
Xaa₂₇ is Tyr, Phe, homoPhe, or 3-pyridylalanine,
Xaa₂₈ is Ile, Val, Leu, homoleucine, norleucine, (1-aminocyclopentyl) carboxylic acid, (1-aminocyclohexyl) carboxylic acid, or 1-aminobutyric acid,

### Xaa₂₉ is Asn or Gln,

Xaa₃₀ is Met, Leu, Val, Ile, homoleucine, norleucine, (1-aminocyclopentyl) carboxylic acid, (1-aminocyclohexyl) carboxylic acid, or 1-aminobutyric acid,
Xaa₃₁ is Leu, Val, Ile, homoleucine, norleucine, (1-aminocyclopentyl) carboxylic acid, (1-aminocyclohexyl) carboxylic acid, or 1-aminobutyric acid,

### Xaa₃₄ is Gln, Asn, or His,

Xaa₃₆ is Tyr or 3-pyridylalanine;
wherein the compound is modified with a serum albumin binding side chain comprising a distal carboxylic acid or tetrazole group.
6. A PYY or PP peptide derivate or analogue thereof according to any of the preceding embodiments, wherein the peptide may be truncated by deletion of a consecutive sequence of one or more amino acids from the N-terminal end.
7. A PYY or PP peptide derivate or analogue thereof according to embodiment 6, wherein the consecutive sequence of one or more amino acids is selected from the group consisting of position 1 to 25 in PYY or position 1 to 2 in PP.
8. A PYY or PP peptide derivate or analogue thereof according to embodiment 6, wherein the consecutive sequence of one or more amino acids is selected from the group consisting of position 1, position 1 to 2, and position 1 to 17 in PYY.
9. A PYY or PP peptide derivate or analogue thereof according to embodiment 6, wherein the consecutive sequence of one or more amino acids is selected from the group consisting of position 1 in PP.10. A PYY or PP peptide derivate or analogue thereof according to any of the preceding embodiments, wherein the serum albumin binding side chain is attached to the side chain of an amino acid of the peptide backbone.
11. A PYY or PP peptide derivate or analogue thereof according to any of the preceding embodiments, wherein the serum albumin binding side chain is attached to an amino group of the side chain of an amino acid of the peptide backbone.
12. A PYY or PP peptide derivate or analogue thereof according to any of the preceding embodiments, wherein the serum albumin binding side chain is attached to the aminoterminal position or position 18 of PP.
13. A PYY or PP peptide derivate or analogue thereof according to any of the preceding embodiments, wherein the serum albumin binding side chain is attached to the aminoterminal position, position 18 or position 22 of PYY.
14. A PYY or PP peptide derivate or analogue thereof according to any of the preceding embodiments, wherein the serum albumin binding side chain is attached to an amino group of the side chain of an amino acid of the peptide backbone selected from the group consisting of 2,3-diaminopropionic acid, 2,4-diaminobutyric acid, ornithine, and Lys.
15. A PYY or PP peptide derivative or analogue thereof according to any of the preceding embodiments, wherein the spacer, -D-, comprises one or more 8-amino-3,6-dioxaoctanoic acid (Oeg) molecules, such as two 8-amino-3,6-dioxaoctanoic acid (Oeg) molecules.
16. A PYY or PP peptide derivate or analogue thereof according to any of the preceding embodiments, wherein A-B-C-D- is selected from the group consisting of [2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl], [2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl], and [4-(16-(1H-Tetrazol-5-yl)hexadecanoylsulfamoyl)butyryl]ethoxy}ethoxy)acetylamino]ethoxy}ethoxy)acetyl].
17. A PYY or PP peptide derivate or analogue thereof according to any of the preceding embodiments, wherein the PYY or PP peptide derivate or analogue thereof is selected from the group consisting of SEQ ID NO: 23, SEQ ID NO: 57, SEQ ID NO: 58, SEQ ID NO: 43, and SEQ ID NO: 55.
18. A PYY or PP peptide derivative or analogue thereof according to any of the preceding embodiments, wherein said derivative is selective for the Y2 and/or Y4 receptors over the Y1 receptor.
19. A PYY or PP peptide derivative or analogue thereof according to any of the preceding embodiments, wherein said derivative is selective for the Y2 and/or Y4 receptors over the Y5 receptor.
20. A PYY or PP peptide derivative or analogue thereof according to any of the preceding embodiments, wherein said derivative is suitable for administration in a once-daily dosing regime.
21. A PYY or PP peptide derivative or analogue thereof according to any of the preceding embodiments, wherein said derivative is suitable for administration in a once-weekly dosing regime.
22. A PYY or PP peptide derivative or analogue thereof according to any of the preceding embodiments, wherein said derivative is suitable for administration in a twice-monthly dosing regime.
23. A PYY or PP peptide derivative or analogue thereof according to any of the preceding embodiments, wherein said derivative is suitable for administration in a once-monthly dosing regime.
24. A PYY or PP peptide derivative or analogue thereof according to any of the preceding embodiments, wherein said derivative shows improved PK profile compared to human PYY, PYY(3-36), or PP.
25. A PYY or PP peptide derivative or analogue thereof according to any of the preceding embodiments, wherein said derivative shows protracted properties compared to human PYY, PYY(3-36), or PP.
26. A PYY or PP peptide derivative or analogue thereof according to any of the preceding embodiments, wherein said derivative shows improved half life in vivo compared to human PYY, PYY(3-36), or PP.
27. A PYY or PP peptide derivative or analogue thereof according to any of the preceding embodiments, wherein a therapeutically effective dose of said derivative causes less side effects compared to human PYY, PYY(3-36), or PP.
28. A PYY or PP peptide derivative or analogue thereof according to any of the preceding embodiments selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and SEQ ID NO: 13.
29. A PYY or PP peptide derivative or analogue thereof according to any of the preceding embodiments selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56.
30. A PYY or PP peptide derivative or analogue thereof according to any of the preceding embodiments selected from the group consisting of SEQ ID NO: 3 to SEQ ID NO: 72, SEQ ID NO: 74 and SEQ ID NO: 75.
31. A composition comprising a PYY or PP peptide derivative or analogue thereof as defined in any of the preceding embodiments and one or more pharmaceutical excipients.
32. A method of treatment of a condition responsive to Y receptor modulation by administration of a PYY or PP peptide derivative or analogue thereof as defined in any of the embodiments 1-30.
33. A method of treatment according to embodiment 32, wherein the condition responsive to Y receptor modulation is obesity.
34. A method of treatment according to embodiment 32 or 33, wherein the condition responsive to Y receptor modulation is obesity-related diseases, such as reduction of food intake, Syndrome X (metabolic syndrome), diabetes, type 2 diabetes mellitus or Non Insulin Dependent Diabetes Mellitus (NIDDM), hyperglycemia, insulin resistance, or impaired glucose tolerance.
35. A method of treatment according to embodiment 32 or 33, wherein the condition responsive to Y receptor modulation is an obesity-related cardiovascular disease such as hypertension, atherosclerosis, coronary artery disease, myocardial infarction, peripheral vascular disease, stroke, thromboembolic diseases, hypercholesterolemia, or hyperlipidemia.
36. A method of treatment according to embodiment 32, wherein the condition responsive to Y receptor modulation is diarrhoea such as infectious diarrhoea, inflammatory diarrhoea, chemotherapy-induced diarrhoea, short bowel syndrome, or the diarrhoea which typically occurs following surgical procedures, e.g., ileostomy.
37. A method of treatment according to embodiment 32, wherein the condition responsive to Y receptor modulation is a condition characterized by damage to the intestine such as chemotherapy-induced diarrhoea, ulcerative colitis, Crohns disease, bowel atrophy, loss of bowel mucosa, and/or loss of bowel mucosal function.
38. A method of treatment according to embodiment 32, wherein the condition responsive to Y receptor modulation is an intestinal inflammatory condition such as ulcerative colitis or Crohns disease.
39. A method of treatment according to embodiment 32, wherein the condition responsive to Y receptor modulation is allergic or non-allergic rhinitis.
40. A method of treatment according to embodiment 32, wherein the condition responsive to Y receptor modulation is anxiety.
41. A method of treatment according to any of the embodiments 32-40, wherein the administration regime is selected from the group consisting of once-daily, once-weekly, twice-monthly, or once-monthly.
42. A method of treatment according to any of the embodiments 32-41, wherein said derivative shows improved PK profile compared to human PYY, PYY(3-36), or PP.
43. A method of treatment according to any of the embodiments 32-42, wherein said derivative shows protracted properties compared to human PYY, PYY(3-36), or PP.
44. A method of treatment according to any of the embodiments 32-43, wherein said derivative shows improved half life in vivo compared to human PYY, PYY(3-36) or PP.
45. A method of treatment according to any of the embodiments 32-44, wherein a therapeutically effective dose of said derivative causes less side effects compared to human PYY, PYY(3-36), or PP.
46. Use of a PYY or PP peptide derivative or analogue thereof as defined in any of embodiments 1-30 for the preparation of a medicament for the treatment of a condition responsive to Y receptor modulation, such as obesity or obesity-related diseases, e.g., reduction of food intake.
47. Use of a PYY or PP peptide derivative or analogue thereof as defined in any of embodiments 1-30 for administration in a mammal, wherein said derivative shows protracted properties compared to human PYY, PYY(3-36), or PP.

### EXAMPLES

### Abbreviations Used

r.t: Room temperature
AcCN: acetonitrile
DIPEA: diisopropylethylamine
H₂O: water
CH₃CN: acetonitrile
DMF: NN dimethylformamide
HBTU: 2-(1H-Benzotriazol-1-yl-)-1,1,3,3 tetramethyluronium hexafluorophosphate
Fmoc: 9H-fluoren-9-ylmethoxycarbonyl
Boc: tert butyloxycarbonyl
OtBu: tert butyl ester
tBu: tert butyl
Trt: triphenylmethyl
Pmc: 2,2,5,7,8-Pentamethyl-chroman-6-sulfonyl
Dde: 1-(4,4-Dimethyl-2,6-dioxocyclohexylidene)ethyl

### HFIP: Hexafluoroisopropanol

ivDde: 1-(4,4-Dimethyl-2,6-dioxocyclohexylidene)-3-methylbutyl
Mtt: 4-methyltrityl
Mmt: 4-methoxytrityl
DCM: dichloromethane
TIPS: triisopropylsilane
TFA: trifluoroacetic acid
Et2O: diethylether

### NMP: 1-Methyl-pyrrolidin-2-one

### DIPEA: Diisopropylethylamine

HOAc: acetic acid
HOAt: 1-Hydroxy-7-azabenzotriazole

### HOBt: 1-Hydroxybenzotriazole

### DIC: Diisopropylcarbodiimtde

MW: Molecular weight

### Synthesis of Resin Bound Peptide

### SPPS Method I

The protected peptidyl resin was synthesized according to the Fmoc strategy on an Advanced ChemTech Synthesiser (APEX 348) 0.25 mmol scale using the manufacturer supplied protocols which employ DIC (dicyclohexylcarbodiimide) and HOBt (1-Hydroxybenzotriazole) mediated couplings in NMP(N-methylpyrrolidone). The starting resin used for the synthesis of the peptide amides was Tentagel RAM (Rapp Polymere, Germany), Rink amid ChemMatrix resin (Matrix Innovation, Canada) Rink-Amide resin (Merck/Novabiochem) and either Wang or chlorotrityl resin was used for peptides with a carboxy C-terminal. The protected amino acid derivatives used were standard Fmoc-amino acids (supplied from e.g. Advanced Chemtech, or Novabiochem. The epsilon amino group of lysine to be derivatised was protected with Mtt. The synthesis of the peptides may in some cases be improved by the use of dipeptides, e.g., pseudoprolines from Novabiochem, Fmoc-Ser(tbu)-ψSer(Me,Me)-OH, see, e.g., catalogue from Novobiochem 2002/2003 or newer version, or W. R. Sampson (1999), J. Pep. Sci. 5, 403.

### SPPS Method II

The protected peptidyl resin was synthesized according to the Fmoc strategy on a Liberty from CEM corporation USA. Either 0.25 mmol or 0.5 mmol scale using the manufacturer supplied protocols which employ DIC (dicyclohexylcarbodiimide) and HOBt (1-Hydroxybenzotriazole) mediated couplings in NMP(N-methylpyrrolidone) was used. The starting resin used for the synthesis of the peptide amides was Tentagel RAM (Rapp Polymere, Germany), Rink amid ChemMatrix resin (Matrix Innovation, Canada) or Rink-Amide resin (Merck/Novabiochem) and either Wang or chlorotrityl resin was used for peptides with a carboxy C-terminal. The protected amino acid derivatives used were standard Fmoc-amino acids (supplied from e.g. Advanced Chemtech, or Novabiochem. The epsilon amino group of lysine in position 13 was protected with Mtt. The synthesis of the peptides may in some cases be improved by the use of dipeptides, e.g., pseudoprolines from Novabiochem, Fmoc-Ser(tbu)-ψSer(Me,Me)-OH, see, e.g., catalogue from Novobiochem 2002/2003 or newer version, or W. R. Sampson (1999), J. Pep. Sci. 5, 403.

Procedure for removal of Mtt-protection: The resin was placed in a syringe and treated with hexafluoroisopropanol for 2×10 min to remove the Mtt group. The resin was then washed with DCM and NMP as described above and neutralized with 5% DIPEA in NMP before coupling the albumin handles.

Procedure for attachment of sidechains to Lysine residue: The albumin binding residue A-B-C-D-, A-C-D-, A-B-C-, or A-B- can be attached to the peptide either by stepwise acylation to resin bound peptide or acylation in solution to the unprotected peptide using standard acylation reagent such as but not limited to DIC, HOBt/DIC, HOAt/DIC, or HBTU.

### Solid Phase Method III

The protected peptidyl resin was synthesized on a Prelude (Protein technologies) according to the instructions from the manufacture. Typically 300 mg resin (Tentagel S Ram, Rapp Polymere) was used in the 10 ml reaction vessel or 1 gram of Tentage S RAM resin was used in the 40 ml reaction vessel according to manufactor. The step-wise assembly of the peptide was done using standard Fmoc/t-Bu strategy according to the manufacture of Prelude.

### Manuel Synthesis of Peptidyl Resin

1 g Tentagel S Ram 0.25 mmol/g (Rapp Polymere, Germany) was swelled in NMP for 30 min in a 50 ml syringe with polypropylene frit. Then the resin was deprotected with 20% piperidin in NMP for 20 min and washed with approx. with NMP. Then amino acid 5 mmol Fmoc-Tyr(tbu)-OH was solubilised in 10 ml 0.5M HOAt in NMP and added to the resin. Then followed by addition of 5 mmol DIC and 1 mmol collidine and coupled for 30 min. Then excess amino acid was removed by washing with NMP and the Fmoc-group was removed by 20% piperidine in NMP for 15 min. Then the piperidin was removed by washing with NMP and the resin was ready for the next amino acid. The amino acids were added in a stepwise manner according to the previously described SPPS synthesis method to give the final peptide sequence. Finally, the N-alpha amino was protected with a Boc group. Optionally, for PYY(3-36) in position 13 the Ser was replaced by a Lys(Mtt) onto which the albumin handles were attached.

### Synthesis of Albumin Handles on Peptide

The protected peptidyl resin was swelled in neat hexafluoroisopropanol (HFIP) approx. 30 ml for 2 min followed by another addition of HFIP and let stand for 5 min. A third addition was performed and let stand for 20 min. Then the resin was washed with NMP and briefly with 20% piperidine in NMP and the again NMP to remove piperidine. Then Fmoc-Oeg (NeoMPS) was added 3 mmol in 6 ml 0.5M HOAt solution in NMP and 3 mmol DIC was added and let stand for 2 hours. Then washed and deprotected with 20% piperidine in NMP and washed followed by another addition of Fmoc-Oeg as mentioned above. Then after deprotection and washing 3 mmol Fmoc-L-Glu-tBu (IRIS-Biotech, Germany) in 6 ml 0.5 M HOAt solution in NMP was added followed by addition of 3 mmol DIC and let stand for approx. 19 hours. The after removing the Fmoc-group 3 mmol of the residue Fmoc-tranexamic acid (NeoMPS) in 6 ml 0.5M HOAt solution was added followed by 3 mmol DIC and let stand for >2 hours. After coupling the resin was washed and Fmoc was removed by 20% piperidine in NMP and after NMP washing 3 mmol mono-tertbutyl-dodecanedioic acid in 6 ml 0.5 M HOAt solution was added followed by 3 mmol DIC and let stand for >16 hours. The resin was washed with NMP and diethylether and dried.

### Final Deprotection and Isolation

The peptide and side chain protection groups were removed by addition of 30 ml 92% TFA, 5% TIPS and 3% ethanol for approx. 2 hours. Then TFA was collected and concentrated by a stream of argon and diethylether was added to precipitate the peptide. The peptide was washed five times with ether and dried.

### HPLC Analysis

### HPLC Analysis Method I:

Buffer A: 0.1% TFA in water

### Buffer B: 0.1% in AcCN

Gradient: 0% buffer B to 90% buffer B in 50 min.
Flow: 0.5 ml/min

### Column: Jubitor Proteo C12, 4.6×250 mm,

Column temperature: 42° C.

### HPLC Analysis Method II

Buffer A: 0.5M ammoniumbicarbonate in 90% water/10% AcCN
Buffer B: 70% AcCN/30% water
Gradient 25% buffer B to 55% in 16 min.
Flow: 0.4 ml/min

### Column: Acquity UPLC HSS T3, 1.8 um, 2.1×150 mm

Column temperature: 30° C.

### Example 1

### Manuel Synthesis of SEQ ID NO: 1, SEQ ID NO: 3, SEQ ID NO: 4, SEQ ID NO: 13

The syntheses were carried out using the above mentioned methods “Synthesis of peptidyl resin”, “Synthesis of albumin handles on peptide” and “Final deprotection and isolation”.

### Analytical Data

### SEQ ID NO: 1

Retention time HPLC method I: 25.9 min
Retention time HPLC method II: 5.6 min
Mw calculated: 4049.6 g/mol
MALDI MS: 4046.4 g/mol

### SEQ ID NO: 3

Retention time HPLC method I: 33.1 min
Retention time HPLC method II: 11.5 min
Mw calculated: 4973.8 g/mol
MALDI MS: 4972.3 g/mol

### SEQ ID NO: 13

Retention time HPLC method I: 34.0 min
Retention time HPLC method II: 10.2 min
Mw calculated: 4932.7 g/mol
MALDI MS: 4931.7 g/mol

### Example 2

### Automated Synthesis of SEQ ID NO: 2, SEQ ID NO: 6, SEQ ID NO: 7, SEQ ID NO: 8 and SEQ ID NO: 9

The syntheses were carried out as described in SPPS Method II using 0.5 g Tentagel HL RAM resin (Rapp Polymere, Germany) on a Liberty peptide synthesizer. After synthesis on the Liberty apparatus, the resin was transferred to a 50 ml syringe with filter frit. The albumin handles was synthesised using the above mentioned method “Synthesis of albumin handles on peptide”. The resin was then treated with 90% TFA, 5% TIPS and 5% water and precipitated in Et2O as described before.

### Biological Assays

The utility of the PYY or PP peptide derivatives or analogues thereof of the present invention as pharmaceutically active agents in the reduction of weight gain and treatment of obesity in mammals (such as humans), may be demonstrated by the activity of the agonists in conventional assays and in the in vitro and in vivo assays described below.

Such assays also provide a means whereby the activities of the PYY or PP peptide derivatives or analogues thereof of this invention can be compared with the activities of known compounds.

### Example 3

### Receptor Potency of PYY and PP Analogues

Receptor potency of PYY and PP derivatives and analogues thereof was determined using the method “Measuring Y2 or Y4 Receptor Activity Using ACTOne Based FLIPR Assay” as described herein. Results are shown in Table 1 and Table 2.

TABLE 1
The activity of PYY analogues in Y2 and Y4 receptor ACTOne assays
as a function of acylation position and type of albumin handle.

| | Acylation position, | | |
|---|---|---|---|
| | fatty acid chain | Y2 cAMP | Y4 cAMP |
| Compound | length | EC50 (nM) | EC50 (nM) |
| SEQ ID NO: 1 | none | 0.7 | 500 |
| SEQ ID NO: 3 | K13, C20 | 24 | >1000 |
| SEQ ID NO: 12 | K4, C20 | 10 | 235 |
| SEQ ID NO: 19 | K13, C20 | 9 | 67 |
| SEQ ID NO: 20 | K13, C20 | 118 | 25 |
| SEQ ID NO: 21 | K13, 20 | 42 | 53 |
| SEQ ID NO: 22 | K13, C20 | 79 | 9 |
| SEQ ID NO: 23 | Nalfa, C20 | 1 | 80 |
| SEQ ID NO: 27 | K11, C18 | 6 | >1000 |
| SEQ ID NO: 28 | K11; C20 | 24 | >1000 |
| SEQ ID NO: 33 | Nalfa, C20 | 97 | >1000 |
| SEQ ID NO: 34 | K25, C20 | 46 | ND |
| SEQ ID NO: 35 | K24, C20 | 12 | ND |
| SEQ ID NO: 40 | K19, C20 | 4 | 690 |
| SEQ ID NO: 51 | K13, Tetrazole | 29 | >1000 |
| SEQ ID NO: 52 | K25, Tetrazole | 71 | >1000 |
| SEQ ID NO: 53 | Nalfa, Tetrazole | 1.2 | >1000 |
| SEQ ID NO: 54 | K19, Tetrazole | 2 | >1000 |
| SEQ ID NO: 57 | K18, C20 | 4.3 | 100 |
| SEQ ID NO: 58 | K22, C20 | 5 | >1000 |
| SEQ ID NO: 59 | K26, C20 | 19 | >1000 |
| SEQ ID NO: 60 | K29, C20 | >1000 | >1000 |
| SEQ ID NO: 61 | K36, C20 | >1000 | >1000 |
| SEQ ID NO: 62 | K21, C20 | 5.2 | >1000 |
| SEQ ID NO: 63 | K30, C20 | 5.8 | 53 |
| SEQ ID NO: 64 | K31, C20 | 7.1 | >1000 |
| SEQ ID NO: 66 | K15, C18 | 14 | >1000 |
| SEQ ID NO: 67 | K16, C18 | 12 | >1000 |
| SEQ ID NO: 68 | K20, C18 | 16 | >1000 |
| SEQ ID NO: 69 | K21, C18 | 21 | >1000 |
| SEQ ID NO: 70 | K32, C18 | 49 | >1000 |

ND = Not determined.

TABLE 2
The activity of PP analogues in Y2 and Y4 receptor ACTOne assays
as a function of acylation position and type of albumin handle.

| | Acylation position, | | |
|---|---|---|---|
| | fatty acid chain | Y2 cAMP | Y4 cAMP |
| Compound | length | EC50 (nM) | EC50 (nM) |
| SEQ ID NO: 2 | none | >1000 | 0.6 |
| SEQ ID NO: 4 | K13, C20 | 54 | 18 |
| SEQ ID NO: 15 | K13, C20 | 73 | 7 |
| SEQ ID NO: 16 | K13, C20 | 63 | 65 |
| SEQ ID NO: 17 | K13, C20 | 130 | 7 |
| SEQ ID NO: 14 | K13, C20 | 92 | 18 |
| SEQ ID NO: 18 | K13, C20 | >1000 | 130 |
| SEQ ID NO: 24 | K13, C18 | 32 | 2 |
| SEQ ID NO: 29 | K11, C20 | >1000 | 2 |
| SEQ ID NO: 30 | K11, C18 | >1000 | 1 |
| SEQ ID NO: 31 | K13, C18 | >1000 | 4 |
| SEQ ID NO: 32 | K18, C18 | >1000 | 0.4 |
| SEQ ID NO: 39 | K10, C20 | 25 | ND |
| SEQ ID NO: 41 | K33, C18 | >1000 | >1000 |
| SEQ ID NO: 42 | K33, C20 | >1000 | >1000 |
| SEQ ID NO: 43 | K18, C20 | >1000 | 0.8 |
| SEQ ID NO: 44 | K29, C20 | >1000 | >890 |
| SEQ ID NO: 45 | K26, C20 | >1000 | 7 |
| SEQ ID NO: 46 | K26, C18 | >1000 | 5 |
| SEQ ID NO: 47 | K35, C18 | >1000 | >1000 |
| SEQ ID NO: 48 | K35, C20 | >1000 | >1000 |
| SEQ ID NO: 49 | K25, C18 | >1000 | >1000 |
| SEQ ID NO: 50 | K25, C20 | >1000 | >1000 |
| SEQ ID NO: 55 | Nalfa, C18 | >1000 | 1 |
| SEQ ID NO: 56 | Nalfa, C20 | >1000 | 4 |
| SEQ ID NO: 73 | none | >1000 | 0.9 |
| SEQ ID NO: 74 | K13, C20 | 29 | 11 |
| SEQ ID NO: 75 | Nalfa, C20 | >1000 | 3.2 nM |

ND = Not determined.

### Example 4

### Quantitative Assay for Plasma Samples

For determination of plasma concentration of the PYY and PP peptide derivative or analogues thereof the following Methods 1-4 were used. Table 3 shows which method was used for which compound.

TABLE 3

| | Method used for determination |
|---|---|
| Compound | of plasma concentration |
| SEQ ID NO: 1 | Method 1 |
| SEQ ID NO: 3 | Method 4 |
| SEQ ID NO: 12 | Method 2 |
| SEQ ID NO: 23 | Method 2 |
| SEQ ID NO: 27 | Method 2 |
| SEQ ID NO: 28 | Method 2 |
| SEQ ID NO: 34 | Method 3 |
| SEQ ID NO: 35 | Method 3 |
| SEQ ID NO: 40 | Method 2 |
| SEQ ID NO: 51 | Method 2 |
| SEQ ID NO: 52 | Method 2 |
| SEQ ID NO: 53 | Method 2 |
| SEQ ID NO: 54 | Method 2 |
| SEQ ID NO: 57 | Method 3 |
| SEQ ID NO: 58 | Method 3 |
| SEQ ID NO: 59 | Method 3 |
| SEQ ID NO: 2 | Method 4 |
| SEQ ID NO: 24 | Method 2 |
| SEQ ID NO: 29 | Method 2 |
| SEQ ID NO: 30 | Method 2 |
| SEQ ID NO: 32 | Method 3 |
| SEQ ID NO: 39 | Method 3 |
| SEQ ID NO: 41 | Method 3 |
| SEQ ID NO: 42 | Method 3 |
| SEQ ID NO: 43 | Method 2 |
| SEQ ID NO: 44 | Method 3 |
| SEQ ID NO: 45 | Method 2 |
| SEQ ID NO: 46 | Method 2 |
| SEQ ID NO: 47 | Method 2 |
| SEQ ID NO: 48 | Method 2 |
| SEQ ID NO: 49 | Method 2 |
| SEQ ID NO: 50 | Method 2 |
| SEQ ID NO: 55 | Method 2 |
| SEQ ID NO: 56 | Method 2 |
| SEQ ID NO: 71 | Method 2 |
| SEQ ID NO: 72 | Method 2 |
| SEQ ID NO: 73 | Method 2 |
| SEQ ID NO: 74 | Method 2 |
| SEQ ID NO: 75 | Method 2 |

### Method 1

Plasma samples were analysed by LC-MS on an LTQ-Orbitrap (ThermoFisher Scientific, Bremen) to which Accela HPLC pumps and an autosampler were connected (both from ThermoFisher). The mass spectrometer was equipped with an electrospray interface, which was operated in positive ionisation mode. Analysis was conducted in selected ion monitoring mode at m/z 829.8±1.5 Da. The compound was detected at 829.4529 Da, which corresponded to [M+6H]6+ with an accuracy of 3.6 ppm. For quantification purposes, the six most intense isotope peaks were extracted with an accuracy of 5 ppm. HPLC was performed on a Jupiter Proteo column (4μ) 90A (50×2.0 mm ID). Mobile phases consisted of A. 0.1% formic acid and B. 0.1% formic acid in acetonitrile. A gradient was run from 10% B to 20% B from 0 to 0.2 min and then from 20% B to 34% B from 0.2 min to 6 min. The flow rate was 0.3 ml/min. For analysis of plasma samples, 30 μl plasma was precipitated with 90 μl ethanol. To 100 μl of the supernatant, 20 μl 95% acetonitrile (containing 5% formic acid) and 200 μl heptane were added. The heptane phase was removed after 5 min and the remaining solution was analysed by LC-MS as described above. For construction of plasma standards, compound was spiked to plasma (minipig) at the following concentrations: 1 nM, 2 nM, 5 nM, 10 nM, 20 nM, 50 nM, 100 nM, 200 nM. The plasma standards were treated as the samples. The lower limit of quantification was estimated to 2 nM.

### Method 2

The test substances (various PYY and PP compounds) were assayed in plasma by Turbulent Flow Chromatography coupled to Liquid Chromatography with subsequent Tandem Mass Spectrometric Detection (TFC/LC/MS/MS). Positive mode ionization and Multiple Reaction Monitoring (MRM) of a multiple protonated species fragmented to a singly charged ion was employed for selectivity. The selectivity of the method allows up to four compounds to be quantified in one sample, e.g. cassette dosing of up to four per animal.

The concentrations of the test substance in unknown samples were calculated using the peak area as a function of amount. Calibration graphs based on plasma samples spiked with the analyte were constructed by regression analysis. Typical dynamic range for standard assay was 1-2,000 nmol/l. The method performance was assured by co-assaying quality control (QC) samples in duplicate at three concentration levels.

Stock and working solutions of analytes were prepared in plasma and incubated by 37° C. for 1 hour.

Sample Preparation: 40.0 μl EDTA-plasma was added 160 μl 50% methanol, 1% formic acid, then centrifuged at 14300 rpm (16457 g) at 4° C. for 20 minutes. The supernatant was transferred to a 96 well plate, plates incubated with 0.4% BSA, 37° C. for ½ hour. Injection volume was 25 μl.

The analysis was carried out on a Sciex API 3000 mass spectrometer (MDS/Sciex, Concord, ON, Canada) using a TurbolonSpray interface. The TFC/LC system consisted of two Flux Rheos 2000 quaternary pumps, a Cohesive VIM module (Cohesive Technologies, Franklin, Mass., USA) and a CTC LC/PAL auto sampler (CTC Analytics, Zingen, Switzerland). For sample clean up a TurboFlow C8 column (0.5×50 mm) (Thermo Scientific, Franklin, Mass., USA) was used and the LC separation was done on a Proteo 4 μm column (2.0×50 mm) (Phenomenex, Torrance, Calif., USA). Eluents were isocratic and gradient combinations of methanol, acetonitril, Milli-Q water and formic acid.

### Method 3

The test substances (various PYY and PP compounds) were assayed in plasma by Turbulent Flow Chromatography coupled to Liquid Chromatography with subsequent Orbitrap Mass Spectrometric Detection (TFC/LC/MS). Positive mode ionization and accurate mass acquisition of a multiple protonated species was employed for selectivity. The selectivity of the method allows up to four compounds to be quantified in one sample, e.g. cassette dosing of up to four per animal.

The concentrations of the test substance in unknown samples were calculated using the peak area as a function of amount. Calibration graphs based on plasma samples spiked with the analyte were constructed by regression analysis. Typical dynamic range for standard assay was 1-2,000 nmol/l. The method performance was assured by co-assaying quality control (QC) samples in duplicate at three concentration levels.

Stock and working solutions of analytes were prepared in plasma and incubated by 37° C. for 1 hour.

Sample Preparation: 40.0 μl EDTA-plasma was added 160 μl 50% methanol, 1% formic acid, then vortexed and centrifuged at 14300 rpm (16457 g) at 4° C. for 20 minutes. The supernatant was transferred to a 96 well plate, plates incubated with 0.4% BSA, 37° C. for ½ hour. Injection volume was 25 μl.

The analysis was carried out on a LTQ Orbitrap Discovery mass spectrometer (Thermo Scientific, Bremen, Germany) using electrospray interface with heated probe. The TFC/LC system consisted of two Flux Rheos Allegro quaternary pumps, a VIM module (Thermo Scientific, Franklin, Mass., USA) and a CTC LC/PAL auto sampler (CTC Analytics, Zingen, Switzerland). For sample clean up a TurboFlow C8 column (0.5×50 mm) (Thermo Scientific, Franklin, Mass., USA) was used and the LC separation was done on a Proteo 4 μm column (2.0×50 mm) (Phenomenex, Torrance, Calif., USA). Eluents were isocratic and gradient combinations of methanol, acetonitril, Milli-Q water and formic acid.

### Method 4

Plasma samples were analysed by LC-MS on an LTQ-Orbitrap (ThermoFisher Scientific, Bremen) to which Accela HPLC pumps and an autosampler were connected (both from ThermoFisher). The mass spectrometer was equipped with an electrospray interface, which was operated in positive ionisation mode. Analysis was conducted in selected ion monitoring mode with a window of 5 Da of the most intense ion. For quantification purposes, the most intense isotope peaks were extracted with an accuracy of 5 ppm. HPLC was performed on a Jupiter Proteo column (4μ) 90A (50×2.0 mm ID). Mobile phases consisted of A. 0.1% formic acid and B. 0.1% formic acid in acetonitrile. A gradient was run from 5% B to 30% B (or 35% B) from 0-6 min. The flow rate was 0.3 ml/min. For analysis of plasma samples, 30 μl plasma was precipitated with 60 μl acetonitrile containing 1% formic acid. For construction of plasma standards, compound was spiked to plasma (minipig) at the following concentrations: 1 nM, 2 nM, 5 nM, 10 nM, 20 nM, 50 nM, 100 nM, 200 nM. The plasma standards were treated as the samples. The lower limit of quantification was estimated to about 1-2 nM.

### Example 5

### Mice Studies

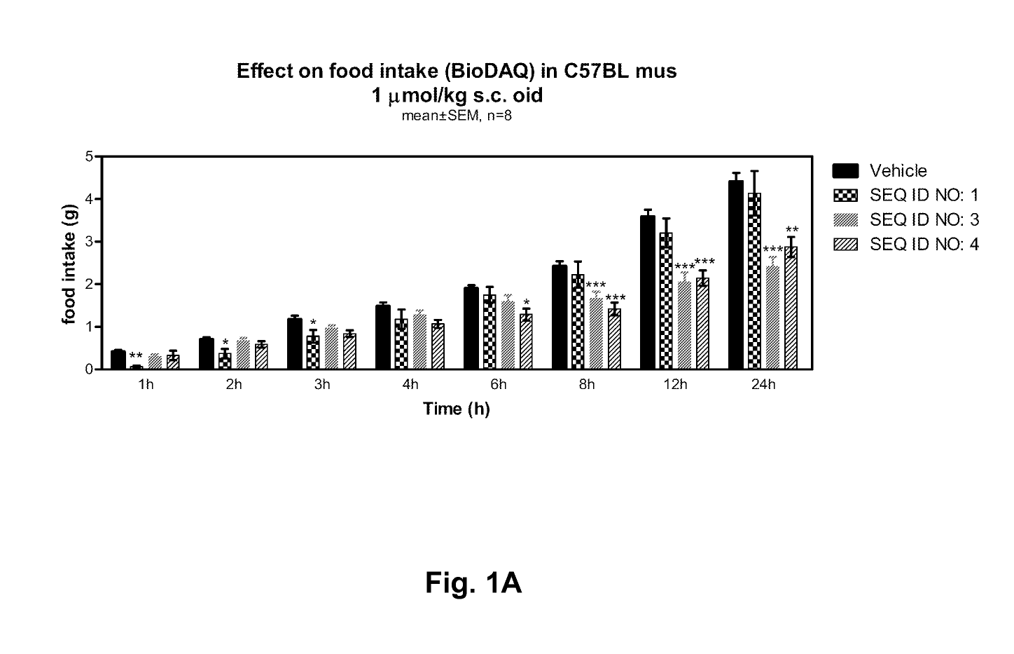

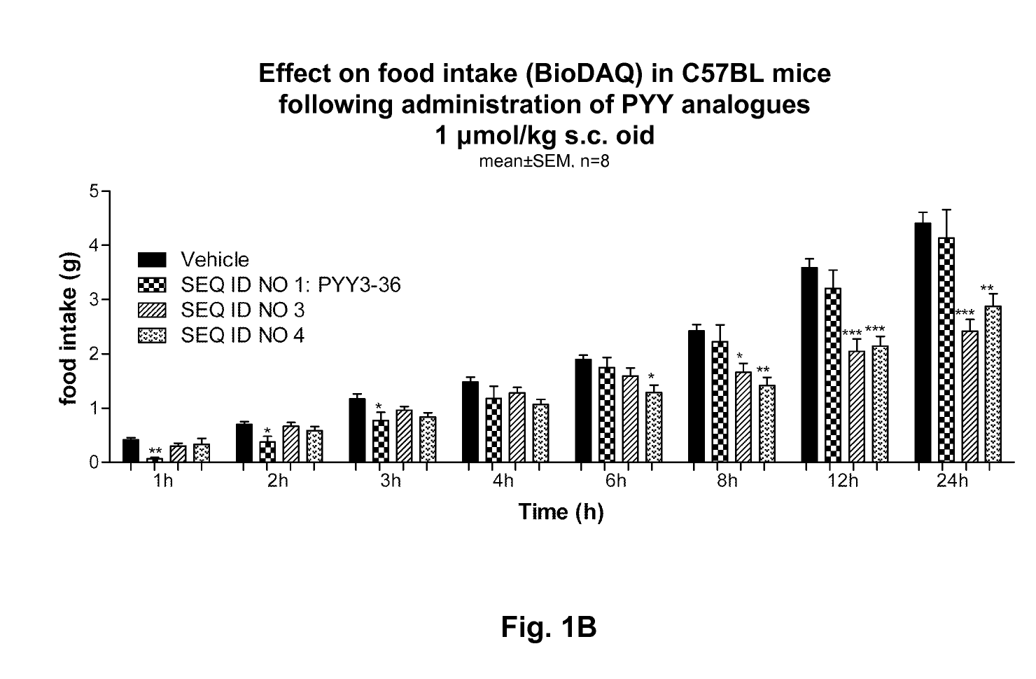

The effect on food intake of vehicle, SEQ ID NO: 1, SEQ ID NO: 3, SEQ ID NO: 4 were monitored in lean fasted-refed C57BL/6 mice. Mice were administrated a single dose of the peptides (1 μmol/kg s.c.) 30 min before food return and cumulative food intake was measured over 24 h. The results are shown in Table 4, FIGS. 1A and 1B. As seen in Table 4, FIGS. 1A and 1B the effect of protracted PYY(3-36) and PP analogues in reducing food-intake is prolonged compared to the effect of unmodified human PYY(3-36). Specifically, the effect of SEQ ID NO: 3 and SEQ ID NO: 4 (protracted PYY(3-36) and PP analogues, respectively) in reducing food-intake is prolonged compared to the effect of unmodified human PYY(3-36) (SEQ ID NO: 1). Whereas the effect of unmodified human PYY(3-36) in reducing food intake has disappeared 6 hours after administration the effect of the protracted PYY(3-36) and PP analogues persists 24 hours after administration of the peptides. Furthermore, an apparent delay in the onset of effect is observed for the protracted PYY(3-36) and PP analogues compared to unmodified human PYY(3-36) which may be consistent with a different pharmacokinetic profile for the protracted peptides. One way ANOVA was carried for each time point out using the software Graph-Pad Prism, version 5.0. The statistical method used for the data in FIG. 1A was an unpaired t-test using the software Graph-Pad Prism, version 5.0. The statistical method used for the data in FIG. 1B and Table 4 was ANOVA, Dunnetts post hoc. Stars in FIGS. 1A and 1B indicate significance versus the vehicle group *) p<0.05, **) p<0.01, ***) p<0.001.

TABLE 4

| | Mean cumulative food intake [g] | | | |
|---|---|---|---|---|
| | | SEQ ID | SEQ ID | SEQ ID |
| Time after | | NO: 1 | NO: 3 | NO: 4 |
| injection | vehicle | 1.0 μmol/kg | 1.0 μmol/kg | 1.0 μmol/kg |
| 1 hour | 0.41 | 0.06* | 0.30 | 0.33 |
| 2 hours | 0.70 | 0.37* | 0.67 | 0.59 |
| 3 hours | 1.17 | 0.78* | 0.97 | 0.84 |
| 4 hours | 1.48 | 1.18 | 1.28 | 1.07 |
| 6 hours | 1.90 | 1.75 | 1.59 | 1.29* |
| 8 hours | 2.42 | 2.23 | 1.67* | 1.42** |
| 12 hours | 3.59 | 3.21 | 2.05*** | 2.14*** |
| 24 hours | 4.41 | 4.14 | 2.42*** | 2.88** |

*p < 0.05,
**p < 0.01,
***p < 0.001 (ANOVA, Dunnetts post hoc)

### Example 6

### Mice Studies; Acute

Studies were conducted to evaluate the acute effects of PP and PYY analogues on food intake compared to vehicle. Fasted lean C57BL/6 mice were administrated a single subcutaneous injection of vehicle or peptide approximately 30 min before food return and the cumulative food intake was measured subsequently. One way ANOVA was carried out for each time point using the software Graph-Pad Prism, version 5.0.

### Study 6A

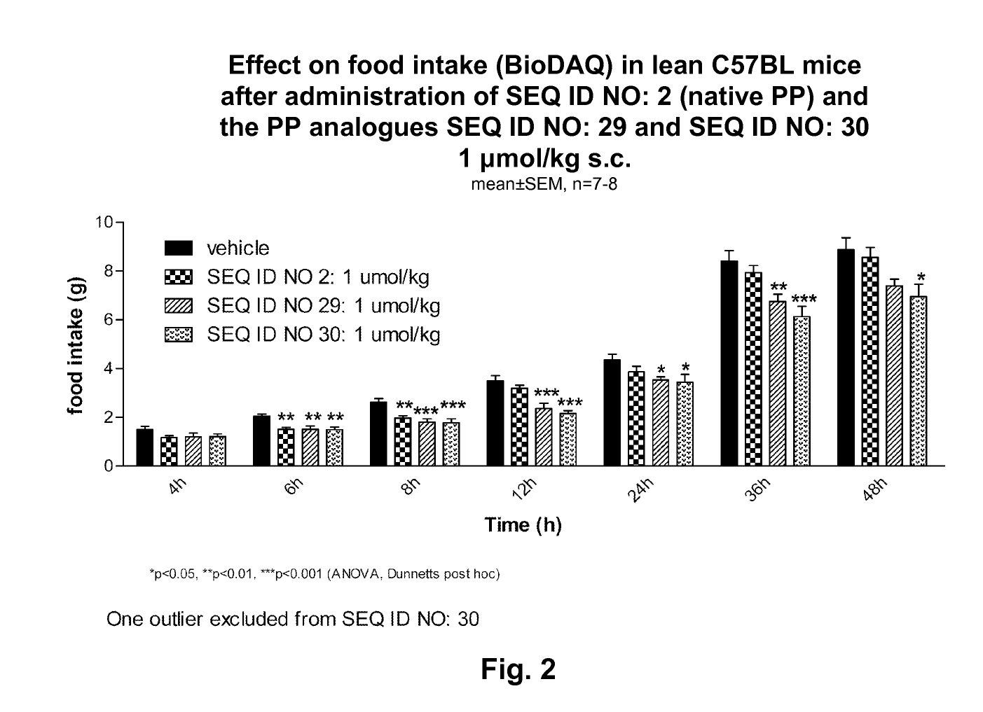

Mice were administered vehicle, hPP(1-36) or one of the two PP analogues SEQ ID NO: 29 and SEQ ID NO: 50. The peptide dose was (1.0 μmol/kg). Results are shown in Table 5 and in FIG. 2. As seen in Table 5 and in FIG. 2 the effect of protracted PP analogues in reducing food-intake is prolonged compared to the effect of unmodified human PP(1-36) SEQ ID NO: 2. Whereas the effect of unmodified human PP in reducing food intake has disappeared 12 hours after administration the effect of the protracted PP analogues persists 36 hours after administration of the peptides.

TABLE 5

| | Mean cumulative food intake [g] | | | |
|---|---|---|---|---|
| | | SEQ ID | SEQ ID | SEQ ID |
| Time after | | NO: 2 | NO: 29 | NO: 30 |
| injection | Vehicle | 1.0 μmol/kg | 1.0 μmol/kg | 1.0 μmol/kg |
| 4 hours | 1.51 | 1.18 | 1.20 | 1.21 |
| 6 hours | 2.04 | 1.51** | 1.51** | 1.50** |
| 8 hours | 2.61 | 1.98** | 1.81*** | 1.78*** |
| 12 hours | 3.49 | 3.19 | 2.36*** | 2.15*** |
| 24 hours | 4.35 | 3.87 | 3.53* | 3.44* |
| 36 hours | 8.41 | 7.93 | 6.78** | 6.13*** |
| 48 hours | 8.87 | 8.56 | 7.39 | 6.95* |

*p < 0.05,
**p < 0.01,
***p < 0.001 (ANOVA, Dunnetts post hoc)

### Study 6B

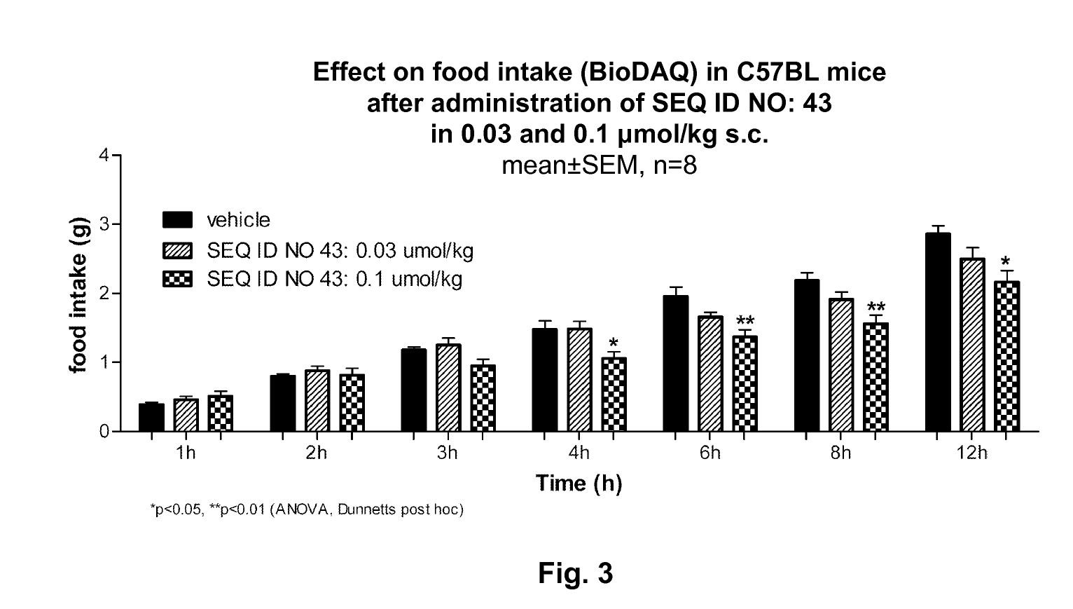

Mice were administered vehicle or the PP analogue SEQ ID NO: 43 in two different doses (0.03 μmol/kg and 0.1 μmol/kg). Results are shown in Table 6 and in FIG. 3. As seen in Table 6 and FIG. 3 the protracted PP analogue SEQ ID NO: 43 reduces food intake dose dependently resulting in reduced cumulative food intake 4-12 hours after injection. However, only the highest dose (0.1 μmol/kg) reached statistical significance.

TABLE 6

| | Mean cumulative food intake [g] | | |
|---|---|---|---|
| Time after | | SEQ ID NO: 43 | SEQ ID NO: 43 |
| injection | vehicle | 0.03 μmol/kg | 0.1 μmol/kg |
| 1 hour | 0.39 | 0.46 | 0.51 |
| 2 hours | 0.80 | 0.88 | 0.82 |
| 3 hours | 1.18 | 1.25 | 0.95 |
| 4 hours | 1.47 | 1.48 | 1.06* |
| 6 hours | 1.96 | 1.66 | 1.37** |
| 8 hours | 2.18 | 1.91 | 1.56** |
| 12 hours | 2.86 | 2.50 | 2.16* |

*p < 0.05,
**p < 0.01 (ANOVA, Dunnetts post hoc)

### Study 6C

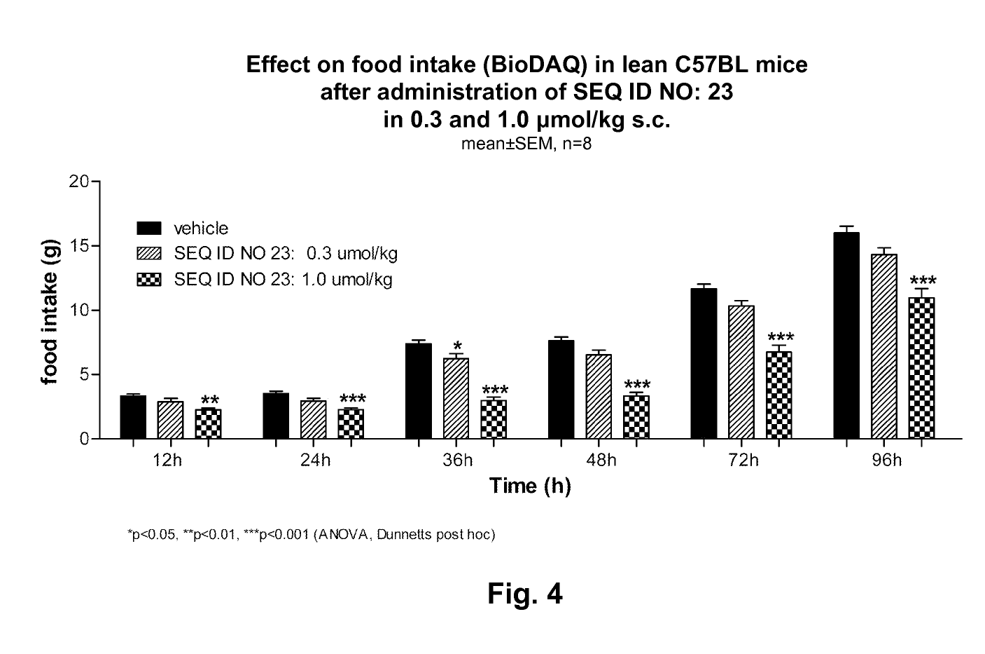

Mice were administered vehicle or the protracted PYY analogue SEQ ID NO: 23 in two different doses (0.3 μmol/kg and 1.0 μmol/kg). Results are shown in Table 7 and in FIG. 4. As seen in Table 7 and FIG. 4 the PYY analogue SEQ ID NO: 23 dose dependently reduces food intake resulting in statistical significant reduced cumulative food intake for up to 96 hours after injection.

TABLE 7

| | Mean cumulative food intake [g] | | |
|---|---|---|---|
| Time after | | SEQ ID NO: 23 | SEQ ID NO: 23 |
| injection | vehicle | 0.3 μmol/kg | 1.0 μmol/kg |
| 12 hours | 3.32 | 2.88 | 2.26** |
| 24 hours | 3.55 | 2.92 | 2.28*** |
| 36 hours | 7.37 | 6.24* | 2.98*** |
| 48 hours | 7.61 | 6.52 | 3.33*** |
| 72 hours | 11.64 | 10.34 | 6.74*** |
| 96 hours | 16.00 | 14.34 | 10.95*** |

*p < 0.05,
**p < 0.01,
***p < 0.001 (ANOVA, Dunnetts post hoc)

### Study 6D

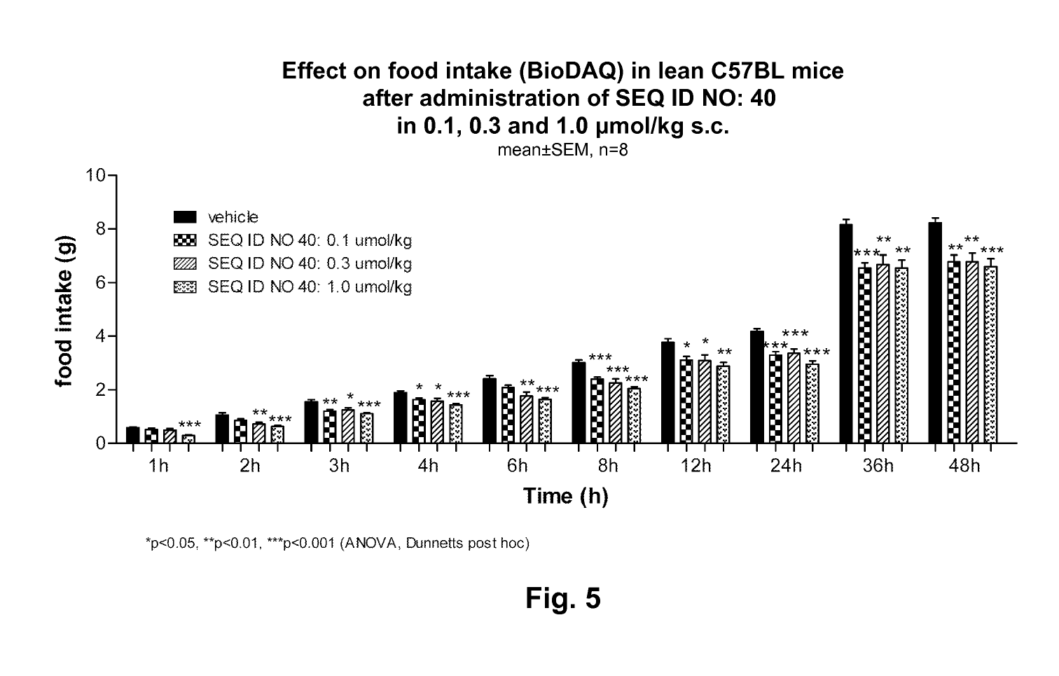

Mice were administered vehicle or the PYY analogue SEQ ID NO: 40 in three different doses (0.1, 0.3 μmol/kg and 1.0 μmol/kg). Results are shown in Table 8 and in FIG. 5. As seen in Table 8 and FIG. 5 the PYY analogue SEQ ID NO: 40 effectively reduced food intake in all three doses.

TABLE 8

| | | Mean cumulative food intake [g] | | | |
|---|---|---|---|---|---|
| | | | SEQ ID | SEQ ID | SEQ ID |
| Time after | | | NO: 40 | NO: 40 | NO: 40 |
| injection | | Vehicle | 0.1 μmol/kg | 0.3 μmol/kg | 1.0 μmol/kg |
| 1 | hour | 0.59 | 0.52 | 0.50 | 0.30*** |
| 2 | hours | 1.06 | 0.85 | 0.72** | 0.63*** |
| 3 | hours | 1.55 | 1.21** | 1.25* | 1.11*** |
| 4 | hours | 1.90 | 1.63* | 1.58* | 1.44*** |
| 6 | hours | 2.41 | 2.09 | 1.77** | 1.65*** |
| 8 | hours | 3.02 | 2.41*** | 2.26*** | 2.05*** |
| 12 | hours | 3.76 | 3.11* | 3.10* | 2.88** |
| 24 | hours | 4.18 | 3.29*** | 3.36*** | 2.97*** |
| 36 | hours | 8.15 | 6.55** | 6.68** | 6.55*** |
| 48 | hours | 8.23 | 6.78** | 6.78** | 6.60*** |

*p < 0.05,
**p < 0.01,
***p < 0.001 (ANOVA, Dunnetts post hoc)

### Study 6E

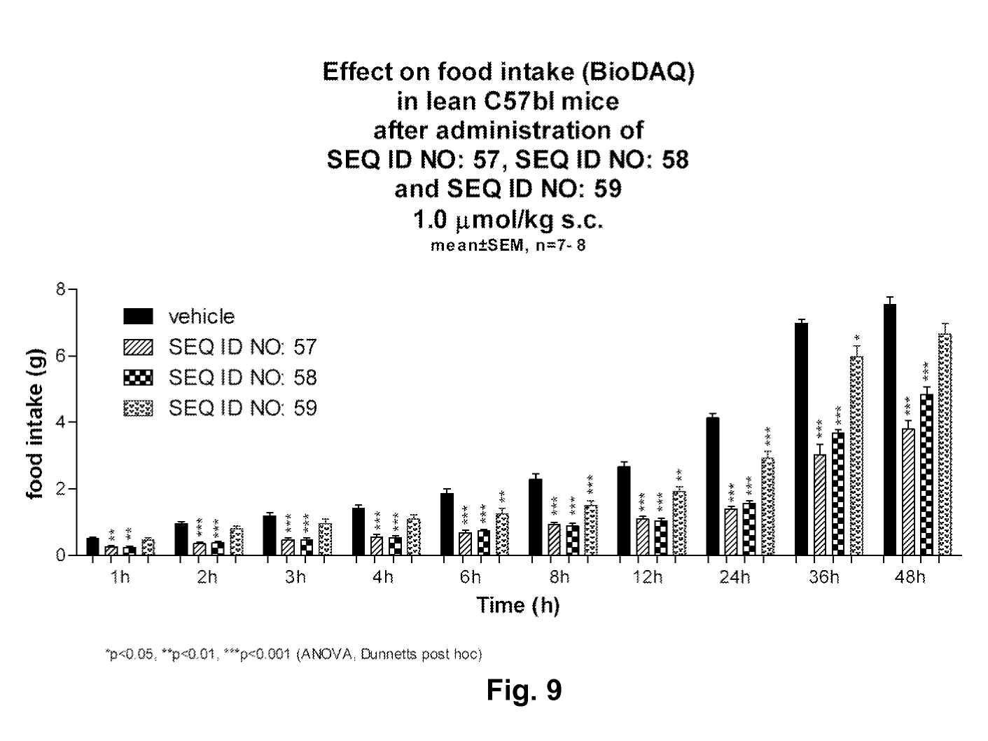

Mice were administered vehicle (n=8) or one of the PYY analogues SEQ ID NO: 57 (n=8), SEQ ID NO: 58 (n=7) and SEQ ID NO: 59 (n=8). The peptide dose was 1.0 μmol/kg. Results are shown in Table 9 and FIG. 9, as seen herein the PYY analogue SEQ ID NO: 57 and SEQ ID NO: 58 effectively reduced food intake resulting in statistical significant reduced cumulative food intake 1-48 hours after injection. The effect of the analogue SEQ ID NO: 59 was less pronounced resulting in statistical significant reduced cumulative food intake 6-36 hours after injection.

TABLE 9

| | | Mean cumulative food intake [g] | | | |
|---|---|---|---|---|---|
| | | | SEQ ID | SEQ ID | SEQ ID |
| Time after | | | NO: 57 | NO: 58 | NO: 59 |
| injection | | vehicle | 1.0 μmol/kg | 1.0 μmol/kg | 1.0 μmol/kg |
| 1 | hour | 0.50 | 0.25** | 0.22** | 0.47 |
| 2 | hours | 0.94 | 0.35*** | 0.37*** | 0.79 |
| 3 | hours | 1.18 | 0.46*** | 0.46*** | 0.95 |
| 4 | hours | 1.40 | 0.54*** | 0.52*** | 1.09 |
| 6 | hours | 1.85 | 0.68*** | 0.72*** | 1.25** |
| 8 | hours | 2.27 | 0.92*** | 0.88*** | 1.50*** |
| 12 | hours | 2.65 | 1.08*** | 1.03*** | 1.92** |
| 24 | hours | 4.12 | 1.39*** | 1.56*** | 2.91*** |
| 36 | hours | 6.96 | 3.02*** | 3.67*** | 5.98* |
| 48 | hours | 7.53 | 3.79*** | 4.83*** | 6.66 |

*p < 0.05,
**p < 0.01,
***p < 0.001 (ANOVA, Dunnetts post hoc)

### Study 6F

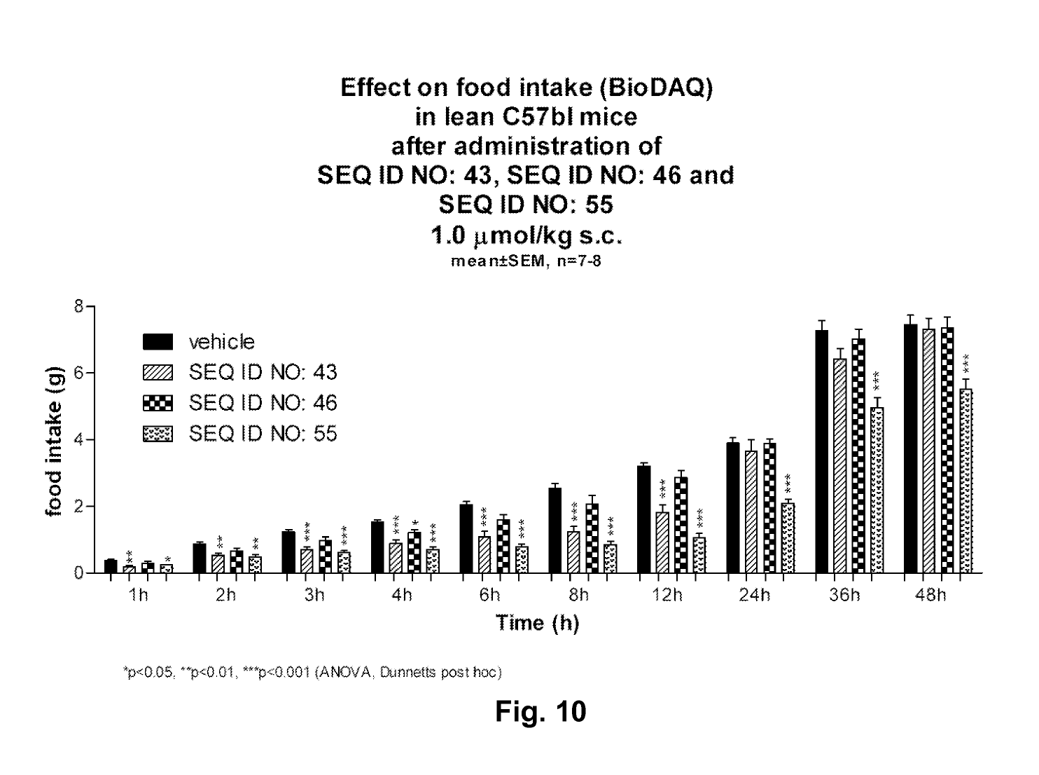

Mice were administered vehicle (n=8) or one of the PP analogues SEQ ID NO: 43 (n=8), SEQ ID NO: 46 (n=7), and SEQ ID NO: 55 (n=8). The peptide dose was 1.0 μmol/kg. Results are shown in Table 10 and in FIG. 10, as seen herein the protracted PP analogue SEQ ID NO: 43 and SEQ ID NO: 55 effectively reduced food intake resulting in statistical significant reduced cumulative food intake 1-12 hours (SEQ ID NO: 43) and 1-48 hours (SEQ ID NO: 55) after injection. The effect of the analogue SEQ ID NO: 46 was less pronounced resulting in statistical significant reduced cumulative food intake 4 hours after injection.

TABLE 10

| | | Mean cumulative food intake [g] | | | |
|---|---|---|---|---|---|
| | | | SEQ ID | SEQ ID | SEQ ID |
| Time after | | | NO: 43 | NO: 46 | NO: 55 |
| injection | | vehicle | 1.0 μmol/kg | 1.0 μmol/kg | 1.0 μmol/kg |
| 1 | hour | 0.38 | 0.20** | 0.30 | 0.24* |
| 2 | hours | 0.87 | 0.53** | 0.66 | 0.49** |
| 3 | hours | 1.23 | 0.70*** | 0.98 | 0.61*** |
| 4 | hours | 1.53 | 0.89*** | 1.20* | 0.70*** |
| 6 | hours | 2.05 | 1.09*** | 1.61 | 0.79*** |
| 8 | hours | 2.54 | 1.24*** | 2.07 | 0.86*** |
| 12 | hours | 3.21 | 1.81*** | 2.86 | 1.06*** |
| 24 | hours | 3.91 | 3.66 | 3.89 | 2.09*** |
| 36 | hours | 7.29 | 6.42 | 7.02 | 4.96*** |
| 48 | hours | 7.45 | 7.33 | 7.37 | 5.51*** |

*p < 0.05,
**p < 0.01,
***p < 0.001 (ANOVA, Dunnetts post hoc)

### Example 7

### Mice Studies; Chronic

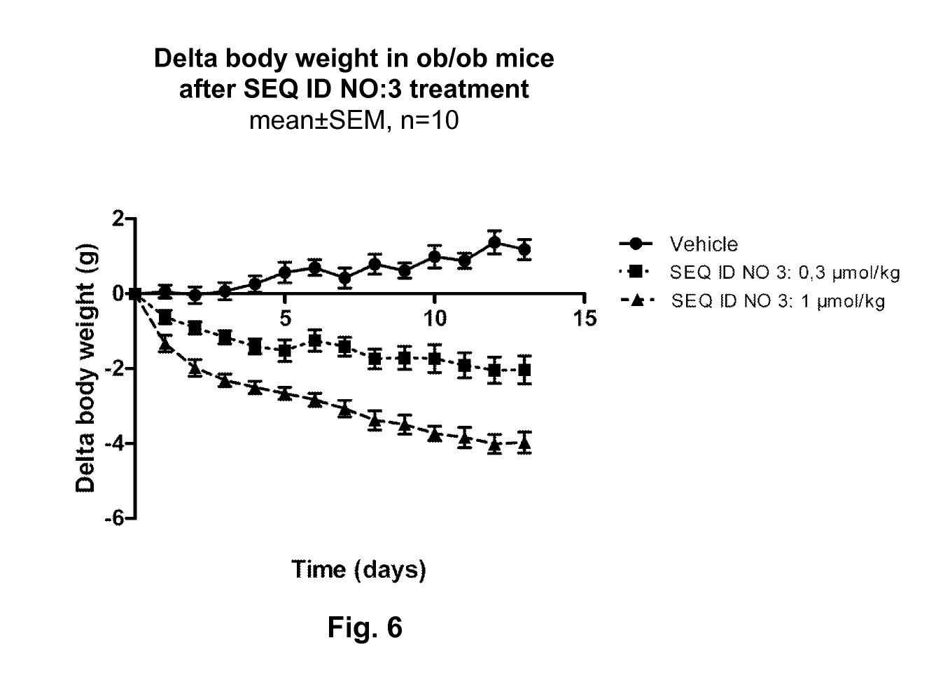

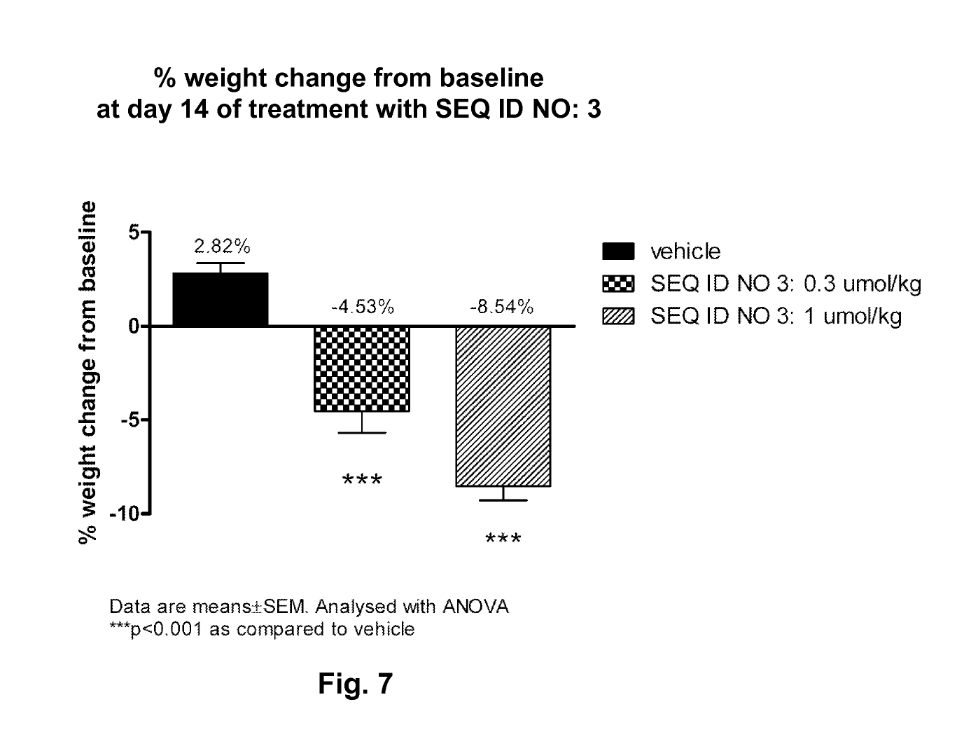

A chronic study was conducted to determine the effect of SEQ ID NO: 3 in two doses (0.3 μmol/kg and 1.0 μmol/kg) on body weight. Ob/ob mice were treated with one daily subcutaneous injection for two weeks. Results are shown in FIGS. 6 and 7. As seen in FIG. 6 body weight decreased dose dependently during the study period. After two weeks the body weight was statistical significantly reduced with 4.5% and 8.5% for animals treated with 0.3 and 1.0 μmol/kg, respectively (FIG. 7). The body weight in vehicle treated animals was increased by 2.8% during the study period (FIG. 7).

### Example 8

### Rat Studies; Acute

### Study 8A

A study was conducted to evaluate the acute effects of hPYY(3-36) (SEQ ID NO: 1) and a PYY analogue (SEQ ID NO: 3) on food intake compared to vehicle. Lean rats were dosed with a single subcutaneous injection of vehicle or peptide approximately 30 min before the light is turned off and the cumulative food intake was measured subsequently. Results are shown in Table 11. As seen in Table 11, treatment with the PYY analogue SEQ ID NO: 3 resulted in a statistical significant reduction in acute food intake in lean rats. In contrast, the effect of hPYY(3-36) was not statistically significant.

TABLE 11
Mean cumulative food intake

| | Mean cumulative food intake [g] | | |
|---|---|---|---|
| Time after | | SEQ ID NO: 3 | SEQ ID NO: 1 |
| injection | vehicle | 1.0 μmol/kg | 1.0 μmol/kg |
| 16 hours | 24.46 | 18.98*** | 22.74 |

***p < 0.001 (ANOVA, Dunnetts post hoc)

### Study 8B

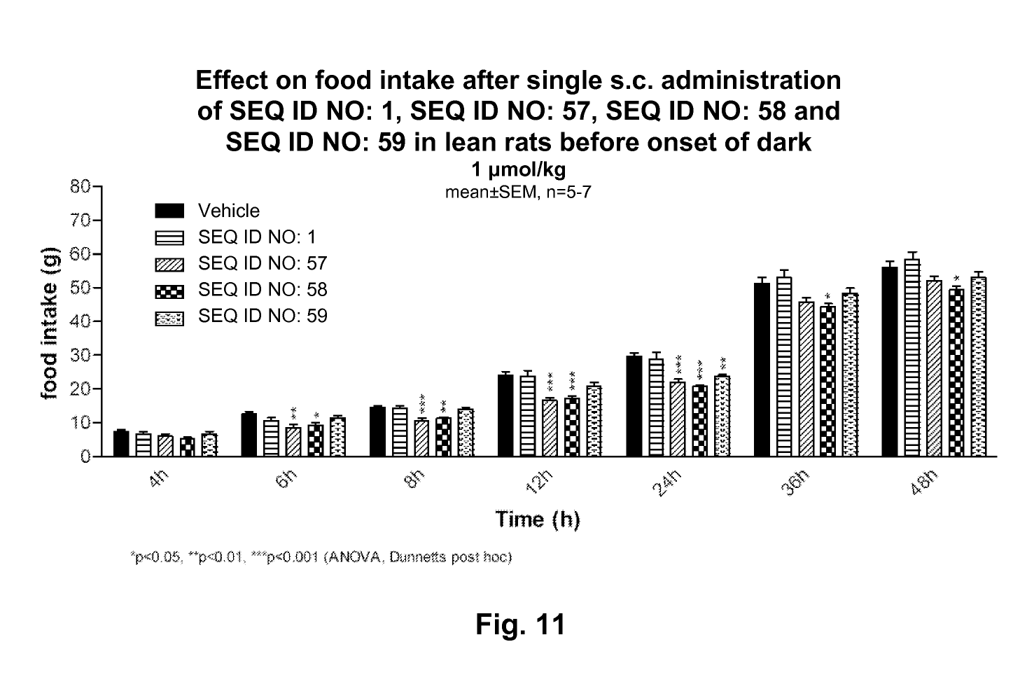

A study was conducted to evaluate the acute effect of native PYY 3-36 (SEQ ID NO: 1, n=5) and the PYY analogues SEQ ID NO: 57 (n=6), SEQ ID NO: 58 (n=5), and SEQ ID NO: 59 (n=5) on food intake compared to vehicle (n=7). Lean rats were dosed with a single subcutaneous injection of vehicle or peptide approximately 30 min before the light is turned off and the cumulative food intake was measured subsequently. Results are shown in Table 12 and FIG. 11, as seen herein treatment with native PYY (SEQ ID NO: 1) had no effect on food intake in lean rats. In contrast, treatment with the PYY analogues SEQ ID NO: 57 and SEQ ID NO: 58 resulted in statistical significant reductions in acute food intake. The cumulative food intake was reduced in 6-24 hours and in 6-48 hours after dosing of SEQ ID NO: 57 and SEQ ID NO: 58 respectively. The effect of the analogue SEQ ID NO: 59 was less pronounced resulting in statistical significant reduced cumulative food intake 24 hours after injection.

TABLE 12

| | Mean cumulative food intake [g] | | | | |
|---|---|---|---|---|---|
| Time after | | SEQ ID NO: 1 | SEQ ID NO: 57 | SEQ ID NO: 58 | SEQ ID NO: 59 |
| injection | vehicle | 1.0 μmol/kg | 1.0 μmol/kg | 1.0 μmol/kg | 1.0 μmol/kg |
| 4 hours | 7.38 | 6.72 | 5.97 | 5.17 | 6.71 |
| 6 hours | 12.65 | 10.72 | 8.54** | 9.17* | 11.45 |
| 8 hours | 14.57 | 14.36 | 10.63*** | 11.24** | 13.98 |
| 12 hours | 24.14 | 23.86 | 16.59*** | 17.23*** | 20.97 |
| 24 hours | 29.68 | 28.88 | 22.01*** | 20.79*** | 23.86** |
| 36 hours | 51.26 | 52.97 | 45.76 | 44.36* | 48.50 |
| 48 hours | 56.08 | 58.53 | 52.10 | 49.27* | 53.04 |

*p < 0.05,
**p < 0.01,
***p < 0.001 (ANOVA, Dunnetts post hoc)

### Example 9

### Pig Studies; Acute

A study was conducted to evaluate the acute effect of the SEQ ID NO: 23 on food intake in pigs. Pigs were dosed with a single subcutaneous injection of vehicle (n=3) or peptide (n=4) and the cumulative food intake was measured subsequently. Results are shown in Table 13, as seen herein treatment with 30 nmol/kg of SEQ ID NO: 23 resulted in a statistical significant reduction in cumulative food intake 12 hours after dosing.

TABLE 13

Mean cumulative food intake [kg]

| Time after | | SEQ ID NO: 23 |
|---|---|---|
| injection | vehicle | 30 nmol/kg |
| 12 hours | 1.34 | 0.98* |

*p < 0.05 (t-test)

### Example 10

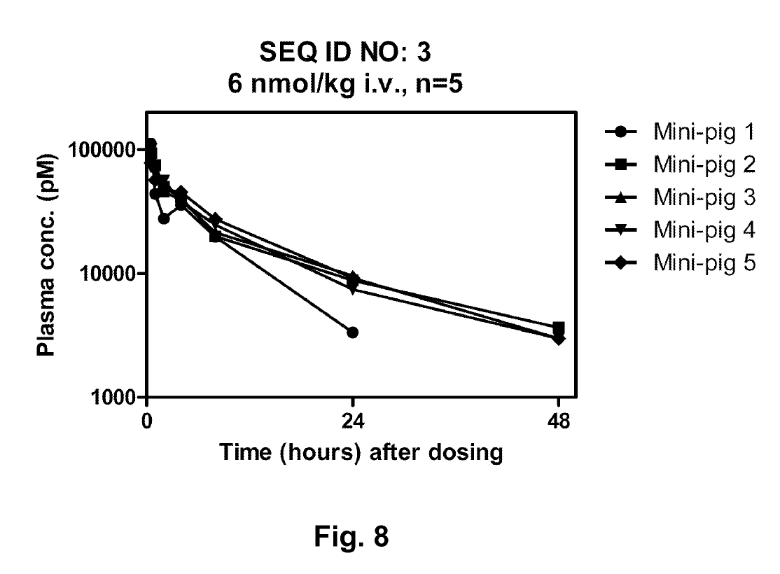

Determination of the PK profile of SEQ ID NO: 3 in mini-pigs. Five Göttingen mini-pigs were administrated a single i.v. bolus dose of SEQ ID NO: 3; blood samples were taken at the indicated time points and the plasma concentration of SEQ ID NO: 3 was determined by LC/MS as described herein. The mean terminal plasma half-life (t1/2) of SEQ ID NO: 3 was calculated to be 12±4 hours by non-compartmental analysis of the plasma concentration-time profiles as described herein. The results are shown in FIG. 8. Thus, the half-life of SEQ ID NO: 3 is considerably prolonged compared to the reported half-life of <30 minutes for unmodified PYY(3-36) in pigs (Ito T et al, Journal of Endocrinology (2006), 191, pp 113-119).

### Example 11

An assay useful for measuring PK of the compounds of the invention is the minipig PK assay. Male Göttingen mini-pigs (n≧3) weighing approximately 15 to 35 kg from Ellegaard Göttingen Minipigs A/S, Denmark were included in the study. The mini-pigs had two central venous catheters inserted which were used for intra venous (i.v.) dosing and bloodsampling. The compound was dissolved in 50 mM K₂HPO₄, 0.05% tween 80, pH=8.0 to an appropriate concentration (e.g. 25-500 nmol/mL). The pigs were dosed intravenously (i.v.) or subcutaneously (s.c.) with between 1 and 30 nmol compound/kg body weight. Blood samples were taken at the following at appropriate time points, such as pre-dose, 30 minutes, 1, 2, 4, 8, 24, 48, 72, 96, 120, 168, 240 and 288 hours post dosing. The blood samples were collected into test tubes containing EDTA buffer (with Aprotinin 15000 KIE/mL and Val-Pyr 0.30 mM) for stabilization and kept on ice for max. 20 minutes before centrifugation. The centrifugation procedure to separate plasma was; 4° C., 3000 rpm for 10 minutes. Plasma were collected and immediately transferred to Micronic tubes stored at −20° C. until assayed.

An additional mini-pig PK assay was used for measuring PK of the compounds of the invention. Mini-pigs weighing 15 to 35 kg from Ellegaard Göttingen Minipigs A/S were included in the studies. The animals had two central venous catheters inserted which were used for intra venous (i.v.) dosing and blood sampling. Compounds were dissolved in 10 mM Na₂HPO₄, 150 mM NaCl, 0.01% tween 80, pH=4.0 to concentrations in the range of 40 nmol/ml to 200 nmol/ml. The mini-pigs were dosed i.v. with 10 nmol compound/kg body weight, occasionally other doses such as 4 nmol/kg, 30 nmol/kg or 50 nmol/kg were administered. Each compound was dosed to 3 or 4 mini-pigs, and two compounds may be given simultaneously to the same animal. Blood were sampled pre-dose and 12 times during the first 10 hours post-dose. Blood were furthermore sampled once daily up till 13 days post dosing. The blood samples were collected into test tubes containing EDTA buffer, trasylol and Val-Pyr for stabilization and kept on ice for max. 20 minutes. Samples were centrifuged at 4° C., 2000G for 10 minutes to separate plasma. Plasma were collected and immediately transferred to Micronic tubes stored at −20° C. until assayed.

Results are shown in Table 14 for PYY analogues or derivatives thereof.

Results are shown in Table 15 for PP analogues or derivatives thereof.

TABLE 14
Half-life (t½) of PYY analogues or derivatives thereof tested
in minipigs as a function of acylation position and type of albumin handle

| | Acylation position, | | | Dose | t½ |
|---|---|---|---|---|---|
| Compound | fatty acid chain length | RoA¹ | n | (nmol/kg) | (hr) |
| SEQ ID NO: 1 | Native (3-36) | i.v. | 4 | 16 | 0.20 |
| | | i.v. | 3 | 47 | 0.49 |
| SEQ ID NO: 3 | K13, C20 | i.v. | 5 | 6 | 13 |
| | | s.c. | 4 | 25 | 23 |
| SEQ ID NO: 12 | K4, C20 | i.v. | 3 | 8 | 8.0 |
| SEQ ID NO: 23 | Nα, C20 | i.v. | 3 | 9.4 | 8.5 |
| | | s.c. | 4 | 28 | 30 |
| SEQ ID NO: 27 | K11, C18 | i.v. | 3 | 5.6 | 4.3 |
| SEQ ID NO: 28 | K11, C20 | i.v. | 3 | 5.6 | 2.0 |
| SEQ ID NO: 34 | K25, C20 | i.v. | 4 | 4 | 45 |
| | | i.v. | 4 | 31 | 66 |
| SEQ ID NO: 35 | K24, C20 | i.v. | 4 | 4 | 50 |
| SEQ ID NO: 40 | K19, C20 | i.v. | 4 | 29 | 27 |
| SEQ ID NO: 51 | K13, tetrazol | i.v. | 3 | 10 | 18 |
| SEQ ID NO: 52 | K25, tetrazol | i.v. | 3 | 10 | 65 |
| SEQ ID NO: 53 | Nα, tetrazol | i.v. | 3 | 10 | 11 |
| SEQ ID NO: 54 | K19, tetrazol | i.v. | 3 | 10 | 30 |
| SEQ ID NO: 57 | K18, C20 | i.v. | 4 | 10 | 22 |
| SEQ ID NO: 58 | K22, C20 | i.v. | 4 | 10 | 34 |
| SEQ ID NO: 59 | K26, C20 | i.v. | 4 | 10 | 30 |

TABLE 15
Half-life (t½) of PP analogues or derivatives thereof tested
in minipigs as a function of acylation position and type of albumin handle

| | Acylation position, | | | | |
|---|---|---|---|---|---|
| | fatty acid chain | | | Dose | t½ |
| Compound | length | RoA¹ | n | (nmol/kg) | (hr) |
| SEQ ID NO: 2 | Native PP(1-36) | i.v. | 4 | 15 | 0.035 |
| | | i.v. | 3 | 45 | 0.031 |
| SEQ ID NO: 29 | K10, C20 | i.v. | 3 | 1 | 23 |
| | | i.v. | 3 | 30 | 27 |
| SEQ ID NO: 74 | K13, C20 | i.v. | 4 | 16 | 8 |
| SEQ ID NO: 24 | K13 | i.v. | 4 | 20 | 3.5 |
| SEQ ID NO: 30 | K10, C18 | i.v. | 3 | 4.4 | 22 |
| SEQ ID NO: 73 | none | i.v. | 4 | 50 | 0.55 |
| SEQ ID NO: 32 | K18, C18 | i.v. | 4 | 12 | 46 |
| SEQ ID NO: 71 | K25, C20 | i.v. | 4 | 13 | 13 |
| SEQ ID NO: 72 | K15, C20 | i.v. | 1 | 8 | 14 |
| SEQ ID NO: 39 | K10, C20 | i.v. | 2 | 4 | 32 |
| SEQ ID NO: 41 | K33, C18 | i.v. | 1 | 1.4 | 105 |
| | | s.c. | 2 | 1.4 | 93 |
| SEQ ID NO: 42 | K33, C20 | i.v. | 2 | 7 | 116 |
| | | s.c. | 2 | 7 | 167 |
| SEQ ID NO: 43 | K18, C20 | i.v. | 4 | 13.5 | 55 |
| | | s.c. | 2 | 1.7 | Below |
| | | | | | LoQ² |
| SEQ ID NO: 44 | K29, C20 | i.v. | 2 | 4 | 74 |
| | | s.c. | 2 | 4 | 93 |
| SEQ ID NO: 45 | K26, C20 | i.v. | 3 | 4 | 58 |
| SEQ ID NO: 46 | K26, C18 | i.v. | 3 | 4 | 62 |
| SEQ ID NO: 47 | K35, C18 | i.v. | 4 | 10 | 78 |
| SEQ ID NO: 48 | K35, C20 | i.v. | 3 | 10 | 93 |
| SEQ ID NO: 49 | K25, C18 | i.v. | 3 | 10 | 88 |
| SEQ ID NO: 50 | K25, C20 | i.v. | 3 | 10 | 82 |
| SEQ ID NO: 55 | Nα, C18 | i.v. | 4 | 7 | 29 |
| SEQ ID NO: 56 | Nα, C20 | i.v. | 1 | 6 | 22 |
| SEQ ID NO: 75 | Nα, C20 | i.v. | 3 | 10 | 48 |

¹RoA: route of administration
²LoQ: limit of quantification

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

1. A PYY or PP peptide derivative or analogue thereof, wherein at least one amino acid residue and/or the N- and/or C-terminus of the peptide backbone is derivatised with a serum albumin binding side chain defined by A-B-C-D-, A-C-D-, A-B-C-, or A-C-, wherein
A- is 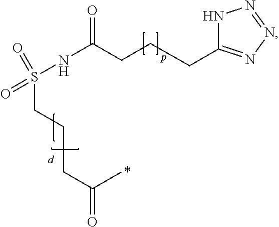
wherein p is selected from the group consisting of 10, 11, 12, 13, 14, 15 and 16 and d is selected from the group consisting of 0, 1, 2, 3, 4 and 5,
and -B- is selected from the group consisting of 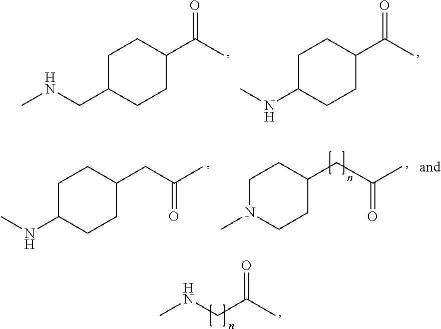
wherein x is selected from the group consisting of 0, 1, 2, 3 and 4, and y is selected from the group consisting of 1, 2, 3, 4, 5, 6, 7, 8, 9, 10, 11 and 12;
or A- is 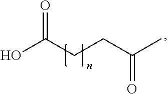
wherein n is selected from the group consisting of 12, 13, 14, 15, 16 17, 18 and 19,
and -B- is selected from the group consisting of 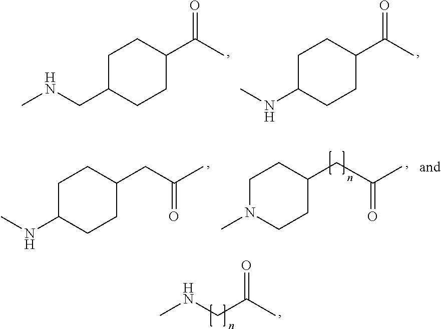
wherein x is selected from the group consisting of 0, 1, 2, 3 and 4; and
-C- is selected from the group consisting of 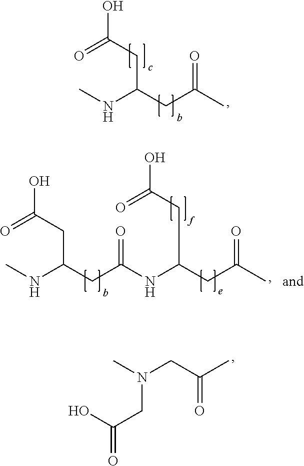
wherein b and e are each independently selected from the group consisting of 0, 1, and 2,
and c and f are each independently selected from the group consisting of 0, 1, and 2
with the proviso that when
c is 0 b is 1 or 2,
c is 1 or 2 b is 0,
f is 0 e is 1 or 2,
f is 1 or 2 e is 0, and
with the proviso that when A- is 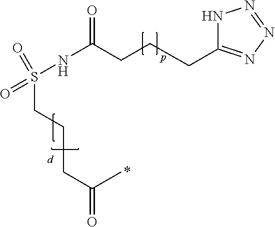
-C- may be deleted; and
-D- is attached to said amino acid residue and is a spacer.

2. A PYY or PP peptide derivative or analogue thereof according to claim 1, wherein the peptide is selected from the group consisting of
a PP analogue according to formula I
(I)
Z-Ala-Pro-Leu-Glu-Pro-Val-Tyr-Pro-Gly-Xaa₁₀-Xaa₁₁-
Xaa₁₂-Xaa₁₃-Xaa₁₄-Xaa₁₅-Xaa₁₆-Xaa₁₇-Xaa₁₈-Xaa₁₉-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Thr-Arg-Xaa₃₄-Arg-Xaa₃₆,
wherein
Z is the side chain A-B-C-D-, A-C-D-, A-B-C-, or A-C- attached to the N-terminal amino group, or not present when A-B-C-D-, A-C-D-, A-B-C-, A-C- is attached to the side chain of an amino acid,
Ala in position 1 may be deleted,
Xaa₁₀ is Asp, Asn, 2,3-diaminopropionic acid, 2,4-diaminobutyric acid, ornithine, or Lys,
Xaa₁₁ is Asp, Asn, 2,3-diaminopropionic acid, 2,4-diaminobutyric acid, ornithine, or Lys,
Xaa₁₂ is Ala, 2,3-diaminopropionic acid, 2,4-diaminobutyric acid, ornithine, or Lys,
Xaa₁₃ is Thr, 2,3-diaminopropionic acid, 2,4-diaminobutyric acid, ornithine, or Lys,
Xaa₁₄ is Pro or hydroxyproline,
Xaa₁₅ is Glu, 2,3-diaminopropionic acid, 2,4-diaminobutyric acid, ornithine, or Lys,
Xaa₁₆ is Gln, 2,3-diaminopropionic acid, 2,4-diaminobutyric acid, ornithine, or Lys,
Xaa₁₇ is Leu, Val, Ile, homoleucine, norleucine, (1-aminocyclopentyl) carboxylic acid, (1-aminocyclohexyl) carboxylic acid, or 1-aminobutyric acid,
Xaa₁₈ is Ala, 2,3-diaminopropionic acid, 2,4-diaminobutyric acid, ornithine, or Lys,
Xaa₁₉ is Gln, 2,3-diaminopropionic acid, 2,4-diaminobutyric acid, ornithine, or Lys,
Xaa₂₀ is Tyr, Phe, or 3-pyridylalanine,
Xaa₂₁ is Ala, 2,3-diaminopropionic acid, 2,4-diaminobutyric acid, ornithine, or Lys,
Xaa₂₂ is Ala, 2,3-diaminopropionic acid, 2,4-diaminobutyric acid, ornithine, or Lys,
Xaa₂₃ is Asp, 2,3-diaminopropionic acid, 2,4-diaminobutyric acid, ornithine, or Lys,
Xaa₂₄ is Leu, Val, Ile, homoleucine, norleucine, (1-aminocyclopentyl) carboxylic acid, (1-aminocyclohexyl) carboxylic acid, or 1-aminobutyric acid,
Xaa₂₅ is Arg, 2,3-diaminopropionic acid, 2,4-diaminobutyric acid, ornithine, or Lys,
Xaa₂₆ is Arg, His, 2,3-diaminopropionic acid, 2,4-diaminobutyric acid, ornithine, or Lys,
Xaa₂₇ is Tyr, Phe, homoPhe, or 3-pyridylalanine,
Xaa₂₈ is Ile, Val, Leu, homoleucine, norleucine, (1-aminocyclopentyl) carboxylic acid, (1-aminocyclohexyl) carboxylic acid, or 1-aminobutyric acid,
Xaa₂₉ is Asn, Gln, or Lys,
Xaa₃₀ is Met, Leu, Val, Ile, homoleucine, norleucine, (1-aminocyclopentyl) carboxylic acid, (1-aminocyclohexyl) carboxylic acid, or 1-aminobutyric acid,
Xaa₃₁ is Leu, Val, Ile, homoleucine, norleucine, (1-aminocyclopentyl) carboxylic acid, (1-aminocyclohexyl) carboxylic acid, or 1-aminobutyric acid,
Arg in position 33 may be substituted with Lys,
Xaa₃₄ is Gln, Asn, or His,
Arg in position 35 may be substituted with Lys,
Xaa₃₆ is Tyr, 3-pyridylalanine;
a PYY analogue according to formula II
(II)
Z-Tyr-Pro-Xaa₃-Xaa₄-Pro-Glu-Ala-Pro-Gly-Xaa₁₀-
Xaa₁₁-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Thr-Arg-Xaa₃₄-Arg-
Xaa₃₆,
wherein
Z is the side chain A-B-C-D-, A-C-D-, A-B-C-, or A-C- attached to the N-terminal amino group, or not present when A-B-C-D-, A-C-D-, A-B-C-, A-C- is attached to the side chain of an amino acid,
Tyr-Pro in position 1 and 2 may be deleted,
Tyr in position 1 may be substituted with Ala or may be deleted,
Xaa₃ is Ile, Val, Leu, (1-aminocyclopentyl) carboxylic acid, (1-aminocyclohexyl) carboxylic acid, or 1-aminobutyric acid,
Xaa₄ is Glu, 2,3-diaminopropionic acid, 2,4-diaminobutyric acid, ornithine, or Lys, Glu in position 6 may be substituted with Val,
Ala in position 7 may be substituted with Tyr,
Xaa₁₀ is Glu, 2,3-diaminopropionic acid, 2,4-diaminobutyric acid, ornithine, or Lys,
Xaa₁₁ is Asp, 2,3-diaminopropionic acid, 2,4-diaminobutyric acid, ornithine, or Lys,
Xaa₁₂ is Ala, 2,3-diaminopropionic acid, 2,4-diaminobutyric acid, ornithine, or Lys,
Xaa₁₃ is Ser, 2,3-diaminopropionic acid, 2,4-diaminobutyric acid, ornithine, or Lys,
Xaa₁₄ is Pro, hydroxyproline, or Lys,
Xaa₁₅ is Glu, 2,3-diaminopropionic acid, 2,4-diaminobutyric acid, ornithine, or Lys,
Xaa₁₆ is Glu, 2,3-diaminopropionic acid, 2,4-diaminobutyric acid, ornithine, or Lys,
Xaa₁₇ is Leu, Val, Ile, homoleucine, norleucine, (1-aminocyclopentyl) carboxylic acid, (1-aminocyclohexyl) carboxylic acid, or 1-aminobutyric acid,
Xaa₁₈ is Asn, 2,3-diaminopropionic acid, 2,4-diaminobutyric acid, ornithine, or Lys,
Xaa₁₉ is Arg, 2,3-diaminopropionic acid, 2,4-diaminobutyric acid, ornithine, or Lys,
Xaa₂₀ is Tyr, Phe, 3-pyridylalaine, 2,3-diaminopropionic acid, 2,4-diaminobutyric acid, ornithine, or Lys,
Xaa₂₁ is Tyr, Phe, 3-pyridylalaine, 2,3-diaminopropionic acid, 2,4-diaminobutyric acid, ornithine, or Lys,
Xaa₂₂ is Asp, 2,3-diaminopropionic acid, 2,4-diaminobutyric acid, ornithine, or Lys,
Xaa₂₃ is Ala, 2,3-diaminopropionic acid, 2,4-diaminobutyric acid, ornithine, or Lys,
Xaa₂₄ is Leu, Ile, Val, homoleucine, norleucine, (1-aminocyclopentyl) carboxylic acid, (1-aminocyclohexyl) carboxylic acid, 1-aminobutyric acid, or Lys,
Xaa₂₅ is Arg, 2,3-diaminopropionic acid, 2,4-diaminobutyric acid, ornithine, or Lys,
Xaa₂₆ is His, Arg, 2,3-diaminopropionic acid, 2,4-diaminobutyric acid, ornithine, or Lys,
Xaa₂₇ is Tyr, Phe, homoPhe, or 3-pyridylalanine,
Xaa₂₈ is Ile, Val, Leu, homoleucine, norleucine, (1-aminocyclopentyl) carboxylic acid, (1-aminocyclohexyl) carboxylic acid, 1-aminobutyric acid, or Lys,
Xaa₂₉ is Asn, Gln, or Lys,
Xaa₃₀ is Met, Leu, Val, Ile, homoleucine, norleucine, (1-aminocyclopentyl) carboxylic acid, (1-aminocyclohexyl) carboxylic acid, 1-aminobutyric acid, or Lys,
Xaa₃₁ is Leu, Val, Ile, homoleucine, norleucine, (1-aminocyclopentyl) carboxylic acid, (1-aminocyclohexyl) carboxylic acid, 1-aminobutyric acid, or Lys,
Thr in position 32 may be substituted with Lys,
Xaa₃₄ is Gln, Asn, or His,
Xaa₃₆ is Tyr, 3-pyridylalanine, or Lys;
wherein the compound is modified with a serum albumin binding side chain comprising a distal carboxylic acid or tetrazole group.

3. A PYY or PP peptide derivate or analogue thereof according to claim 1, wherein the serum albumin binding side chain is attached to an amino group of the side chain of an amino acid of the peptide backbone selected from the group consisting of 2,3-diaminopropionic acid, 2,4-diaminobutyric acid, ornithine, and Lys.

4. A PYY or PP peptide derivative or analogue thereof according to claim 1, wherein the spacer, -D-, comprises one or more 8-amino-3,6-dioxaoctanoic acid (Oeg) molecules.

5. A PYY or PP peptide derivative or analogue thereof according to claim 1, wherein said derivative is selective for the Y2 and/or Y4 receptors over the Y1 receptor.

6. A PYY or PP peptide derivative or analogue thereof according to claim 1, wherein said derivative is selective for the Y2 and/or Y4 receptors over the Y5 receptor.

7. A PYY or PP peptide derivative or analogue thereof according to claim 1, wherein said derivative shows improved PK profile compared to human PYY, PYY(3-36), or PP.

8. A PYY or PP peptide derivative or analogue thereof according to claim 1, wherein said derivative shows protracted properties compared to human PYY, PYY(3-36), or PP.

9. A PYY or PP peptide derivative or analogue thereof according to claim 1, wherein said derivative shows improved half life in vivo compared to human PYY, PYY(3-36), or PP.

10. A PYY or PP peptide derivative or analogue thereof according to claim 1, selected from the group consisting of
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13]hPYY(3-36) 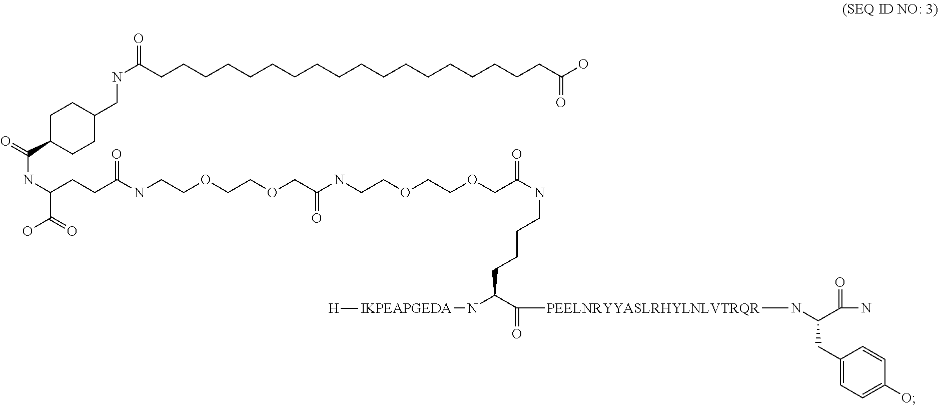
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,Leu17,Leu30,Gln34]hPP(1-36) 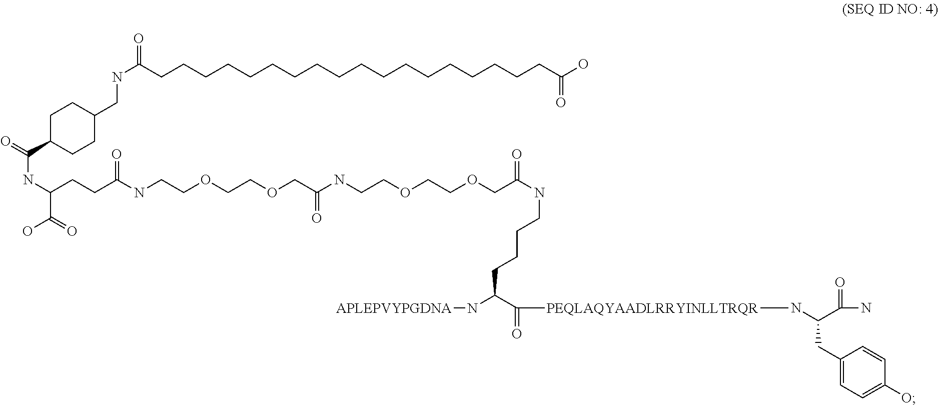
N-epsilon13-[2-(2-{2-[2-(2-{2-[(S)-4-Carboxy-4-(17-carboxyheptadecanoylamino)butyrylamino]ethoxy}ethoxy)-acetylamino]ethoxy}ethoxy)acetyl][Lys13]hPYY(3-36) 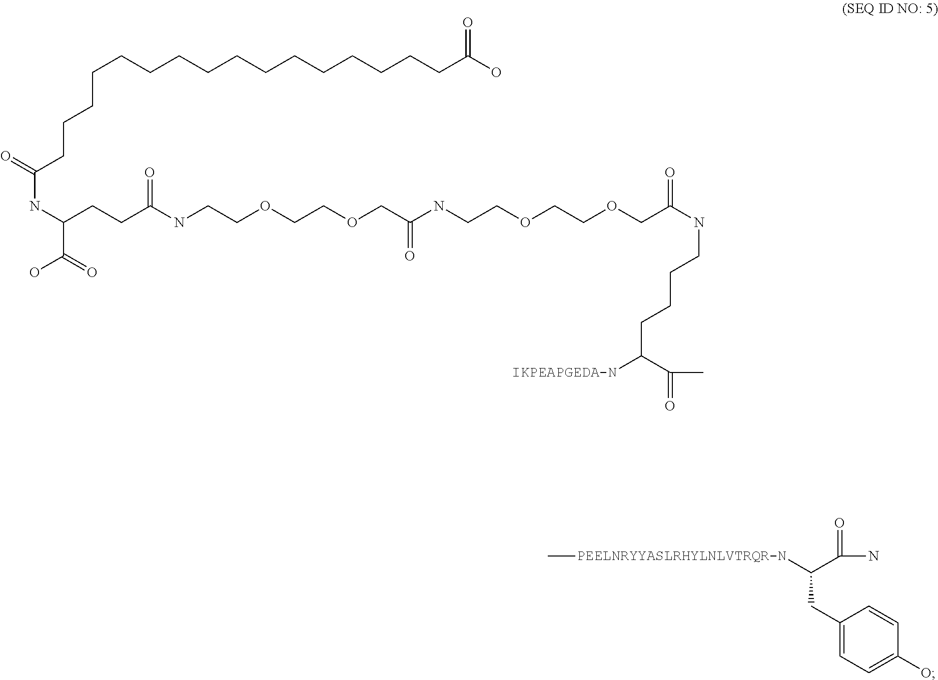
N-epsilon10-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys10,Leu17,Leu30]hPP2-36 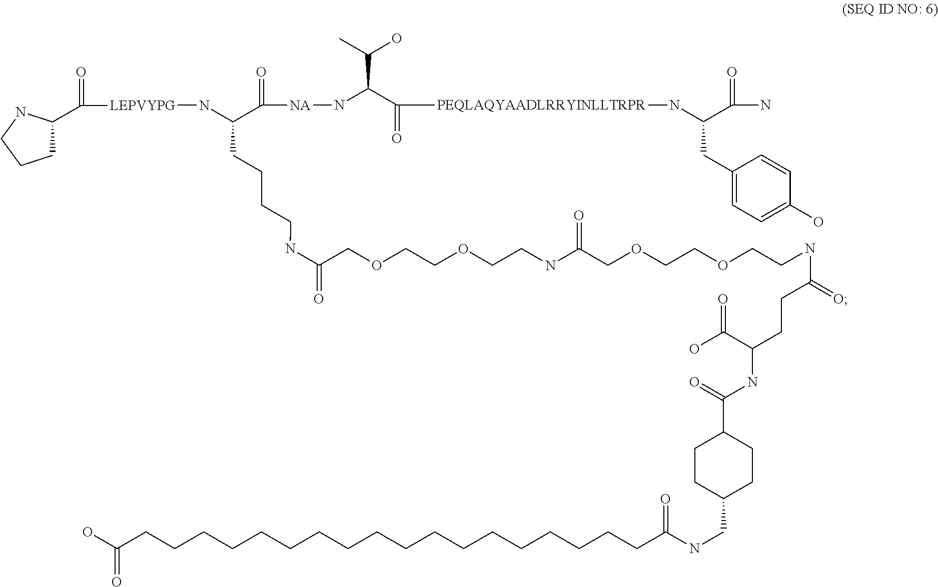
N-epsilon10-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys10,Leu17,Leu30]hPP2-36 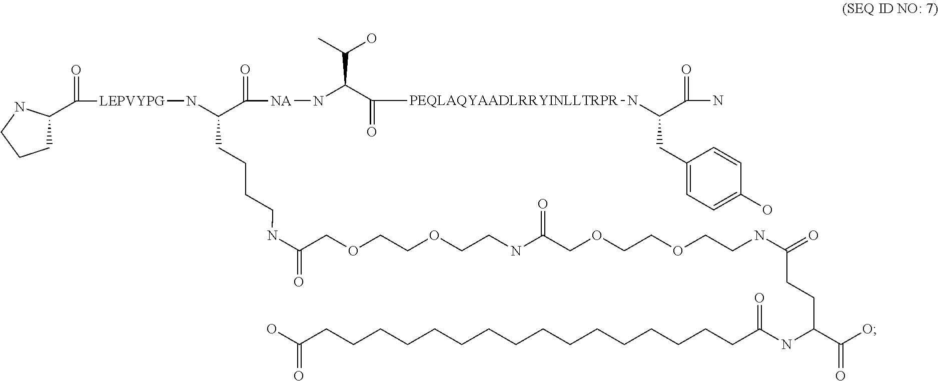
N-epsilon11-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys11]hPYY(3-36) 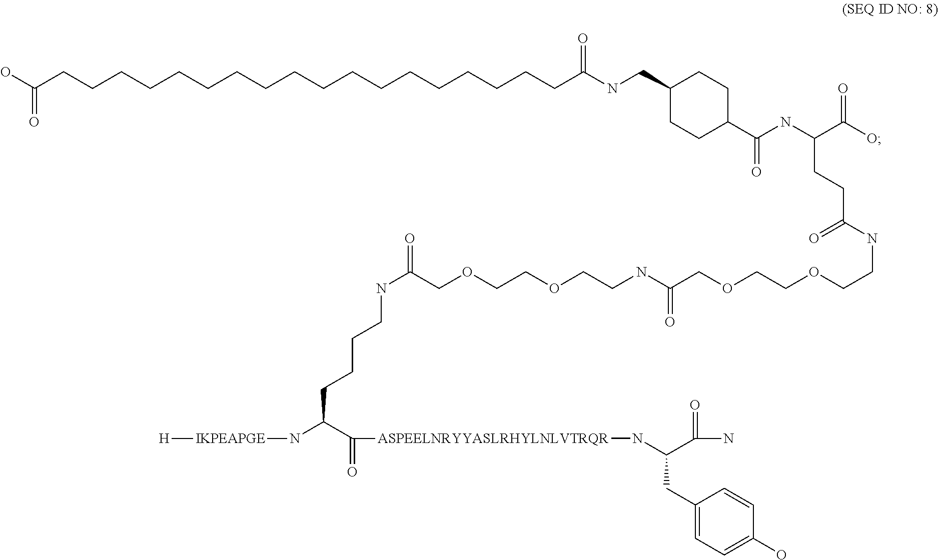
N-epsilon11-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys 11]hPYY(3-36) 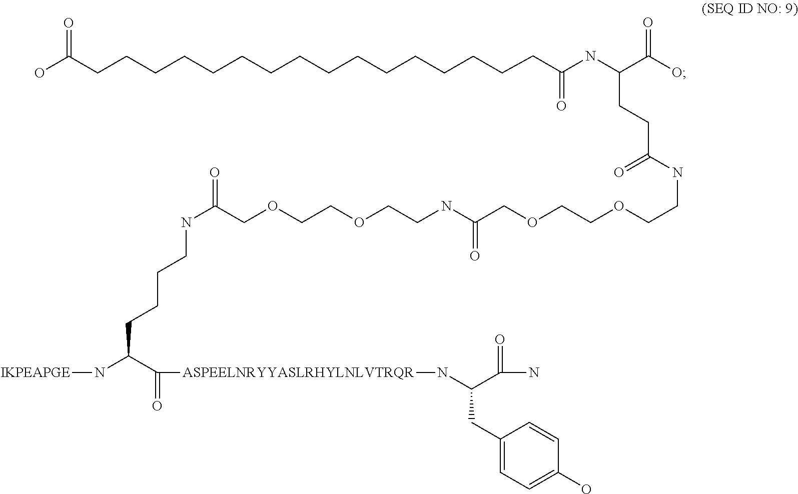
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,Leu17,Nle30,Gln34]hPP(1-36) 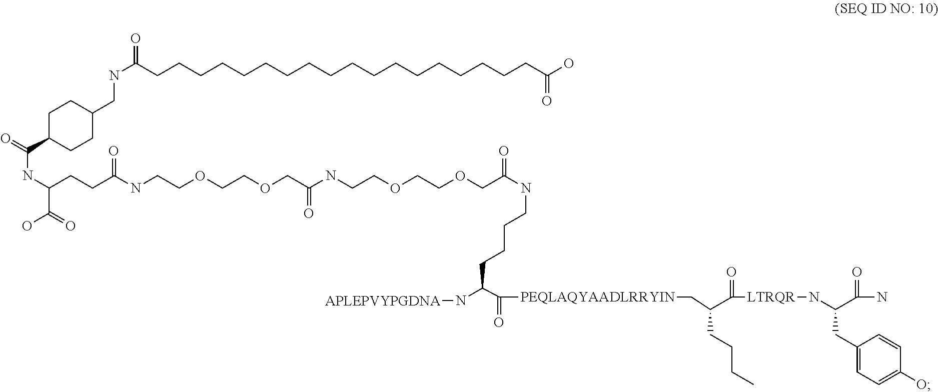
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13]hPYY2-36 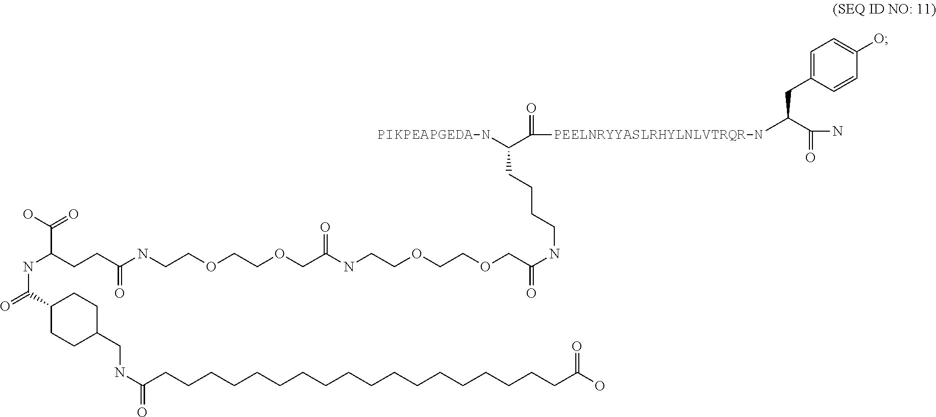
N-epsilon4-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY(3-36) 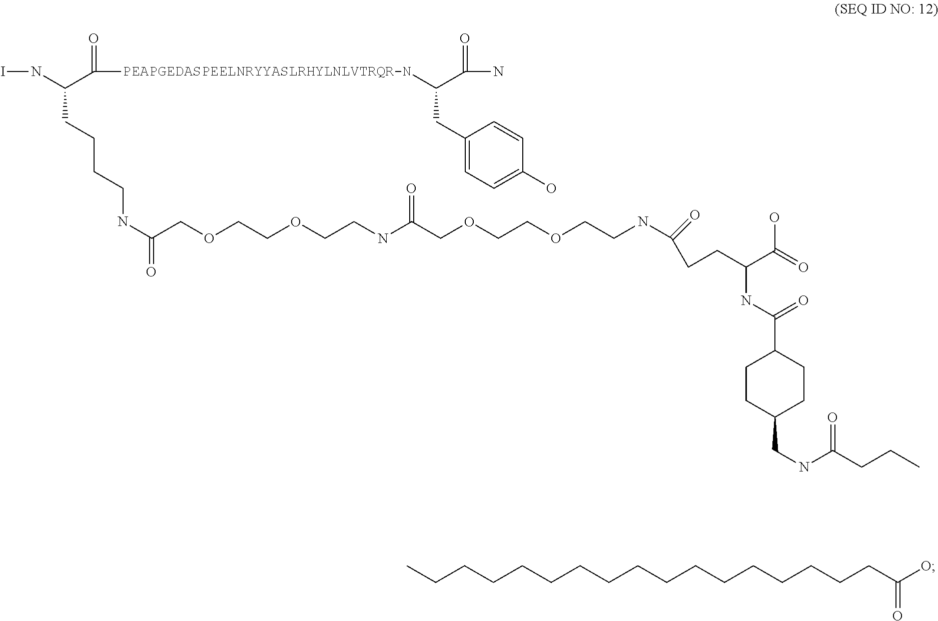
N-epsilon4-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl]hPYY(3-36) 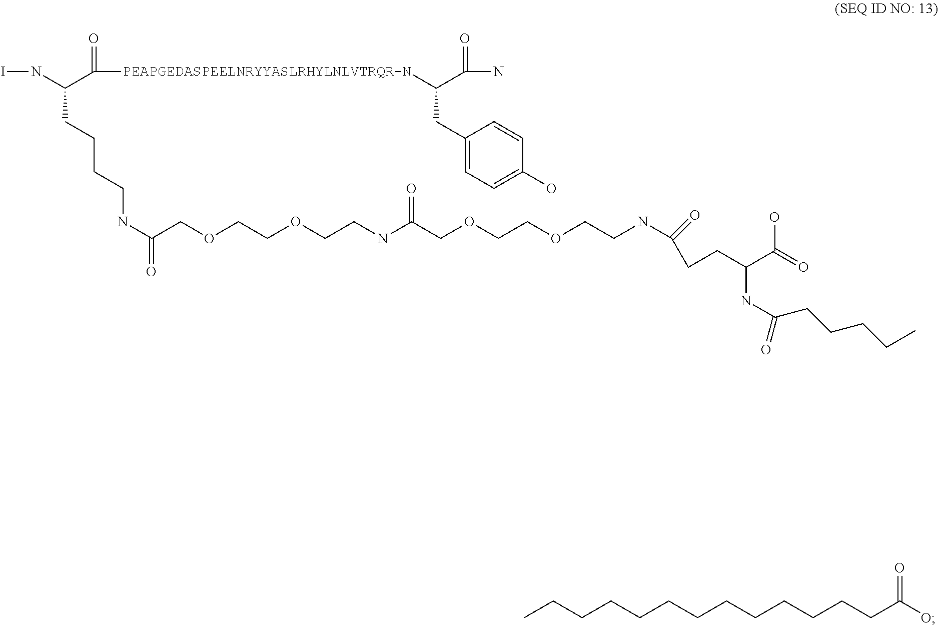
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Asn10,Asp11,Lys13,Leu17,Leu30, Val31]hPP(1-36) 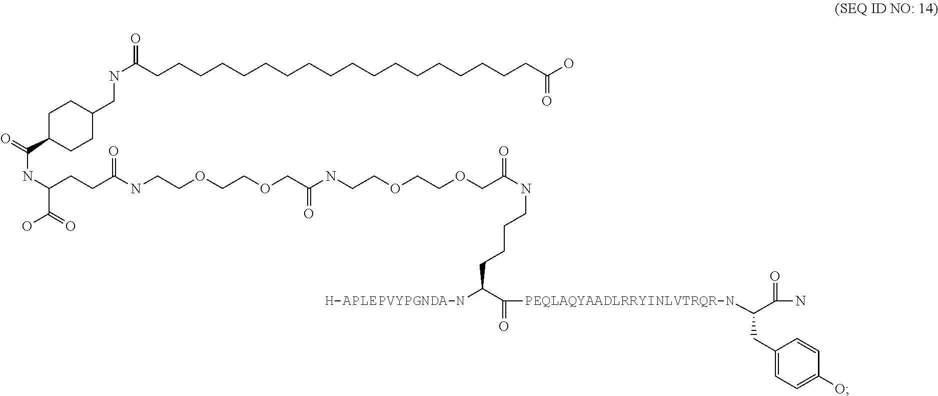
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,Leu17,Leu28,Val30,Gln34]hPP(1-36) 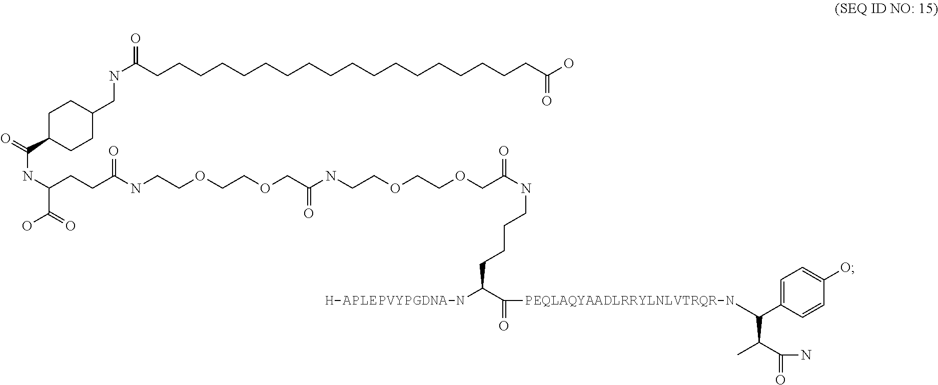
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,Leu17,Val28,Leu30,Gln34]hPP(1-36) 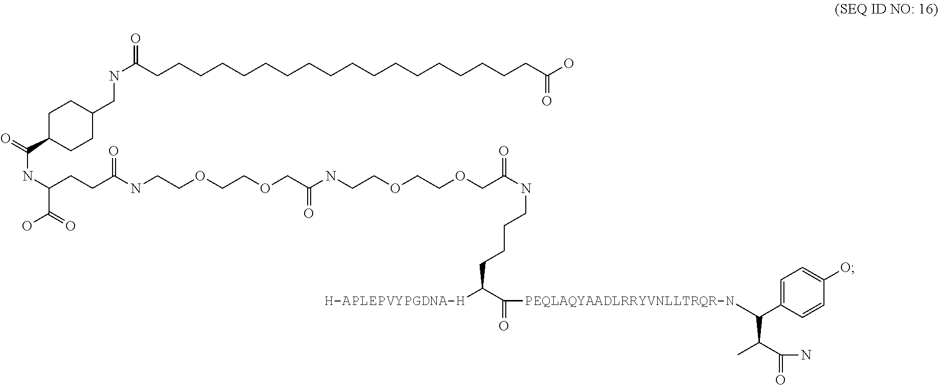
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,Leu17,Val30,Gln34]hPP(1-36) 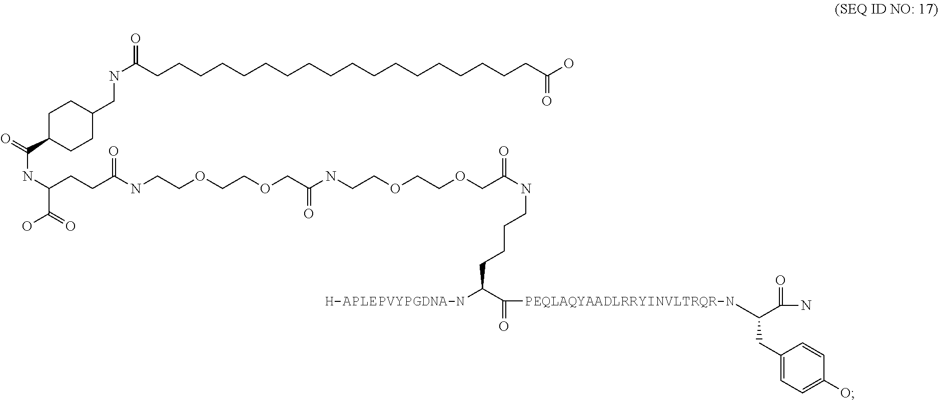
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,Leu17,Gln29,Leu30]hPP(1-36) 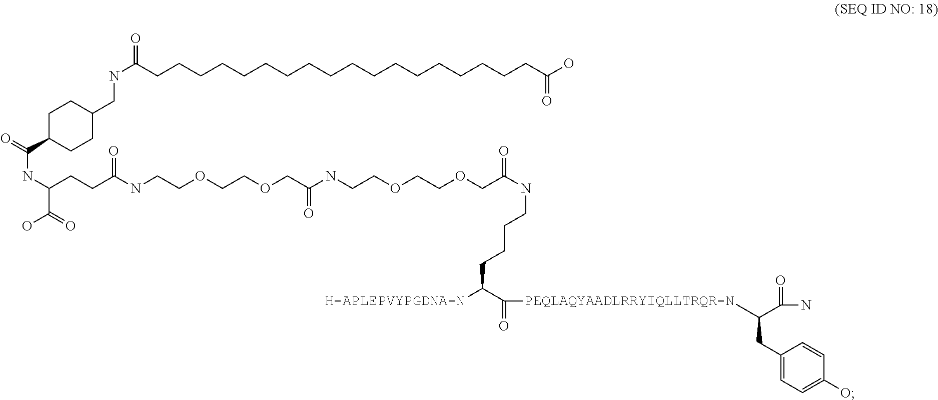
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,Arg26]hPYY(3-36) 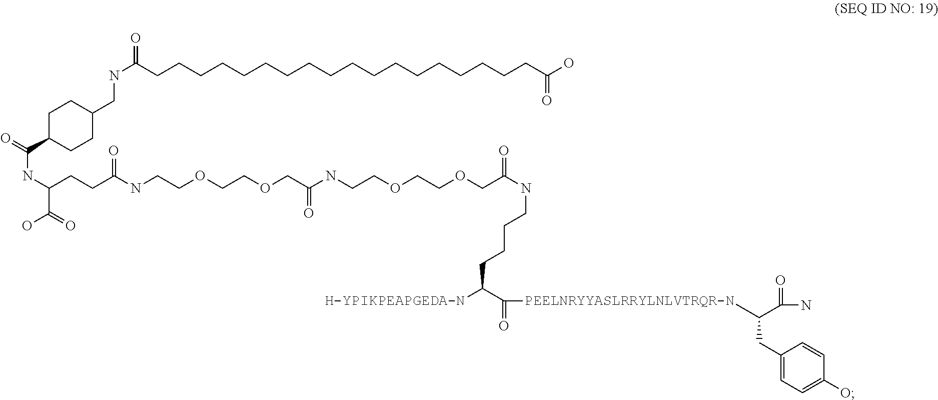
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Leu3,Glu4,Val6,Tyr7,Lys13, Arg26]hPYY(1-36) 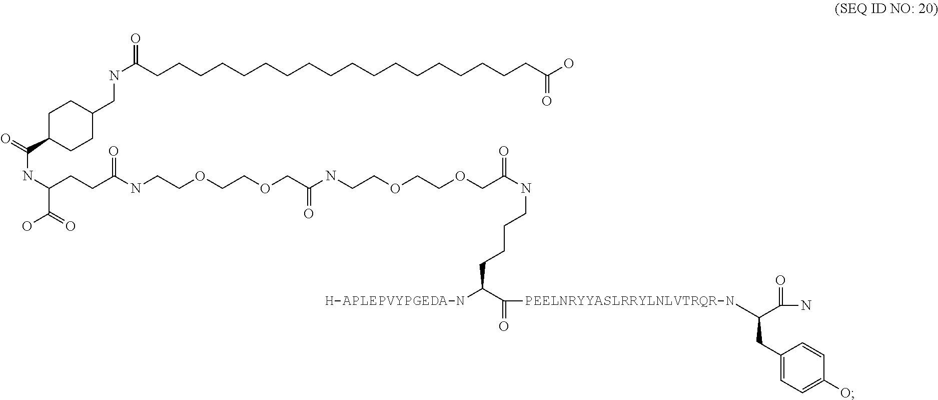
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Glu4, Lys13,Arg26]hPYY(1-36) 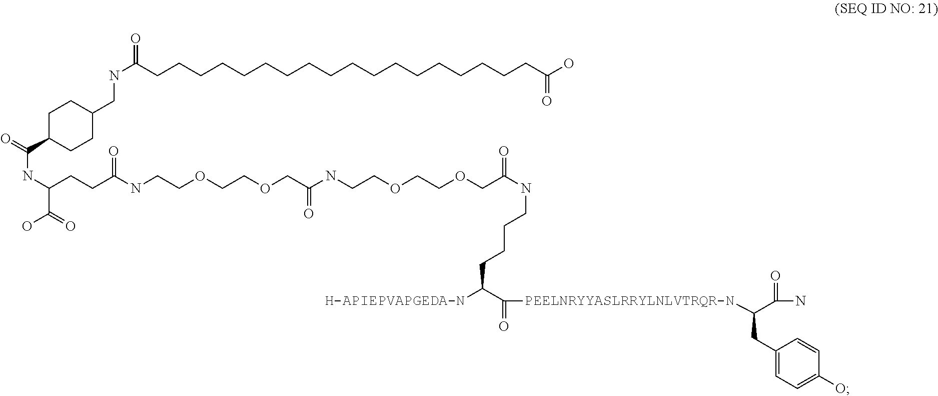
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Ala1,Glu4,Tyr7,Lys13,Arg26]hPYY(1-36) 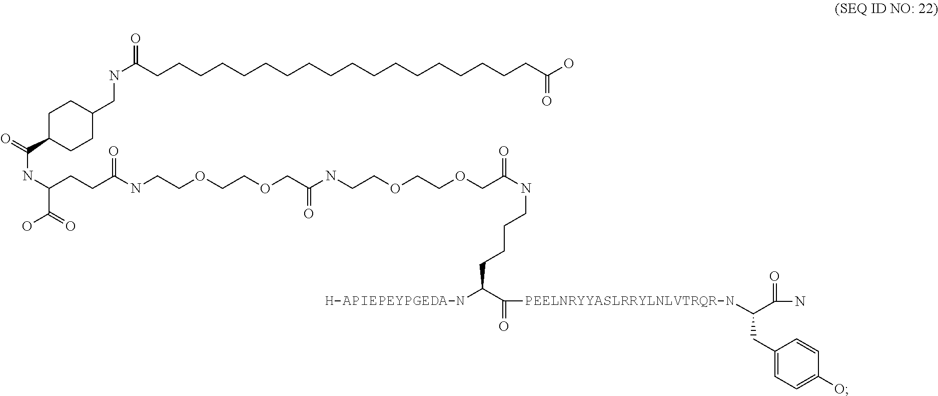
N-alfa-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY(3-36) 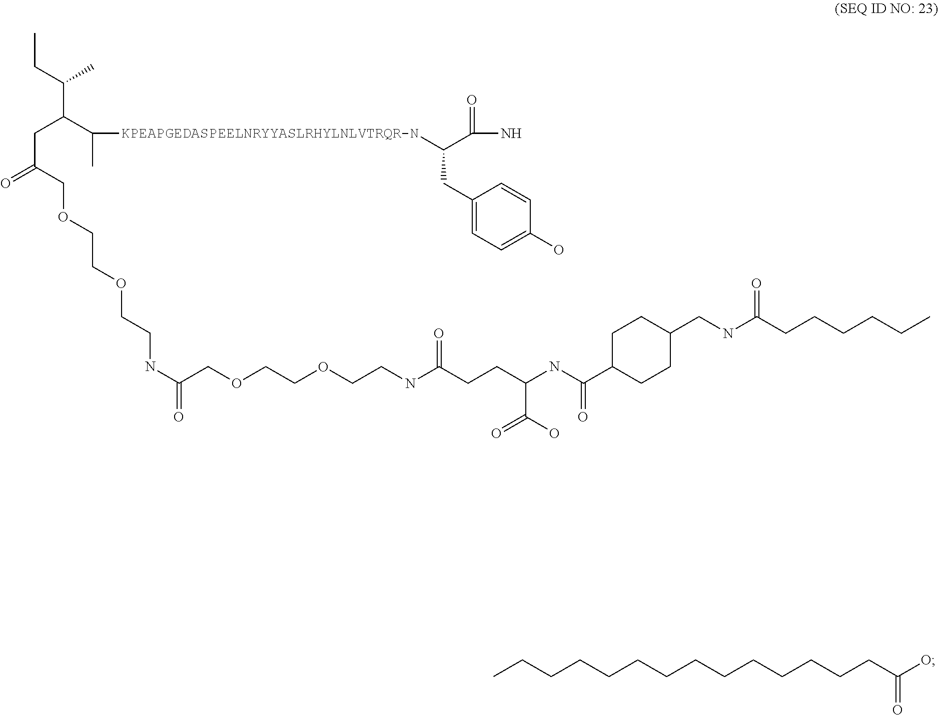
N-epsilon13-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys13]hPP(1-36) 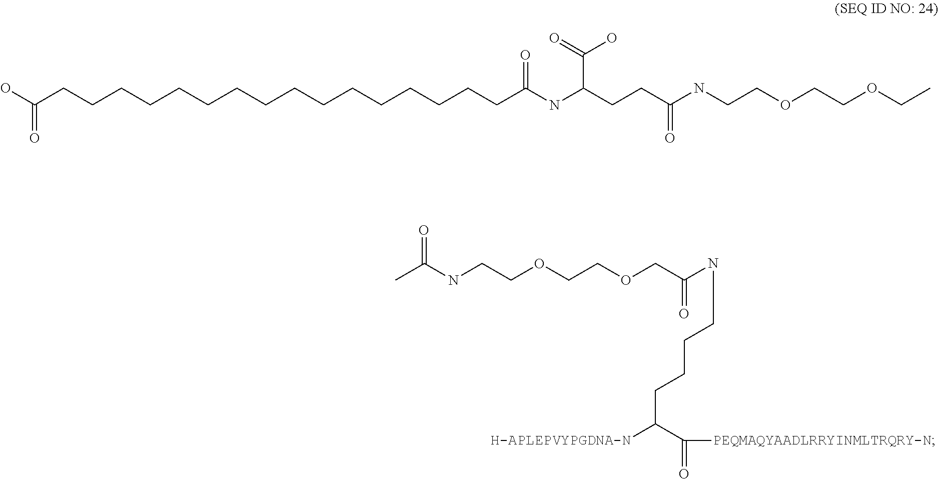
N-epsilon4-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys4]hPYY(3-36) 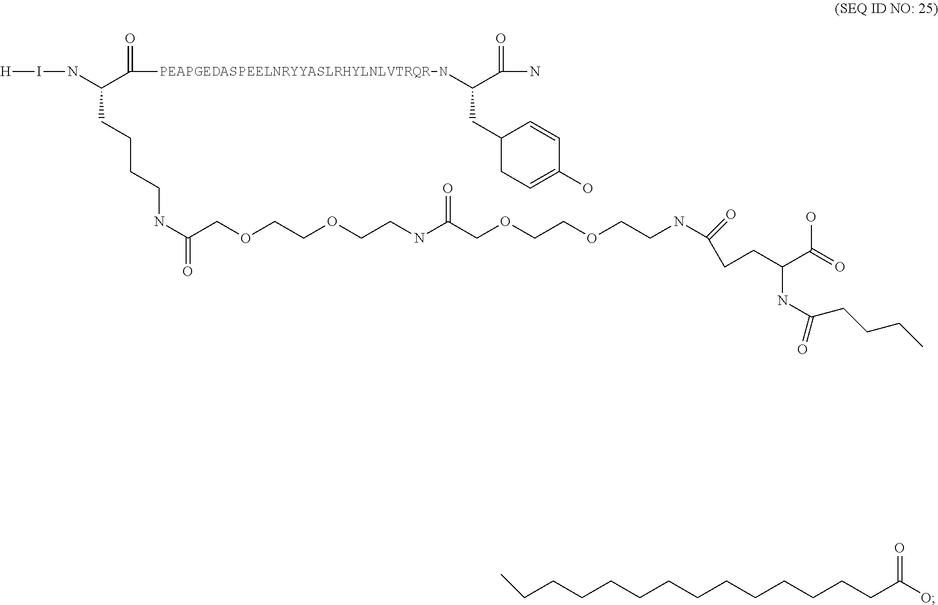
N-epsilon13-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys13,Gln34]hPP(1-36) 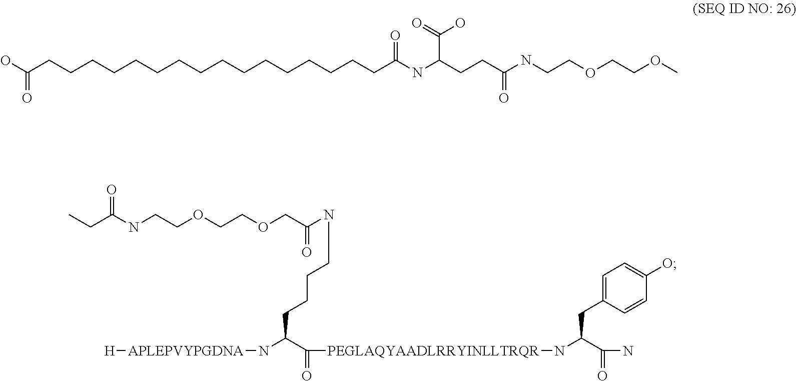
N-epsilon11-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys 11]hPYY(3-36) 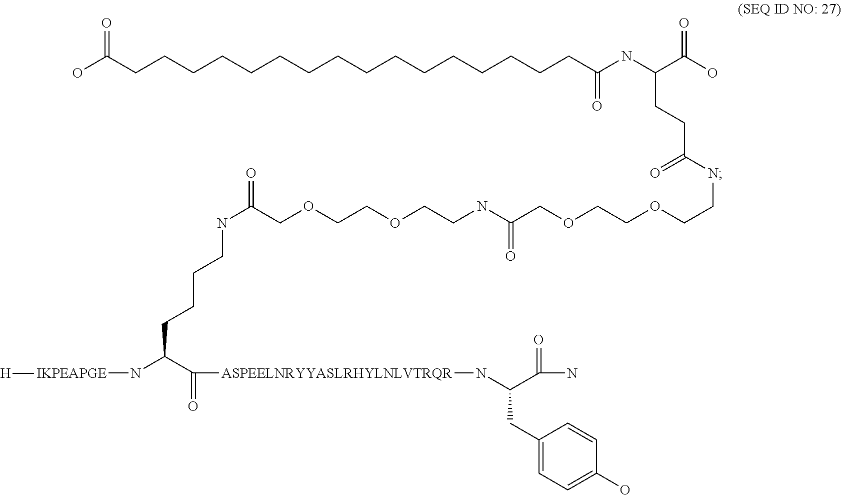
N-epsilon11-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys11]hPYY(3-36) 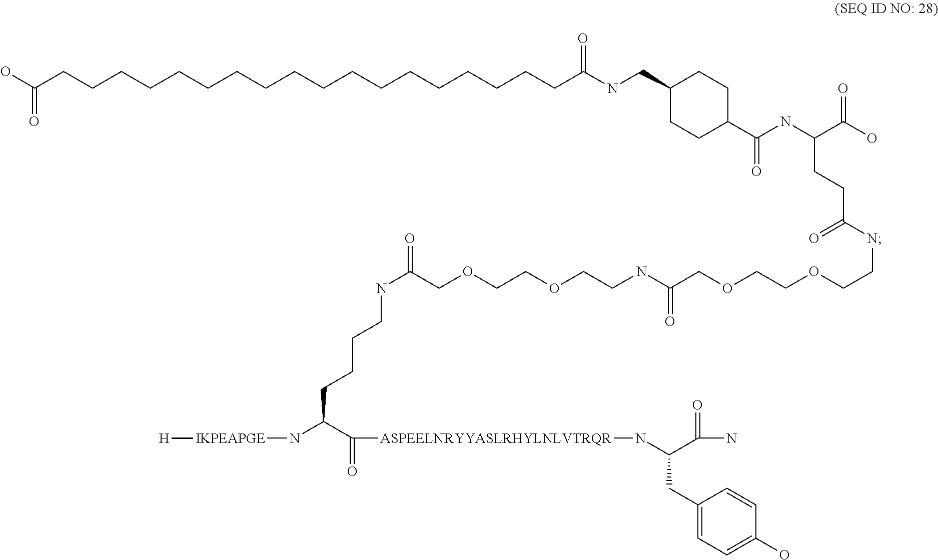
N-epsilon11-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys11,Leu17,Leu30]hPP2-36 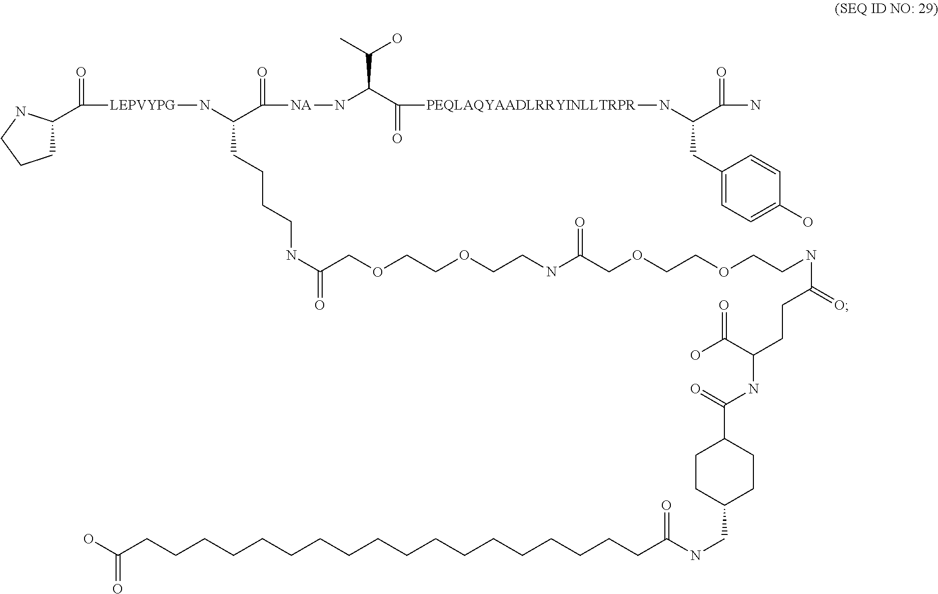
N-epsilon11-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys11,Leu17,Leu30]hPP2-36 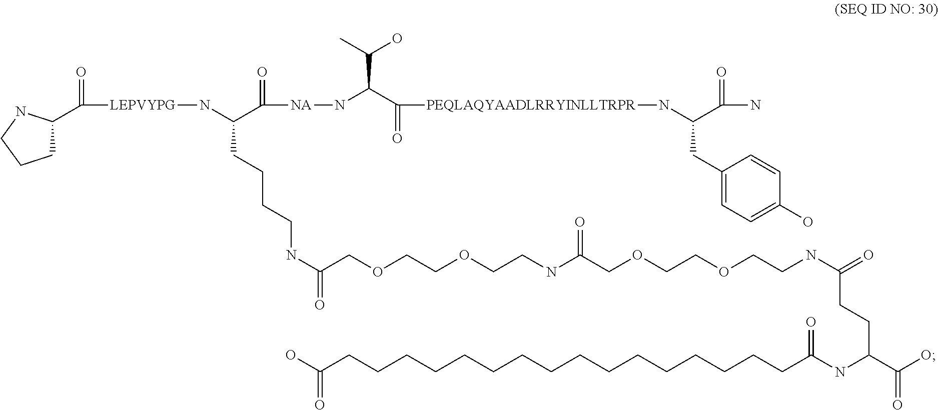
N-epsilon13-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys13]hPP(1-36) 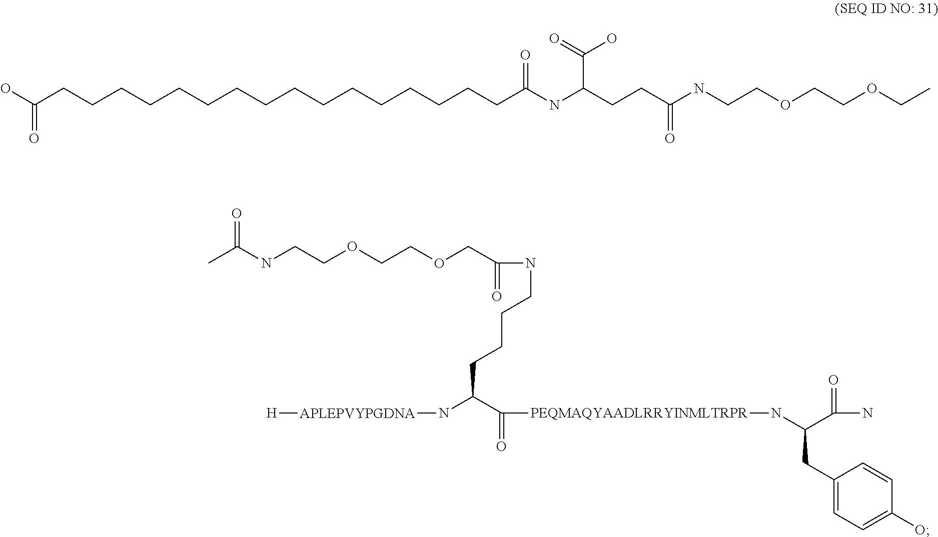
N-epsilon18-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys18,Leu17,Leu30]hPP2-36 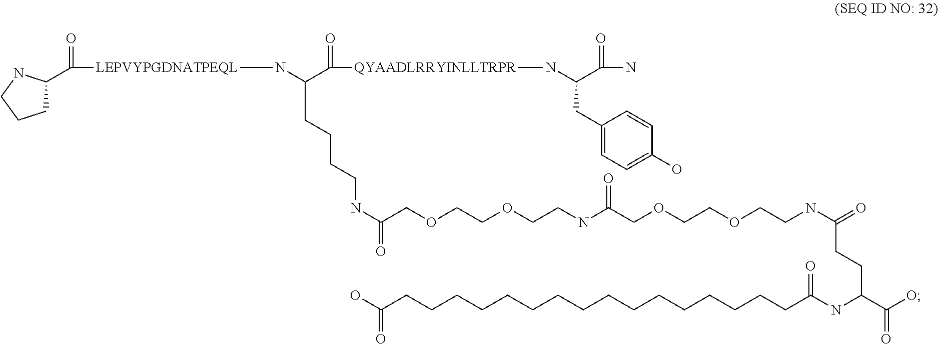
N-alfa-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl]hPYY18-36 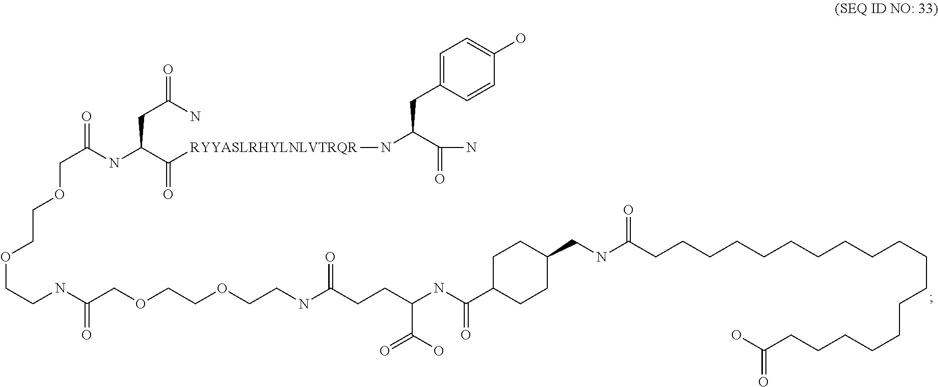
N-epsilon25-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys25]hPYY(3-36) 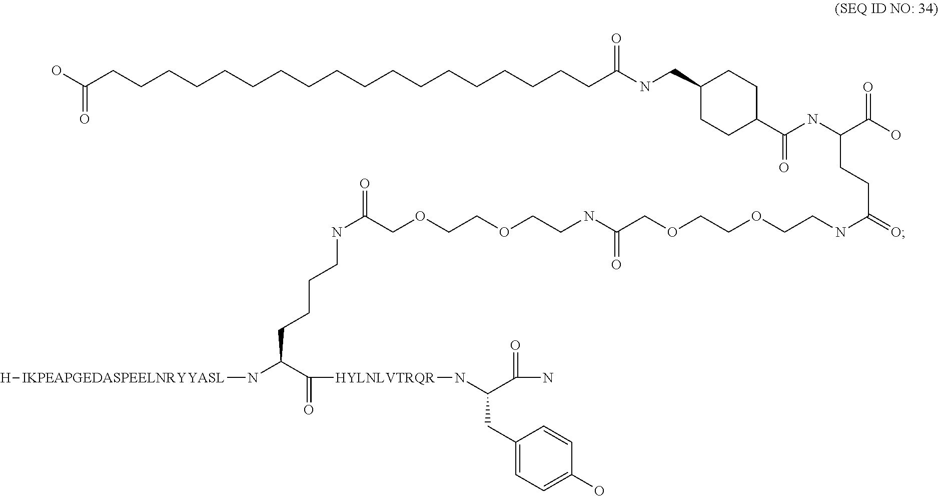
N-epsilon24-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys24]hPYY(3-36) 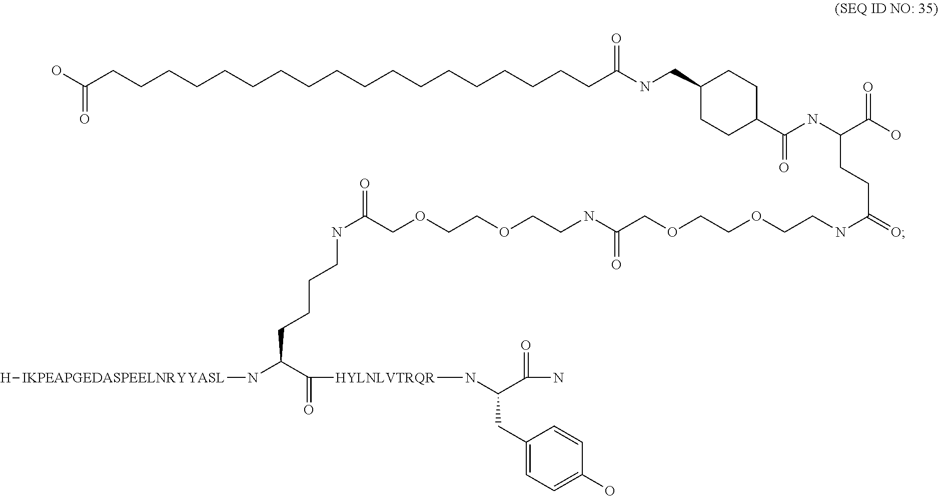
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys13,Leu17,Leu30]hPP(1-36) 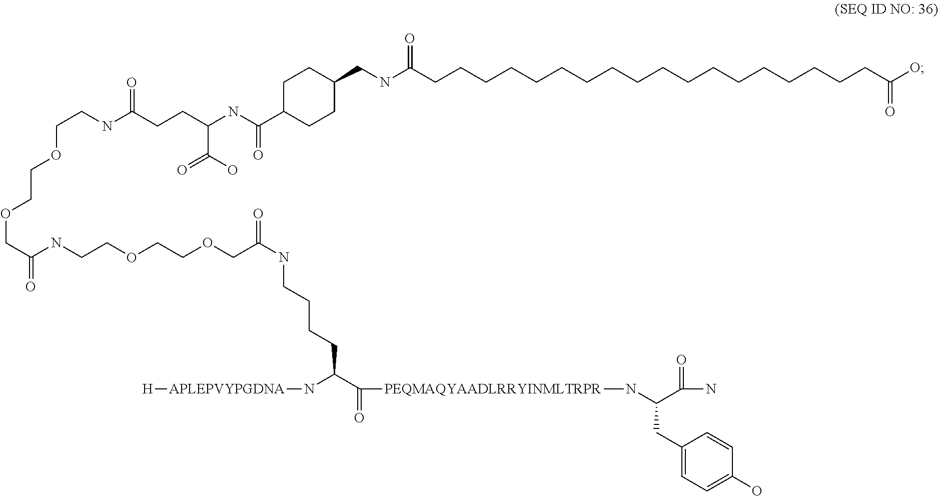
N-epsilon25-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Leu17,Lys25,Leu30]hPP(1-36) 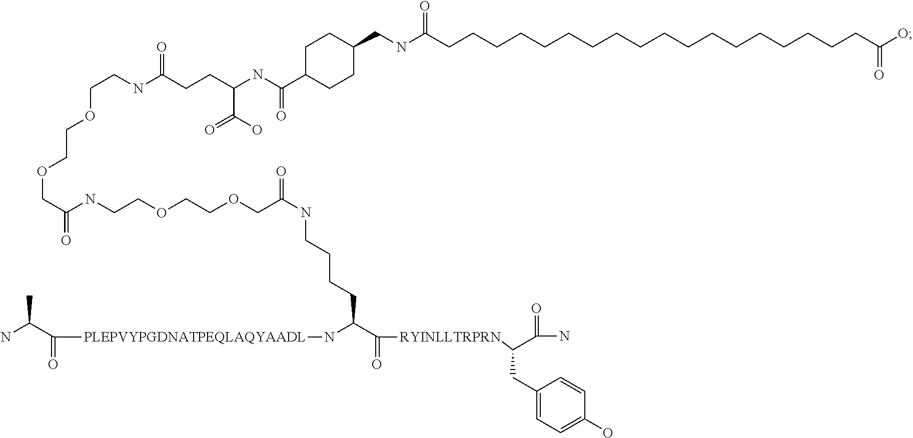
N-epsilon15-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys15,Leu17,Leu30]hPP(1-36) 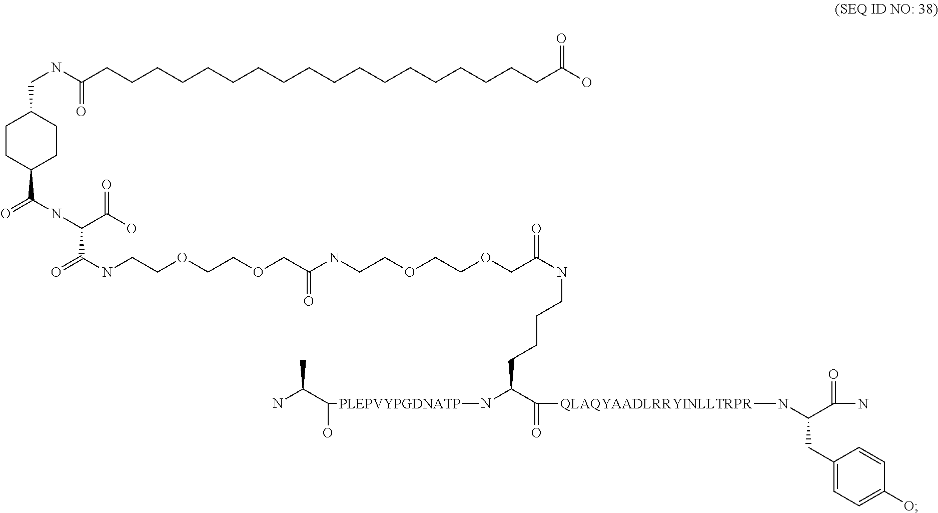
N-epsilon10-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys10,Leu17,Leu30,Gln34]hPP2-36 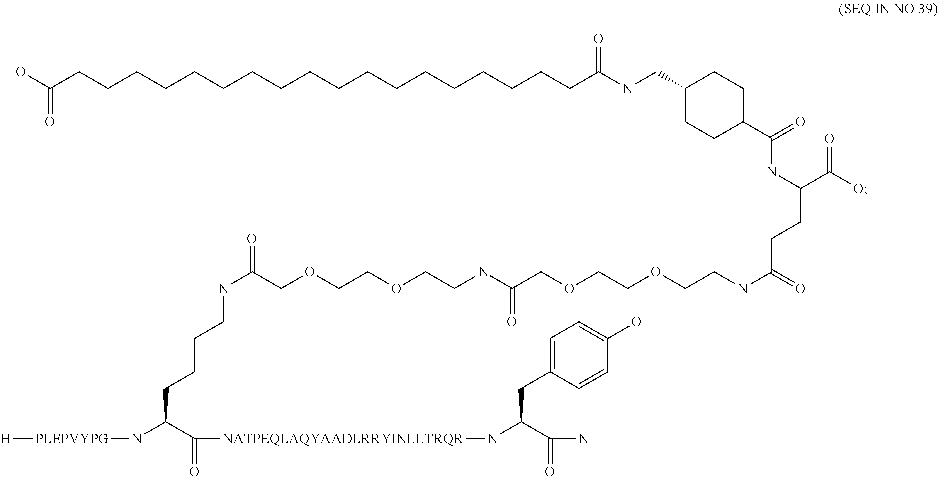
N-epsilon19-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys19]hPYY(3-36) 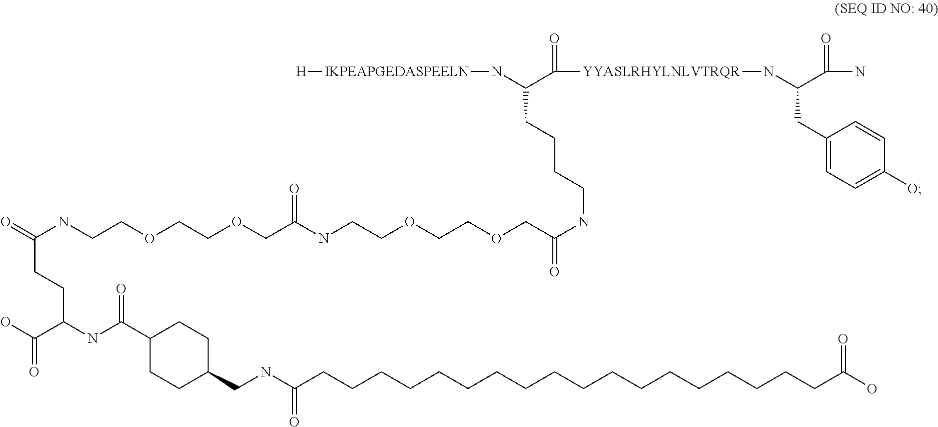
N-epsilon33-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Leu17,Leu30,Lys33]hPP2-36 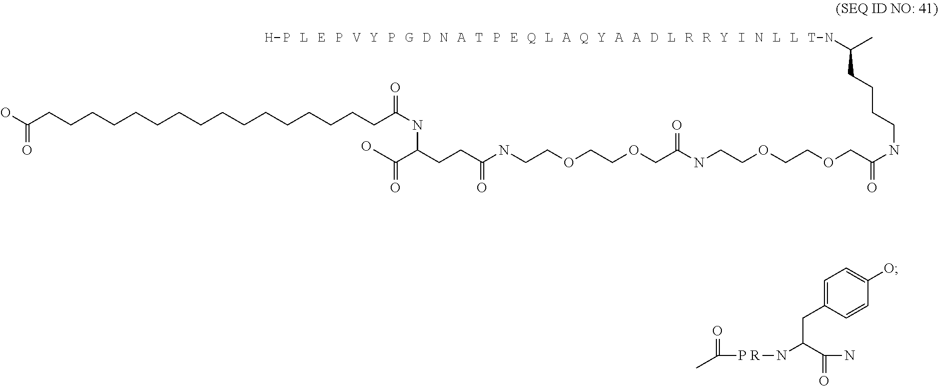
N-epsilon33-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Leu17,Leu30,Lys33]hPP2-36 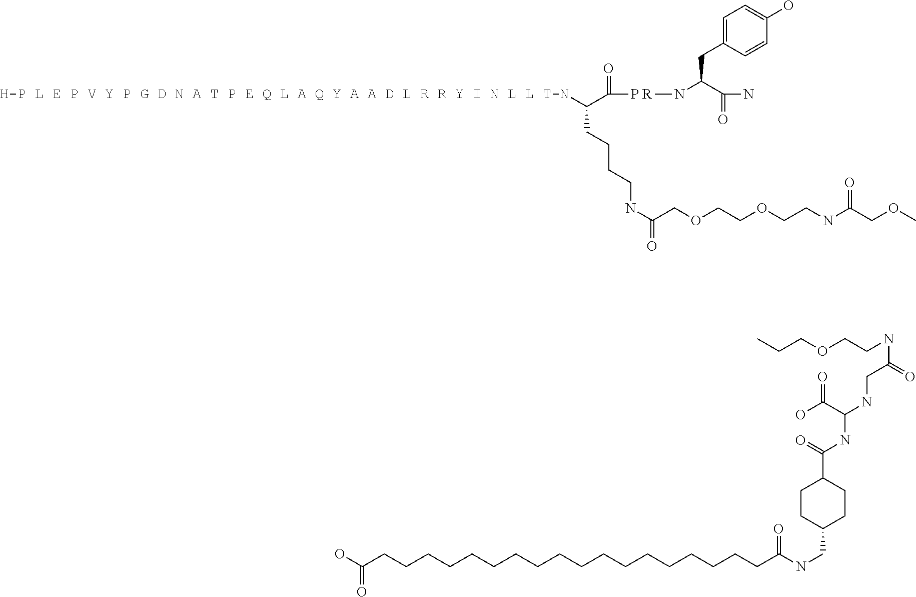
(SEQ ID NO: 42);
N-epsilon18-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Leu17,Lys18,Leu30]hPP2-36 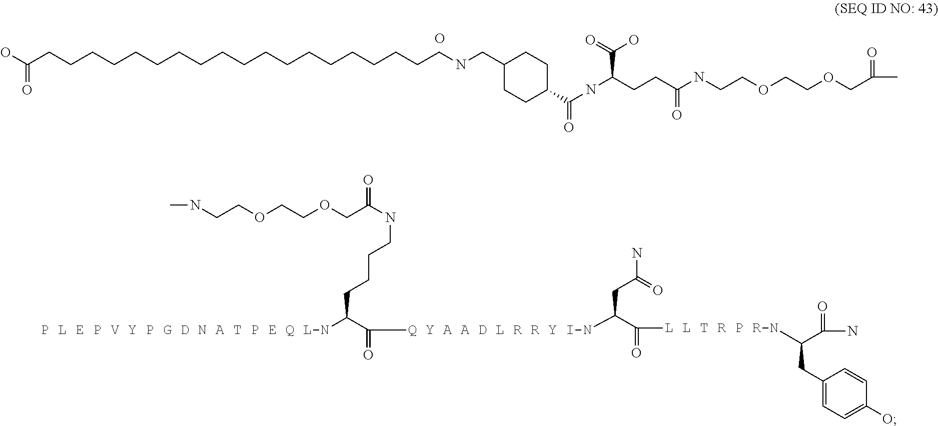
N-epsilon29-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Leu17,Lys29,Leu30]hPP2-36 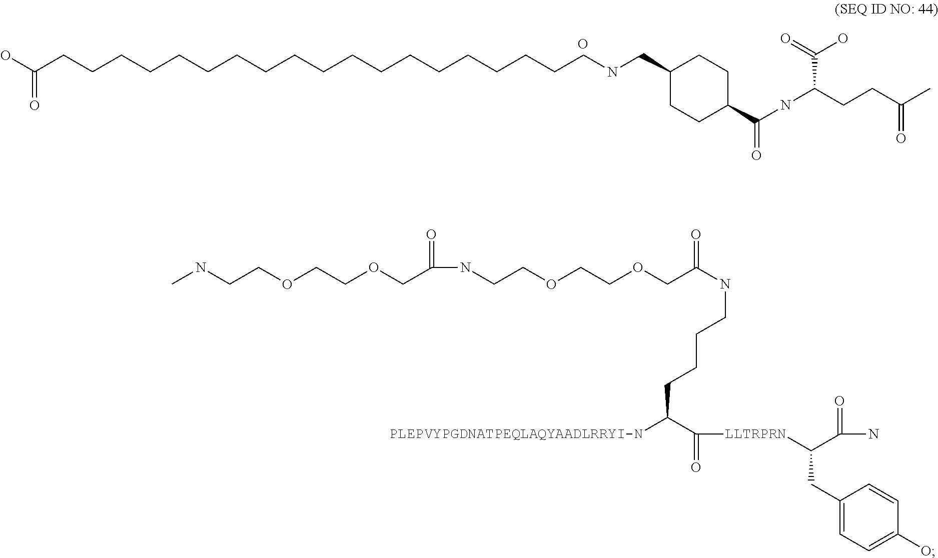
N-epsilon26-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Leu17,Lys26,Leu30]hPP2-36 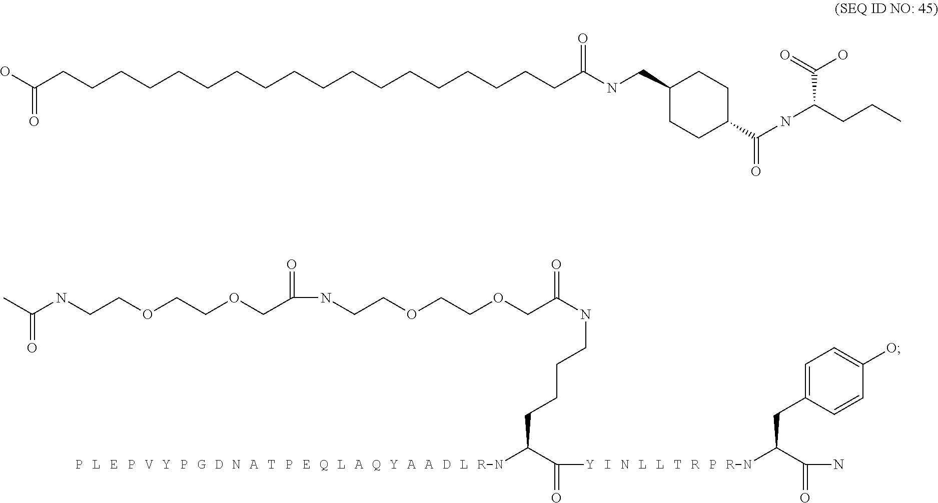
N-epsilon26-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Leu17,Lys26,Leu30]hPP2-36 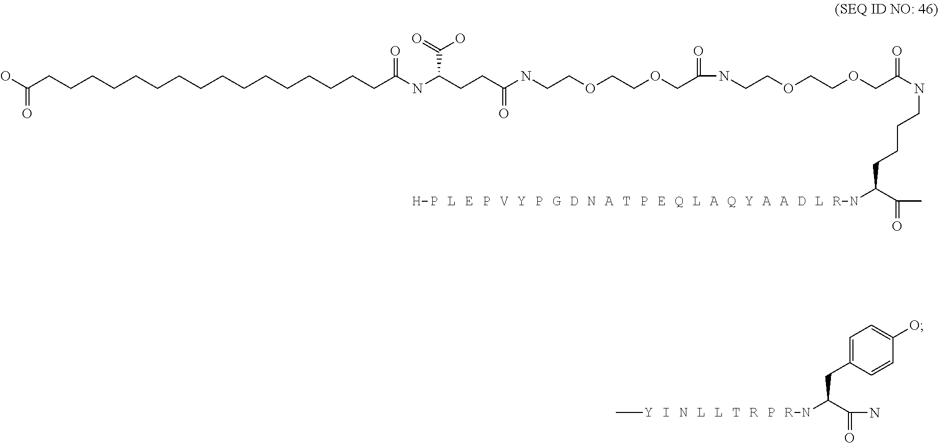
N-epsilon35-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Leu17,Leu30,Lys35]hPP2-36 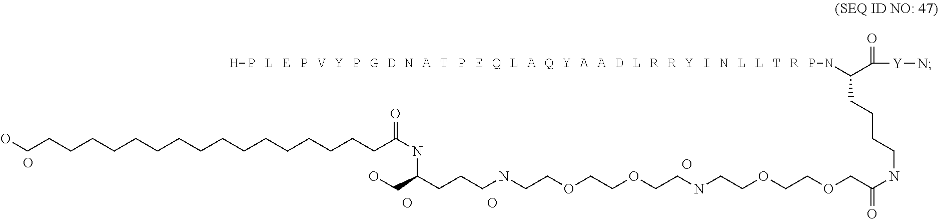
N-epsilon35-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Leu17,Leu30,Lys35]hPP2-36 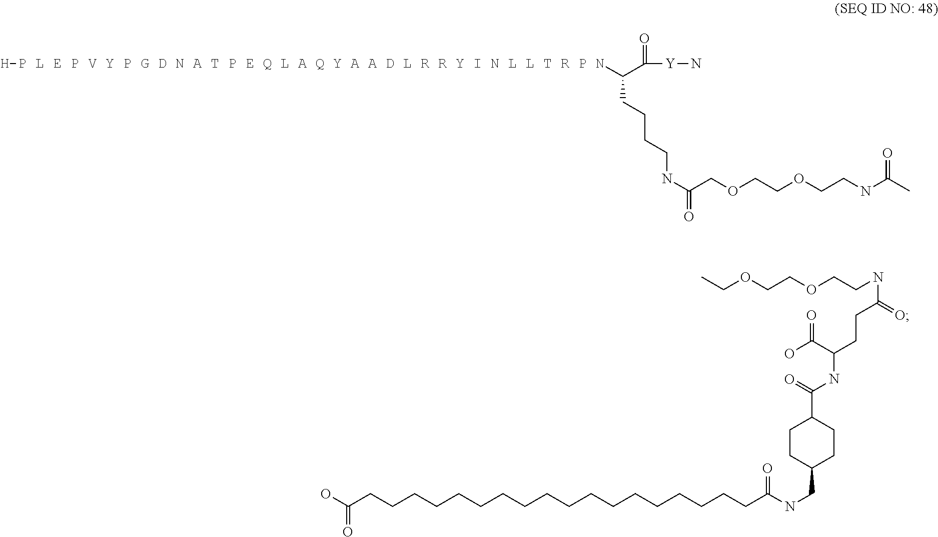
N-epsilon25-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Leu17,Lys25,Leu30]hPP2-36 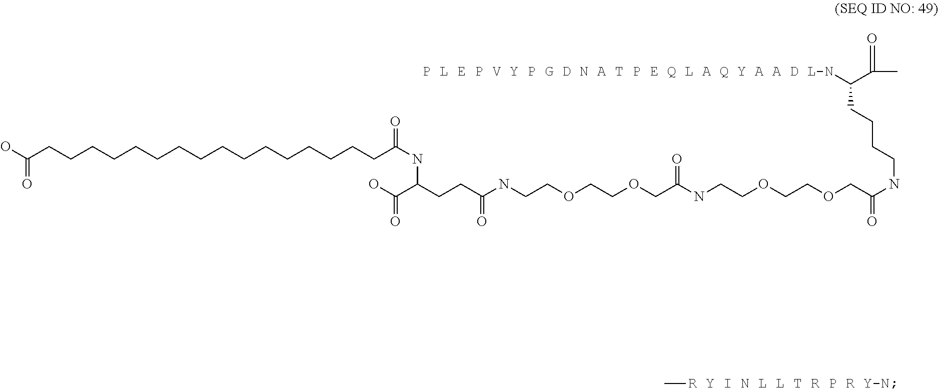
N-epsilon25-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Leu17,Lys25, Leu30]hPP2-36 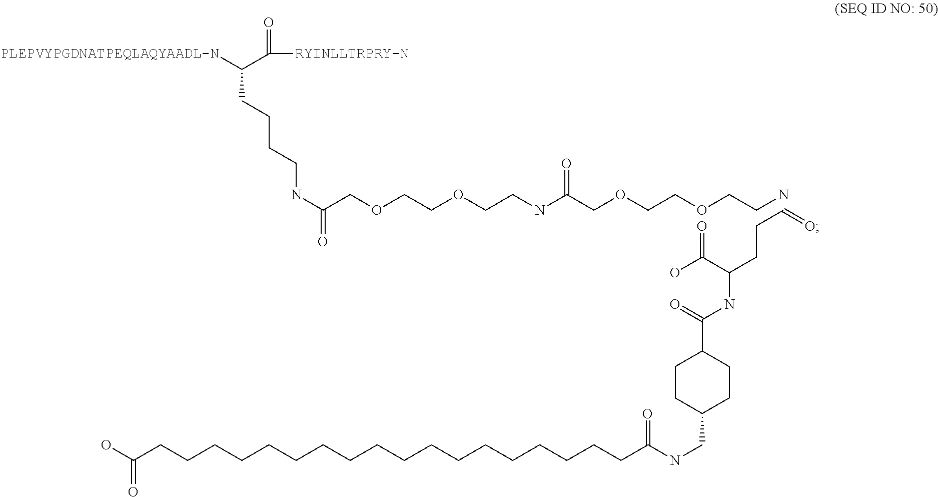
N-epsilon13-[4-(16-(1H-Tetrazol-5-yl)hexadecanoylsulfamoyl)butyryl]ethoxy}ethoxy)acetylamino]ethoxy}ethoxy)acetyl][Lys13[PYY(3-36) 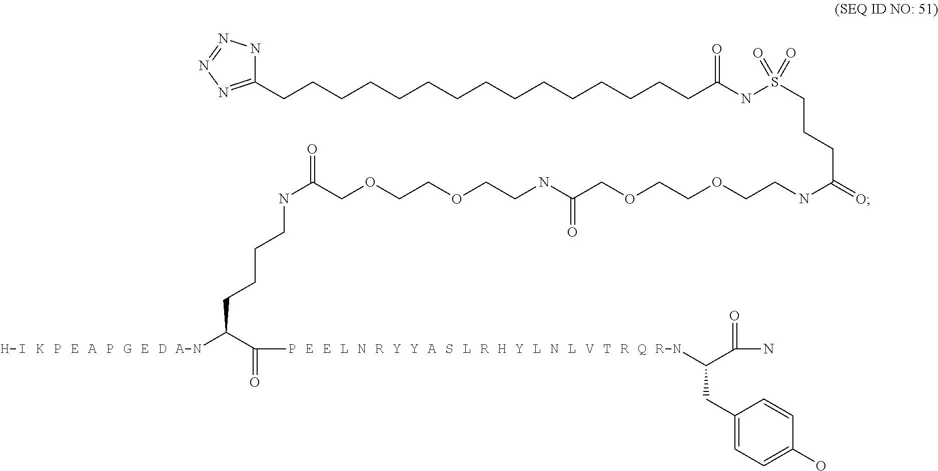
N-epsilon25-[4-(16-(1H-Tetrazol-5-yl)hexadecanoylsulfamoyl)butyryl]ethoxy}ethoxy)acetylamino]ethoxy}ethoxy)acetyl][Lys25[PYY(3-36) 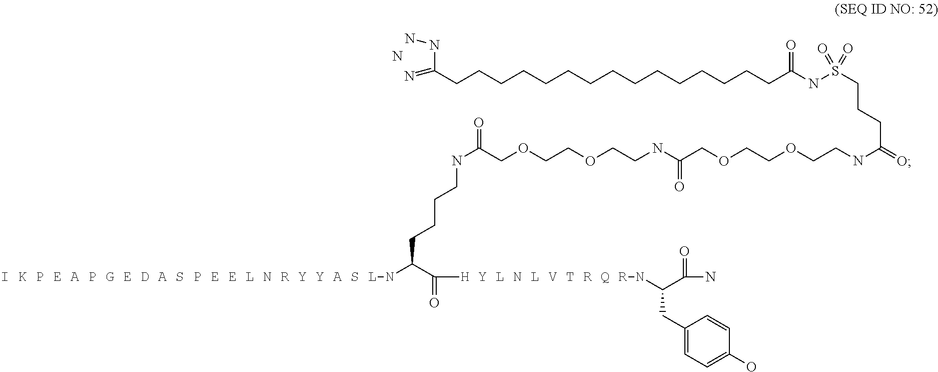
N-alfa-[4-(16-(1H-Tetrazol-5-yl)hexadecanoylsulfamoyl)butyryl]ethoxy}ethoxy)acetylamino]ethoxy}ethoxy)acetyl]PYY(3-36) 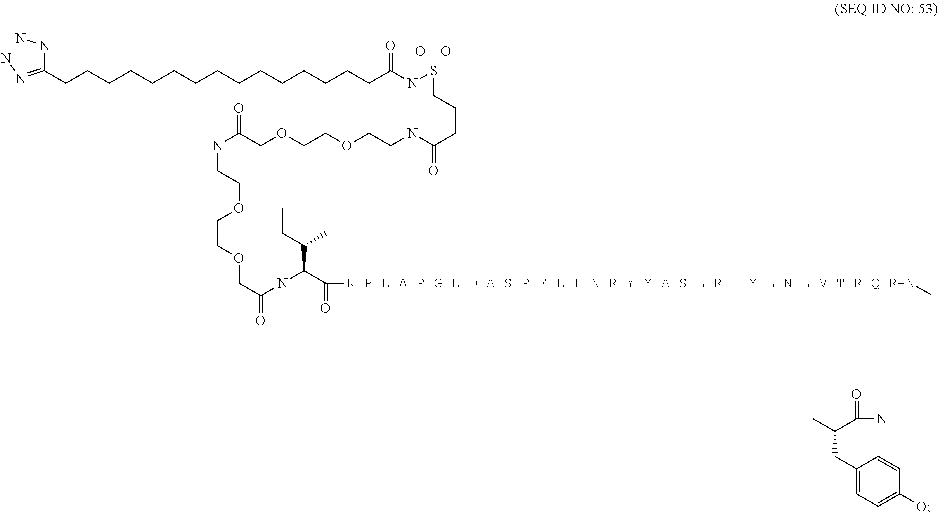
N-alfa-[4-(16-(1H-Tetrazol-5-yl)hexadecanoylsulfamoyl)butyryl]ethoxy}ethoxy)acetylamino]ethoxy}ethoxy)acetyl]PYY(3-36) 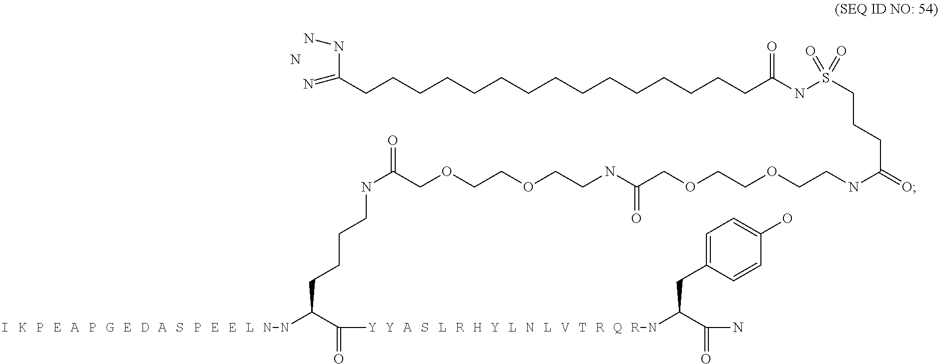
N-alfa-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Leu17,Leu30]hPP2-36 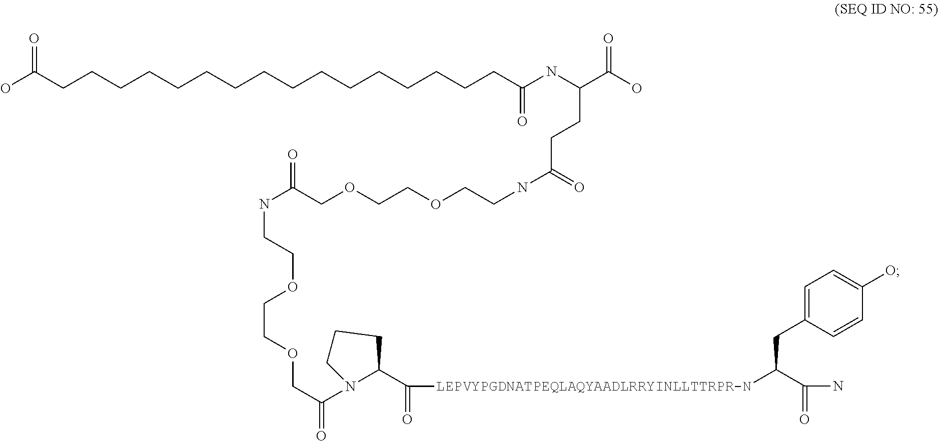
N-alfa-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Leu17,Leu30]hPP2-36 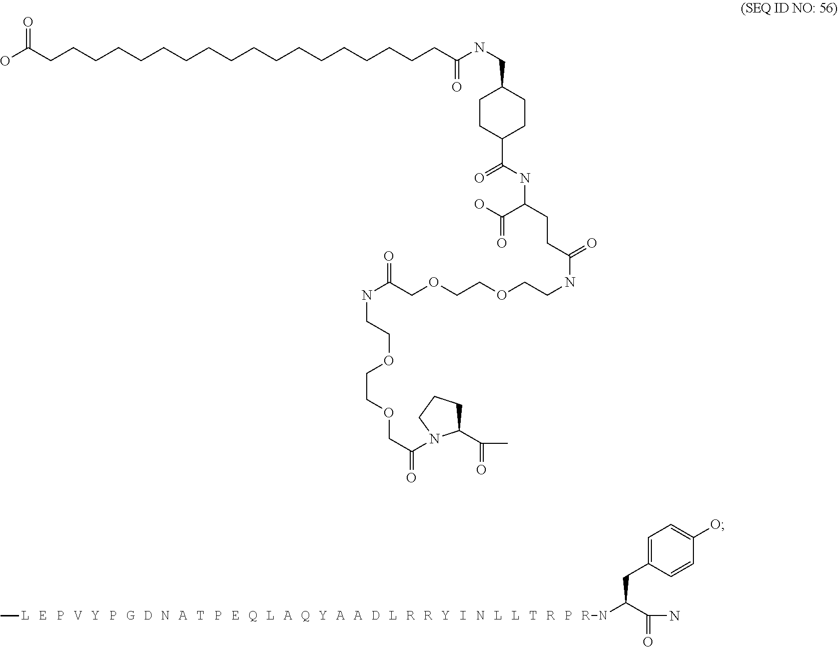
N-epsilon18-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys18]PYY3-36 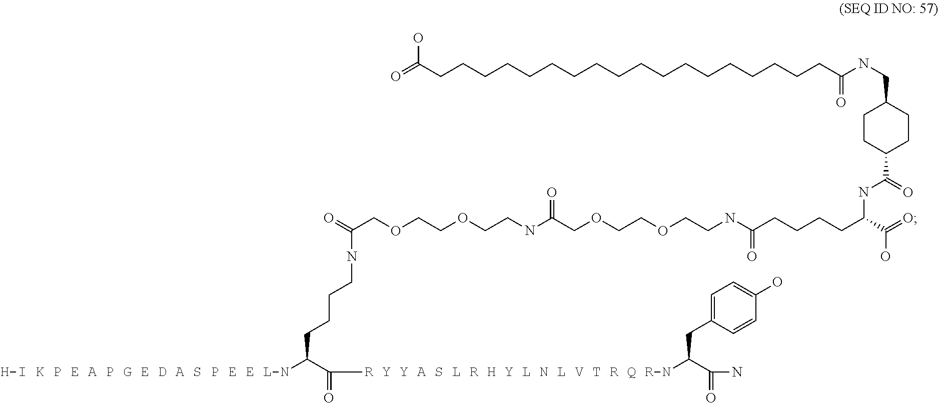
N-epsilon22-[2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl][Lys22]PYY3-36 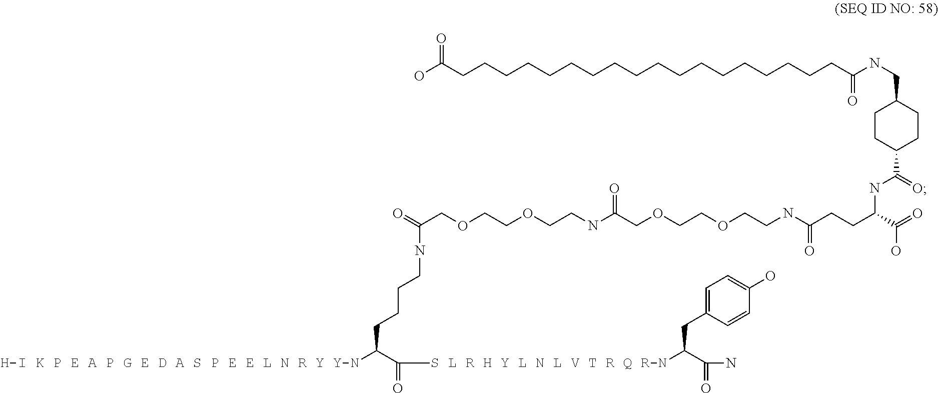
N-epsilon26-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys26]PYY3-36 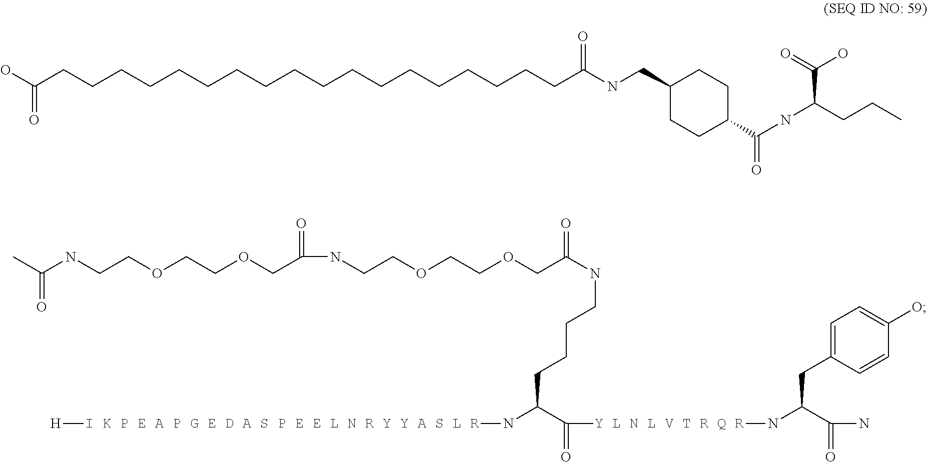
N-epsilon29-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys29]PYY3-36 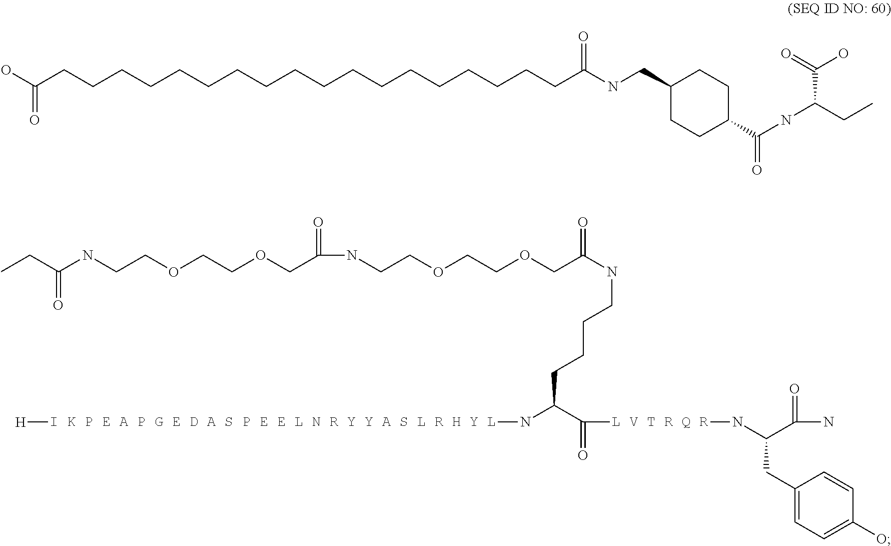
N-epsilon36-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Lys36]PYY3-36 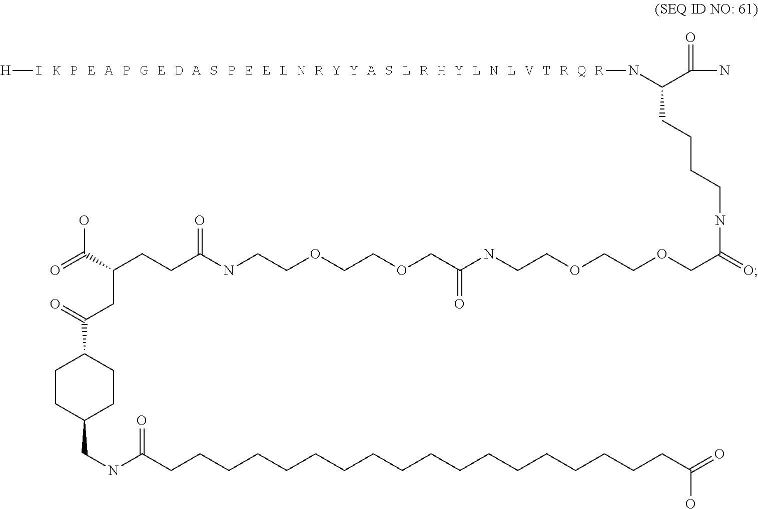
N-epsilon21-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys21]PYY3-36 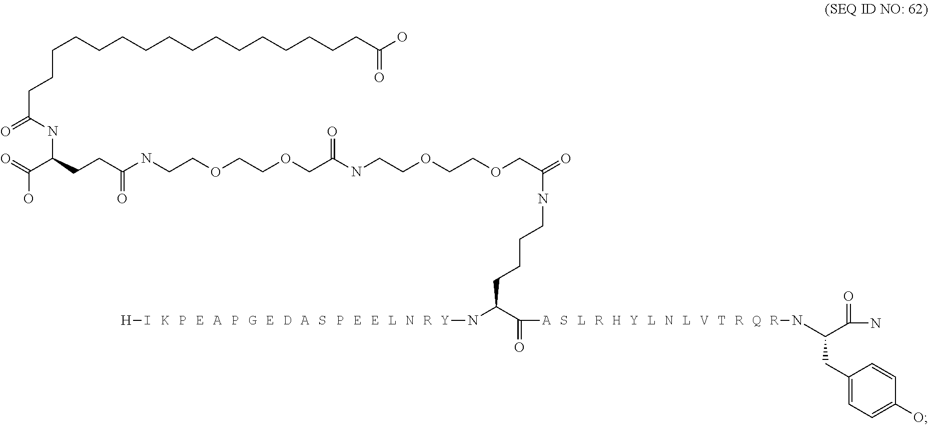
N-epsilon30-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys30]PYY3-36 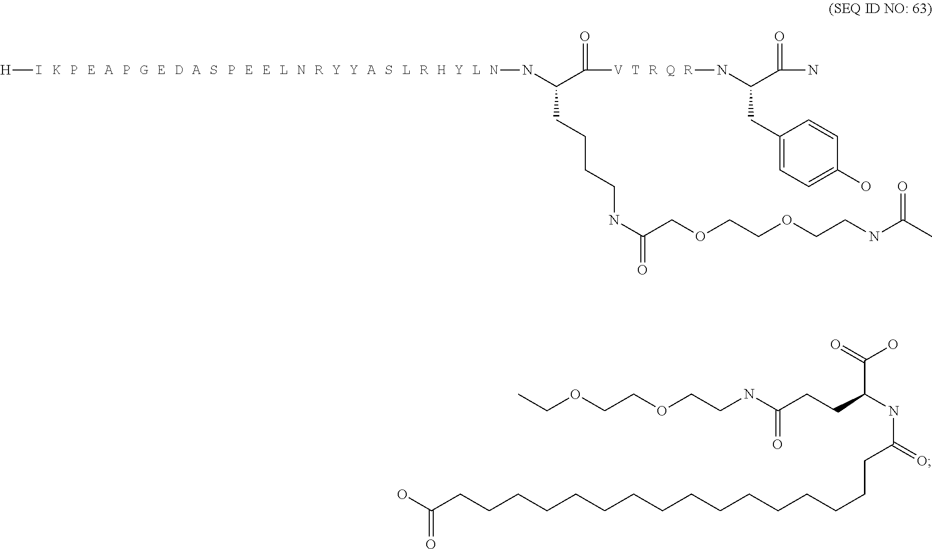
N-epsilon31-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys31]PYY3-36 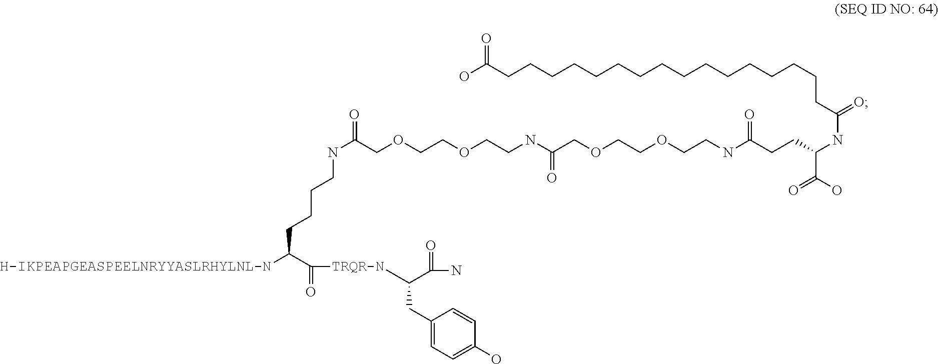
N-epsilon14-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys14]PYY3-36 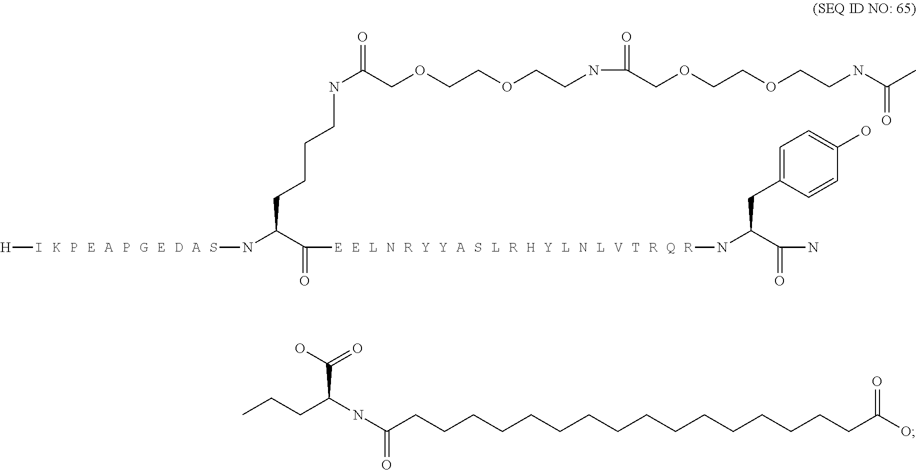
N-epsilon15-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys15[PYY3-36 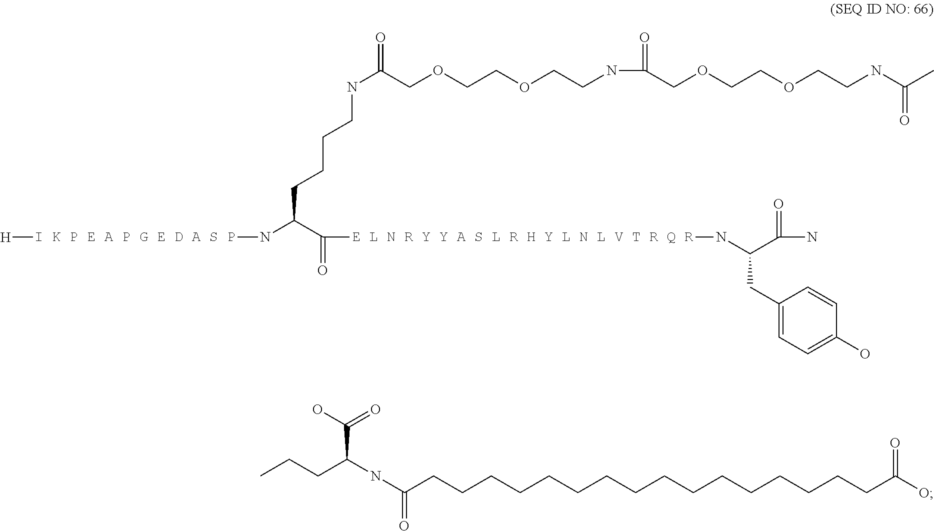
N-epsilon16-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys16]PYY3-36 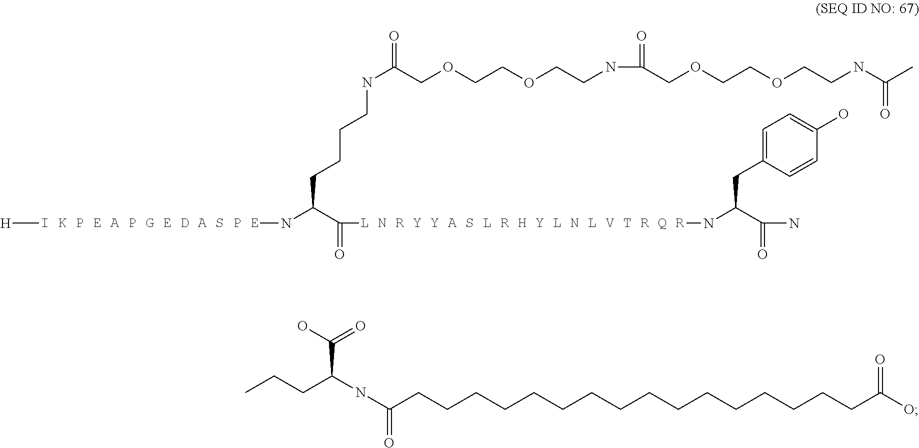
N-epsilon20-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys20]PYY3-36 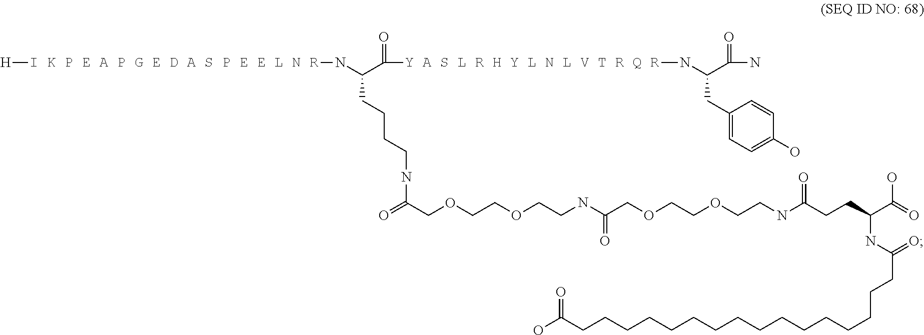
N-epsilon28-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys28]PYY3-36 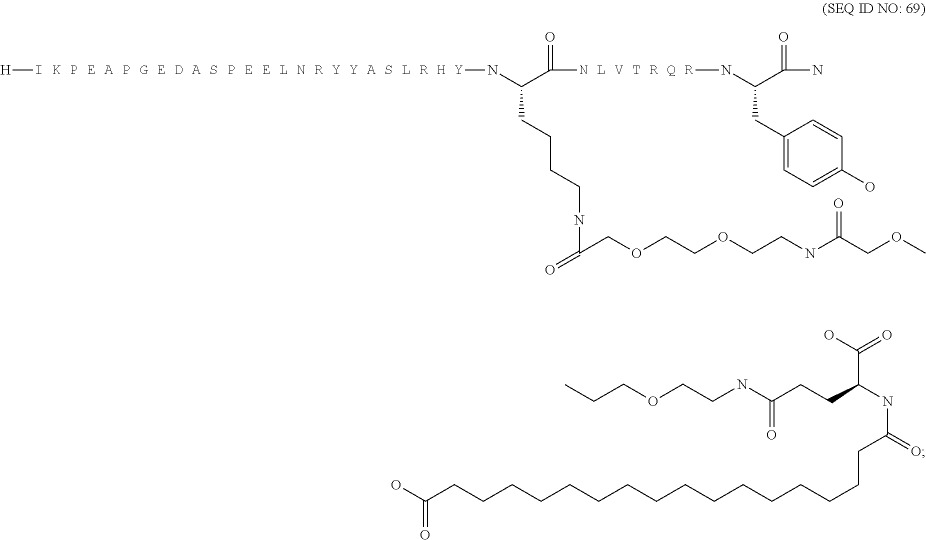
N-epsilon32-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Lys32]PYY3-36 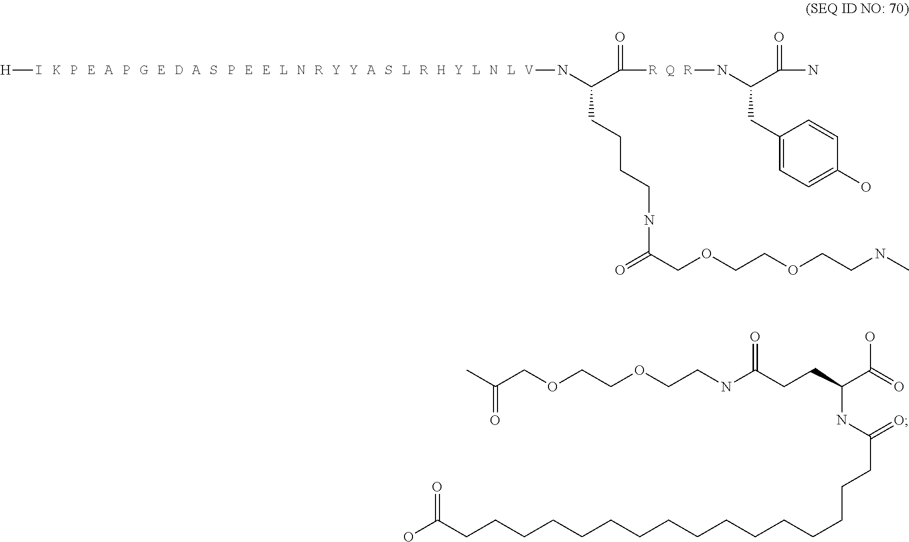
N-epsilon25-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Leu17,Lys25,Leu30]PP1-36 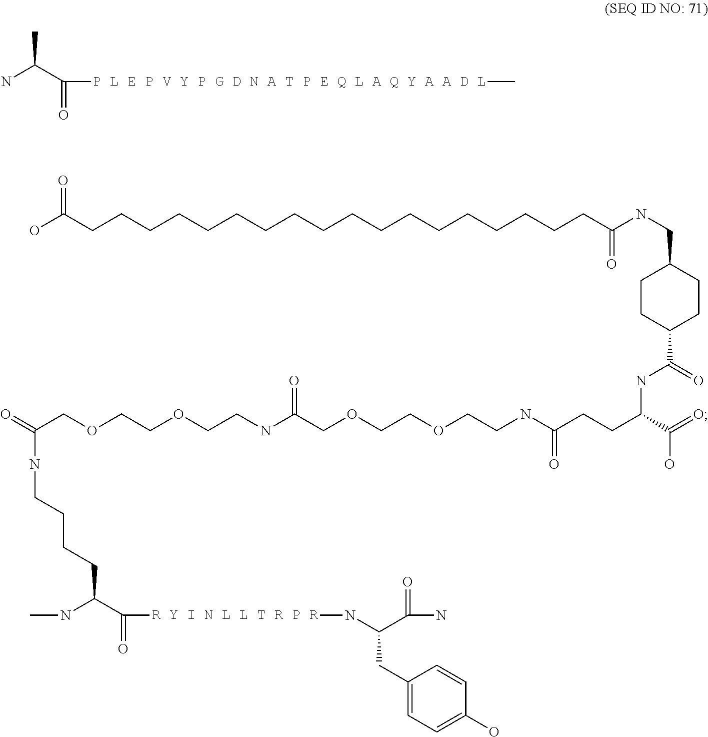
N-epsilon15-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Leu17,Lys15,Leu30]PP1-36 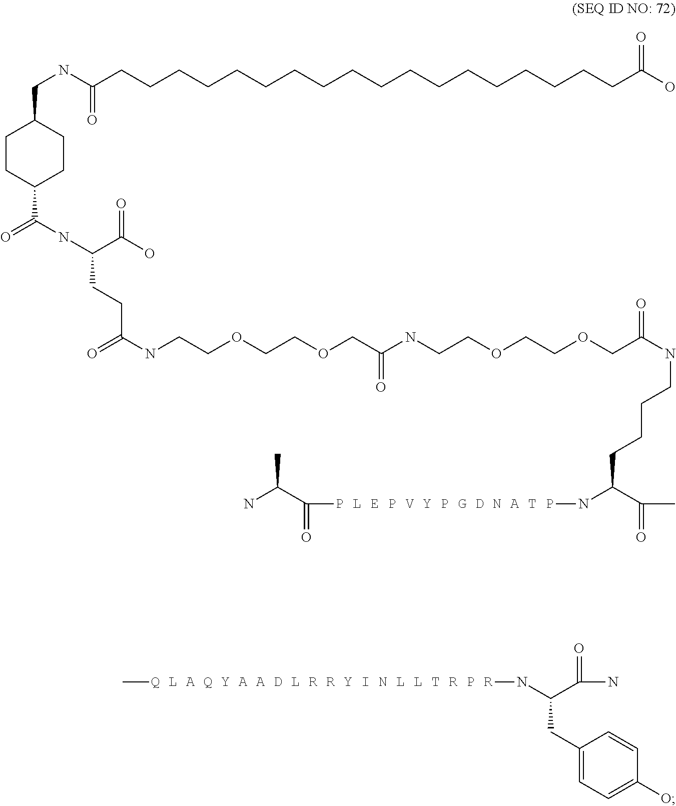
N-epsilon13-[2-(2-{2-[2-(2-{2-[(S)-4-Carboxy-4-({trans-4-[(19-carboxynonadecanoylamino)methyl]cyclohexanecarbonyl}amino)butyrylamino]-ethoxy}ethoxy)acetylamino]ethoxy}ethoxy)acetyl][Leu17,Lys13,Leu30,Gln34]PP1-36 acid 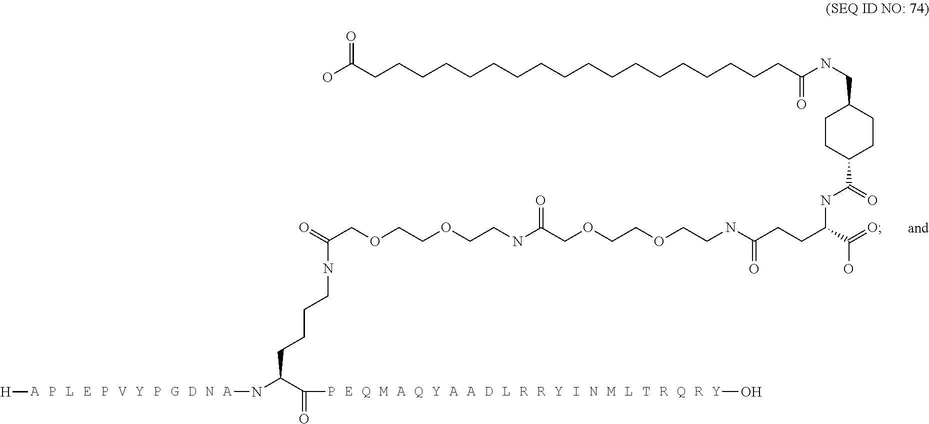
N-alfa-[2-(2-{2-[2-(2-{2-[(S)-4-Carboxy-4-({trans-44(19-carboxynonadecanoylamino)methyl]cyclohexanecarbonyl}amino)butyrylamino]-ethoxy}ethoxy)acetylamino]ethoxy}ethoxy)acetyl][Leu17, Leu30]PP1-36

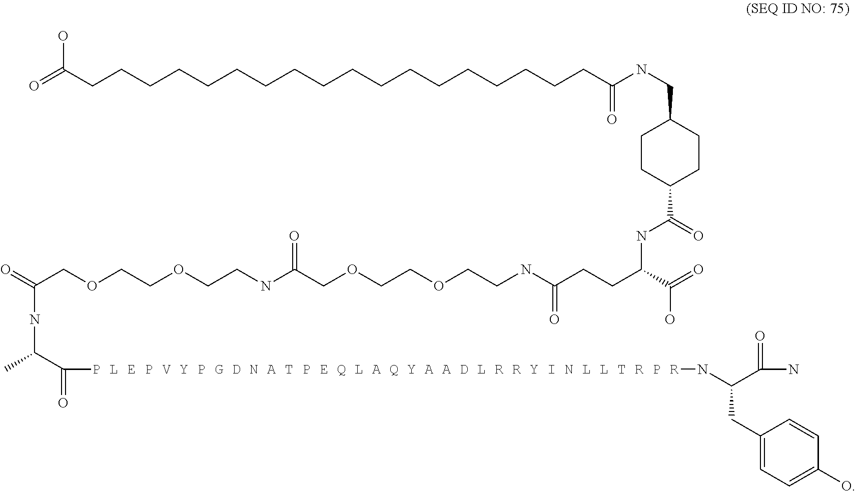

11. A composition comprising a PYY or PP peptide derivative or analogue thereof according to claim 1 and one or more pharmaceutical excipients.

12. A method of treatment of a condition responsive to Y receptor modulation by administration to a subject in need of such treatment of a PYY or PP peptide derivative or analogue thereof according to claim 1.

13. A method of treatment according to claim 12, wherein the condition responsive to Y receptor modulation is obesity.

14. A method of treatment according to claim 12, wherein the administration regime is selected from the group consisting of once-daily, once-weekly, twice-monthly, or once-monthly.

15. (canceled)


SEQUENCE LISTING (75 sequences)

SEQ ID NO: 1 - protein, 34 aa, ARTIFICIAL
  artificial protein based on human PYY or PP
IKPEAPGEDASPEELNRYYASLRHYLNLVTRQRY

SEQ ID NO: 2 - protein, 36 aa, Homo sapiens
APLEPVYPGDNATPEQMAQYAADLRRYINMLTRPRY

SEQ ID NO: 3 - protein, 34 aa, ARTIFICIAL
  artificial protein based on human PYY or PP
IKPEAPGEDAKPEELNRYYASLRHYLNLVTRQRY

SEQ ID NO: 4 - protein, 36 aa, ARTIFICIAL
  artificial protein based on human PYY or PP
APLEPVYPGDNAKPEQLAQYAADLRRYINLLTRQRY

SEQ ID NO: 5 - protein, 34 aa, ARTIFICIAL; identical to SEQ ID NO: 3
  artificial protein based on human PYY or PP

SEQ ID NO: 6 - protein, 35 aa, ARTIFICIAL
  artificial protein based on human PYY or PP
PLEPVYPGKNATPEQLAQYAADLRRYINLLTRPRY

SEQ ID NO: 7 - protein, 35 aa, ARTIFICIAL; identical to SEQ ID NO: 6
  artificial protein based on human PYY or PP

SEQ ID NO: 8 - protein, 34 aa, ARTIFICIAL
  artificial protein based on human PYY or PP
IKPEAPGEKASPEELNRYYASLRHYLNLVTRQRY

SEQ ID NO: 9 - protein, 34 aa, ARTIFICIAL; identical to SEQ ID NO: 8
  artificial protein based on human PYY or PP

SEQ ID NO: 10 - protein, 36 aa, ARTIFICIAL
  artificial protein based on human PYY or PP
APLEPVYPGDNAKPEQLAQYAADLRRYINXLTRQRY

SEQ ID NO: 11 - protein, 35 aa, ARTIFICIAL
  artificial protein based on human PYY or PP
PIKPEAPGEDAKPEELNRYYASLRHYLNLVTRQRY

SEQ ID NO: 12 - protein, 34 aa, ARTIFICIAL; identical to SEQ ID NO: 1
  artificial protein based on human PYY or PP

SEQ ID NO: 13 - protein, 34 aa, ARTIFICIAL; identical to SEQ ID NO: 1
  artificial protein based on human PYY or PP

SEQ ID NO: 14 - protein, 36 aa, ARTIFICIAL
  artificial protein based on human PYY or PP
APLEPVYPGNDAKPEQLAQYAADLRRYINLVTRPRY

SEQ ID NO: 15 - protein, 36 aa, ARTIFICIAL
  artificial protein based on human PYY or PP
APLEPVYPGDNAKPEQLAQYAADLRRYLNVLTRQRY

SEQ ID NO: 16 - protein, 36 aa, ARTIFICIAL
  artificial protein based on human PYY or PP
APLEPVYPGDNAKPEQLAQYAADLRRYVNLLTRQRY

SEQ ID NO: 17 - protein, 36 aa, ARTIFICIAL
  artificial protein based on human PYY or PP
APLEPVYPGDNAKPEQLAQYAADLRRYINVLTRQRY

SEQ ID NO: 18 - protein, 36 aa, ARTIFICIAL
  artificial protein based on human PYY or PP
APLEPVYPGDNAKPEQLAQYAADLRRYIQLLTRPRY

SEQ ID NO: 19 - protein, 34 aa, ARTIFICIAL
  artificial protein based on human PYY or PP
IKPEAPGEDAKPEELNRYYASLRRYLNLVTRQRY

SEQ ID NO: 20 - protein, 36 aa, ARTIFICIAL
  artificial protein based on human PYY or PP
APLEPVYPGEDAKPEELNRYYASLRRYLNLVTRQRY

SEQ ID NO: 21 - protein, 36 aa, ARTIFICIAL
  artificial protein based on human PYY or PP
APIEPEAPGEDAKPEELNRYYASLRRYLNLVTRQRY

SEQ ID NO: 22 - protein, 36 aa, ARTIFICIAL
  artificial protein based on human PYY or PP
APIEPEYPGEDAKPEELNRYYASLRRYLNLVTRQRY

SEQ ID NO: 23 - protein, 34 aa, ARTIFICIAL; identical to SEQ ID NO: 1
  artificial protein based on human PYY or PP

SEQ ID NO: 24 - protein, 36 aa, ARTIFICIAL
  artificial protein based on human PYY or PP
APLEPVYPGDNAKPEQMAQYAADLRRYINMLTRPRY

SEQ ID NO: 25 - protein, 34 aa, ARTIFICIAL; identical to SEQ ID NO: 1
  artificial protein based on human PYY or PP

SEQ ID NO: 26 - protein, 36 aa, ARTIFICIAL
  artificial protein based on human PYY or PP
APLEPVYPGDNAKPEQMAQYAADLRRYINMLTRQRY

SEQ ID NO: 27 - protein, 34 aa, ARTIFICIAL; identical to SEQ ID NO: 8
  artificial protein based on human PYY or PP

SEQ ID NO: 28 - protein, 34 aa, ARTIFICIAL; identical to SEQ ID NO: 8
  artificial protein based on human PYY or PP

SEQ ID NO: 29 - protein, 35 aa, ARTIFICIAL
  artificial protein based on human PYY or PP
PLEPVYPGDKATPEQLAQYAADLRRYINLLTRPRY

SEQ ID NO: 30 - protein, 35 aa, ARTIFICIAL; identical to SEQ ID NO: 29
  artificial protein based on human PYY or PP

SEQ ID NO: 31 - protein, 36 aa, ARTIFICIAL; identical to SEQ ID NO: 24
  artificial protein based on human PYY or PP

SEQ ID NO: 32 - protein, 35 aa, ARTIFICIAL
  artificial protein based on human PYY or PP
PLEPVYPGDNATPEQLKQYAADLRRYINLLTRPRY

SEQ ID NO: 33 - protein, 19 aa, ARTIFICIAL
  artificial protein based on human PYY or PP
NRYYASLRHYLNLVTRQRY

SEQ ID NO: 34 - protein, 34 aa, ARTIFICIAL
  artificial protein based on human PYY or PP
IKPEAPGEDASPEELNRYYASLKHYLNLVTRQRY

SEQ ID NO: 35 - protein, 34 aa, ARTIFICIAL
  artificial protein based on human PYY or PP
IKPEAPGEDASPEELNRYYASKRHYLNLVTRQRY

SEQ ID NO: 36 - protein, 36 aa, ARTIFICIAL
  artificial protein based on human PYY or PP
APLEPVYPGDNAKPEQLAQYAADLRRYINLLTRPRY

SEQ ID NO: 37 - protein, 36 aa, ARTIFICIAL
  artificial protein based on human PYY or PP
APLEPVYPGDNATPEQLAQYAADLKRYINLLTRPRY

SEQ ID NO: 38 - protein, 36 aa, ARTIFICIAL
  artificial protein based on human PYY or PP
APLEPVYPGDNATPKQLAQYAADLRRYINLLTRPRY

SEQ ID NO: 39 - protein, 35 aa, ARTIFICIAL
  artificial protein based on human PYY or PP
PLEPVYPGKNATPEQLAQYAADLRRYINLLTRQRY

SEQ ID NO: 40 - protein, 34 aa, ARTIFICIAL
  artificial protein based on human PYY or PP
IKPEAPGEDASPEELNKYYASLRHYLNLVTRQRY

SEQ ID NO: 41 - protein, 35 aa, ARTIFICIAL
  artificial protein based on human PYY or PP
PLEPVYPGDNATPEQLAQYAADLRRYINLLTKPRY

SEQ ID NO: 42 - protein, 35 aa, ARTIFICIAL; identical to SEQ ID NO: 41
  artificial protein based on human PYY or PP

SEQ ID NO: 43 - protein, 35 aa, ARTIFICIAL; identical to SEQ ID NO: 32
  artificial protein based on human PYY or PP

SEQ ID NO: 44 - protein, 35 aa, ARTIFICIAL
  artificial protein based on human PYY or PP
PLEPVYPGDNATPEQLAQYAADLRRYIKLLTRPRY

SEQ ID NO: 45 - protein, 35 aa, ARTIFICIAL
  artificial protein based on human PYY or PP
PLEPVYPGDNATPEQLAQYAADLRKYINLLTRPRY

SEQ ID NO: 46 - protein, 35 aa, ARTIFICIAL; identical to SEQ ID NO: 45
  artificial protein based on human PYY or PP

SEQ ID NO: 47 - protein, 35 aa, ARTIFICIAL
  artificial protein based on human PYY or PP
PLEPVYPGDNATPEQLAQYAADLRRYINLLTRPKY

SEQ ID NO: 48 - protein, 35 aa, ARTIFICIAL; identical to SEQ ID NO: 47
  artificial protein based on human PYY or PP

SEQ ID NO: 49 - protein, 35 aa, ARTIFICIAL
  artificial protein based on human PYY or PP
PLEPVYPGDNATPEQLAQYAADKRRYINLLTRPRY

SEQ ID NO: 50 - protein, 35 aa, ARTIFICIAL
  artificial protein based on human PYY or PP
PLEPVYPGDNATPEQLAQYAADLKRYINLLTRPRY

SEQ ID NO: 51 - protein, 34 aa, ARTIFICIAL; identical to SEQ ID NO: 3
  artificial protein based on human PYY or PP

SEQ ID NO: 52 - protein, 34 aa, ARTIFICIAL; identical to SEQ ID NO: 34
  artificial protein based on human PYY or PP

SEQ ID NO: 53 - protein, 34 aa, ARTIFICIAL; identical to SEQ ID NO: 1
  artificial protein based on human PYY or PP

SEQ ID NO: 54 - protein, 34 aa, ARTIFICIAL; identical to SEQ ID NO: 1
  artificial protein based on human PYY or PP

SEQ ID NO: 55 - protein, 35 aa, ARTIFICIAL
  artificial protein based on human PYY or PP
PLEPVYPGDNATPEQLAQYAADLRRYINLLTRPRY

SEQ ID NO: 56 - protein, 35 aa, ARTIFICIAL; identical to SEQ ID NO: 55
  artificial protein based on human PYY or PP

SEQ ID NO: 57 - protein, 34 aa, ARTIFICIAL
  artificial protein based on human PYY or PP
IKPEAPGEDASPEELKRYYASLRHYLNLVTRQRY

SEQ ID NO: 58 - protein, 34 aa, ARTIFICIAL
  artificial protein based on human PYY or PP
IKPEAPGEDASPEELNRYYKSLRHYLNLVTRQRY

SEQ ID NO: 59 - protein, 34 aa, ARTIFICIAL
  artificial protein based on human PYY or PP
IKPEAPGEDASPEELNRYYASLRKYLNLVTRQRY

SEQ ID NO: 60 - protein, 34 aa, ARTIFICIAL
  artificial protein based on human PYY or PP
IKPEAPGEDASPEELNRYYASLRHYLKLVTRQRY

SEQ ID NO: 61 - protein, 34 aa, ARTIFICIAL
  artificial protein based on human PYY or PP
IKPEAPGEDASPEELNRYYASLRHYLNLVTRQRK

SEQ ID NO: 62 - protein, 34 aa, ARTIFICIAL
  artificial protein based on human PYY or PP
IKPEAPGEDASPEELNRYKASLRHYLNLVTRQRY

SEQ ID NO: 63 - protein, 34 aa, ARTIFICIAL
  artificial protein based on human PYY or PP
IKPEAPGEDASPEELNRYYASLRHYLNKVTRQRY

SEQ ID NO: 64 - protein, 34 aa, ARTIFICIAL
  artificial protein based on human PYY or PP
IKPEAPGEDASPEELNRYYASLRHYLNLKTRQRY

SEQ ID NO: 65 - protein, 34 aa, ARTIFICIAL
  artificial protein based on human PYY or PP
IKPEAPGEDASKEELNRYYASLRHYLNLVTRQRY

SEQ ID NO: 66 - protein, 34 aa, ARTIFICIAL
  artificial protein based on human PYY or PP
IKPEAPGEDASPKELNRYYASLRHYLNLVTRQRY

SEQ ID NO: 67 - protein, 34 aa, ARTIFICIAL
  artificial protein based on human PYY or PP
IKPEAPGEDASPEKLNRYYASLRHYLNLVTRQRY

SEQ ID NO: 68 - protein, 34 aa, ARTIFICIAL
  artificial protein based on human PYY or PP
IKPEAPGEDASPEELNRYYASLRHYKNLVTRQRY

SEQ ID NO: 69 - protein, 34 aa, ARTIFICIAL; identical to SEQ ID NO: 68
  artificial protein based on human PYY or PP

SEQ ID NO: 70 - protein, 34 aa, ARTIFICIAL
  artificial protein based on human PYY or PP
IKPEAPGEDASPEELNRYYASLRHYLNLVKRQRY

SEQ ID NO: 71 - protein, 36 aa, ARTIFICIAL; identical to SEQ ID NO: 37
  artificial protein based on human PYY or PP

SEQ ID NO: 72 - protein, 36 aa, ARTIFICIAL; identical to SEQ ID NO: 38
  artificial protein based on human PYY or PP

SEQ ID NO: 73 - protein, 35 aa, ARTIFICIAL; identical to SEQ ID NO: 55
  artificial protein based on human PYY or PP

SEQ ID NO: 74 - protein, 36 aa, ARTIFICIAL; identical to SEQ ID NO: 4
  Artificial protein based on human PYY or PP

SEQ ID NO: 75 - protein, 36 aa, ARTIFICIAL
  Articificial protein based on human PYY or PP
APLEPVYPGDNATPEQLAQYAADLRRYINLLTRPRY